SEQUENCE LISTING (40 sequences)

SEQ ID NO: 1 - protein, 535 aa, Artificial Sequence
  Description of Artificial Sequence Synthetic polypeptide
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNDEGTYSEPRPIGTRYLTRNL

SEQ ID NO: 2 - protein, 535 aa, Artificial Sequence
  Description of Artificial Sequence Synthetic polypeptide
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 3 - protein, 534 aa, Unknown
  Description of Unknown wt-rh74-VP3 sequence
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGWADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPRPIGTRYLTRNL

SEQ ID NO: 4 - DNA, 1611 bp, Artificial Sequence
  Description of Artificial Sequence Synthetic polynucleotide
atggctgctggcggaggtgcccctatggccgataacaatgaaggtgctgatggcgtgggctccagcagcggaaattggcactgtgatagcacctggctgggcgacagagtgatcaccaccagcacaagaacatgggccctgcctacctacaacaaccacctgtacaagcagatcagcaacggcacaagcggcggctccaccaacgataacacctactttggctacagcaccccttggggctacttcgacttcaaccggttccactgccacttcagccccagagactggcagcggctgatcaacaacaactggggcttcagacccaagaggctgaacttcaagctgttcaacatccaagtgaaagaagtgacccagaacgagggcaccaagacaatcgccaacaacctgaccagcaccatccaggtgttcaccgacagcgagtaccagctgccttatgtgctgggatctgcccaccaaggctgtctgcctccatttcctgccgacgtgttcatgatccctcagtacggctacctgacactgaacaacggatctcaggccgtgggcagaagctccttctactgcctggaatacttccctagccagatgctgcggaccggcaacaacttcgagttcagctacaatttcgaggacgtgcccttccacagcagctacgcccactctcaaagcctggacagactgatgaaccctctgatcgaccagtacctgtactacctgagcagaacccagagcacaggcggaacagctggaacacagcaactgctgtttagccaggctggccccaacaatatgagcgcccaggccaaaaattggctgcccggaccttgctacagacagcagagagtgtctaccacactgagccagaacaacaacagcaactttgcctggaccggcgccaccaagtaccacctgaatggcagagacagcctggtcaatcctggcgtggccatggctacccacaaggacgacgaggaaagattcttcccaagcagcggcgtgctgatgttcggaaagcaaggcgccggaaaggacaacgtggactacagcagcgtgatgctgaccagcgaggaagagatcaagaccaccaatcctgtggccaccgagcagtacggagtggtggccgataatctgcaacagcagaacgccgctcctatcgtgggagctgtgaattctcaaggcgccctgcctggcatggtctggcagaatagagatgtgtacctgcaaggccccatctgggccaagattccccacaccgatggcaactttcacccctctccactgatgggcggcttcggacttaaacaccctccacctcagatcctgatcaagaatacccctgtgcctgccgatcctcctaccaccttcaatcaggccaaactggccagcttcatcacccagtactctaccggacaggtgtccgtggaaatcgagtgggaactccagaaagaaaacagcaagcggtggaaccccgagatccagtacaccagcaactactacaagagcaccaacgtcgacttcgccgtgaacaccgagggcacatacagcgagcctagacctatcggcacccggtatctgacccggaacctgtaatga

SEQ ID NO: 5 - DNA, 1611 bp, Artificial Sequence
  Description of Artificial Sequence Synthetic polynucleotide
atggctgcaggaggaggtgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggcgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaatga

SEQ ID NO: 6 - DNA, 2217 bp, Artificial Sequence
  Description of Artificial Sequence Synthetic polynucleotide
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacagtggctgcaggcggtggcgcaccagtggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 7 - protein, 738 aa, Artificial Sequence
  Description of Artificial Sequence Synthetic polypeptide
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTVAAGGGAPVADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 8 - protein, 601 aa, Artificial Sequence
  Description of Artificial Sequence Synthetic polypeptide
TAPGKKRPVEPSPQRSPDSSTGIGKKGQQPARKRLNFGQTGDSESVPDPQPLGEPPAAPSGVGPNTVAAGGGAPVADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 535 aa, Artificial Sequence
  Description of Artificial Sequence Synthetic polypeptide
VAAGGGAPVADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 10 - DNA, 9690 bp, Artificial Sequence
  Description of Artificial Sequence Synthetic polynucleotide
ggccgcgaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttgtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgggcctcggcggccaagcttggcaatccggtactgttggtaaaactagtccaccatggctgctggcggaggtgcccctatggccgataacaatgaaggtgctgatggcgtgggctccagcagcggaaattggcactgtgatagcacctggctgggcgacagagtgatcaccaccagcacaagaacatgggccctgcctacctacaacaaccacctgtacaagcagatcagcaacggcacaagcggcggctccaccaacgataacacctactttggctacagcaccccttggggctacttcgacttcaaccggttccactgccacttcagccccagagactggcagcggctgatcaacaacaactggggcttcagacccaagaggctgaacttcaagctgttcaacatccaagtgaaagaagtgacccagaacgagggcaccaagacaatcgccaacaacctgaccagcaccatccaggtgttcaccgacagcgagtaccagctgccttatgtgctgggatctgcccaccaaggctgtctgcctccatttcctgccgacgtgttcatgatccctcagtacggctacctgacactgaacaacggatctcaggccgtgggcagaagctccttctactgcctggaatacttccctagccagatgctgcggaccggcaacaacttcgagttcagctacaatttcgaggacgtgcccttccacagcagctacgcccactctcaaagcctggacagactgatgaaccctctgatcgaccagtacctgtactacctgagcagaacccagagcacaggcggaacagctggaacacagcaactgctgtttagccaggctggccccaacaatatgagcgcccaggccaaaaattggctgcccggaccttgctacagacagcagagagtgtctaccacactgagccagaacaacaacagcaactttgcctggaccggcgccaccaagtaccacctgaatggcagagacagcctggtcaatcctggcgtggccatggctacccacaaggacgacgaggaaagattcttcccaagcagcggcgtgctgatgttcggaaagcaaggcgccggaaaggacaacgtggactacagcagcgtgatgctgaccagcgaggaagagatcaagaccaccaatcctgtggccaccgagcagtacggagtggtggccgataatctgcaacagcagaacgccgctcctatcgtgggagctgtgaattctcaaggcgccctgcctggcatggtctggcagaatagagatgtgtacctgcaaggccccatctgggccaagattccccacaccgatggcaactttcacccctctccactgatgggcggcttcggacttaaacaccctccacctcagatcctgatcaagaatacccctgtgcctgccgatcctcctaccaccttcaatcaggccaaactggccagcttcatcacccagtactctaccggacaggtgtccgtggaaatcgagtgggaactccagaaagaaaacagcaagcggtggaaccccgagatccagtacaccagcaactactacaagagcaccaacgtcgacttcgccgtgaacaccgagggcacatacagcgagcctagacctatcggcacccggtatctgacccggaacctgtaatgacttaagcctaggctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaacgttaatattttgttaaaattcgcgttaaatttttgttaaatcagctcattttttaaccaataggccgaaatcggcaaaatcccttataaatcaaaagaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacagtggctgcaggcggtggcgcaccagtggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggc

SEQ ID NO: 11 - DNA, 7293 bp, Artificial Sequence
  Description of Artificial Sequence Synthetic polynucleotide
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgggcctcggcggccaagcttggcaatccggtactgttggtaaaactagtccaccatggctgctggcggaggtgcccctatggccgataacaatgaaggtgctgatggcgtgggctccagcagcggaaattggcactgtgatagcacctggctgggcgacagagtgatcaccaccagcacaagaacatgggccctgcctacctacaacaaccacctgtacaagcagatcagcaacggcacaagcggcggctccaccaacgataacacctactttggctacagcaccccttggggctacttcgacttcaaccggttccactgccacttcagccccagagactggcagcggctgatcaacaacaactggggcttcagacccaagaggctgaacttcaagctgttcaacatccaagtgaaagaagtgacccagaacgagggcaccaagacaatcgccaacaacctgaccagcaccatccaggtgttcaccgacagcgagtaccagctgccttatgtgctgggatctgcccaccaaggctgtctgcctccatttcctgccgacgtgttcatgatccctcagtacggctacctgacactgaacaacggatctcaggccgtgggcagaagctccttctactgcctggaatacttccctagccagatgctgcggaccggcaacaacttcgagttcagctacaatttcgaggacgtgcccttccacagcagctacgcccactctcaaagcctggacagactgatgaaccctctgatcgaccagtacctgtactacctgagcagaacccagagcacaggcggaacagctggaacacagcaactgctgtttagccaggctggccccaacaatatgagcgcccaggccaaaaattggctgcccggaccttgctacagacagcagagagtgtctaccacactgagccagaacaacaacagcaactttgcctggaccggcgccaccaagtaccacctgaatggcagagacagcctggtcaatcctggcgtggccatggctacccacaaggacgacgaggaaagattcttcccaagcagcggcgtgctgatgttcggaaagcaaggcgccggaaaggacaacgtggactacagcagcgtgatgctgaccagcgaggaagagatcaagaccaccaatcctgtggccaccgagcagtacggagtggtggccgataatctgcaacagcagaacgccgctcctatcgtgggagctgtgaattctcaaggcgccctgcctggcatggtctggcagaatagagatgtgtacctgcaaggccccatctgggccaagattccccacaccgatggcaactttcacccctctccactgatgggcggcttcggacttaaacaccctccacctcagatcctgatcaagaatacccctgtgcctgccgatcctcctaccaccttcaatcaggccaaactggccagcttcatcacccagtactctaccggacaggtgtccgtggaaatcgagtgggaactccagaaagaaaacagcaagcggtggaaccccgagatccagtacaccagcaactactacaagagcaccaacgtcgacttcgccgtgaacaccgagggcacatacagcgagcctagacctatcggcacccggtatctgacccggaacctgtaatgacttaagcctaggctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaacgttaatattttgttaaaattcgcgttaaatttttgttaaatcagctcattttttaaccaataggccgaaatcggcaaaatcccttataaatcaaaagaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacagtggctgcaggcggtggcgcaccagtggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaa

SEQ ID NO: 12 - DNA, 9690 bp, Artificial Sequence
  Description of Artificial Sequence Synthetic polynucleotide
ggccgcgaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttgtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgggcctcggcggccaagcttggcaatccggtactgttggtaaaactagtccaccatggctgcaggaggaggtgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggcgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaatgacttaagcctaggctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaacgttaatattttgttaaaattcgcgttaaatttttgttaaatcagctcattttttaaccaataggccgaaatcggcaaaatcccttataaatcaaaagaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacagtggctgcaggcggtggcgcaccagtggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaagcccgagtgagcacgcagggtctccattttgaagcgggaggtttgaacgcgcagccgccaagccgaattctgcagatatccatcacactggc

SEQ ID NO: 13 - DNA, 7293 bp, Artificial Sequence
  Description of Artificial Sequence Synthetic polynucleotide
gtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatcactagaagctttattgcggtagtttatcacagttaaattgctaacgcagtcagtgggcctcggcggccaagcttggcaatccggtactgttggtaaaactagtccaccatggctgcaggaggaggtgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggcgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaatgacttaagcctaggctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaacgttaatattttgttaaaattcgcgttaaatttttgttaaatcagctcattttttaaccaataggccgaaatcggcaaaatcccttataaatcaaaagaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgatcgaggtcgacggtatcgggggagctcgcagggtctccattttgaagcgggaggtttgaacgcgcagccgccatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacagtggctgcaggcggtggcgcaccagtggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcctgttaatcaataaaccggttgattcgtttcagttgaactttggtctctgcgaagggcgaattcgtttaaacctgcaggactagaggtcctgtattagaggtcacgtgagtgttttgcgacattttgcgacaccatgtggtcacgctgggtatttaa

SEQ ID NO: 14 - protein, 534 aa, Artificial Sequence
  Description of Artificial Sequence Synthetic polypeptide
MAAGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDKERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPHPIGTRYLTRPL

SEQ ID NO: 15 - protein, 534 aa, Artificial Sequence
  Description of Artificial Sequence Synthetic polypeptide
MAAGGGAPMADNNEGADGVGSSSGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYNFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDKERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFNQAKLASFITQYSTGQVSVEIEWELQKENSKRWNPEIQYTSNYYKSTNVDFAVNTEGTYSEPHPIGTRYLTRPL

SEQ ID NO: 16 - DNA, 1608 bp, Artificial Sequence
  Description of Artificial Sequence Synthetic polynucleotide
atggctgctggtggcggagcacctatggccgataacaatgaaggcgccgatggcgtgggcagcagctctggaaattggcactgtgacagcacctggctgggcgacagagtgatcaccaccagcaccagaacatgggccctgcctacctacaacaaccacctgtacaagcagatcagcagcgccagcacaggcgccagcaacgacaatcactacttcggctacagcaccccttggggctacttcgacttcaaccggttccactgccacttcagccccagagactggcagcggctgatcaacaacaactggggcttcagacccaagcggctgaacttcaagctgttcaacatccaagtgaaagaagtgacccagaacgagggcaccaagacaatcgccaacaacctgaccagcacaatccaggtgttcaccgacagcgagtaccagctgccttacgtgctgggatctgcccaccaaggatgcctgcctccatttcctgccgacgtgttcatgatccctcagtacggctacctgacactgaacaacggctctcaggccgtgggcagaagcagcttctactgcctggaatacttccctagccagatgctgcggaccggcaacaacttcgagttcagctaccagttcgaggacgtgcccttccacagcagctatgcccactctcagagcctggaccggctgatgaaccctctgatcgaccagtacctgtactacctgagcagaacccagagcaccggcggaacagctggaacacagcaactgctgtttagccaggctggccccaacaacatgtctgcccaggccaagaattggctgcccggaccttgttaccggcagcagagagtgtctaccacactgagccagaacaacaacagcaacttcgcctggactggcgccaccaagtaccacctgaatggcagagacagcctggtcaatcctggcgtggccatggctacccacaaggacgacaaagagcggttcttcccaagcagcggcgtgctgatgtttggcaaacaaggcgccggaaaggacaacgtggactacagcagcgtgatgctgaccagcgaggaagagatcaagaccaccaatcctgtggccaccgagcagtatggcgtggtggctgataatctgcagcagcagaacgccgctcctatcgtgggagccgtgaattctcaaggcgctctgcctggcatggtctggcagaatagggacgtgtacctgcagggccccatctgggccaagattcctcacaccgacggcaactttcacccctctccactgatgggcggcttcggactgaaacaccctccacctcagatcctgatcaagaacacccctgtgcctgccgatcctcctaccacattcagccaggccaaactggccagcttcatcacccagtacagcaccggacaggtgtccgtggaaatcgagtgggagctgcagaaagaaaacagcaagcggtggaaccccgagatccagtacaccagcaactactacaagagcaccaatgtggacttcgccgtgaacaccgatggcacctacagcgagcctcatcctatcggcaccagatacctgacaagacccctgtaatga

SEQ ID NO: 17 - DNA, 1608 bp, Artificial Sequence
  Description of Artificial Sequence Synthetic polynucleotide
atggctgctggtggcggagcacctatggccgataacaatgaaggcgccgatggcgtgggcagcagctctggaaattggcactgtgacagcacctggctgggcgacagagtgatcaccaccagcaccagaacatgggccctgcctacctacaacaaccacctgtacaagcagatcagcagcgccagcacaggcgccagcaacgacaatcactacttcggctacagcaccccttggggctacttcgacttcaaccggttccactgccacttcagccccagagactggcagcggctgatcaacaacaactggggcttcagacccaagcggctgaacttcaagctgttcaacatccaagtgaaagaagtgacccagaacgagggcaccaagacaatcgccaacaacctgaccagcacaatccaggtgttcaccgacagcgagtaccagctgccttacgtgctgggatctgcccaccaaggatgcctgcctccatttcctgccgacgtgttcatgatccctcagtacggctacctgacactgaacaacggctctcaggccgtgggcagaagcagcttctactgcctggaatacttccctagccagatgctgcggaccggcaacaacttcgagttcagctacaatttcgaggacgtgcccttccacagcagctacgcccattctcagagcctggaccggctgatgaaccctctgatcgaccagtacctgtactacctgagcagaacccagagcaccggcggaacagctggaacacagcaactgctgtttagccaggctggccccaacaacatgtctgcccaggccaagaattggctgcccggaccttgttaccggcagcagagagtgtctaccacactgagccagaacaacaacagcaacttcgcctggactggcgccaccaagtaccacctgaatggcagagacagcctggtcaatcctggcgtggccatggctacccacaaggacgacaaagagcggttcttcccaagcagcggcgtgctgatgtttggcaaacaaggcgccggaaaggacaacgtggactacagcagcgtgatgctgaccagcgaggaagagatcaagaccaccaatcctgtggccaccgagcagtatggagtggtggccgataatctgcagcagcagaacgccgctcctatcgtgggagccgtgaattctcaaggcgctctgcctggcatggtctggcagaatagggacgtgtacctgcagggccccatctgggccaagattcctcacaccgacggcaactttcacccctctccactgatgggcggcttcggactgaaacaccctccacctcagatcctgatcaagaacacccctgtgcctgccgatcctcctaccaccttcaatcaggccaagctggcctccttcatcacccagtactctaccggccaggtgtccgtggaaatcgagtgggagctgcagaaagaaaacagcaagcggtggaaccccgagatccagtacaccagcaactactacaagagcaccaatgtggacttcgccgtgaacaccgagggcacatacagcgagcctcatcctatcggcaccagatacctgacaagacccctgtaatga

SEQ ID NO: 18 - protein, 534 aa, Adeno-associated virus 1
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 19 - protein, 533 aa, Adeno-associated virus 2
MATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 20 - protein, 534 aa, Adeno-associated virus 6
MASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 21 - protein, 534 aa, Adeno-associated virus 6; identical to SEQ ID NO: 20

SEQ ID NO: 22 - protein, 534 aa, Adeno-associated virus 7
VAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 23 - protein, 534 aa, Adeno-associated virus 9
MASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 24 - protein, 535 aa, Adeno-associated virus
MAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 25 - protein, 534 aa, Adeno-associated virus
MAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPVTGSCFWQQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 26 - protein, 5 aa, Artificial Sequence
  Description of Artificial Sequence Synthetic peptide
FVFLP

SEQ ID NO: 27 - protein, 4 aa, Artificial Sequence
  Description of Artificial Sequence Synthetic peptide
RGNR

SEQ ID NO: 28 - protein, 60 aa, Adeno-associated virus 1
ESVPDPQPLGEPPATPAAVGPTTMASGGGAPMADNNEGADGVGNASGNWHCDSTWLGDRV

SEQ ID NO: 29 - protein, 60 aa, Adeno-associated virus 6; identical to SEQ ID NO: 28

SEQ ID NO: 30 - protein, 60 aa, Adeno-associated virus 2
DSVPDPQPLGQPPAAPSGLGTNTMATGSGAPMADNNEGADGVGNSSGNWHCDSTWMGDRV

SEQ ID NO: 31 - protein, 60 aa, Adeno-associated virus 3B
ESVPDPQPLGEPPAAPTSLGSNTMASGGGAPMADNNEGADGVGNSSGNWHCDSQWLGDRV

SEQ ID NO: 32 - protein, 60 aa, Adeno-associated virus 8
ESVPDPQPLGEPPAAPSGVGPNTMAAGGGAPMADNNEGADGVGSSSGNWHCDSTWLGDRV

SEQ ID NO: 33 - protein, 60 aa, Adeno-associated virus
ESVPDPQPIGEPPAGPSGLGSGTMAAGGGAPMADNNEGADGVGSSSGNWHCDSTWLGDRV

SEQ ID NO: 34 - protein, 60 aa, Adeno-associated virus 9
ESVPDPQPIGEPPAAPSGVGSLTMASGGGAPVADNNEGADGVGSSSGNWHCDSQWLGDRV

SEQ ID NO: 35 - DNA, 37 bp, Adeno-associated virus 8
tacaatggctgcaggcggtggcgcaccaatggcagac

SEQ ID NO: 36 - DNA, 37 bp, Artificial Sequence
  Description of Artificial Sequence Synthetic oligonucleotide
tacagtggctgcaggcggtggcgcaccagtggcagac

SEQ ID NO: 37 - protein, 12 aa, Adeno-associated virus 8
TMAAGGGAPMAD

SEQ ID NO: 38 - protein, 12 aa, Artificial Sequence
  Description of Artificial Sequence Synthetic peptide
TVAAGGGAPVAD

SEQ ID NO: 39 - protein, 11 aa, Adeno-associated virus 8
QWLQAVAHQWQ

SEQ ID NO: 40 - protein, 11 aa, Artificial Sequence
  Description of Artificial Sequence Synthetic peptide
QWLQAVAHQWQ

(19) United States
(12) Patent Application Publication
(10) Pub. No.: US 2024/0189447 A1
(43) Pub. Date: Jun. 13, 2024

(54) RATIONAL POLYPLOID AAV VIRIONS THAT CROSS THE BLOOD BRAIN BARRIER AND ELICIT REDUCED HUMORAL RESPONSE

(71) Applicant: ASKLEPIOS BIOPHARMACEUTICAL, INC., Research Triangle Park, NC (US)

(72) Inventors: Lester Suarez, Research Triangle Park, NC (US); Amaury Pupo Merino, Research Triangle Park, NC (US); Audry Fernandez, Research Triangle Park, NC (US)

(73) Assignee: ASKLEPIOS BIOPHARMACEUTICAL, INC., Research Triangle Park, NC (US)

(21) Appl. No.: 18/286,709

(22) PCT Filed: Apr. 14, 2022
(86) PCT No.: PCT/US2022/024809

§ 371 Date: Oct. 12, 2023

Related U.S. Application Data
(60) Provisional application No. 63/175,954, filed on Apr. 16, 2021.
(60) Provisional application No. 63/180,414, filed on Apr. 27, 2021.

(51) Int. Cl. A61K 48/00; C12N 7/00; C12N 15/86

(52) U.S. Cl. CPC A61K 48/0041; C12N 7/00; C12N 15/86; C12N 2750/14122; C12N 2750/14143; C12N 2750/14145

(57) ABSTRACT

The present invention relates to a substantially homogenous population of a rational polyploid adeno-associated virus (AAV) virons that cross the blood brain barrier (BBB), where the rational polyploid comprises a VP3 viral structural protein from any AAV serotype that cross the BBB. In some embodiments, the rational polyploid crosses the BBB upon systemic or intrathecal administration to a subject. In some embodiments, a rational polyploid AAV virion comprises at least one VP1 and/or VP2 viral structural protein in addition to the VP3 protein. In some embodiments, the VP3 capsid protein is from a non-human primate, and in some embodiments the VP3 capsid protein is a AAV rhesus monkey serotype. In specific embodiments, rational polyploid AAV virion comprises a VP1 capsid protein from AAV8, and at least a VP3 capsid protein from any AAV serotype that cross the BBB.

### CROSS-REFERENCED APPLICATIONS

This invention claims benefit under 35 U.S.C. § 119(e) of U.S. Provisional Application 63/175,954 filed on Apr. 16, 2021, and U.S. Provisional Application 63/180,414 filed on Apr. 27, 2021, the contents of each are incorporated herein in their entirety by reference.

### SEQUENCE LISTING

The instant application contains a Sequence Listing which has been submitted electronically in ASCII format and is hereby incorporated by reference in its entirety. Said ASCII copy, created on Apr. 26, 2021, is named 046192-098020PL02_SL.txt and is 139,904 bytes in size.

### FIELD OF THE INVENTION

The present invention is directed to methods for production of a group of rational polyploid adeno-associated virus (AAV) particles, virions and virus capsids with desired properties, the virions, substantially homogenous populations of such virions, methods of producing substantially homogenous populations, and uses thereof. More specifically, the invention is directed to polyploid AAV virion particles comprising a VP3 structural protein from any AAV serotype which crosses the blood brain barrier (BBB) and wherein the polyploid AAV virion crosses the BBB and/or transduces a cell of the BBB upon systemic or, intrathecal administration.

### BACKGROUND OF THE INVENTION

Central nervous system (CNS) diseases are some of the most difficult to treat because the blood-brain barrier (BBB) almost entirely limits the passage of many therapeutic drugs into the CNS. Adeno-associated virus (AAV) vector has been widely used in the treatment of various central nervous system (CNS) diseases. Due to the presence of the blood-brain barrier (BBB), early attempts at AAV-based CNS diseases treatment were mainly performed through intracranial injections. For example, in treating disorders of the central nervous systems (CNS; i.e., brain and spinal cord), delivery of the AAV-based therapy is complicated, with direct administration generally involving invasive surgeries, with the blood brain barrier impeding the access of the AAV-based therapy to the CNS if administered systemically. Further, high doses of AAV-based therapies are necessary to yield sufficient transduction of target CNS tissue, giving rise to enhanced risk of side effects and/or production difficulties given the high volumes needed. Though the peripheral nervous system (PNS; i.e., nervous tissue outside the brain and spinal cord) may be thought of as more accessible for therapeutic intervention, some PNS tissues, such as dorsal root ganglia remain difficult to target. The AAV serotype AAV9 has been widely studied for its ability to cross the BBB to transduce astrocytes, but its efficiency is limited. For example, systemic injections of AAV9 has been assessed in clinical trials for multiple CNS diseases. However, the development of systemic AAV injections to treat CNS diseases is still associated with many challenges, such as the efficiency of AAV in crossing the BBB, the peripheral toxicity caused by the expression of AAV-delivered genes, and the immune barrier against AAV in the blood.

To date, 12 AAV serotypes and more than 100 variants have been identified, as well as animal and non-human primate AAV serotypes, including Rhesus monkey (AAVrh) and chimpanzees. Different serotype capsids have different infectivity in tissues or culture cells, which depend on the primary receptor and co-receptors on the cell surface or the intracellular trafficking pathway itself. Although the application of AAV vectors has been proven safe and shown therapeutic effect, there have been differences between serotypes, including different transduction and biodistribution profiles.

In some clinical trials, there have been report of distinct species-specific differences in transgene expression between mice, non-human primates and humans, showing high transgene expression in pre-clinical mice studies but lacking similar gene expression in human clinical trials, including potential capsid specific cytotoxic T lymphocyte (CTL) response that eradicates AAV transduced hepatocytes, resulting in therapeutic failure. Therefore, the results from these clinical trials highlight the necessity to explore effective approaches for enhancement of AAV transduction without increasing vector capsid burden. In addition, the majority of people have been naturally exposed to AAVs. As a result, a large portion of the population has developed neutralizing antibodies (Nabs) in the blood and other bodily fluids against certain serotypes of AAV.

A full spectrum of immune responses to adeno-associated viruses has been assessed to include innate immunity, cytotoxic T-cell (CTL) responses and humoral responses. The pre-existing anti-AAV immunity, in particular, neutralizing antibodies (NAbs) to AAV serotypes has emerged as a significant challenge for clinical applications of AAV vector mediated gene delivery. Several studies have discussed the prevalence of pre-existing NAbs against the commonly used AAV serotypes (e.g. AAV serotypes 1-9). Several studies have shown that the induction of antibodies by natural exposure to AAV early in life can compromise the subsequent use of AAV as a gene therapy vector and/or, potent humoral response induced by AAV vector can compromise the potential requirement of repeat dosing with the same AAV vector (Hurlbut et al., Mol Ther, 2010, 18(11):1983; Manno et al., Nat Med, 2006, 12(3):342; and Wang et al., Blood, 2006, 107(5):1810; Calcedo et al., Front Immunol, 2013, 4, 341; the contents of each of which are incorporated herein by reference in their entirety).

Therefore, humoral immunity against AAV vectors represents a significant barrier to effective gene transfer, resulting in clearance of the AAV vector before it enters the target cell. Antibodies directed against the AAV capsid are highly prevalent in humans, a natural host for this virus, and cross-react with a wide range of serotypes because of the degree of homology of capsid protein sequence. As NAb can efficiently block AAV-mediated transduction in vivo, strategies to overcome humoral immunity to the viral capsid are of great importance to achieve successful gene transfer.

As such, there is a need to tailor AAV vectors that efficiently cross the BBB and have a combined set of desirable features, including different transduction efficiency and reduced immune responses would be highly beneficial in gene therapy drug development.

### SUMMARY OF THE INVENTION

The technology herein relates to a substantially homogenous population of rational polyploid or haploid AAV vectors, virions or pharmaceutical compositions thereof, that cross the blood brain barrier (BBB) and/or has reduced antigenicity. It is well known that a barrier of delivering therapeutic genes via gene therapy and viral vectors to the CNS is the blood brain barrier. The blood brain barrier is a semipermeable border of endothelial cells that prevents certain chemicals and molecules in the bloodstream from crossing into the extracellular fluid of the central nervous system. Herein, the inventors have rationally designed a AAV virion to comprise structural VP proteins from more than one serotype to increase the vector transduction efficiency to the CNS and PNS after systemic and intrathecal delivery. Embodiments of the present invention are based upon the surprising discovery that the VP3 viral structural protein from an AAV serotype known to efficiently cross the blood brain is responsible for the blood brain crossing phenotype. Thus, rational polyploids can be designed to cross the blood brain barrier as well as to avoid immune responses to parental serotypes, e.g., evading neutralizing antibodies and eliciting less humoral response to parental serotypes that create the rational polyploid.

Previous reports show that designing AAV vectors which are composed of capsids from two or more AAV serotypes, such as rational polyploids can take advantages from individual serotypes for an altered behavior such as tropism, transduction or antigenicity. Some of these polyploid viruses have the ability to change the tropism and transduction efficiency, as well as escape the neutralization by neutralizing antibodies (Nabs).

The inventors have previously discovered methodology that permits the rational design and production of virions where the virions are sometimes referred to as rationalpolyploid virions to refer to the fact that the capsid proteins VP1, VP2, and VP3 come from at least two different serotypes, but not all the same serotype. The term haploid is sometimes used to refer to a virion where the capsid proteins VP1, VP2 and VP3 are from at least two different serotypes, and the term triploid is used to commonly refer to a virion where the capsid proteins VP1, VP2 and VP3 are from three different serotypes. In particular, such rational polyploid, e.g., rational haploid virions and their method of production are disclosed in U.S. Pat. No. 10,550,405, which is incorporated herein in its entirety by reference.

Herein, the technology generally relates to a homogenous population of rational polyploid adeno-associated virus (AAV) particles, virions and virus capsids comprising a VP3 structural protein from any AAV serotype which crosses the blood brain barrier (BBB) and wherein the polyploid AAV virion crosses the BBB, and/or transduces a blood component to allow delivery via the cerebral circulation to the brain, upon systemic or intrathecal administration. In some embodiments, the VP3 capsid protein is from a non-human primate such as an AAV rhesus monkey (rhAAV) serotype. A rational polyploid AAV virion can also comprise at least one VP1 and/or VP2 viral structural protein from a different serotype from the VP3 protein. In some embodiments, rational polyploid AAV virion comprises a VP1 capsid protein from AAV8, and at least a VP3 capsid protein from any AAV serotype that cross the BBB. In some embodiments, the viral structural proteins (e.g., any one or more of VP1, VP2 or VP3) can be modified, e.g., by changes to the nucleotides, chemically modified, mixed serotypes, etc.

Using, for example, a VP3 structural protein from any AAV serotype that efficiently crosses the BBB changes the biodistribution and transduction efficiency of the vector after systemic or intrathecal administration, and in particular, shows an increased ability of the AAV vector to cross the BBB and transduce one or more tissues in the CNS or peripheral nervous system (PNS). In some embodiments, the AAV polyploid virions disclosed herein show altered biodistribution and increased ability of the AAV vector to transduce brain blood vessels (BBV) and/or a blood component, e.g., a cell in the blood, to allow delivery of the AAV transduced cell to the brain (or CNS or PNS) via the cerebral circulation. In some embodiments, the rational polyploid vector comprises a VP3 viral structural protein is from any serotype selected from the group consisting of AAV1, AAV6, AAV6.2, AAV7, AAV9, rh10, rh74, rh39, and rh43. In some embodiments, the rational polyploid vector comprises a VP3 viral structural protein from any non-human primate AAV serotype, for example, a rhesus monkey AAV serotype.

In some embodiments, the rational polyploid vector comprises a VP1 or VP2 structural protein, or both VP1 and VP2 structural protein from a serotype that efficiently crosses the BBB, such as e.g., AAV1, AAV6, AAV6.2, AAV7, AAV9, rh10, rh74, rh39, and rh43. In other embodiments, the rational polyploid vector disclosed herein comprises a VP1 or VP2, or both VP1 and VP2 structural protein from a serotype that does not cross the BBB. In some embodiments, the rational polyploid vector comprises a VP1 or VP2, or both VP1 and VP2 structural protein from any non-human primate AAV serotype, for example, a rhesus monkey AAV serotype (rhAAV or, AAV rh), as long as at least one of the rhesus serotypes is different. Alternatively, in some embodiments, the rational polyploid vector comprises a VP1 or VP2, or both VP1 and VP is not from a non-human primate AAV serotype. Non limiting examples of AAV serotypes, from which VP3 of the rational polyploid population of the present invention can be selected, are described in PCT/US2018/066551 (WO2019126356A1), filed Dec. 19, 2018; or PCT/US2014/055490 (WO2015038958) filed Sep. 12, 2014; or, Molecular Therapy: Methods & Clinical Development Vol. 20 Mar. 2021; each of which are herein incorporated by reference in their entirety. Non limiting examples of AAV rhesus monkey serotypes, from which VP3 of the rational polyploid population of the present invention can be selected are AAVrh10, AAV rh74, AAV rh39, AAV rh43, AAV rh38, AAV rh40, AAV rh2, AAV rh25, AAV rh57, AAV rh50, AAV rh49, AAV rh58, AAV rh61, AAV rh52, AAV rh53, AAV rh51, AAV rh64, AAV rh8, AAV rh1, AAV rh62, AAV rh48, AAV rh54, AAV rh55, AAV rh35, AAV rh37, AAV rh36, AAV rh13, AAV rh32, AAV rh33, AAV rh34 e.g., as described in Gao et al., Journal of Virology, June 2004, pg 6381-6388 which is incorporated herein by reference in its entirety

In some embodiments, the rational polyploid vector disclosed herein has enhanced binding to the brain microvascular endothelial cell (BMVEC) relative to a AAV8 vector. In some embodiments, the population of rational polyploid AAV virions has enhanced binding to brain microvascular endothelial cell (BMVEC) relative to AAV8, AAV9, PHP.B or, PHP.eB. In some embodiments, the population has at least 2 fold enhanced binding, at least 3 fold enhanced binding, at least 4 fold enhanced binding, at least 5 fold enhanced binding, at least 6 fold enhanced binding, at least 7 fold enhanced binding, at least 8 fold enhanced binding, at least 9 fold enhanced binding, at least 10 fold or, more enhanced binding relative to AAV8. In some embodiments the population of the rational polyploid AAV virions has equivalent binding to BMVEC as compared to AAV9, PHP.B, or, PHP.eB. In some embodiments, the population of haploid AAV virion has enhanced penetration of brain microvascular endothelial cells (BMVEC) relative to an AAV that does not efficiently cross the blood brain barrier e.g. AAV 8 or AAV 2 or AAV 5. In some embodiments, the rational polyploid vector disclosed herein has enhanced transduction to one or more of cortex, striatum, thalamus, medulla, hippocampus, cerebellum and spinal cord of a subject relative to a non-rational polyploid AAV particle that lacks ability to efficiently cross blood brain barrier. In some embodiment, the rational polyploid vector disclosed herein has enhanced transduction relative to any one of AAV2, AAV8 or, AAV5 in one or more of CNS regions selected from the group consisting of medulla, cervical, thoracic, lumbar, and choroid plexus.

In some embodiments, the rational polyploid vectors disclosed herein transduces a cell or tissue of the CNS. The cell of the CNS may be, but is not limited to, neurons (e.g., excitatory, inhibitory, motor, sensory, autonomic, sympathetic, parasympathetic, Purkinje, Betz, etc.), glial cells (e.g., microglia, astrocytes, oligodendrocytes) and/or supporting cells of the brain such as immune cells (e.g., T cells). The tissue of the CNS may be, but is not limited to, the cortex (e.g., frontal, parietal, occipital, temporal), thalamus, hypothalamus, striatum, putamen, caudate nucleus, hippocampus, entorhinal cortex, basal ganglia, or deep cerebellar nuclei. In some embodiments, the rational polyploid vectors disclosed herein transduce a cell or tissue of the PNS. The cell or tissue of the PNS may be, but is not limited to, a dorsal root ganglion (DRG).

In some embodiments, the rational polyploid vector disclosed herein has biodistribution in CNS, and in some embodiments, the biodistribution in the CNS is the same as (i.e., equivalent), or more (i.e., increased) than the biodistribution of AAV9 in the CNS.

In some embodiments, the rational polyploid vector disclosed herein has at least 0.05 vg/cell, 0.1 vg/cell, at least 0.2 vg/cell, at least 0.4 vg/cell, at least 0.6 vg/cell, at least 0.8 vg/cell, at least 1 vg/cell, at least 5 vg/cell, at least 10 vg/cell, at least 20 vg/cell, at least 25 vg/cell, or preferably more.

In some embodiments, the rational polyploid vector disclosed herein elicits less, or a lower, humoral immune response as compared to the humoral response as elicited by the parental AAV VP1 or, AAV VP2 serotype—that is, for example, if the rational polyploid vector comprises a VP1 and/or VP2 from AAV8 serotype and a VP3 from a serotype that crosses the BBB, the humoral response elicited by the rational polyploid vector is less as compared to the AAV8 parental virion. The reduction in the humoral response can be at least 10%, 20%, 30%, 40% or more than 40% as compared to the parental AAV VP1 or, AAV VP2 serotype. In one embodiment, humoral response is reduced as compared to the humoral response elicited by parental AAV VP3 serotype. In some embodiments, the rational polyploid vector disclosed herein elicits less, or a lower, humoral immune response as compared to the humoral response as elicited by the parental AAV VP3 serotype—that is, for example, if the rational polyploid vector comprises a VP1 and/or VP2 from AAV8 serotype and a VP3 from a serotype that crosses the BBB, including a AAVrh serotype such as, e.g., AAVrh10 or AAVrh74 serotype, the humoral response elicited by the rational polyploid vector is less as compared to the AAVrh10 or AAVrh74 parental virion. In some embodiments, the reduction in the humoral response can be at least 10%, 20%, 30%, 40% or more than 40% as compared to the parental AAV VP3 serotype. In certain aspect of the embodiment, VP3 is selected from a non-human parvovirus serotype e.g., AAVrh, such as, AAVrh10, or AAVrh74 as disclosed herein and in the Examples. In some embodiments, due to the reduced humoral immune response as compared to humoral immune response to the parental serotypes, it allows for repeat dosing, for example, the rational polyploid vectors as disclosed herein can be administered multiple times, e.g., an initial dose followed by one or more subsequent doses (e.g., boosters).

In some embodiments, the rational polyploid vector disclosed herein evades the neutralizing antibodies against the parental serotype of the VP1 or VP2 or VP3 viral structural proteins—that is, for example, if the rational polyploid vector comprises a VP1 and/or VP2 from AAV8 serotype and a VP3 from a serotype that crosses the BBB, e.g., from the AAV9 serotype, the rational polyploid vector evades the neutralizing antibodies to parental AAV8 and/or AAV9 serotypes. In some embodiments, the amount of neutralization of the rational polyploid vector from anti-AAV neutralizing antibodies to the parental serotype is less than 30%, or less than 20%, or less than 10% or, even less than 10%. For illustrative purposes only, in some embodiments, if the anti-AAV antibodies to the parental serotype neutralize the parental AAV serotype by 50%, the anti-AAV antibodies to the parental serotype neutralize or inactivate the rational polyploid by 40%, or 30%, or 20% or 10%, or less than 10%.

In some embodiments, the disclosed herein relates to a population of rational polyploid AAV virions that allow repeat dosing, the population comprising: at least one of AAV VP1, or, VP2 viral structural proteins and a AAV VP3 viral structural protein; where the VP1 and VP2 viral structural proteins are each from any AAV viral serotype, and the VP3 viral structural protein is selected from a rhesus monkey AAV serotype; and where the population of rational polyploid AAV virions elicits a reduced humoral response as compared to the humoral response elicited by the parental AAV serotype of the VP1 or VP2 viral structural proteins, wherein, the VP1 and VP2 are not from a Rhesus AAV serotype, and wherein, the repeat dosing comprises a first administration of the population of rational polyploid AAV virions and a second administration of a parental AAV serotype of the VP1 structural viral protein or, VP2 structural viral protein.

Due to the rational polyploid virions have a reduced humoral immune response, the rational polyploid virions, as described in the present invention, allows repeat dosing with parental AAV serotype e.g., repeat dosing comprises first administration with rational polyploid virion and a second administration of a parental AAV serotype which was used to provide structural protein for VP1 or, VP2 of the rational polyploid virion. For illustrative purposes only, a rational polyploid vector of AAV8-8-rh10 is administered as a first dose, a second dose can be a AAV8-8-8 serotype. In some embodiments, the rational polyploid virion allows repeat dosing wherein repeat dosing comprises a first administration of rational polyploid virion and a second administration of a parental AAV serotype VP3 viral structural protein wherein VP3 is from AAV rhesus monkey serotype. For illustrative purposes only, a rational polyploid vector of AAV8-8-rh10 is administered as a first dose, a second dose can be a AAVrh10-rh10-rh10 serotype.

In some embodiments, at least one of the viral capsid protein is a modified viral capsid protein. In some embodiments, at least one of the viral capsid protein is a chimeric viral capsid protein. The viral capsid protein can be modified by substitution, insertion or, deletion of one or, more amino acids. In some embodiments, at least one of the VP capsid viral proteins is not a chimeric. In some embodiments, VP1 is a chimeric VP1 protein. In some embodiments, VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from any other non-AAV8 vector, e.g., rh10, rh74 etc. In another embodiment only VP3 is chimeric and VP1 and VP2 are non-chimeric. In another embodiment at least one of the viral proteins is from a completely different serotype. In another example, no chimeric is present.

In one embodiment an AAV rational polyploid virion described herein that encapsidates an AAV genome (including a heterologous gene located between 2 AAV ITRs) can be formed with only two of the viral structural proteins, VP1 and VP3. In one embodiment such a AAV rational polyploid virion is conformationally correct, i.e., has T=1 icosahedral symmetry. In one embodiment, the AAV haploid virions described herein are infectious. The ITR can be an ITR from any serotype, e.g., AAV8 or AAV2, or from any of the 12 serotypes of AAV isolated for gene therapy, other species, mutant serotypes, shuffled serotypes of such genes, e.g., AAV1, AAV2, VP1.5, AAV4 VP2, AAV4 VP3, Rh10 VP3, Rh74 VP3, Rh74 VP2 or any other AAV serotype desired, for example as disclosed in Table 1.

In some embodiments, a substantially pure population of AAV rational polyploid virions disclosed herein is at least 10¹ virions, at least 10² virions, at least 10³ virions, at least 10⁴ virions, at least 10⁵ virions, at least 10⁶ virions, at least 10⁷ virions, at least 10⁸ virions, at least 10⁹ virions, at least 10¹⁰ virions, at least 10¹¹ virions, at least 10¹² virions, at least 10¹⁵virions, at least 10¹⁷ virions. In one embodiment, the population is at least 100 viral particles. In one embodiment, the population of AAV rational polyploid virions disclosed herein is from 10⁹ to 10¹² virions

In one embodiment, the population is at least 1×10⁴ viral genomes (vg)/ml, is at least 1×10⁵ viral genomes (vg)/ml, is at least 1×10⁶ viral genomes (vg)/ml, at least 1×10⁷ viral genomes (vg)/ml, at least 1×10⁸ viral genomes (vg)/ml, at least 1×10⁹ viral genomes (vg)/ml, at least 1×10¹⁰ vg/ml, at least 1×10¹¹ vg/ml, at least 1×10¹² vg/ml. In one embodiment, the population ranges from about 1×10⁵ vg/ml to about 1×10¹³ vg/ml.

In some embodiments, a polyploid AAV vector as disclosed herein useful for the methods to treat a disease or disorder of the brain or spinal cord, or a neuronal or neurodegenerative disease, exemplary doses for achieving therapeutic effects are titers of at least about 1.0E12 to 4.0E12 vg/kg, or about 1.2E12 to 3.0E12 vg/kg, or about 1.2E12 to 2.5E12 vg/kg, or about 2.5E12 to 4.0E12 vg/kg.

A substantially homogenous population is at least 90% of only the desired AAV rational polyploid described herein, at least 91%, at least 93%, at least 95%, at least 97%, at least 99%, at least 99.5%, or at least 99.9%. In one embodiment, the population is completely homogenous. Accordingly, some aspects of the technology described herein relates to a system for producing a substantially homogenous haploid or rational polyploid AAV virions, comprising a vector comprising the nucleic acid encoding a VP1 only from one AAV serotype selected from Table 1, and, optionally VP2 only from one AAV serotype selected from Table 1, and VP3 from any AAV serotype that efficiently crosses the BBB, e.g., AAV1, AAV6, AAV6.2, AAV7, AAV9, rh10, rh74, rh39, and rh43. In some embodiments, the VP3 protein is from a non-human primate, e.g., a chimpanzee or rhesus monkey AAV (AAVrh) serotype, e.g., AAVrh.10, AAVrh.74, AAVrh.73, AAVrh.75, AAVrh.76, rAAVrh.39, rAAVrh.43, as disclosed herein. In some embodiments, an exemplary system comprises (i) a promoter operatively linked to a nucleic acid encoding VP1 and VP2 from a first AAV serotype only, but does not express VP3 from the first AAV serotype, where the first AAV serotype is selected from any serotype listed in Table 1, and (ii) a promoter operatively linked to a nucleic acid encoding a VP3 protein from any serotype that efficiently crosses the BBB as disclose herein, e.g., AAV1, AAV6, AAV6.2, AAV7, AAV9, rh10, rh74, rh39, and rh43.

One aspect provided herein provides a population of rational polyploid AAV virions suitable for use in crossing the blood brain barrier, wherein, the rational polyploid AAV virions comprise at least one of AAV VP1, or, VP2 viral structural proteins and an AAV VP3 viral structural protein; wherein, the VP1 and VP2 viral structural proteins are each from any AAV serotype, and the VP3 viral structural protein is from an AAV serotype that efficiently crosses the blood brain barrier and is different from the serotype of at least one of VP1 or VP2, and wherein, the population of rational polyploid AAV virions crosses the blood brain barrier (BBB) and/or transduces an endothelial cell of the BBB, and/or is a blood component that crosses the BBB upon systemic or, intrathecal administration.

In one embodiment of any aspect herein, the population exhibits enhanced transduction activity across the blood brain barrier (BBB) relative to a non-rational polyploid AAV particle that lacks ability to cross blood brain barrier.

In one embodiment of any aspect herein, the VP3 viral structural protein is an AAV rhesus monkey serotype.

In one embodiment of any aspect herein, the VP3 viral structural protein is from a serotype that efficiently crosses the blood brain barrier selected from the group consisting of AAV1, AAV6, AAV6.2, AAV7, AAV9, AAVrh10, AAVrh74, AAVrh.39, and AAVrh43.

In one embodiment of any aspect herein, the population has enhanced transduction to one or more of cortex, striatum, thalamus, medulla, hippocampus, cerebellum and spinal cord of a subject relative to a non-rational polyploid AAV particle that lacks ability to efficiently cross blood brain barrier.

In one embodiment of any aspect herein, said rational polyploid AAV has enhanced transduction relative to AAV2 in one or more of CNS regions selected from the group consisting of medulla, cervical, thoracic, lumbar, and choroid plexus.

In one embodiment of any aspect herein, said rational polyploid AAV has enhanced binding to brain microvascular endothelial cell (BMVEC) relative to AAV8.

In one embodiment of any aspect herein, the population has biodistribution in CNS.

In one embodiment of any aspect herein, the CNS biodistribution is at least 0.05 vg/cell, 0.1 vg/cell, at least 0.2 vg/cell, at least 0.4 vg/cell, at least 0.6 vg/cell, at least 0.8 vg/cell, at least 1 vg/cell, at least 5 vg/cell, at least 10 vg/cell, at least 20 vg/cell, at least 25 vg/cell, or preferably more.

In one embodiment of any aspect herein, either VP1 or, VP2 selected from AAV serotype that crosses blood brain barrier.

In one embodiment of any aspect herein, either VP1 or, VP2 selected from an AAV serotype that do not cross blood brain barrier.

In one embodiment of any aspect herein, VP1 or, VP2 not selected from AAV rhesus monkey serotype.

In one embodiment of any aspect herein, either VP1 or, VP2 selected from AAV rhesus monkey serotype.

In one embodiment of any aspect herein, the population elicits less humoral immune response as compared to the humoral response as elicited by the parental AAV VP1 or, AAV VP2 serotype.

In one embodiment of any aspect herein, the population evades neutralizing antibodies against the parental serotypes of AAV VP1, VP2 or, VP3 viral structural proteins.

One aspect provided herein provides a method for delivering a transgene across the blood brain barrier comprising administering a population of any of the rational polyploid AAV virions described herein.

One aspect provided herein provides a method for repeat dosing comprising a first and second administrations, wherein, the repeat dosing comprises the first administration of any of the rational polyploid AAV virions described herein, and the second administration of parental AAV serotypes of VP1 or VP2 viral structural protein, wherein the population of rational polyploid AAV virion elicits a reduced humoral response as compared to the humoral response as elicited by the parental AAV serotypes of VP1 or VP2 viral structural protein, and wherein, VP1 or, VP2 is not from a Rhesus AAV serotype.

One aspect provided herein provides a population of rational polyploid AAV virions that allow repeat dosing, the population comprising: at least one of AAV VP1, or, VP2 viral structural proteins and a AAV VP3 viral structural protein; wherein, the VP1 and VP2 viral structural proteins are each from any AAV viral serotype, and the VP3 viral structural protein is selected from a rhesus monkey AAV serotype; wherein, the population of rational polyploid AAV virions elicits a reduced humoral response as compared to the humoral response elicited by the parental AAV serotype of the VP1 or VP2 viral structural proteins, wherein, the VP1 and VP2 are not from a Rhesus AAV serotype, and wherein, the repeat dosing comprises a first administration of the population of rational polyploid AAV virions and a second administration of a parental AAV serotype of the VP1 structural viral protein or, VP2 structural viral protein.

One aspect provided herein provides a population of rational polyploid AAV virion, wherein, the population comprises (a) VP1 and VP2 of AAV viral structural protein selected from AAV8 viral serotype, and (b) VP3 selected from AAV rhesus monkey serotype, AAV rh10 or, AAVrh74
wherein, said population of rational polyploid AAV virion elicit reduced humoral response than elicited by parental AAV8 serotype.

One aspect provided herein provides a method for repeat dosing comprising a first and second administrations, wherein, the first administration is a population of any of the rational polyploid AAV virions described herein, and the second administration is the parental AAV serotype of VP1 or VP2 viral structural protein, wherein, the first administration elicits a reduced humoral response as compared to the humoral response as elicited by the parental AAV serotypes of VP1 or VP2 viral structural protein, and wherein, VP1 or VP2 are not from a Rhesus AAV serotype.

In one embodiment of any aspect herein, the population evades neutralizing antibodies against the parental serotypes of AAV VP1, VP2 or, VP3 viral structural proteins.

One aspect provided herein provides a method for delivering a transgene across the blood brain barrier comprising administering a population of any of the rational polyploid AAV virions described herein.

In one embodiment of any aspect herein, the VP3 protein is a mutated VP3 protein from AAVrh10 or AAVrh74 serotype.

In one embodiment of any aspect herein, the mutated AAVrh74 VP3 protein has the amino acid sequence of SEQ ID NO: 2 or a protein having at least 85% sequence identity to SEQ ID NO: 2, or wherein the mutated AAVrh74 VP3 comprises at least one of the following modifications of SEQ ID NO: 2: N263S, G264A, T265S, S266T, G268A, T270del, T274H, E533K, R726H, N736P.

In one embodiment of any aspect herein, the mutated AAVrh10 VP3 protein is encoded by a nucleic acid of SEQ ID NO: 5 that comprises at least one or more of: Q214N, S462N and D517E mutations as compared to AAVrh10_VP3 nucleic acid of SEQ ID NO: 5, or comprises a nucleic acid sequence at least 85% sequence identity to SEQ ID NO: 5 comprising at least one mutation selected from Q214N, S462N and D517E.

In one embodiment of any aspect herein, the VP3 protein is a AAVrh74 VP3 protein comprising the amino acid sequences of SEQ ID NO: 2 or 3 or a protein having at least 85% sequence identity to SEQ ID NO: 2 or SEQ ID NO: 3, or comprises at least one of the following amino acid modifications of N263S, G264A, T265S, S266T, G268A, T270del, T274H, E533K, R726H, N736P of SEQ ID NO: 2.

One aspect provided herein provides a substantially homogenous population of any of the virions described herein, wherein the population is at least 10¹ virions.

One aspect provided herein provides a nucleic acid comprising, in a 5′ to 3′ direction: (a) a first nucleic acid encoding an AAVrh10 VP3 capsid protein operatively linked to a first promoter; (b) a first poly A sequence (c) a second nucleic acid encoding a rep protein (d) a third nucleic acid encoding AAV8 VP1 and VP2 viral structural proteins and wherein the third nucleic acid sequence is not capable of expressing an AAV8 VP3 viral structural protein, and (e) a second poly A sequence.

One aspect provided herein provides a nucleic acid comprising, in a 5′ to 3′ direction: (a) a first nucleic acid encoding a AAVrh74 VP3 capsid protein operatively linked to a first promoter; (b) a first poly A sequence (c) a second nucleic acid encoding a rep protein (d) a third nucleic acid encoding AAV8 VP1 and VP2 viral structural proteins and wherein the third nucleic acid sequence is not capable of expressing a AAV8 VP3 viral structural protein, and (e) a second poly A sequence.

One aspect provided herein provides a viral vector comprising: (a) any of the AAV virions described herein; and (b) a nucleic acid comprising at least one terminal repeat sequence, and a heterologous gene, wherein the nucleic acid is encapsulated by the AAV capsid.

In one embodiment of any aspect herein, the population comprises chimeric or, modified viral structural protein wherein the modified viral structural protein comprises insertion, deletion or, substitution of one or more amino acids.

In one embodiment of any aspect herein, the substantially homogenous population produces a significantly less anti-AAV IgG antibodies against parental AAV serotypes of VP1 or VP2 structural proteins in the serum in vivo as compared to a substantially homogenous population of virions comprising parental AAV serotype.

In one embodiment of any aspect herein, the parental AAV serotype is AAV8.

One aspect provided herein provides a population of rational polyploid AAV virions that allow repeat dosing, the population comprising: at least one of AAV VP1, or, VP2 viral structural proteins and a AAV VP3 viral structural protein; wherein, the VP1 and VP2 viral structural proteins are each from any AAV viral serotype, and the VP3 viral structural protein is selected from a rhesus monkey AAV serotype; wherein, the population of rational polyploid AAV virions evade neutralizing antibodies against parental AAV rhesus monkey serotype of VP3 viral structural protein, wherein, the VP1 and VP2 are not from a Rhesus AAV serotype, wherein, the repeat dosing comprises a first administration of the population of the parental AAV rhesus monkey serotype of VP3 structural protein and a second administration of the population of rational polyploid AAV virions, and wherein, the VP3 structural protein of the rational polyploid virions is a AAV rhesus monkey mutated viral structural protein VP3.

In one embodiment of any aspect herein, the AAV rhesus monkey mutated viral structural protein VP3 is from a mutated AAV rh10 VP3 viral structural protein or from a mutated AAV rh74 VP3 viral structural protein.

In one embodiment of any aspect herein, the mutated viral structural protein VP3 comprises a mutation at an amino acid that corresponds to an amino acid selected from the group consisting of N263, G264, T265, S266T, G268, T270, T274, E533 wherein all amino acid positions correspond to native VP1 sequence numbering of AAV rh10 or AAVrh74.

In one embodiment of any aspect herein, the mutation is selected from the group consisting of N263S, G264A, T265S, S266T, G268A, T270del, T274H, E533K

In one embodiment of any aspect herein, the mutated viral structural protein VP3 further comprises a mutation at an amino acid that corresponds to an amino acid selected from the group consisting of R727 and N737 wherein all amino acid positions correspond to native VP1 sequence numbering of AAVrh10.

In one embodiment of any aspect herein, the mutation is selected from the group consisting of R727H and N737P.

In one embodiment of any aspect herein, the mutated viral structural protein VP3 further comprises a mutation at an amino acid that corresponds to an amino acid selected from the group consisting of R726 and N736 wherein all amino acid positions correspond to native VP1 sequence numbering of AAV rh74.

In one embodiment of any aspect herein, the mutation is selected from the group consisting of R726H and N736P.

In one embodiment of any aspect herein, the mutated viral structural protein VP3 further comprises a mutation at an amino acid that corresponds to W at 581, wherein W is replaced by two subsequent V residues (VV) and wherein all amino acid positions correspond to native VP1 sequence numbering of AAV rh74.

In one embodiment of any aspect herein, AAV VP1 or VP2 viral structural protein is any AAV serotype selected from Table 1.

In one embodiment of any aspect herein, AAV VP1 or VP2 structural protein is AAV8.

In additional embodiments, the present invention provides a AAV rational polyploid virus vector comprising: (a) a rational polyploid AAV vector as disclosed herein; and (b) a nucleic acid comprising at least one terminal repeat sequence, wherein the nucleic acid is encapsidated by the AAV rational polyploid virus. The AAV rational polyploid virus vector can be an AAV haploid particle and the AAV rational polyploid virus vector protein, capsid, virus vector and/or AAV haploid particle as disclosed herein can be present in a composition that further comprises a pharmaceutically acceptable carrier.

In further embodiments, the present invention provides a method of administering a nucleic acid to a cell, the method comprising contacting the cell with the AAV haploid virus vector of this invention and/or a composition of this invention.

Also provided herein is a method of delivering a nucleic acid to a subject, the method comprising administering to the subject the AAV haploid virus vector and/or a composition of this invention.

Additionally, provided herein is the AAV8 haploid capsid protein, capsid, virus vector, AAV particle and/or composition of this invention for use as a medicament in the beneficial treatment of a disorder or disease.

In further embodiments, the present invention provides a population of rational polyploid AAV virions that allow repeat dosing (i.e., an initial dose, and one, or 2, or 3, or 4 or 5 or more than 5 subsequent doses or boosters), the population comprising: at least one of AAV VP1, or, VP2 viral structural proteins and a AAV VP3 viral structural protein; where the VP1 and VP2 viral structural proteins are each from any AAV viral serotype, and the VP3 viral structural protein is selected from a rhesus monkey AAV serotype (rhAAV) and where the population of rational polyploid AAV virions evade neutralizing antibodies against parental AAV rhesus monkey serotype of VP3 viral structural protein, where the VP1 and VP2 are not from a Rhesus AAV serotype and the repeat dosing comprises a first administration of the population of the parental AAV rhesus monkey serotype of VP3 structural protein and a second administration of the population of rational polyploid AAV virions, and wherein the VP3 structural protein of the rational polyploid virions is a AAV rhesus monkey mutated viral structural protein. In some embodiments, the VP3 structural protein is a modified VP3 protein from a rhAAV, e.g., a modified VP3 protein from rh10 or rh74. In some embodiments, the modified VP3 protein is a rh10-LP2 VP3 protein as disclosed herein. In some embodiments, the modified VP3 protein is a rh74-LP2 VP3 protein as disclosed herein.

In some embodiments, the population of rational polyploid virions that allow repeat dosing the repeat dosing comprises a first administration of the rational polyploid AAV virion and the second administration of parental AAV VP3 viral structural protein of rhesus monkey serotype and wherein, the VP3 structural protein of the rational polyploid virions is a AAV rhesus monkey mutated viral structural protein VP3, wherein the population comprises at least one of AAV VP1, or, VP2 viral structural proteins and a AAV VP3 viral structural protein; wherein, the VP1 and VP2 viral structural proteins are each from any AAV viral serotype, and the VP3 viral structural protein is selected from a rhesus monkey AAV serotype; wherein, the population of rational polyploid AAV virions evade neutralizing antibodies against parental AAV rhesus monkey serotype of VP3 viral structural protein, and wherein, at least one of the VP1 and VP2 are not from a Rhesus AAV serotype. In some embodiments, both the VP1 and VP2 structural proteins are not from a Rhesus AAV serotype.

Rational polyploid comprising rhesus monkey modified VP3 protein (e.g., a rh10-VP3 protein comprising any one or more of modifications: N263S, G264A, T265S, S266T, G268A, T270del, T274H, E533K, R727H, N737P as disclosed herein, or a rh74-VP3 protein comprising any one or more of N263S, G264A, T265S, S266T, G268A, T270del, T274H, E533K, R726H, N736P) that can evade neutralizing Ab against parental AAV rhesus monkey serotype. In some embodiments, the population can escape neutralizing Ab against parental AAV VP1 serotype, or, AAV VP2serotype wherein VP1 or VP2 not from rhesus monkey serotype.

These and other aspects of the invention are addressed in more detail in the description of the invention set forth below.

### BRIEF DESCRIPTION OF THE DRAWINGS

This application file contains at least one drawing executed in color. Copies of this patent application publication with color drawings will be provided by the Office upon request and payment of the necessary fee. The accompanying drawings illustrate aspects of the present invention.

The following Detailed Description, given by way of example, but not intended to limit the invention to specific embodiments described, may be understood in conjunction with the accompanying drawings, incorporated herein by reference. Various preferred features and embodiments of the present invention will now be described by way of non-limiting example and with reference to the accompanying drawings, in which:

FIGS. 1A-1B show schematics of an all-in-one construct for generation of a AAV8 haploid as disclosed herein. FIG. 1A shows a schematic of a construct for generation of AAV8-8-rh10 or AAV8-8-rh74 vectors, comprising (i) a promoter operatively linked to a nucleic acid encoding a VP3 protein from the AAVrh10 serotype or a AAVrh74 serotype followed by a poly A sequence, (ii) a nucleic acid sequence encoding Rep 2 genes (e.g., comprising promoters p5, p15 and p40), and (iii) a nucleic acid encoding VP1 and VP2 from AAV8 serotype, where the start initiation codon for VP3 capsid protein expression is inactivated. This construct can be used for the generation of haploid AAV8 viruses in vitro. FIG. 1B shows an exemplary plasmid map for the generation of an AAV8-8-rh74 haploid vector.

FIG. 2 shows structural models of AAV8-8-rh10 (left) and AAV8-8-rh74 (right) haploid viruses (four random capsids from a population, as VP subunits can combine in different ways), where red is the surface of the virion due to AAV8 VP1 or VP2 capsid proteins, green shows representation of VP3 capsid protein from AAVrh10 serotype, and blue shows surface representation from the VP3 capsid protein from AAVrh74 serotype.

FIG. 3 shows stimulation and modeling of the tertiary protein structure of VP3 capsid protein from AAVrh10 serotype (left) or AAVrh74 serotype (right).

FIG. 4 Interface analysis of parental serotypes, AAV88rh.10 and AAV88rh.74 haploids, the mutants replacing AAVrh.10 residues by the corresponding residues in AAVrh.74 and the haploids resulting of the use of these mutants instead of the parental AAVrh.10 VP3. Y axis shows the calculated interaction energy between a VP3 subunit and all the neighboring subunits making direct contact. Each complex was minimized before the calculation of the interaction energy, and this process repeated 30 times per complex. The mean values for the interaction energies were compared by a Wilcoxon pairwise test. Different labeling letters on top of the boxplots represent statistically significant differences between the groups.

FIG. 5 shows a schematic of a construct for the generation of a AAV8 haploid comprising VP1 and VP2 from the AAV8 serotype, showing the modification of two ATG initiation codons to GTG to result in two amino acid substitution M203V and M211V, to prevent translation of the VP3 AAV8 capsid protein. FIG. 5 discloses SEQ ID NOS 28-40, respectively, in order of appearance.

FIG. 6 shows results from a western blot showing expression of VP1, VP2 and VP3 proteins from AAV8-8-rh10 or AAV8-8-rh74 haploids, as compared to AAV8, or where AAV8-8-VP3KO. Top panel is a schematic shown in FIG. 1 of the construct for production of AAV8-8-rh10 or AAV8-8-rh74 haploids, and bottom panel shows a western blot analysis of Pro10 cells infected with AAV8 vector, or AAV8-8-rh10 or AAV8-8-rh74 haploids, and an anti-CAP antibody used to detect VP1 and VP2 from AAV8 serotype, and VP3 from AAVrh74 (lane 1) or AAVrh10 (lane 2) or AAV8 (lane 4). Lane 3 shows absence of VP3 capsid protein expression, indicating VP3 is not expressed from the construct comprising a nucleic acid where two ATG initiation codons are changed to GTG to result in amino acid substitutions M203V and M211V, which prevent translation and expression of the VP3 AAV8 capsid protein.

FIG. 7 shows results of genome protection assay of AAV8-8-rh10 or AAV8-8-rh74 haploids as compared to AAV8 vector (AAV8-8-8[WT]) or AAV8-VP3KO (AAV8-8-VP3KO).

FIG. 8 is a table of the specific productivity of AAV8-8-rh10 or AAV8-8-rh74 haploids as determined by qPCR and ELISA, showing AAV8-8-rh10 productivity was comparable to AAV8 and AAVrh10 control. AAV8-8-rh74 was at a lower productivity level as compared to AAV8-8-rh10 and controls. Comparison of means statistically insignificant.

FIG. 9A-9B shows Affinity Chromatography results and AEX chromatography of AAV8-8-rh10 or AAV8-8-rh74 haploids. FIG. 9A shows Affinity Chromatography results, showing lower SEC 260/280 value of AAV8-8-rh74 haploids (see arrow) suggesting a lower packaging efficiency during production. FIG. 9B shows AEX chromatography, where after Affinity Chromatography, Iodixanol density gradient ultracentrifugation (DGUC) process step is performed to separate empty capsids from full capsids, and the Iodixanol pool is diluted and loaded onto the AEX column. The SEC 260/280 value closer to 1.3 (˜75% or greater) suggest successful enrichment of full capsids. AAV8-8-rh74 value (arrow) is 1.1 indicating lower enrichment.

FIGS. 10A-10D shows results for product recovery as determined by qPCR and ELISA from AAV8-8-rh10 or AAV8-8-rh74 haploids as compared to AAV8 and AAVrh10 controls. FIG. 10A shows product recovery from AAV8 control, and FIG. 10B shows product recovery from AAVrh10 control. The current generation process product recovery for AAV8 and AAVrh10 is 8-12% based on qPCR assay, and the recovery of the AAV8 and AAVrh10 controls is comparable to each other. FIG. 10C shows product recovery from AAV8-8-rh10 vector comprising Luciferase gene, which shows the overall recovery is comparable to AAV8 and AAVrh10 controls (FIGS. 10A and 10B respectively). FIG. 10D shows product recovery from AAV8-8-rh74 vector comprising Luciferase gene, showing overall recovery below 5%, with a lower initial productivity which results in lower recovery, however, DSP unit operations report lower recovery starting with affinity chromatography (˜5-12%).

FIG. 11A-11C show electropherogram analysis of the AAV8-8-rh10 or AAV8-8-rh74 haploids as compared to AAV8 and AAVrh10 controls. FIG. 11A shows the ratio of VP1, VP2 and VP3 of AAV8-8-rh10 or AAV8-8-rh74 haploids as compared to AAV8 and AAVrh10 controls. FIG. 11B shows electropherogram analysis of AAV8 control (overlay of 3 individual preparations). FIG. 11C shows electropherogram analysis of AAV8-8-rh10 (bottom) and AAV8-8-rh74 (top) haploids.

FIG. 12A-12B show representative electropherogram analysis of the AAV8-8-rh10 or AAV8-8-rh74 haploids. FIG. 12A show two representative electropherogram graphs of the AAV8-8-rh10 or AAV8-8-rh74 haploids, showing the AAV8 reference material and each of the AAV8-8-rh10 or AAV8-8-rh74 virions. FIG. 12B shows representative electropherogram graphs of AAV5 and AAV9 serotypes, showing that different serotypes have exhibited different relative ratios of the three capsid proteins.

FIG. 13A-13B shows results of transduction efficiency of the AAV8-8-rh10 or AAV8-8-rh74 haploids in Pro10 cells as compared to AAV8 and AAVrh10 controls. FIG. 13A is a schematic of the protocol to determine transduction efficacy of each AAV8 haploid vector, where the plasmids for generation of the vectors are used to transduce Pro10 production cell line. FIG. 13B shows results of efficiency of transduction at 100K MOI of two experiments (left and middle graphs) and combined meta analysis (right graph) of AAV8-8-rh10 or AAV8-8-rh74 haploids as compared to AAV8 and AAVrh10 controls, which showed that there were some differences in the ability of the haploid vectors to transduce Pro10 cells: AAV8-8-Rh74 haploid transduced Pro10 cells similar to AAV8 control. Statistics was performed by ANOVA+Tukey tests.

FIG. 14A-14B shows escape from AAV8 neutralizing antibodies by AAV8-8-rh10 or AAV8-8-rh74 haploids as compared to AAV8 and AAVrh10 controls. FIG. 14A shows results from two experiments (left and middle graphs) and combined meta analysis (right graphs) suggesting AAV8-8-rh10 or AAV8-8-rh74 haploids can efficiently escape neutralizing Ab AAV8 1/100 as compared to AAV8 and AAVrh10 controls and thus exhibit lower % luciferase inhibition compared to controls. FIG. 14B shows results from two experiments (left and middle graphs) and combined meta analysis (right graphs) showing higher efficiency of AAV8-8-rh10 or AAV8-8-rh74 haploids in escaping neutralizing Ab AAV8 1/200 as compared to AAV8 and AAVrh10 controls as suggested by lower % luciferase inhibition compared to controls.

FIG. 15 (experiment 1, experiment 2 and combined meta analysis) show in presence of AAV8 neutralizing Ab (serum from mice inoculated with AAV8), AAV8 mediated luciferase expression was affected in a dose dependent manner whereas, AAV8-8-rh74 mediated luciferase expression was unaffected in presence or in absence (black bar) of AAV8 neutralizing antibodies. Statistics was performed by ANOVA+Tukey tests.

FIG. 16 (experiment 1, experiment 2 and combined meta-analysis) shows in presence of serum from AAV8-inoculated mice comprising AAV8 neutralizing Ab, AAV8-8-rh10 mediated luciferase expression was affected almost similarly to the AAV8 control (as shown in FIG. 15). As suggested by the lower panel of FIG. 16, AAV8 neutralizing Ab (AAV8 serum) showed cross reactivity against AAVrh10. Statistics was performed by ANOVA+Tukey tests.

FIG. 17A-17B shows results of transduction efficiency of the AAV8-8-rh10 or AAV8-8-rh74 haploids in the human fibroblast cell line GM16095 cells as compared to AAV8 and AAVrh10 controls. FIG. 17A is a schematic of the protocol to determine transduction efficacy of each AAV8 haploid vector, where the plasmids for generation of the vectors are used to transduce the neuronal cell line GM16095.

FIG. 17B shows results of efficiency of transduction at 100K MOI of AAV8-8-rh10 or AAV8-8-rh74 haploids as compared to AAV8 and AAVrh10 controls, which showed that there were some differences in the ability of the haploid vectors to transduce GM16095 cells: AAV8-8-Rh74 haploid transduced GM16095cells significantly more efficiently than AAV8 or AAVrh10 control, whereas AAVrh10 is more efficient than AAV8 in this GM16095 cell line, Statistics was performed by ANOVA+Tukey tests.

FIG. 18A-18B shows escape from AAV8 neutralizing antibodies (Nab) in GM16095 cells by AAV8-8-rh74 haploid as compared to AAV8 and AAVrh10 controls. FIG. 18A shows that NAb in the serum of mice inoculated with AAV8 inhibited the transduction of GM16095 cells with AAV8 in a dose-dependent manner. AAV8-8-Rh74 haploid vector efficiently escaped from anti-AAV8 NAb, and no differences were found in the luciferase transgene expression in the presence and absence of NAb. Transduction with AAV8-8-Rh10 haploid was inhibited by the NAb in similar extent than AAV8 control. Cross-reactivity of the anti-AAV8 NAb was detected against AAVRh10. FIG. 18B shows that NAb in the serum of mice inoculated with AAV8 at 1/100 or 1/200 inhibited the transduction of GM16095 cells with AAV8 and AAV8-8-rh10, but did not inhibit the transduction of GM16095 cells with AAV8-8-rh74 at either 1/100 or 1/200 concentrations, demonstrating that AAV8-8-Rh74 haploid vector was able to escape from anti-AAV8 NAb, whereas transduction with AAV8-8-Rh10 haploid was inhibited by the NAb in similar extent than AAV8 control. Cross-reactivity of the anti-AAV8 NAb was detected against AAVRh10.

FIG. 19A-19B shows efficiency of transduction of Pro10 cells in vitro by the AAV8 haploid vectors and escape from neutralizing antibodies (Nab) in Pro10 cells by AAV8-8-rh74 haploid as compared to AAV8 and AAVrh10 controls. FIG. 19A shows the higher efficacy of AAV8-8-rh74 transduction of Pro10 cells compared to AAV8 or, AAVrh10 at 100K MOI (left) and in the presence of AAV8 serum at 1/100 (middle) and 1/200 concentrations. FIG. 19B shows that NAb in the serum of mice inoculated with AAV8 inhibited the transduction of Pro10 cells with AAV8 in a dose-dependent manner. AAV8-8-Rh74 haploid vector was able to escape from anti-AAV8 NAb, and no differences were found in the AAV8-8-rh74 mediated luciferase transgene expression in the presence and absence of NAb. Transduction with AAV8-8-Rh10 haploid was inhibited by the NAb in similar extent than AAV8 control. Cross-reactivity of the anti-AAV8 NAb was detected against AAVRh10.

FIG. 20 is a schematic showing the protocol for analysis of the biodistribution of transduction of the AAV8-8-rh10 or AAV8-8-rh74 haploids as compared to AAV8 and AAVrh10 controls in mice in vivo. The experiment was performed with four groups of mice each group having 5 in them; in each group, 4 mice were injected with experimental vector (experimental mice), and one was left untreated (control mouse). The mice were injected intravenously with 5×10¹⁰ vg/mouse of control AAV8 (control mouse) or, AAVrh10 or haploid AAV8-8-rh10 or AAV8-8-rh74 (experimental mice).

FIG. 21A-21D shows the biodistribution of the transduction from the AAV8 haploids in vivo as determined by luciferase expression. FIG. 21A shows the ventral biodistribution of luciferase expression after 30 second exposure at 7 days post injection (dpi) of AAV8-8-rh10 (Group 2) or AAV8-8-rh74 (Group 3) haploids as compared to AAV8 (Group 1) and AAVrh10 (Group 4) controls, showing a different biodistribution of the AAV8-8-Rh74 vector, indicating both systemic biodistribution as well as significant distribution of AAV8-8-Rh74 in the brain and spinal cord and crossing blood brain barrier.

FIG. 21B shows the ventral biodistribution of luciferase expression after 1 minute exposure at 7 days post injection (7 dpi) of AAV8-8-rh10 (Group 2) or AAV8-8-rh74 (Group 3) haploids as compared to AAV8 (Group 1) and AAVrh10 (Group 4) controls, showing significantly higher distribution of AAV8-8-Rh74 and corroborates the result shown in 21A. FIG. 21C shows the ventral biodistribution of luciferase expression after auto-exposure at 7 days post injection (7 dpi) of AAV8-8-rh10 (Group 2) or AAV8-8-rh74 (Group 3) haploids as compared to AAV8 (Group 1) and AAVrh10 (Group 4) controls, showing the basal bioluminescence level in all four groups and thus further confirming the significantly enhanced biodistribution of AAV8-8-rh74 compared to other groups. FIG. 21D is the graphical representation of the total Flux (p/s) of the dorsal vs. ventral view of luciferase expression after 7 days. Statistics was performed by ANOVA+Tukey tests (n=4 mice/group). Similar result is obtained at 14 days and 21 days post injection wherein AAV 8-8-rh74 consistently show enhanced systemic biodistribution e.g., in CNS and other tissues compared to other groups. in vivo. Statistics was performed by ANOVA+Tukey tests (n=4 mice/group). Similar result is obtained at 14 days and 21 days post injection wherein AAV 8-8-rh74 consistently show enhanced systemic biodistribution e.g., in CNS and other tissues compared to other groups.

FIG. 22 shows the amino acid sequence alignment of the VP3 capsid protein from AAVrh10 (top sequence) compared to the VP3 capsid protein from AAVrh74 (bottom sequence), showing these sequence differ only in 4 regions.

FIG. 23A-23C show AAVrh.10 (green) vs AAVrh.74 (cyan). VP3 domain from both serotypes differ only in 5 positions: Q417N (red), VV581W (blue), S665N (magenta) and D720E (orange) [using the nomenclature/numbering from the amino acid sequence of the VP1 capsid protein]. FIG. 23A shows the superimposed tertiary structure of VP3 capsid protein from AAVrh.10 vs AAVrh.74, with the residues of interest colored as described, while FIG. 23B shows surface of AAVrh10, and FIG. 23C shows AAVrh74 capsids, with the previously described colors convention. FIGS. 23B and 23C clearly show that all positions, except for 417, are accessible to the solvent (and NAbs). Both Q417 in AAVrh.10 and N417 in AAVrh.74 are buried (no red patches on the surfaces), so they are likely not responsible for any difference in the recognition by NAbs. Positions 581 and/or 665 and/or 720 could be responsible of a different recognition by AcNs of AAVrh.10 vs. AAVrh74, which is extensible to the haploids containing them

FIG. 24 results from the protease cleavage profiles between AAV8-8-rh10 or AAV8-8-rh74 haploids. The number of cleavages variate in 19, from the 49 proteases was analyzed. From these, the following group can be highlighted: (i) Cathepsin K (the only lysosomal enzyme in the prediction set). AAVrh.74 has an additional cleavage site, (ii) Matrix Metallopeptidase-1, -2, -3 and -9. AAVrh.74 loses a site for -1 and -3 and gains one for MMP-2 and -9. As with Cathepsin K is probable that the capsids could have contact with these enzymes in their normal biodistribution and infection process. (iii) Granzymes, Elastase, Cathepsin G and Caspases: these enzymes could be relevant for the immune response to the virus. (iv) Pepsin and Chymotrypsin are digestive enzymes.

FIG. 25A-25D show the humoral immune response of AAV8-8-rh10 or AAV8-8-rh74 haploids as compared to AAV8 and AAVrh10 controls. FIG. 25A shows anti-AAV8 IgG levels (1/1000 serum dilution) and FIG. 25B shows anti-AAV8 IgG levels (1/5000 serum dilution), showing significantly reduced anti-AAV8 IgG levels were detected in the serum from mice inoculated with both haploid vectors, in comparison to the mice injected with AAV8. No cross-reactivity against AAV8 was found with serum from the mice inoculated with AAVrh10 at the serum dilutions tested. FIG. 25C shows anti-AAVrh10 IgG levels (1/1000 serum dilution) and FIG. 25D shows anti-AAVrh10 IgG levels (1/5000 serum dilution), and shows that AAVrh10 was significantly less immunogenic than AAV8, and no significant differences were observed in the anti-AAVrh10 IgG levels between the mice inoculated with AAVrh10 and the rest of the experimental groups at the serum dilution tested. Statistics was performed by ANOVA+Tukey tests (n=4 mice/group).

FIG. 26A-26B show the humoral immune response to AAV8, AAVrh10, haploids AAV8-8-rh10 and AAV8-8-rh74; and AAV8 mediated GM16095 cell transduction in presence of Nabs e.g. in presence of serum from mice inoculated with AAV8 (group 1), AAV8-8-rh10 (group 2), AAV8-8-rh74 (group 3), AAVrh10 (group 4), and no treatment control (group 5). FIG. 26A shows neutralization of AAV8 mediated transduction of GM16095 cells by AAV8 serum in a dose dependent manner whereas AAV8 mediated transduction was not neutralized in presence of serum (1/100, or, 1/200, or, 1/400 dilution) from mice inoculated with AAV8-8-rh10 (Group 2) or AAV8-8-rh74 (Group 3) haploids or, AAVrh10 (Group 4) or non-treated controls (group 5). FIG. 26B shows a graph of the neutralization by AAV8 serum at 1/100 dilution (left) or 1/200 dilution (right) showing inhibition of luciferase expression from AAV8 only, but not from AAV8-8-rh10 or AAV8-8-rh74 haploids or AAVrh10, showing AAV8 serum at these concentrations does not neutralize transfection or transduction efficiency by the AAV8-8-rh10 or AAV8-8-rh74 haploid.

FIG. 27A-27B show the humoral immune response to AAV8, AAVrh10, haploids AAV8-8-rh10 and AAV8-8-rh74; AAVrh10 mediated GM16095 cell transduction in presence of Nabs e.g., serum from mice inoculated with AAV8 (group 1), AAV8-8-rh10 (group 2), AAV8-8-rh74 (group 3), AAVrh10 (group 4), and no treatment control (group 5). FIG. 27A shows. no neutralization of GM16095 cell transduction with AAVrh10 was detected with the serum from mice inoculated with AAVrh10 (group 4) at any of dilutions tested (1/100, 1/200 and 1/400). FIG. 27B shows a graph of the neutralization by AAVrh10 serum at 1/100 dilution (left) or 1/200 dilution (right) showing inhibition of luciferase expression from AAV8, AAV8-8-rh10 and AAV8-8-rh74, suggesting their cross-reactivity against AAVrh10 serum. shows a graph of the neutralization by AAVrh10 serum at 1/100 dilution (left) or 1/200 dilution (right) showing inhibition of luciferase expression from AAV8, AAV8-8-rh10 and AAV8-8-rh74, suggesting their cross-reactivity against AAVrh10 serum.

FIG. 28 shows a schematic of the construct for generation of the AAV8-8-rh74 haploid (top construct) or AAV8-8-rh10 haploid (bottom construct). Also shown is the amino acid sequence alignment of the VP3 capsid protein from AAVrh10 (top sequence) compared to the VP3 capsid protein from AAVrh74 (bottom sequence), showing these sequence differ only in 4 regions; Q214N, VV378W, S462N, D517E when using the nomenclature/numbering from the amino acid sequence of the VP3 capsid protein from AAVrh10 (also shown in FIG. 22). The corresponding amino acid positions according to VP1 capsid protein of AAVrh10 are Q417N, V581(del)V582W, S665N and D720E.

FIG. 29A-29C shows production yield of AAV8-8-rh10 or AAV8-8-rh74 haploid comprising amino acid modifications. FIG. 29A shows more than a 4-fold increased yield of AAV8-8-rh74(W581VV) (also referred to AAV8-8-rh74vv) as compared to the yield from AAV8-8-rh74 (unmodified) and this yield is similar to the yield of AAV8-8-rh10. In the FIG. 29 A, AAVrh74vv is represented as AAV8-8rh74 581-W582V (interchangeably used as W581VV throughout the application). Modifications of N417Q and N664S and E719D of rh74 VP3 protein did not improve yield (see AAV8-8-rh74 (N417Q), AAV8-8-rh74 (N664S), AAV8-8-rh74 (E719D). Results are shown from 3 independent transduction experiments. FIG. 29B show AAV8-8-rh10 (V581del) and AAV8-8-rh10 (V582W) significantly reduced yield as compared to unmodified AAV8-8-rh10, whereas AAV8-8-rh10(S665N) and AAV8-8-rh10(D720E) did not significantly affect the yield as compared to unmodified AAV8-8-rh10. Together FIG. 29A and FIG. 29B confirm by forward and reverse mutations, that amino acid positions 581 and 582 of the VP3 capsid protein from Rh10 are important for virus production. FIG. 29C using an ITR-qPCR analysis after DNase and proteinase K treatment, further confirmed that Q417N, S665N and D720E did not strongly affect virus production, whereas V581del and V582W reduced virus production as compared to unmodified AAV8-8-rh10.

FIG. 30A-30C show gene expression of luciferase transgene in brain and spinal cord tissue from mice intravenously administered the AAV8-8-rh74 vector, demonstrating that it crosses the blood brain barrier (BBB). FIG. 30A shows the protocol for assessing biodistribution of haploid AAV8-8-Rh74 or AAV8-8-Rh10 vectors in the brain, spinal cord and small intestine, showing that 28-days after i.v. administration of 2.5×10¹² vg/kg of the AAV vector, the tissues are collected and luciferase transcript assessed by RT-PCR. FIG. 30B shows results of RT-PCR for luciferase in the brain tissue of mice 28-days after i.v. administration of 2.5×10¹² vg/kg of either AAVRh10, AAV8, AAV8-8-Rh74 or AAV8-8-Rh10 (n=4). FIG. 30C shows results of RT-PCR for luciferase in the spinal cord of mice 28-days after i.v. administration of 2.5×10¹ vg/kg of either AAVRh10, AAV8, AAV8-8-Rh74 or AAV8-8-Rh10 (n=4).

FIG. 31 shows results of RT-PCR to determine gene expression of the luciferase transgene in the small intestine of mice 28-days after intravenous administration of AAVRh10, AAV8, AAV8-8-Rh74 or AAV8-8-Rh10 (n=4), demonstrating that the AAV8-8-rh74 vector has significant tropism and efficiently transduces the small intestine as compared to AAVRh10, AAV8, and AAV8-8-Rh10.

FIG. 32 shows Tables 9, 10, 11 and 12.

### DETAILED DESCRIPTION OF THE INVENTION

The present invention will now be described with reference to the accompanying drawings, in which representative embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention. All publications, patent applications, patents, accession numbers and other references mentioned herein are incorporated by reference herein in their entirety.

The designation of all amino acid positions in the AAV capsid viral structural proteins in the description of the invention and the appended claims is with respect to VP1 capsid subunit numbering (native AAV8 VP1 capsid protein: GenBank Accession No. AF513852.1, protein ID: AAN03856.1). It will be understood by those skilled in the art that the modifications described herein if inserted into the AAV cap gene may result in modifications in the structural viral proteins VP1, VP2 and/or VP3 which make up the capsid subunits. Alternatively, the capsid subunits can be expressed independently to achieve modification in only one or two of the capsid subunits (VP1, VP2, VP1+VP2).

In particular, in previous studies, the inventors have demonstrated that polyploid (e.g., haploid) AAV vectors by using the VPs from multiple serotypes result in different biodistribution, a higher transduction in specific tissues e.g. liver

Herein, the technology generally relates to a homogenous population of rational polyploid adeno-associated virus (AAV) particles, virions and virus capsids comprising a VP3 structural protein from any AAV serotype which crosses the blood brain barrier (BBB) and wherein the polyploid AAV virion crosses the BBB and/or transduces a cell of the BBB or a brain blood vessel (BBV) endothelial cell (BBV-EC) and/or a blood component that crosses the BBB upon systemic or intrathecal administration. In some embodiments, the VP3 capsid protein is from a non-human primate, and in some embodiments the VP3 capsid protein is a AAV rhesus monkey serotype. In some embodiments, a rational polyploid AAV virion comprises at least one VP1 and/or VP2 viral structural protein in addition to the VP3 protein. In specific embodiments, rational polyploid AAV virion comprises a VP1 capsid protein from AAV8, and at least a VP3 capsid protein from any AAV serotype that cross the BBB.

In some embodiments, a homogenous population of rational polyploid adeno-associated virus (AAV) particles disclosed herein that can cross the BBB and/or transduces a cell of the BBB or a brain blood vessel (BBV) endothelial cell (BBV-EC) and/or a blood component that crosses the BBB can be selected from any of: AAV8-8-rh10, AAV8-8-rh74, AAV8-8-rh74vv, AAV8-8-rh10LP2, AAV8-8-rh74LP2, AAV8-8-rh74vvLP2. In some embodiments, such AAV haploid virions that cross the BBB and/or transduce a cell of the BBB or a BBV-EC or a cell that crosses the BBB can comprise AAV8 VP1 and VP2 structural proteins (e.g., SEQ ID NO: 7 and 8) or comprise a modified proteins of SEQ ID NO: 7 or 8, and a VP3 protein selected from any of: rh10 VP3 (SEQ ID NO: 1), rh74 VP3 (SEQ ID NO: 3), rh74vv VP3 (SEQ ID NO: 2), rh10-LP2 VP3 protein (SEQ ID NO: 14), rh74-LP2 VP3 protein (SEQ ID NO: 17), and rh74vv-LP2 VP3 protein (SEQ ID NO: 15), or a VP3 protein having an amino acid sequence that is at least 85% sequence identity to any of SEQ ID NO: 1, 2, 3, 14, 15 and 18.

The technology described herein is based on, in part, the discovery that using a VP3 structural protein, herein also referred to as a “capsid protein” from any AAV serotype that efficiently crosses the BBB changes the biodistribution and transduction efficiency of the vector after systemic or intrathecal administration, and in particular, in some embodiments, shows an increased the ability of the AAV vector to cross the BBB and transduce one or more tissues in the CNS or peripheral nervous system (PNS). In some embodiments, it shows increased transduction of a BBB endothelial cell (BBB EC) and/or component of the BBB, or increased transduction of an endothelial cell of a brain blood vessel (BBV), or increased transduction of a blood component that crosses the BBB. In some embodiments, the rational polyploid vector comprises a VP3 viral structural protein is from any serotype selected from the group consisting of AAV1, AAV6, AAV6.2, AAV7, AAV9, rh10, rh74, rh39, and rh43. In some embodiments, the rational polyploid vector comprises a VP3 viral structural protein from any non-human primate AAV serotype, for example, a rhesus monkey AAV serotype.

For illustrative purposes only, the Examples demonstrate exemplary polyploid e.g., haploid vectors that have increased ability to cross the BBB upon systemic or intrathecal administration. For exemplary purposes only, such haploid vectors comprise a VP3 from any AAV serotype that efficiently crosses the BBB and a VP1 and/or VP2 from the AAV8 serotype. The AAV VP1 and/or VP2 structural protein can be a VP1 or VP2 from any AAV serotype selected from Table 1. Accordingly, exemplary haploid vectors are described herein, comprise a capsid protein VP1, wherein said capsid protein VP1 is from AAV8 serotype and at least a capsid protein VP3, wherein said capsid protein VP3 is from any AAV serotype which crosses the BBB and/or a non-human primate, and is not the AAV8 serotype.

Preferably, such population of rational polyploid virions is substantially homogenous. In some embodiments, a rational polyploid virions of this invention can comprise a VP2 capsid protein, wherein said VP2 capsid protein is from any serotype, or a chimeric VP2 protein thereof, or where the VP2 capsid protein is from any serotype that is the same as the serotype from which VP3 comes, or alternatively, wherein the VP2 capsid proteins is from different serotype as the serotype from which VP3 comes from.

In some embodiments, a rational polyploid virions disclosed herein contain VP1 from AAV8 serotype and at least a VP3 capsid protein, where VP3 is not from AAV8 and is selected from any serotypes which cross the BBB and/or is a non-human primate AAV serotypes, is produced.

### I. Definitions

The following terms are used in the description herein and the appended claims:

The singular forms “a,” “an” and “the” are intended to include the plural forms as well, unless the context clearly indicates otherwise.

Furthermore, the term “about,” as used herein when referring to a measurable value such as an amount of the length of a polynucleotide or polypeptide sequence, dose, time, temperature, and the like, is meant to encompass variations of 20%, +10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

As used herein, the transitional phrase “consisting essentially of” means that the scope of a claim is to be interpreted to encompass the specified materials or steps recited in the claim, “and those that do not materially affect the basic and novel characteristic(s)” of the claimed invention. See, In re Herz, 537 F.2d 549, 551-52, 190 USPQ 461,463 (CCPA 1976) (emphasis in the original); see also MPEP § 2111.03. Thus, the term “consisting essentially of” when used in a claim of this invention is not intended to be interpreted to be equivalent to “comprising.” Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc. as if each such subcombination is expressly set forth herein. Moreover, such language also indicates that one or more of the specified amino acids can be disclaimed (e.g., by negative proviso). For example, in particular embodiments the amino acid is not A, G or I; is not A; is not G or V; etc. as if each such possible disclaimer is expressly set forth herein.

As used herein, the terms “reduce,” “reduces,” “reduction” and similar terms mean a decrease of at least about 25%, 35%, 50%, 75%, 80%, 85%, 90%, 95%, 97% or more. As used herein, the terms “enhance,” “enhances,” “enhancement” and similar terms indicate an increase of at least about 25%, 50%, 75%, 100%, 150%, 200%, 300%, 400%, 500% or more. Enhanced transduction ability of the rational polyploid virions of the invention across blood brain barrier is relative to AAV virions that lack ability to cross blood brain barrier. The enhanced transduction ability of the rational polyploid virions across blood brain barrier is at least 25%, 50%, 60%, 70%, 80%, 90% or 95%, or 100% or more, or at least 1.2 fold, or at least 1.5 fold, or at least 2 fold, at least 5 fold, at least 10 fold, at least 20 fold, at least 50 fold or more compared to that of AAV virion that lack ability to cross blood brain barrier. Non limiting examples of AAV serotypes that lack the ability to cross blood brain barrier include AAV2, AAV5, AAV8. In one embodiment, the population exhibits enhanced transduction activity across the blood brain barrier (BBB) relative to an AAV serotype that does not efficiently cress the blood brain barrier, e.g. AAV8, AAV2 or, AAV5. In one embodiment the population exhibits enhanced transduction activity across the blood brain barrier (BBB) relative to AAV8. In one embodiment, the population exhibits enhanced transduction activity across the blood brain barrier (BBB) relative to AAV2. In another embodiment, the population exhibits enhanced transduction activity across the blood brain barrier (BBB) relative to AAV5. In some embodiments, the population of rational polyploid AAV virions has enhanced biodistribution in brain and spinal cord relative to AAV8. The term “parvovirus” as used herein encompasses the family Parvoviridae, including autonomously replicating parvoviruses and dependoviruses. The autonomous parvoviruses include members of the genera Parvovirus, Erythrovirus, Densovirus, Iteravirus, and Contravirus. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, Muscovy duck parvovirus, B19 virus, and any other autonomous parvovirus now known or later discovered. Other autonomous parvoviruses are known to those skilled in the art. See, e.g., BERNARD N. FIELDS et al., VIROLOGY, volume 2, chapter 69 (4th ed., Lippincott-Raven Publishers).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, avian AAV, bovine AAV, canine AAV, equine AAV, ovine AAV, and any other AAV now known or later discovered. See, e.g., BERNARD N. FIELDS et al., VIROLOGY, volume 2, chapter 69 (4th ed., Lippincott-Raven Publishers). A number of relatively new AAV serotypes and clades have been identified (see, e.g., Gao et al., (2004) 1 Virology 78:6381-6388; Moris et al., (2004) Virology 33-375-383; and Table 3).

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as GenBank. See, e.g., GenBank Accession Numbers NC 002077, NC_001401, NC 001729, NC 001863, NC_001829, NC 001862, NC_000883, NC 001701, NC 001510, NC 006152, NC_006261, AF063497, U89790, AF043303, AF028705, AF028704, J02275, J01901, J02275, X01457, AF288061, AH009962, AY028226, AY028223, NC_001358, NC 001540, AF513851, AF513852, AY530579; the disclosures of which are incorporated by reference herein for teaching parvovirus and AAV nucleic acid and amino acid sequences. See also, e.g., Srivistava et al., (1983) J. Virology 45:555; Chiarini et al., (1998) J. Virology 71:6823; Chiarini et al., (1999) J. Virology 73:1309; Bantel-Schaal et al., (1999) J. Virology 73:939; Xiao et al., (1999) J. Virology 73:3994; Muramatsu et al., (1996) Virology 221:208; Shade et al., (1986) J. Virol. 58:921; Gao et al., (2002) Proc. Nat. Acad. Sci. USA 99:11854; Moris et al., (2004) Virology 33-:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 1.

The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99:10405-10), AAV4 (Padron et al., (2005) J. Virol. 79: 5047-58), AAVS (Walters et al., (2004) 0. 78: 3361-71) and CPV (Xie et al., (1996) J. Mol. Biol. 6:497-520 and Tsao et al., (1991) Science 251: 1456-64).

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a heterologous nucleic acid(s) of interest.

As used here, “systemic tropism” and “systemic transduction” (and equivalent terms) indicate that the virus capsid or virus vector of the invention exhibits tropism for and/or transduces tissues throughout the body (e.g., brain, lung, skeletal muscle, heart, liver, kidney and/or pancreas). In embodiments of the invention, systemic transduction of the central nervous system (e.g., brain, neuronal cells, etc.) is observed.

As used herein, “selective tropism” or “specific tropism” means delivery of virus vectors to and/or specific transduction of certain target cells and/or certain tissues.

Unless indicated otherwise, “efficient transduction” or “efficient tropism,” or similar terms, can be determined by reference to a suitable control (e.g., at least about 50%, 60%, 70%, 80%, 85%, 90%, 95%, 100%, 125%, 150%, 175%, 200%, 250%, 300%, 350%, 400%, 500% or more of the transduction or tropism, respectively, of the control). In some embodiments, the control that serves as a reference to determine whether an AAV virion can ‘efficiently cross blood brain barrier’, is AAV virions that lack ability to cross blood brain barrier. In particular embodiments, the rational polyploid virus vectors described herein efficiently transduces or has efficient tropism for the CNS or peripheral nervous system (PNS), including neuronal cells and non-neuronal cells. Suitable controls will depend on a variety of factors including the desired tropism and/or transduction profile. A person of skill in the art can determine if a rational polyploid AAV vector disclosed herein can efficiently cross blood brain barrier by monitoring transduction across CNS regions e.g., brain regions as better than AAV2 under similar conditions. E.g., as described in: Molecular Therapy vol. 19 no. 8 Aug. 2011, herein incorporated by reference in its entirety. Monitoring can be by any method known to one of ordinary skilled art, and includes RT-PCR analysis of brain and spinal cord tissue, western blots of brain and spinal cord tissue or immunohistochemistry of brain and spinal cord tissue of animal models, as well as in vivo analysis of bioluminescence of a luciferase expressing rational polyploid in an animal model. It is preferred that the serotype that efficiently crosses blood brain barrier is better than AAV6 in crossing blood brain barrier and transducing CNS regions e.g., brain regions. In one embodiment, the population can cross blood brain barrier and transduce CNS regions better than AAV5. In some embodiments, the population has enhanced transduction to one or more of cortex, striatum, thalamus, medulla, hippocampus, cerebellum and spinal cord of a subject relative to AAV8, a non-haploid AAV particle that lacks ability to efficiently cross blood brain barrier. In some embodiments, the rational polyploid population is administered in a subject by intravenous injection, or, intrathecal injection or, intravascular injection in brain. An enhanced transduction ability of the rational polyploid virions disclosed herein to transduce the CNS or a brain region is at least 20% or more, or 30% or more, or 40% or more, or 50% or more, 60% or more, 70% or more, 80% or more, 90% or more or 95% or more, or 100% or more, or at least 1.2 fold, or at least 1.5 fold, or at least 2 fold, at least 5 fold, at least 10 fold, at least 20 fold, at least 50 fold or more compared to that of a AAV2 or AAV5 virion under similar conditions.

Similarly, it can be determined if a virus “does not efficiently transduce” or “does not have efficient tropism” for a target tissue, or similar terms, by reference to a suitable control. In particular embodiments, the virus vector does not efficiently transduce (i.e., does not have efficient tropism) for liver, kidney, gonads and/or germ cells. In particular embodiments, transduction (e.g., undesirable transduction) of tissue(s) (e.g., liver) is 20% or less, 10% or less, 5% or less, 1% or less, 0.1% or less of the level of transduction of the desired target tissue(s) (e.g., skeletal muscle, diaphragm muscle, cardiac muscle and/or cells of the central nervous system).

In some embodiments of this invention, an AAV particle comprising a capsid of this invention can demonstrate multiple phenotypes of efficient transduction of certain tissues/cells and very low levels of transduction (e.g., reduced transduction) for certain tissues/cells, the transduction of which is not desirable.

As used herein, the term “polypeptide” encompasses both peptides and proteins, unless indicated otherwise.

A “polynucleotide” is a sequence of nucleotide bases, and may be RNA, DNA or DNA-RNA hybrid sequences (including both naturally occurring and non-naturally occurring nucleotides), but in representative embodiments are either single or double stranded DNA sequences.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In representative embodiments an “isolated” nucleotide is enriched by at least about 10-fold, 100-fold, 1000-fold, 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In representative embodiments an “isolated” polypeptide is enriched by at least about 10-fold, 100-fold, 1000-fold, 10,000-fold or more as compared with the starting material.

An “isolated cell” refers to a cell that is separated from other components with which it is normally associated in its natural state. For example, an isolated cell can be a cell in culture medium and/or a cell in a pharmaceutically acceptable carrier of this invention. Thus, an isolated cell can be delivered to and/or introduced into a subject. In some embodiments, an isolated cell can be a cell that is removed from a subject and manipulated as described herein ex vivo and then returned to the subject.

A population of virions can be generated by any of the methods described herein. In one embodiment, the population is at least 10¹virions. In one embodiment, the population is at least 10² virions, at least 10³ virions, at least 10⁴ virions, at least 10⁵virions, at least 10⁶ virions, at least 10⁷ virions, at least 10⁸ virions, at least 10⁹ virions, at least 10¹⁰ virions, at least 10¹¹ virions, at least 10¹² virions, at least 10¹³ virions, at least 10¹⁴ virions, at least 10¹⁵ virions, at least 10¹⁶ virions, or at least 10¹⁷ virions. A population of virions can be heterogeneous or can be homogeneous (e.g., substantially homogeneous or completely homogeneous).

A “substantially homogeneous population” as the term is used herein, refers to a population of virions that are mostly identical, with few to no contaminant virions (those that are not identical) therein. A substantially homogeneous population is at least 90% of identical virions (e.g., the desired virion), and can be at least 91%, at least 92%, at least 93%, at least 94%, at least 95%, at least 96%, at least 97%, at least 98%, at least 99%, at least 99.5%, at least 99.9% of identical virions.

A population of virions that is completely homogeneous contains only identical virions.

As used herein, by “isolate” or “purify” (or grammatical equivalents) a virus vector or virus particle or population of virus particles, it is meant that the virus vector or virus particle or population of virus particles is at least partially separated from at least some of the other components in the starting material. In representative embodiments an “isolated” or “purified” virus vector or virus particle or population of virus particles is enriched by at least about 10-fold, 100-fold, 1000-fold, 10,000-fold or more as compared with the starting material.

A “therapeutic polypeptide” is a polypeptide that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability or induction of an immune response.

By the terms “treat,” “treating,” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the methods of the invention. The prevention can be complete, e.g., the total absence of the disease, disorder and/or clinical symptom(s). The prevention can also be partial, such that the occurrence of the disease, disorder and/or clinical symptom(s) in the subject and/or the severity of onset is substantially less than what would occur in the absence of the present invention.

A “treatment effective” amount as used herein is an amount that is sufficient to provide some improvement or benefit to the subject. Alternatively stated, a “treatment effective” amount is an amount that will provide some alleviation, mitigation, decrease or stabilization in at least one clinical symptom in the subject. Those skilled in the art will appreciate that the therapeutic effects need not be complete or curative, as long as some benefit is provided to the subject.

A “prevention effective” amount as used herein is an amount that is sufficient to prevent and/or delay the onset of a disease, disorder and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some preventative benefit is provided to the subject.

The terms “heterologous nucleotide sequence” and “heterologous nucleic acid molecule” are used interchangeably herein and refer to a nucleic acid sequence that is not naturally occurring in the virus. Generally, the heterologous nucleic acid molecule or heterologous nucleotide sequence comprises an open reading frame that encodes a polypeptide and/or nontranslated RNA of interest (e.g., for delivery to a cell and/or subject).

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone.

A “rAAV vector genome” or “rAAV genome” is an AAV genome (i.e., vDNA) that comprises one or more heterologous nucleic acid sequences. rAAV vectors generally require only the terminal repeat(s) (TR(s)) in cis to generate virus. All other viral sequences are dispensable and may be supplied in trans (Muzyczka, (1992) Curr. Topics Microbiol. Immunol. 158:97). Typically, the rAAV vector genome will only retain the one or more TR sequence so as to maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one TR sequence (e.g., AAV TR sequence), optionally two TRs (e.g., two AAV TRs), which typically will be at the 5′ and 3′ ends of the vector genome and flank the heterologous nucleic acid, but need not be contiguous thereto. The TRs can be the same or different from each other.

The term “terminal repeat” or “TR” includes any viral terminal repeat or synthetic sequence that forms a hairpin structure and functions as an inverted terminal repeat (i.e., mediates the desired functions such as replication, virus packaging, integration and/or provirus rescue, and the like). The TR can be an AAV TR or a non-AAV TR. For example, a non-AAV TR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as a TR, which can further be modified by truncation, substitution, deletion, insertion and/or addition. Further, the TR can be partially or completely synthetic, such as the “double-D sequence” as described in U.S. Pat. No. 5,478,745 to Samulski et al.

An “AAV terminal repeat” or “AAV TR” may be from any AAV, including but not limited to serotypes 1, 2, 3, 4, 5, 6, 7, 8, 9, 10, 11 or 12 or any other AAV now known or later discovered (see, e.g., Table 1). An AAV terminal repeat need not have the native terminal repeat sequence (e.g., a native AAV TR sequence may be altered by insertion, deletion, truncation and/or missense mutations), as long as the terminal repeat mediates the desired functions, e.g., replication, virus packaging, integration, and/or provirus rescue, and the like. AAV proteins VP1, VP2 and VP3 are capsid proteins that interact together to form an AAV capsid of an icosahedral symmetry. VP1.5 is an AAV capsid protein described in US Publication No. 2014/0037585. In the present invention, in some embodiments, AAV ITR is 145 bp. In some embodiments, AAV ITR is smaller than 145 bp. In some embodiments, AAV ITR is 130 bp.

The virus vectors of the invention can further be “targeted” virus vectors (e.g., having a directed tropism) and/or a “hybrid” parvovirus (i.e., in which the viral TRs and viral capsid are from different parvoviruses) as described in international patent publication WO 00/28004 and Chao et al., (2000) Molecular Therapy 2:619.

The virus vectors of the invention can further be duplexed parvovirus particles as described in international patent publication WO 01/92551 (the disclosure of which is incorporated herein by reference in its entirety). Thus, in some embodiments, double stranded (duplex) genomes can be packaged into the virus capsids of the invention.

Further, the viral capsid or genomic elements can contain other modifications, including insertions, deletions and/or substitutions.

A “chimeric” viral structural protein as used herein means an AAV viral structural protein (capsid) that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.,) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. In other embodiments the substitutions are all from the same serotype. In other embodiments the substitutions are all from AAV or synthetic. Production of a chimeric capsid protein can be carried out according to protocols well known in the art and a large number of chimeric capsid proteins are described in the literature as well as herein that can be included in the capsid of this invention. In some embodiments, the rational polyploid comprises at least one chimeric viral structural protein. In this aspect, the viral structural protein is generated by the N terminus of one AAV serotype and the C terminus of another AAV serotype. In some embodiments, the rational polyploid comprises no chimeric viral structural protein.

A “modified” viral structural protein can be a structural capsid protein that comprises a non-capsid protein or modification, or is a chemically modified viral structural protein, e.g., the addition of non-naturally occurring or synthetic amino acids, or substitution of an amino acid with a non-naturally occurring amino acid, as well as chemical modifications to one or more existing amino acids of the AAV capsid protein. Surface modification to capsid proteins is disclosed in U.S. Pat. Nos. 10,294,281, 9,409,953 and US Application 2018/0105559 which is incorporated herein in its entirety by reference. In some embodiments, the surface modification can include a targeting protein to target the CNS e.g., as disclosed in WO2020028751, in particular, Table 2 of WO2020028751, which is incorporated herein in its entirety by reference. In certain embodiments, rational engineering and/or mutational methods are used to modifications and/or targeting peptides having enhanced transduction of a target tissue (e.g., CNS or PNS). Targeting peptides of for use and modification of one or more capsid proteins of a rational polyploid vector disclosed herein can be identified and/or designed by any method known in the art, for example, using the CREATE system as described in Deverman et al., (Nature Biotechnology 34(2):204-209 (2016)) and in International Patent Application Publication Nos. WO2015038958 and WO2017100671, which are incorporated herein in their entirety.

In an alternative embodiment, a virion particle can be constructed wherein at least one viral protein from the group consisting of AAV capsid proteins, VP1, VP2 and VP3, is different from at least one of the other viral proteins, required to form the virion particle capable of encapsidating an AAV genome. For each viral protein present (VP1, VP2, and/or VP3), that protein is the same type (e.g., all AAV2 VP1). In one instance, at least one of the viral proteins is a chimeric viral protein and at least one of the other two viral proteins is not a chimeric. In one embodiment VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from AAV2; or only the chimeric VP1/VP2 28m-2P3 (the N-terminal from AAV8 and the C-terminal from AAV2 without mutation of VP3 start codon) paired with only VP3 from AAV2. In another embodiment only VP3 is chimeric and VP1 and VP2 are non-chimeric. In another embodiment at least one of the viral proteins is from a completely different serotype. For example, only the chimeric VP1/VP2 28m-2P3 paired with VP3 from only AAV3. In another example, no chimeric is present.

As used herein, the term “amino acid” encompasses any naturally occurring amino acid, modified forms thereof, and synthetic amino acids. Naturally occurring, levorotatory (L-) amino acids are shown in Table 10.

Alternatively, the amino acid can be a modified amino acid residue (nonlimiting examples are shown in Table 12) and/or can be an amino acid that is modified by post-translation modification (e.g., acetylation, amidation, formylation, hydroxylation, methylation, phosphorylation or sulfatation).

Further, the non-naturally occurring amino acid can be an “unnatural” amino acid as described by Wang et al., Annu Rev Biophys Biomol Struct. 35:225-49 (2006). These unnatural amino acids can advantageously be used to chemically link molecules of interest to the AAV capsid protein.

As used herein, the term “homologous recombination” means a type of genetic recombination in which nucleotide sequences are exchanged between two similar or identical molecules of DNA. Homologous recombination also produces new combinations of DNA sequences. These new combinations of DNA represent genetic variation. Homologous recombination is also used in horizontal gene transfer to exchange genetic material between different strains and species of viruses.

Viral infection to a host can stimulate the host's immune defense system to protect the infected host from the virus. One of the immune responses a host activates to defend itself from the attack of a foreign agent is the humoral immune response, which produces antibody-mediated immunity.

As used herein, the term “humoral immunity” refers to the antibody-mediated beta cellular immune system, which is mediated by macromolecules (as opposed to cell-mediated immunity) found in extracellular fluids such as secreted antibodies, complement proteins and certain antimicrobial peptides. In particular, it refers to the antibody mediated immune response of a host.

As used herein “antigenic” or “antigenicity” is used interchangeably with “immunogenicity” and refer to the ability of a substance, e.g., a rational polyploid virion to induce a specific immune response. It is also referred to the degree to which the substance can stimulate an immune response, for instance, the ability of the substance that is capable of binding to an antibody or to a T-cell receptor or to a B cell.

As used herein, the term “Neutralizing antibody” is used interchangeably with “Nab” and refers to antibodies that specifically bind to epitopes crucial for viral function and interfere with viral infectivity, for example, blocking AAV virion entry into the host cell. Neutralizing antibodies (NAbs) encompassed in the definition refers to antibodies that defend a cell from an antigen or infectious agent by inhibiting or neutralizing any effect it has biologically. In general, an antibody binds to an antigen and signals to white blood cells that this antigen has been targeted (i.e. flagged). The flagged antigen is processed and consequently destroyed, while neutralizing antibodies neutralize the biological effect of the antigen itself. A NAb may be a broadly neutralizing antibody (bNAb) that works on multiple serotypes of a virus, or a specific NAb that specifically recognizes one serotype.

“Neutralization” to viruses, in particular to AAV capsids and AAV serotypes, is defined here as the abrogation of virus infectivity in vitro or in vivo by the binding of a neutralizing compound (e.g., antibody) to the virus serotype and/or the binding of a cell surface and preventing the interaction with AAV. In the context of the present invention, the definition does not include the blocking of infection by a neutralizing antibody that binds to a receptor for the virus on the (host) cell surface.

In some examples, neutralizing capacity is determined by measuring the activity of a reporter gene product (e.g., luciferase, GFP). The neutralizing capacity of an antibody to a specific AAV serotype may be at least 50%, e.g., at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%.

As used herein “Blood brain barrier” or “BBB” refers to a highly selective semipermeable border of endothelial cells that prevents solutes in the circulating blood from non-selectively crossing into the extracellular fluid of the central nervous system where neurons reside. The blood-brain barrier is formed by endothelial cells of the capillary wall, astrocyte end-feet ensheathing the capillary, and pericytes embedded in the capillary basement membrane. This system allows the passage of some molecules by passive diffusion, as well as the selective and active transport of various nutrients, ions, organic anions, and macromolecules such as glucose, water and amino acids that are crucial to neural function.

As used herein “brain blood vessels” or “BBV” refer to blood vessels and capillaries in the brain that are part of the cerebral circulation and have BBB function.

As used herein, the phrase “cell of the BBB” refers to any cells that is part of, or a component of the BBB, and includes endothelial cells of the capillary wall of the BBB, astrocyte end-feet ensheathing the capillary, and pericytes embedded in the capillary basement membrane.

As used herein, the phrase “endothelial cell of the BBB” can be used interchangeably herein with “BBB ECs” or “BBB endothelial cells” and refers to an endothelial cell of the capillary wall of the BBB.

As used herein, the phrase “cell that crosses the BBB” refers to any cell that can cross an intact BBB, i.e., where the BBB is not leaky or compromised. Cells that can cross the BBB include, but are not limited to perivascular pericytes, macrophages, T cells and monocytes.

As used herein, a “blood component” as used herein refers to a cell in the blood, and can includes platelets (thrombocytes), red blood cells (rbc) (erythrocytes), leukocytes, including, lymphocytes, monocytes, eosinophils, basophils and neutrophils.

As used herein, the term “gene editing,” “Genome editing,” or “genome engineering” means a type of genetic engineering in which DNA is inserted, deleted or replaced in the genome of a living organism using engineered nucleases, or “molecular scissors.” These nucleases create site-specific double-strand breaks (DSBs) at desired locations in the genome.

As used herein, the term “gene delivery” means a process by which foreign DNA is transferred to host cells for applications of gene therapy.

As used herein, the term “CRISPR” stands for Clustered Regularly Interspaced Short Palindromic Repeats, which are the hallmark of a bacterial defense system that forms the basis for CRISPR-Cas9 genome editing technology.

As used herein, the term “zinc finger” means a small protein structural motif that is characterized by the coordination of one or more zinc ions, in order to stabilize the fold.

### II. Rational Haploid or Polyploid AAV Vectors in General

A rational polyploid as used herein, e.g., rational haploid refer to a virion that is formed from viral structural proteins VP1, VP2 or, VP3 coming from at least two different AAV serotypes and wherein, each of VP1, VP2 or, VP3 is only from one parental AAV serotype. A non-rational haploid can refer to a chimeric or mosaic haploid. In one embodiment, the viral structural proteins of parental serotype can be a modified viral structural protein or, can be chimeric. In one embodiment, the parental serotype is not chimeric serotype. In some embodiments, the modified parental AAV serotype comprise insertion, deletion or, substitution of one or more amino acids. The rational polyploid virions e.g., rational haploid, of this invention are not mosaic virions.

In some embodiments, the AAV hybrid particles as disclosed herein can be synthetic AAV hybrid viral vector designed to display a range of desirable phenotypes that are suitable for different in vitro and in vivo applications. Thus, in one embodiment, the present invention provides an AAV hybrid particle or virion. In one embodiment, the present invention provides a substantially pure population of AAV hybrid particles or virions.

The present invention provides an array of synthetic viral vectors displaying a range of desirable phenotypes that are suitable for different in vitro, in vivo and clinical applications.

In particular, the present invention is based on the unexpected discovery that combining at least a VP3 capsid protein only from any AAV serotype that efficiently crosses the BBB, with a VP1 and/or VP2 capsid protein only from a different serotype (e.g., a VP1 and/or VP2 capsid protein from an AAV serotype selected from Table 1), allows for the development of improved AAV virions that have multiple desirable phenotypes in each individual capsid, including but not limited to increased systemic delivery and tropism as well as a different antigenic profile, such as, e.g., the ability to evade neutralizing antibodies (Nab) after administration in vivo.

In some embodiments, the rational polyploid vector disclosed herein has biodistribution in the CNS and/or PNS due to the ability of the rational polyploid vector to cross the BBB. In some embodiments, the rational polyploid vector disclosed herein has biodistribution in the CNS and/or PNS due to the ability to transduce a brain blood vessel (BBV) and/or a blood component. In some embodiments, the rational polyploid vector disclosed herein has biodistribution in the CNS and/or PNS by transducing a brain blood vessel (BBV) and/or a blood component, e.g., a cell in the blood, which allows delivery of the AAV transduced cell to the brain via the cerebral circulation. In some embodiments, the rational polyploid vector disclosed herein can transduce a cell that crosses the endothelial cell, including perivascular pericytes and macrophages, as well as other immune cells such as T cells and blood monocytes. That is, in some embodiments, the rational polyploid AAV vectors have biodistribution in the brain via an indirect route—the AAV polyploid vectors disclosed herein can reach the brain indirectly via transducing a cell that enters the brain and/or by transducing a brain blood vessel (BBV), including an endothelial cell in the brain. In some embodiments, a rational polyploid vector disclosed herein can be taken up by the neuronal and/or non-neuronal cells contacting the brain blood vessels or other organs, e.g., via retrograde transport from the peripheral organ to the CNS via autonomic or peripheral nerve fibers. In some embodiments, the rational polyploid of the invention has enhanced binding to brain microvascular endothelial cells (BMVECs) compared to that of AAV8. In some embodiments, the rational polyploid of the invention has enhanced binding to brain microvascular endothelial cells (BMVECs) compared to that of AAV2. Binding to BMVECs is described in Molecular Therapy, Methods & Clinical development, vol 20, March 2021 which is incorporated herein by reference in its entirety. In some embodiments, the rational polyploid of the present invention can diffuse or, trancytose or shuttle across the endothelial barrier, e.g., blood brain barrier, wherein, it does not affect the integrity of blood brain barrier compared to that of AAV2. On the contrary, AAV2 is endocytosed transducing BMVECs with minimal diffusion across the endothelial barrier. The rational polyploid of the present invention engages in transcytosis of BMVECs and transduces CNS regions e.g brain parenchyma cells. Primary BMVECs create an effective endothelial barrier and served as a model relevant to human BBB to test AAV serotypes for transcytosis, endocytosis and transduction as described in J Neurochem 2017 January 140(2), 216-230; and J Neurochem 2017, 140, 192-194 both of which are incorporated by reference in entirety.

Such haploid or polyploid virions are sometimes referred to as triploid virions, to refer to the fact that the capsid proteins VP1, VP2, and VP3 come from at least two different serotypes. Exemplary methods for producing such AAV haploid virions are described herein. By preventing the translation of undesired open reading frames of VP3 from the VP1 or VP2 AAV serotype, these methods result in the production of homogeneous populations of the generated virions.

The ability to generate a homogeneous (e.g., substantially or completely) population of recombinant AAV haploid virions dramatically reduces or eliminates carryover of properties of undesired/contaminating virions (e.g., transduction specificity or antigenicity).

In one embodiment, a AAV haploid virion described herein that encapsidates an AAV genome (including a heterologous gene located between 2 AAV ITRs) can be formed with only two of the viral structural proteins, VP1 and VP3. In one embodiment, such a AAV haploid virion is conformationally correct, i.e., has T=1 icosahedral symmetry. In one embodiment, the AAV haploid virions described herein are infectious. In one embodiment, the AAV haploid virions described herein has a different biodistribution as compared to the native AAV serotype, for example the AAV haploid virions described herein have a systemic transduction as compared to the native AAV serotype. In one embodiment, the AAV haploid virions described herein have a reduced antigenic profile as compared to the native AAV serotype, e.g., the AAV haploid virions described herein has a reduced ability to induce humoral immune response or, increased ability to escape neutralizing antibodies (Nab) as compared to the native AAV serotype.

The AAV virion has T=1 icosahedral symmetry and is composed of the three structural viral proteins, VP1, VP2, and VP3. 60 copies of the three viral proteins in a ratio of 1:1:8 to 10 (VP1:VP2:VP3, respectively) form the virion (Rayaprolu, V., et al., J. Virol. 87(24): 13150-13160 (2013).

In one embodiment, an AAV virion that encapsidates an AAV genome including a heterologous gene between 2 AAV ITRs can be formed with only two of the viral structural proteins, VP1 and VP3. In one embodiment this virion is conformationally correct, i.e., has T=1 icosahedral symmetry. In one embodiment the virions are infectious. Infectious virions include VP1/VP3 or VP1/VP2/VP3. Typically, virions comprising only VP2/VP3 or only VP3 are not infectious.

In some embodiments, the technology herein also provides an AAV capsid wherein the capsid comprises capsid protein VP1, wherein said capsid protein VP1 is from one or more than one first AAV serotype and capsid protein VP2, wherein said capsid protein VP2 is from one or more than one second AAV serotype and wherein at least one of said first AAV serotype is different from at least one of said second AAV serotype, in any combination, and where at least one of VP1, VP2 or VP3 is from a rhesus monkey AAV (AAVrh) serotype. In some embodiments no chimeric viral structural protein is present in the virion.

In some embodiments, the AAV haploid particle as disclosed herein can comprise a capsid protein that comprises capsid protein VP3, wherein said capsid protein VP3 is from one or more than one third AAV serotype and where the third AAV serotype is a AAV serotype which crosses the BBB, wherein at least one of said one or more than one third AAV serotype is different from said first AAV serotype and/or said second AAV serotype, in any combination, and wherein the first or second AAV serotype, but not both, are a rhesus monkey AAV (AAVrh) serotype. In some embodiments, the AAV capsid described herein can comprise capsid protein VP1.5.

The present invention further provides an AAV particle that comprises an adeno-associated virus (AAV) capsid, wherein the capsid comprises capsid protein VP1, wherein said capsid protein VP1 is from one or more than one first AAV serotype (e.g., a serotype selected from Table 1) and capsid protein VP1.5, wherein said capsid protein VP1.5 is from one or more than one second AAV serotype and wherein at least one of said first AAV serotype is different from at least one of said second AAV serotype, in any combination, and the first AAV serotype or the second AAV serotype, but not both, is from a rhesus monkey AAV (AAVrh serotype).

In some embodiments, the capsid comprises capsid protein VP3, wherein said capsid protein VP3 is from one or more than one third AAV serotype which is a AAV serotype which crosses the BBB, wherein at least one of said one or more than one third AAV serotype is different from said first AAV serotype and/or said second AAV serotype, in any combination, and wherein at least one of the first AAV serotype, the second AAV serotype or the third AAV serotype is from a AAV serotype which crosses the BBB, or in some embodiments a non-human AAV serotype, e.g., rhesus monkey AAV (AAVrh) serotype, and the other serotype is selected from any AAV serotype selected from Table 1. In some embodiments, the AAV capsid described herein can comprise capsid protein VP1.5.

In some instances, the VP3 protein can be chemically modified to cross the BBB, or alternatively, in some embodiments, comprise a target peptide to increased its ability to cross the BBB. The present invention further provides an adeno-associated virus (AAV) capsid, wherein the capsid comprises capsid protein VP1, wherein said capsid protein VP1 is from one or more than one first AAV serotype and capsid protein VP1.5, wherein said capsid protein VP1.5 is from one or more than one second AAV serotype and wherein at least one of said first AAV serotype is different from at least one of said second AAV serotype, in any combination, and wherein the first AAV serotype or the second AAV serotype, but not both, is from rhesus monkey AAV (AAVrh) serotype, and the other serotype is selected from any AAV serotype selected from Table 1.

In some embodiments, the AAV capsid of this invention comprises capsid protein VP3, wherein said capsid protein VP3 is from one or more than one third AAV serotype which is a AAV serotype which crosses the BBB, wherein at least one of said one or more than one third AAV serotype is different from said first AAV serotype and/or said second AAV serotype, in any combination, and wherein at least one of the first AAV serotype, or second AAV serotype, or third AAV serotype, is from a AAV serotype which crosses the BBB, and in some embodiments, a non-human primate AAV, e.g., a rhesus monkey AAV (AAVrh) serotype, and the other serotypes can be selected from any AAV serotype selected from Table 1. In some embodiments, the AAV capsid protein described herein can comprise capsid protein VP2.

In some embodiments, the rational polyploid virions disclosed herein can be synthetic rational polyploid AAV viral vector designed to display a range of desirable phenotypes that are suitable for different in vitro and in vivo applications. Thus, in one embodiment, the present invention provides a rational polyploid AAV viral vector or virion. In one embodiment, the present invention provides a substantially pure population of rational polyploid AAV viral vectors or virions.

Viral infection to a host can stimulate the host's immune defense system to protect the infected host from the virus. One of the immune responses a host activates to defend itself from the attack of a foreign agent is the humoral immune response, which produces antibody-mediated immunity. In one embodiment, the rational polyploid virions disclosed herein elicit a reduced humoral response as compared to the parental serotype, or are less effected by anti-AAV neutralizing antibodies as compared to the parental serotype.

For explanation purposes only, neutralizing antibodies are different from non-neutralizing antibodies. Antibody based immunity consists of neutralizing and non-neutralizing antibodies; non-neutralizing antibodies makeup the greater part of the antibody pool generated during the immune response, but only a small fraction is functional and participates in the clearance of infected cells, sometimes through interaction with other immune cells and/or with the complement system. In contrast, neutralizing antibodies specifically bind epitopes crucial for viral function, and interfere with viral infectivity, for example, blocking viral entry to the host cell. Neutralizing antibodies (NAbs) bind and inhibit AAV transduction of target cells through several mechanisms. AAV neutralizing antibodies have been the focus of many studies because of their significant deleterious effect on the efficacy of AAV-mediated gene therapy. Recent studies have shown that AAV binding antibodies may also have an impact on AAV vector distribution and safety (Klasse et al., J Gen Virol, 2002, 83(Pt 9):2091; and Wang et al., Hum Gene Ther, 2011, 22(11):1389; the contents of each of which are incorporated herein by reference in their entirety).

Detection of pre-existing neutralizing antibodies to AAV capsids and AAV serotypes in AAV gene delivery is critical for developing appropriate approaches on how to overcome the challenge posited by these antecedent antibodies. The use of different/alternative AAV capsids and AAV serotypes, to which lower titers or absences of neutralizing antibodies are detected in a patient or a group of patients, may overcome this challenge.

### II. Rational AAV Polyploid Vectors that Cross the BBB

The present invention provides an array of synthetic rational polyploid AAV viral vectors displaying a range of desirable phenotypes that are suitable for different in vitro and in vivo applications. In particular, the present invention is based on the unexpected discovery that combining at least a VP3 structural protein, e.g., capsid protein, that efficiently crosses the BBB, with at least a VP1 capsid protein, and/or VP2 capsid protein from any different AAV serotype in an individual capsid allows for the development of improved AAV capsids that have multiple desirable phenotypes in each individual capsid, such as at least one property selected from, but not limited to, increased tropism for the CNS and/or PNS, ability to cross the blood brain barrier (BBB) and/or transduce a blood brain vessel (BBV) or blood component that allows delivery of the rational AAV capsid to the brain via the cerebral circulation, elicit reduced humoral response, ability to evade neutralizing antibodies (Nab) after administration in vivo. In some embodiments, one desirable property exhibited by the rational polyploid AAV virions is the ability to allow it to be selected as a redosing vector. Such haploid or polyploid virions refer to the fact that the capsid proteins VP1, VP2, and VP3 come from at least two different serotypes. Exemplary methods for producing such rational polyploid AAV virions are described herein. By preventing the translation of undesired open reading frames of VP3 from the AAV serotype of the VP1 and/or VP2 protein, these methods result in the production of homogeneous populations of the generated virions.

The ability to generate a homogeneous (e.g., substantially or completely) population of recombinant rational polyploid AAV virions dramatically reduces or eliminates carryover of properties of undesired/contaminating virions (e.g., transduction specificity or antigenicity).

In some embodiments, the population of rational polyploid AAV virions as disclosed herein has enhanced transduction to one or more of endothelial cells of brain blood vessels (BBV), astrocytes, oligodendrocytes, CC1+ oligodendrocytes, neuronal subtypes including NeuN+ cells throughout the brain, midbrain tyrosine hydroxylase (TH)+ dopaminergic neurons, Calbindin+ cerebellar Purkinje cells, interneuron populations and CD31+ endothelial cells of a subject relative to a non-haploid AAV particle that lacks ability to cross blood brain barrier e.g., AAV2, or AAV5 or, AAV8. In some embodiments, the population of rational polyploid AAV virions has equivalent or, enhanced transduction to one or more of cortex, striatum, thalamus, medulla, hippocampus, cerebellum and spinal cord of a subject relative to a non-haploid AAV particle that has ability to cross blood brain barrier. In some aspects of the embodiment, the population of rational polyploid AAV virions equivalent or, enhanced transduction to one or more of astrocytes, oligodendrocytes, CC1+ oligodendrocytes, neuronal subtypes including NeuN+ cells throughout the brain, midbrain tyrosine hydroxylase (TH)+ dopaminergic neurons, Calbindin+ cerebellar Purkinje cells, interneuron populations and CD31+ endothelial cells of a subject relative to a non-rational polyploid AAV particle that has ability to cross blood brain barrier.

Methods to determine relative transduction efficiencies are described in www.moleculartherapy.org vol. 19 no. 8, 1440-1448, 2011, the non patent literature is incorporated by reference in its entirety. In some embodiments, the population of haploid AAV virion disclosed herein has enhanced transduction throughout CNS relative to AAV2 or, AAV5. In some embodiments, the population of haploid AAV virion has enhanced transduction in striatum relative to AAV2 or, AAV5, e.g., at least 20%, or 30%, or 40%, or 50%, 60%, 70%, 80%, 90% or 95%, or 100% better, or at least 1.2 fold, or at least 1.5 fold, or at least 2 fold, at least 5 fold, at least 10 fold, at least 20 fold, at least 50 fold or more enhanced transduction in the striatum as compared to that of a AAV2 or AAV5 virion under similar conditions.

In one embodiment, the present invention provides an adeno-associated virus (AAV) haploid or polyploid capsid, wherein the capsid comprises a capsid protein VP1, wherein said capsid protein VP1 is from any serotype and at least a capsid protein VP3, wherein said capsid protein VP3 is from any AAV serotype which crosses the BBB and/or a non-human primate, and which is not the same serotype as the serotype of the VP1 (or VP2, if present) AAV serotype. Preferably, such population of rational polyploid virions is substantially homogenous. In some embodiments, a rational polyploid virions disclosed herein can comprise a VP2 capsid protein, wherein said VP2 capsid protein is from any AAV serotype disclosed in Table 1, or a chimeric VP2 protein thereof, or where the VP2 capsid protein is from any serotype that is the same as the serotype from which VP3 comes, or alternatively, wherein the VP2 capsid proteins is from different serotype as the serotype from which VP3 comes from. Exemplary configurations of VP1-VP2-VP3 of a rational polyploid e.g., haploid virion disclosed herein can be represented as follows: X-Y-Z, X-X-Z, Z-X-Z, X-Z-Z, where X, Y and Z each are only from one AAV serotype and which are different serotypes, and where X and Z can be selected from any serotype disclosed in Table 1, and Z is from any serotype that crosses the BBB. That is, in any rational haploid vector, VP3 is only from one Z serotype, VP1 is only from one X serotype, and so forth. In some embodiments, X and Y can be selected from any serotypes which cross the BBB and/or non-human primate AAV serotypes, but in such instances, they are from a different serotype to the serotype for Z. In some embodiments, Z is from any non-human primate AAV serotype, e.g., from a Rhesus monkey serotype. In some embodiments, Z can be selected from any serotype AAV1, AAV6, AAV6.2, AAV7, AAV9, rh10, rh74, rh39, and rh43.

In one embodiment, a rational haploid virion described herein that encapsidates an AAV genome (including a heterologous gene located between 2 AAV ITRs) can be formed with only two of the viral structural proteins, VP1 and VP3. In one embodiment such a rational haploid AAV virions is conformationally correct, i.e., has T=1 icosahedral symmetry. In one embodiment, the rational haploid AAV virions described herein are infectious.

The AAV virion has T=1 icosahedral symmetry and is composed of the three structural viral proteins, VP1, VP2, and VP3. 60 copies of the three viral proteins in a ratio of 1:1:8 to 10 (VP1:VP2:VP3, respectively) form the virion (Rayaprolu, V., et al., J. Virol. 87(24): 13150-13160 (2013).

In one embodiment, the rational polyploid AAV virion is an isolated virion that has at least one of the viral structural proteins, VP1, VP2, and VP3 from a different serotype than the other VPs, and each VP is only from one serotype. For illustrative purposes only, if the VP1 is only from AAV2, the VP2 is only from AAV4, and the VP3 is only from a serotype that crosses the BBB that is not AAV2 or AAV4. In some embodiments, the population of rational polyploid AAV virions has enhanced biodistribution in brain and spinal cord relative to AAV8. In some embodiments, CNS biodistribution is at least 0.1 VG/cell, at least 0.2 vg/cell, at least 0.4 vg/cell, at least 0.6 vg/cell, at least 0.8 vg/cell, at least 1 vg/cell, at least 2 vg/cell, at least 3 vg/cell, at least 4 vg/cell, at least 5 vg/cell, at least 6 vg/cell, at least 7 vg/cell at least 8 vg/cell, at least 9 vg/cell, at least 10 vg/cell, at least between 10-15 vg/cell, at least between 15-20 vg/cell, at least between 20-30 vg/cell, at least between 30-40 vg/cell, at least between 40-50 vg/cell, at least 50VG/cell at least between 50-60 vg/cell, at least between 60-70 vg/cell, at least between 70-80 vg/cell, at least between 80-90 vg/cell, at least between 90-100 vg/cell, at least 100 vg/cell, or more.

CNS biodistribution constitutes biodistribution in regions of brain and regions of spinal cord. Non limiting exemplary CNS biodistribution regions include, olfactory bulb, striatum, hippocampus, cortex, thalamus, hypothalamus, cerebellum, medulla, cervical, thoracic, lumbar, choroid plexus, habenular nucleus, cornu ammonis, dentate gyrus, caudate-putamen, amygdala. In some embodiments, the population of rational polyploid AAV virion has enhanced transduction in neuron than in glial cells. In some embodiments, the population of rational polyploid virion has enhanced transduction in astrocytes. In some embodiments, the population of rational polyploid AAV virion of the invention has significant transduction in tissues other than CNS. In several aspects of the embodiment, the population of rational polyploid AAV virion has transduction in lung, kidney, and supraphysiological levels in the liver, heart, skeletal muscle, intestine, and spleen.

In some embodiments, the population of rational polyploid AAV virion of the invention has significant transduction of endothelial cells of the brain blood vessel (BBV). In some embodiments, the population of rational polyploid AAV virion of the invention has significant transduction of cells that are part of the BBB, including any one or more of: endothelial cells of the BBB, astrocyte cell projections called astrocytic feet (also known as “glia limitans”) surround the endothelial cells of the BBB. In some embodiments, the population of rational polyploid AAV virion of the invention has significant transduction of a blood component that can crosses the BBB, including, but not limited to, activated T cells, blood monocytes, macrophages.

In one embodiment a rational polyploid AAV virion that encapsidates an AAV genome including a heterologous gene between 2 AAV ITRs can be formed with only two of the viral structural proteins, VP1 and VP3, where VP3 is from a serotype that efficiently crosses the BBB. In one embodiment this virion is conformationally correct, i.e., has T=1 icosahedral symmetry. In one embodiment the virions are infectious.

Infectious virions include VP1/VP3 or VP1/VP2/VP3. Typically, virions comprising only VP2/VP3 or only VP3 are not infectious.

In some embodiments, the viral structural proteins VP2 used to generate these populations of rational polyploid AAV virions can be from any of the 12 serotypes of AAV isolated for gene therapy, other species, mutant serotypes, shuffled serotypes of such genes, e.g., AAV1, AAV2, AAV VP1.5, AAV4 VP2, AAV4 VP3, AAV Rh10 VP3, AAV Rh74 VP3, AAV Rh74 VP2 or any other AAV serotype desired, for example as disclosed in Table 1.

As disclosed herein, the VP3 structural capsid protein is selected from any AAV serotype that efficiently crosses the BBB. Such AAV serotypes that cross the BBB are selected from any of AAV1, AAV6, AAV6.2, AAV7, AAV9, rh10, rh74, rh39, and rh43. Exemplary rational polyploid vectors comprising VP1, VP2 and VP3 proteins for use in the methods and compositions disclosed herein include, but are not limited to, AAV-X-Y-1; AAV-X-X-1; AAV-X-Y-6; AAV-X-X-6; AAV-X-Y-6.2; AAV-X-X-6.2; AAV-X-Y-7; AAV-X-X-7; AAV-X-Y-9; AAV-X-X-9; AAV-X-Y-rh10; AAV-X-X-rh10; AAV-X-Y-rh74; AAV-X-X-rh74; AAV-X-Y-rh39; AAV-X-X-rh39; AAV-X-Y-43; AAV-X-X-43, where X and Z are each selected only from one AAV serotype and are different serotypes and are selected from any serotype disclosed in Table 1. In some embodiments, X and/or Y can be from any serotype that crosses the BBB, or alternatively from any non-human primate AAV serotype, and when this occurs, the serotype for X and/or Y is different to the serotype from that of VP3. In some embodiments where the rational polyploid comprises a VP1, VP2 and VP3 protein, the serotype of the VP1 protein serotype is the same as the serotype of the VP3 protein which crosses the BBB, and the serotype of the VP2 protein is from a different serotype. In some embodiments where the rational polyploid comprises a VP1, VP2 and VP3 protein, the serotype of the VP2 protein serotype is the same as the serotype of the VP3 protein which crosses the BBB, and the serotype of the VP1 protein is from a different serotype.

In some embodiments, a rational polyploid vector can comprise only a VP1 and VP3 protein, or only a VP2 and VP3 protein, and such exemplary rational polyploid vectors for use in the methods and compositions disclosed herein include, but are not limited to, AAV-X-1; AAV-X-6; AAV-X-6.2; AAV-X-7; AAV-X-9; AAV-X-rh10; AAV-X-rh74; AAV-X-rh39; AAV-X-43, where X is either VP1 or VP2 and is selected only from one serotype selected from any serotype disclosed in Table 1. In some embodiments, X can be from any serotype that crosses the BBB, or alternatively from any non-human primate AAV serotype, and when this occurs, the serotype for X is different to the AAV serotype of VP3.

In some embodiments, VP3 can be selected from any AAV serotype that crosses the BBB, including but not limited to AAV1, AAV6, AAV6.2, AAV7, AAV9, AAVrh10, AAVrh74, AAVrh39, and AAVrh43 or variants having at least 85% amino acid sequence identity to the native amino acid sequences. AAV serotypes which cross the BBB are disclosed in Zhang et al., Mol. Therapy, 19(8); 2011; 1440-1448, Nonnenmacher et al., Mol Ther: Methods and Clinical development; 2021, 336, and Gao et al., J. Virol, 2004; 6381-6388, which are incorporated herein in their entirety by reference.

In some embodiments, a rational AAV polyploid vector disclosed herein comprises VP1, VP2 or VP3 proteins, where VP1, VP2 or VP3 are each from different serotypes, where serotype X or Y is any AAV serotype selected from Table 1, Serotype Z is from any serotype that crosses the BBB only, and/or alternatively, and non-human primate AAV or any serotype or chimeric or non-naturally occurring serotype that crosses the BBB that is not serotype X or the serotype Y.

In some embodiments, the AAV rational polyploid comprises three serotypes, e.g., X, Y and Z, where VP1, VP2 and VP3 are only from one serotype, each of which are a different serotype, and where X and Y can be selected from any serotype selected from Table 1, and VP3 (serotype Z) is from a is any AAV serotype that crosses the BBB. Such combinations are shown in Table 2.

TABLE 2
Table of combinations of different capsid proteins
of a rational AAV polyploid comprising three different
serotypes; X, Y and Z, were serotype Z is selected
from any AAV serotype that crosses the BBB.

| VP1 | VP2 | VP3 |
|---|---|---|
| Serotype X | Serotype Y | Serotype Z (BBB) |
| Serotype X | Serotype X | Serotype Z (BBB) |
| Serotype Z (BBB) | Serotype Y | Serotype Z (BBB) |
| Serotype X | Serotype Z (BBB) | Serotype Z (BBB) |

In some embodiments, the AAV rational haploid vector comprises only two serotypes, were one of the serotypes is any AAV serotype that crosses the BBB (referred to as serotype Z) and one of the serotypes (serotype X) is from any serotype selected from Table 1. In some embodiments, exemplary serotypes for serotype A include, but are not limited to AAV8, AAV9, AAV3, AAV3b. In some embodiments, exemplary serotypes for serotype Z are AAV serotypes that cross the BBB selected from any of: AAV1, AAV6, AAV6.2, AAV7, AAV9, AAVrh10, AAVrh74, AAVrh39. In some embodiments, serotypes for serotype Z can also be selected from any non-human primate AAV serotype, including rhesus monkey serotypes AAVrh.10, AAVrh.74, AAVrh.73, AAVrh.75, AAVrh.76, rAAVrh39, rAAVrh.43.

### IV. Exemplary AAV8 Haploid Vectors that Cross the BBB

In one embodiment, the rational polyploid vector disclosed herein is an adeno-associated virus 8 (AAV8) haploid or polyploid capsid, wherein the capsid comprises a capsid protein VP1, wherein said capsid protein VP1 is from AAV8 serotype and at least a capsid protein VP3, wherein said capsid protein VP3 is from any AAV serotype which crosses the BBB and/or a non-human primate, and is not the AAV8 serotype. Preferably, such population of AAV8 haploid virions is substantially homogenous. In some embodiments, a AAV8 haploid capsid of this invention can comprise a VP2 capsid protein, wherein said VP2 capsid protein is from AAV8 serotype, or a chimeric VP2 protein thereof, or where the VP2 capsid protein is from any serotype that is the same as the serotype from which VP3 comes, or alternatively, wherein the VP2 capsid proteins is from different serotype as the serotype from which VP3 comes from. Exemplary configurations of VP1-VP2-VP3 in the AAV8 haploid capsids of the invention can be represented as follows: AAV8-8-Y, AAV8-X-Y, AAV8-Y-Y, where X is a VP2 capsid protein from any serotype (except AAV8), and Y is a VP3 protein from any serotype (except AAV8), where X and Y are from different serotypes, and where X and Y can be selected from any serotypes which cross the BBB and/or non-human primate AAV serotypes.

In some embodiments, a AAV8 haploid or polyploid virions disclosed herein contain VP1 from AAV8 serotype and at least a VP3 capsid protein, where VP3 is not from AAV8 and is selected from any serotypes which cross the BBB and/or is a non-human primate AAV serotypes, is produced. For example, only AAV8 haploid or polyploid virions where VP1 and optionally VP2 is from the AAV8 serotype, and VP3 is not from AAV8 and is selected from any AAV serotype which crosses the BBB and/or is a non-human primate AAV serotypes is produced.

In one embodiment, the AAV8 virion is an isolated virion that has at least VP1 from AAV8 serotype and one of the viral structural proteins, VP2 and/or VP3 from a different serotype than AAV8, where either VP2 and/or VP3 is from any AAV serotype where the native AAV vector crosses the BBB. For example, a AA8 haploid virion described herein can comprise VP1 from AAV8, and VP2 or VP3 or both VP2 and VP3 from AAV9, AAV7 (accession number AF513852, which is the whole genome of AAV7), rAAVrh74, rAAVrh.39, rAAVrh.43 or variants thereof.

In one embodiment, the AAV8 virion is an isolated virion that has at least VP1 from AAV8 serotype and one of the viral structural proteins, VP2 and/or VP3 from a different serotype than AAV8, where either VP2 and/or VP3 is from any non-human primate AAV serotype. For example, the VP1 is only from AAV8, and VP2 and/or VP3 is from rhesus monkey. For example, a AA8 haploid virion described herein can comprise VP1 from AAV8, and VP2 or VP3 or both VP2 and VP3 from rAAVrh.10, rAAVrh.74, rAAVrh.39 or rAAVrh.43 or variants thereof. Throughout this application, a virion described herein can be represented as AAV(VP1)-VP2-VP3, or alternatively as “AAVnnn”. For example, for illustrative purposes only, a virion with VP1 from AAV8, VP2 from AAV8 and VP3 from AAVrh.10 could be represented as AAV8-8-rh10 or as AAV88rh10. Accordingly, using this nomenclature, exemplary AAV8 haploid virions herein include, but are not limited to AAV8-8-rh10, AAV8-8-rh74, AAV8-8-rh39, AAV8-8-rh43, or variants thereof, where VP1 and VP2 capsid proteins are from AAV8, and VP3 is from the serotype as indicated. Other exemplary AAV8 haploid vectors include, but are not limited to AAV8-X-rh10, AAV8-X-rh74, AAV8-X-rh39, AAV8-X-rh43, or variants thereof, where X is VP2 from any serotype except AAV8. In some embodiments, X is a VP2 capsid protein from any rhesus monkey AAV serotype, including but not limited to AAVrh10, rAAVrh74, rAAVrh.39 or rAAVrh.43, or variants thereof.

In some embodiments herein, a virion particle can be constructed wherein VP1 capsid proteins is from AAV8 and at least one of VP2 or VP3 viral protein is not from AAV8. In some embodiments, VP1 and VP2 can be from the AAV8 serotype. In all embodiments disclosed herein, VP3 is not from AAV8. In some embodiments, VP2 and VP3 are from the same serotype, e.g., AAV8-rh10-rh10. In some embodiments, VP2 and VP3 are from different serotypes, e.g., AAV8-8-rh10, or AAV8-rh74-rh10. In each aspect of all the embodiments described herein, at least VP1 from AAV8 and a VP3 capsid protein from another serotype are required to form the virion particle capable of encapsidating an AAV genome. For each virion particle, the capsid protein (VP1, VP2, and/or VP3), that protein is the same type (e.g., all virions comprise an AAV8 VP1). In some embodiments, at least one of the viral capsid protein is a chimeric viral protein and at least one of the other two viral proteins is not a chimeric. In some embodiments VP1 is a chimeric AAV8 VP1 protein. In some embodiments, VP1 and VP2 are chimeric and only VP3 is non-chimeric. For example, only the viral particle composed of VP1/VP2 from the chimeric AAV2/8 (the N-terminus of AAV2 and the C-terminus of AAV8) paired with only VP3 from any other non-AAV8 vector, e.g., rh10, rh74 etc. In another embodiment only VP3 is chimeric and VP1 and VP2 are non-chimeric. In another embodiment at least one of the viral proteins is from a completely different serotype. In another example, no chimeric is present.

In some embodiments, a rational polyploid virion disclosed herein is a AAV8 haploid virion, and can be selected from any of: AAV88rh10, AAV88rh74, AAV88rh74vv, AAV88rh10LP2, AAV88rh74LP2, AAV88rh74vvLP2, each of which are described in the EXAMPLES section provided herein. In some embodiments, these AAV8 haploid virions can be represented as AAV8-8-rh10, AAV8-8-rh74, AAV8-8-rh74vv, AAV8-8-rh10LP2, AAV8-8-rh74LP2, AAV8-8-rh74vvLP2. By way of explanation only, these AAV haploid virions comprise AAV8 VP1 and VP2 structural proteins (e.g., SEQ ID NO: 7 and 8) or comprise a modified proteins of SEQ ID NO: 7 or 8, and a VP3 protein selected from any of: rh10 VP3 (SEQ ID NO: 1), rh74 VP3 (SEQ ID NO: 3), rh74vv VP3 (SEQ ID NO: 2), rh10-LP2 VP3 protein (SEQ ID NO: 14), rh74-LP2 VP3 protein, (SEQ ID NO: 17), and rh74vv-LP2 VP3 protein (SEQ ID NO: 15), or a VP3 protein having an amino acid sequence that is at least 85% sequence identity to any of SEQ ID NO: 1, 2, 3, 14, 15 and 18. In some embodiments, the modified AAV8 VP1 or VP2 proteins comprise a peptide insertion at an appropriate site in the VP1 and/or VP2 protein, including but not limited to a targeting peptide or BBB penetrating peptide, as disclosed herein. In some embodiments, a linker, e.g., peptide linker may flank the targeting peptide (i.e., each end of the targeting peptide may comprise a peptide linker), or there is a peptide linker located at one end of the targeting peptide. In some embodiments, the AAV8 VP1 and/or VP2 structural proteins are modified as a double mutant (Y444+ 733F) or a triple mutant ((Y444+ 733F T494V) as disclosed in Gilkes Site-specific modifications to AAV8 capsid yields enhanced brain transduction in the neonatal MPS IIIB mouse. Gene Ther (2020), where this non-patent publication is incorporated herein in its entirety by reference.

In some embodiments only virions that contain VP1 capsid protein from AAV8 and VP3 capsid protein that is from a different serotype than AAV8 are produced. For example, VP1 and VP2 are from AAV8 serotype and VP3 is from an alternative serotype, only. In other embodiments, the VP1 is from AAV8 serotype and the VP2 and VP3 are from another serotype, only, where the VP2 and VP3 capsid proteins are from a serotype that crosses the BBB and/or is a non-human primate AAV serotype. In another embodiment, only particles where VP1 is from AAV8 serotype, VP2 is from a second serotype, and VP3 is from yet another serotype, are produced, and is referred to herein as a polyploid AAV8 virnon.

This can be done by, for example, site specific deletions, and/or additions, changing splice donor sites, splice acceptor sites, start codons and combinations thereof.

This permits methods for producing populations of substantially homogenous populations of the polyploid virions-such as the haploid particles.

In some embodiments, exemplary AAV8 haploid vectors are selected from the following:
a. AAV8-8-rhY (where Y is a VP3 from non-human primate AAV serotype, including rhesus monkey),
b. AAV8-8-rh10,
c. AAV8-X-rh10, where x is any serotype except Rh10;
d. AAV8-X-rh10, where x is any serotype except AAV8;
e. AAV8-8-rh74,
f. AAV8-8-rh74vv, where rh74 VP3 capsid protein comprises a W581VV modification;
g. AAV8-X-rh74 or rh74vv, where x is any serotype except Rh74;
h. AAV8-X-rh74/rh74vv, where x is any serotype except AAV8;
i. AAV8-8-Y, where is Y is a VP3 capsid protein from any serotype that crosses BBB (e.g., exemplary VP3 capsid proteins that cross the BBB can be selected from AAV1, AAV6, AAV6.2, AAV7 (accession number AF513852, which is the whole genome of AAV7), AAV9, rAAVrh10, rAAVrh74, rAAVrh39, rAAVrh43);
j. AAV8-X-Y, where X is any serotype except AAV8 and where Y is a VP3 is from any serotype that crosses BBB and where Y is not from AAV8
k. AAV8-X-Y, where X is any serotype that crosses the BBB, where Y is a VP3 from any serotype that crosses BBB, and where X and Y are not a VP2 or VP3 capsid protein from AAV8 serotype, respectively, where a serotype that crosses the BBB can be selected from AAV1, AAV6, AAV6.2, AAV7 (accession number AF513852, which is the whole genome of AAV7), AAV9, rAAVrh10, rAAVrh74, rAAVrh39, rAAVrh43);
l. AAV8-X-X, where X is any serotype that crosses the BBB, and where X is not a VP2 or VP3 capsid protein from AAV8 serotype, respectively, where a serotype that crosses the BBB can be selected from AAV1, AAV6, AAV6.2, AAV7 (accession number AF513852, which is the whole genome of AAV7), AAV9, rAAVrh10, rAAVrh74, rAAVrh39, rAAVrh43);
m. AAV8-X-Y, where X is any serotype except AAV8 and where Y is a VP3 is from any serotype that crosses BBB and where Y is not from AAV8

In some embodiments, exemplary AAV haploid vector that cross the BBB are disclosed in Table 4.

TABLE 4
Exemplary rational polyploid or polyploid vectors, wherein VP3 is from a serotype
that crosses the BBB (e.g., selected from any of AAV1, AAV6, AAV6.2, AAV7,
AAV9, AAVrh10, AAVrh74, AAVrh39), and VP1 and/or VP2 are from a different
serotype to that of the serotype of VP3, and can also be from serotype that
crosses the BBB, or selected from AAV8 or X, where X is a serotype that is
not AAV8 and can be selected from any serotype from Table 1.

| | VP1 | VP2 | VP3 (crosses BBB) |
|---|---|---|---|
| AAV-8-8-1; | 8 or X | 8 or X | AAV1 |
| AAV-8-X-1; | | | |
| AAV-BBB-8-1; | AAV6, AAV6.2, AAV7, | X or 8 | AAV1 |
| AAV-BBB-X-1; | AAV9, rAAVrh10, | | |
| | rAAVrh74, rAAVrh39, | | |
| | rAAVrh43, AAVRh74vv | | |
| AAV-X-BBB-1; | X or 8 | AAV6, AAV6.2, AAV7, | AAV1 |
| AAV-8-BBB-1; | | AAV9, rAAVrh10, | |
| | | rAAVrh74, rAAVrh39, | |
| | | rAAVrh43, AAVRh74vv | |
| AAV-8-8-6; | 8 or X | 8 or X | AAV6 |
| AAV-8-X-6; | | | |
| AAV-BBB-8-6; | AAVI, AAV6.2, AAV7, | X or 8 | AAV6 |
| AAV-BBB-X-6; | AAV9, rAAVrh10, | | |
| | rAAVrh74, rAAVrh39, | | |
| | rAAVrh43, AAVRh74vv | | |
| AAV-X-BBB-6; | 8 or X | AAV1, AAV6.2, AAV7, | AAV6 |
| AAV-8-BBB-6; | | AAV9, rAAVrh10, | |
| | | rAAVrh74, rAAVrh39, | |
| | | rAAVrh43, AAVRh74vv | |
| AAV-8-8-6.2; | 8 or X | 8 or X | AAV6.2 |
| AAV-8-X-6.2; | | | |
| AAV-BBB-8-6.2; | AAV1, AAV6, AAV7, | X or 8 | AAV6.2 |
| AAV-BBB-X-6.2; | AAV9, rAAVrh10, | | |
| | rAAVrh74, rAAVrh39, | | |
| | rAAVrh43, AAVRh74vv | | |
| AAV-X-BBB-6.2; | 8 or X | AAVI, AAV6, AAV7, | AAV6.2 |
| AAV-8-BBB-6.2; | | AAV9, rAAVrh10, | |
| | | rAAVrh74, rAAVrh39, | |
| | | rAAVrh43, AAVRh74vv | |
| AAV-8-8-7; AAV- | 8 or X | 8 or X | AAV7 |
| 8-X-7; | | | |
| AAV-BBB-8-7; | AAV1, AAV6, AAV6.2, | X or 8 | AAV7 |
| AAV-BBB-X-7; | AAV9, rAAVrh10, | | |
| | rAAVrh74, rAAVrh39, | | |
| | rAAVrh43, AAVRh74vv | | |
| AAV-X-BBB-7; | 8 or X | AAV1, AAV6, AAV6.2, | AAV7 |
| AAV-8-BBB-7; | | AAV9, rAAVrh10, | |
| | | rAAVrh74, rAAVrh39, | |
| | | rAAVrh43, AAVRh74vv | |
| AAV-8-8-9; | 8 or X | 8 or X | AAV9 |
| AAV-8-X-9; | | | |
| AAV-BBB-8-9; | AAVI, AAV6, AAV6.2, | X or 8 | AAV9 |
| AAV-BBB-X-9; | AAV7, rAAVrh10, | | |
| | rAAVrh74, rAAVrh39, | | |
| | rAAVrh43, AAVRh74vv | | |
| AAV-X-BBB-9; | 8 or X | AAV1, AAV6, AAV6.2, | AAV9 |
| AAV-8-BBB-9; | | AAV7, rAAVrh10, | |
| | | rAAVrh74, rAAVrh39, | |
| | | rAAVrh43, AAVRh74vv | |
| AAV-8-8-10; | 8 or X | 8 or X | rAAVrh10 |
| AAV-8-X-10; | | | |
| AAV-BBB-8-rh10; | AAVI, AAV6, AAV6.2, | X or 8 | rAAVrh10 |
| AAV-BBB-X-rh10; | AAV7, AAV9, | | |
| | rAAVrh74, rAAVrh39, | | |
| | rAAVrh43, AAVRh74vv | | |
| AAV-X-BBB-rh10; | 8 or X | AAVI, AAV6, AAV6.2, | rAAVrh10 |
| AAV-8-BBB-rh10; | | AAV7, rAAVrh10, | |
| | | rAAVrh74, rAAVrh39, | |
| | | rAAVrh43, AAVRh74vv | |
| AAV-8-8-rh74; | 8 or X | 8 or X | rAAVrh74 |
| AAV-8-X-rh74; | | | |
| AAV-BBB-8-rh74; | AAVI, AAV6, AAV6.2, | X or 8 | rAAVrh74 |
| AAV-BBB-X-rh74; | AAV7, AAV9, | | |
| | rAAVrh10, rAAVrh39, | | |
| | rAAVrh43, AAVRh74vv | | |
| AAV-X-BBB-rh74; | 8 or X | AAVI, AAV6, AAV6.2, | rAAVrh74 |
| AAV-8-BBB-rh74; | | AAV7, AAV9, rAAVrh10, | |
| | | rAAVrh39, rAAVrh43, | |
| | | AAVRh74vv | |
| AAV-8-8-rh74vv; | 8 or X | 8 or X | AAVRh74vv |
| AAV-8-X-rh74; | | | |
| AAV-BBB-8-rh74vv; | AAVI, AAV6, AAV6.2, | X or 8 | AAVRh74vv |
| AAV-BBB-X-rh74vv; | AAV7, AAV9, | | |
| | rAAVrh10, rAAVrh39, | | |
| | rAAVrh43, | | |
| AAV-X-BBB-74vv; | 8 or X | AAV1, AAV6, AAV6.2, | AAVRh74vv |
| AAV-8-BBB-rh74vv; | | AAV7, AAV9, rAAVrh10, | |
| | | rAAVrh39, rAAVrh43, | |
| AAV-8-8-rh39; | 8 or X | 8 or X | rAAVrh39 |
| AAV-8-X-rh39; | | | |
| AAV-BBB-8-rh39; | AAV1, AAV6, AAV6.2, | X or 8 | rAAVrh39 |
| AAV-BBB-X-rh39; | AAV7, AAV9, | | |
| | rAAVrh10, rAAVrh74, | | |
| | rAAVrh43, AAVRh74vv | | |
| AAV-X-BBB-rh39; | 8 or | AAVI, AAV6, AAV6.2, | rAAVrh39 |
| AAV-8-BBB-rh39; | | AAV7, AAV9, rAAVrh10, | |
| | | rAAVrh74, rAAVrh43, | |
| | | AAVRh74vv | |
| AAV-8-8-rh43; | 8 or X | 8 or X | rAAVrh43 |
| AAV-8-X-rh43; | | | |
| AAV-BBB-8-rh43; | AAV1, AAV6, AAV6.2, | 8 or X | rAAVrh43 |
| AAV-BBB-X-rh43; | AAV7, AAV9, | | |
| | rAAVrh10, rAAVrh74, | | |
| | rAAVrh39, AAVRh74vv | | |
| AAV-X-BBB-rh43; | 8 or X | AAV1, AAV6, AAV6.2, | rAAVrh43 |
| AAV-8-BBB-rh43; | | AAV7, AAV9, rAAVrh10, | |
| | | rAAVrh74, rAAVrh39, | |
| | | AAVRh74vv | |
| AAV8-X-rh10 | 8 or X | AAVrh. 10, AAVrh.74, | AAVrh.10 |
| | | AAVrh.74vv, AAVrh.39, | |
| | | AAVrh.43, AAV7, AAV9 | |
| AAV8-X-rh74 | 8 or X | AAVrh.10, AAVrh.74, | AAVrh.74 |
| | | AAVrh.74vv, AAVrh.39, | |
| | | AAVrh.43, AAV7, AAV9 | |
| AAV8-X-rh74vv | 8 or X | AAVrh.10, AAVrh.74, | AAVRh74vv |
| | | AAVrh.74vv, AAVrh.39, | |
| | | AAVrh.43, AAV7, AAV9 | |
| AAV8-X-rh39 | 8 or X | AAVrh.10, AAVrh.74, | AAVrh.39 |
| | | AAVrh.74vv, AAVrh.39, | |
| | | AAVrh.43, AAV7, AAV9 | |
| AAV8-X-rh43 | 8 or X | AAVrh.10, AAVrh.74, | AAVrh.43 |
| | | AAVrh.74vv, AAVrh.39, | |
| | | AAVrh.43, AAV7, AAV9 | |
| AAV8-X-7 | 8 or X | AAVrh.10, AAVrh.74, | AAV7 |
| | | AAVrh.74vv, AAVrh.39, | |
| | | AAVrh.43, AAV7, AAV9 | |
| AAV8-X-9 | 8 or X | AAVrh.10, AAVrh.74, | AAV9 |
| | | AAVrh.74vv, AAVrh.39, | |
| | | AAVrh.43, AAV7, AAV9 | |
| AAVX-X-rh | X (any AAV serotype | X (any AAV serotype from | AAV Rhesus monkey |
| | from Table 1) | Table 1) | serotype selected from |
| | | VP1 and VP2 selected from | AAVrh10, AAV rh74, |
| | | Table 1 can be same or | AAV rh39, AAV rh43, |
| | | different | AAV rh38, AAV rh40, |
| | | | AAV rh2, AAV rh25, |
| | | | AAV rh57, AAV rh50, |
| | | | AAV rh49, AAV rh58, |
| | | | AAV rh61, AAV rh52, |
| | | | AAV rh 53, AAV |
| | | | rh51, AAV rh64, AAV |
| | | | rh8, AAV rh1, AAV |
| | | | rh62, AAV rh48, AAV |
| | | | rh54, AAV rh55, AAV |
| | | | rh35, AAV rh 37, |
| | | | AAV rh 36, AAV |
| | | | rh13, AAV rh32, AAV |
| | | | rh33, AAV rh34 e.g., |
| | | | as described in Gao et |
| | | | al., Journal of |
| | | | Virology, June 2004, |
| | | | pg 6381-6388 which is |
| | | | incorporated herein by |
| | | | reference in its |
| | | | entirety. |

### (i) AAV8-8-Rh10 Haploid Vectors

In some embodiments, an exemplary AAV haploid vector is AAV8-8-rh10, where VP1 and VP2 are only from AAV8 serotype and VP3 is only from rhesus monkey AAV10 (AAVrh10) serotype. In some embodiments, the VP3 capsid protein is a chimeric VP3 protein from AAVrh10 serotype. In some embodiments, the AAV8-8-rh10 haploid vector comprises a VP3 capsid protein having an amino acid sequence of SEQ ID NO: 1, or an amino acid sequence at least 85%, or at least 90%, or at least 95% or at least 98% sequence identity to SEQ ID NO:1, where SEQ ID NO: 1 is the amino acid of codon optimized VP3 capsid protein from AAVrh10. In some embodiments, the VP3 is a modified VP3 protein comprising at least 1, or at least 2 or at least 3 modifications selected from: Q214N, S462N and D517E of SEQ ID NO: 1. SEQ ID NO: 1 comprising the amino acid of VP3 from the AAVrh10 serotype is encoded by the nucleic acid sequence of SEQ ID NO: 5, or a variant of at least 95%, or at least 98% nucleic acid sequence identity to SEQ ID NO: 5.

In some embodiments, an exemplary AAV8 haploid vector is AAV8-8-rh10, where VP1 and VP2 are encoded by the nucleic acid sequence of SEQ ID NO: 6, which comprises M203V and M211V, M204 and/or M212M204 and/or M212 such that the VP3 protein from AAV8 serotype is not expressed. In some embodiments, the nucleic acid variant comprises a modification of at least one base of ACG at positions 412-414 of SEQ ID NO: 6 to disrupt or render the start codon (the Threonine (T or Thr)) of VP2 inoperable, so that the nucleic acid of SEQ ID NO: 6 encodes only VP1 of the AAV8 serotype, and does not encode either VP2 or VP3 from the AAV8 serotype. In such an embodiment, an AAV8 haploid vector can comprise a VP2 protein from a different serotype, e.g., a AAVrh serotype, or alternatively, VP2 may be absent in the AAV8 haplotype, as discussed herein. such that the VP3 protein from AAV8 serotype is not expressed. In some embodiments, the nucleic acid variant comprises a modification of at least one base of ACG at positions 412-414 of SEQ ID NO: 6 to disrupt or render the start codon (the Threonine (T or Thr)) of VP2 inoperable, so that the nucleic acid of SEQ ID NO: 6 encodes only VP1 of the AAV8 serotype, and does not encode either VP2 or VP3 from the AAV8 serotype. In such an embodiment, an AAV8 haploid vector can comprise a VP2 protein from a different serotype, e.g., a AAVrh serotype, or alternatively, VP2 may be absent in the AAV8 haplotype, as discussed herein.

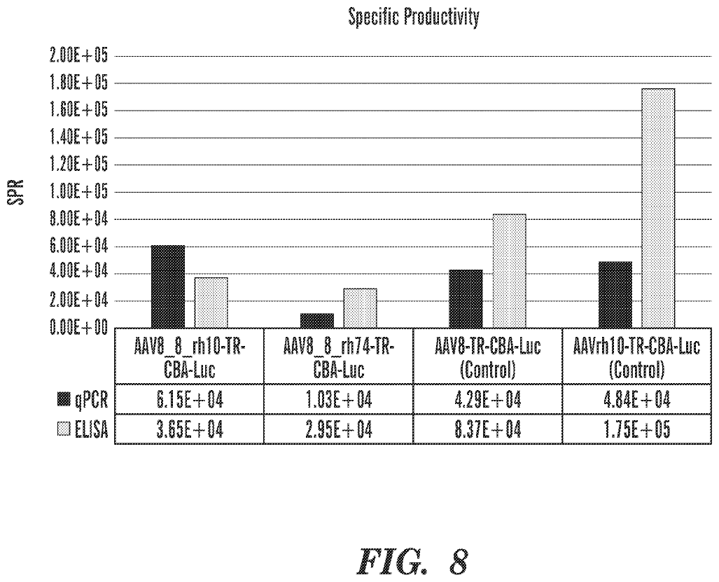

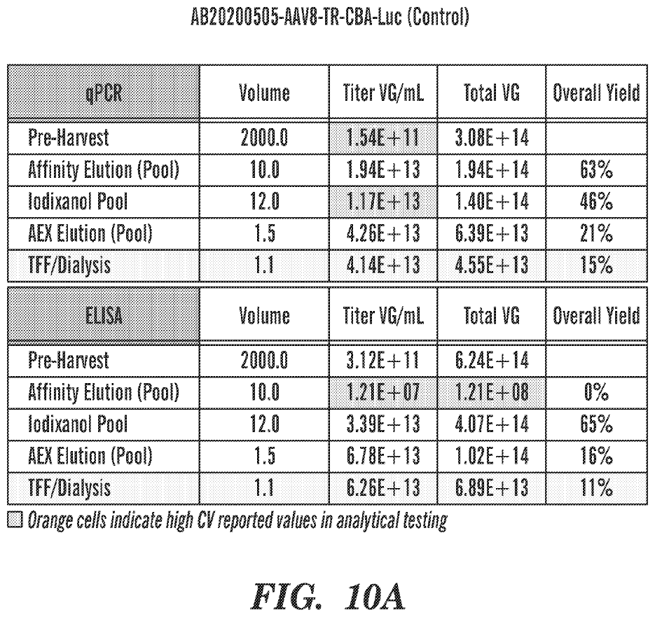

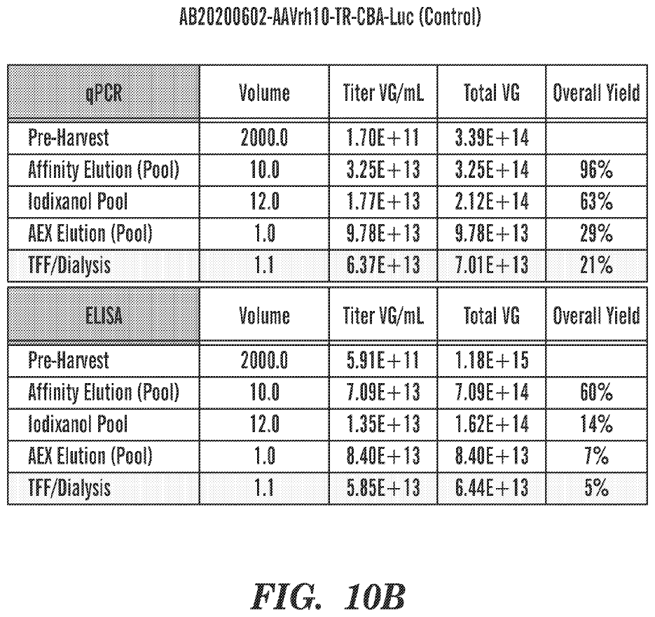

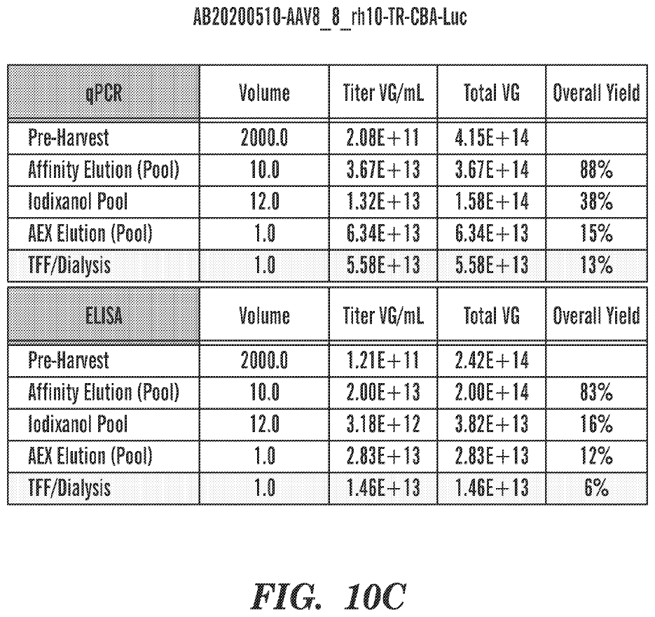

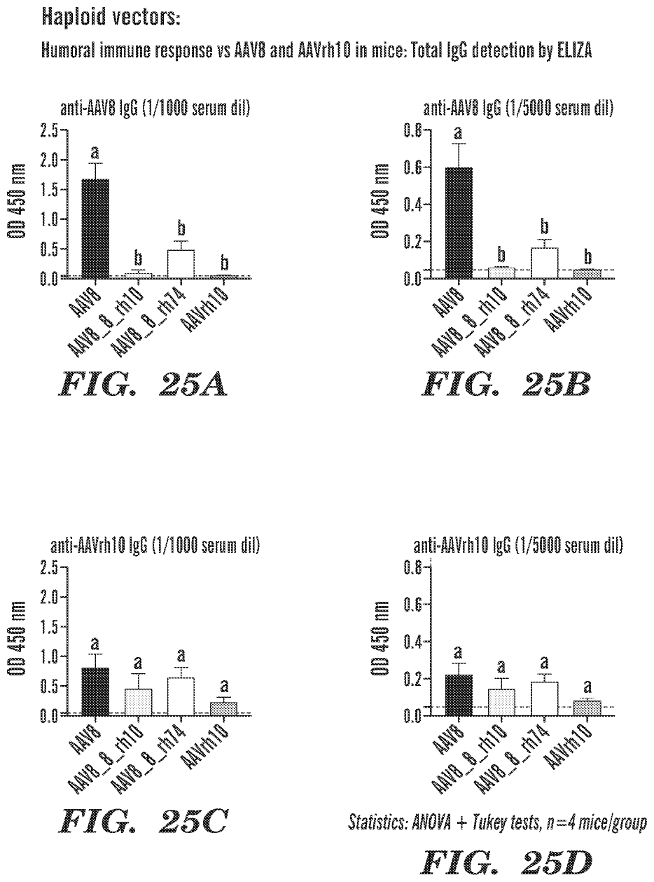

As shown in the Examples herein, rational polyploid AAV8-8-rh10 produced less humoral response compared to parental AAV8 (FIG. 25A) demonstrating that this AAV8-8-rh10 haploid had a different and less antigenic profile as compared to parental AAV8 vectors FIGS. 8B and 10C also demonstrated the production yield and specific productivity from AAV8-8-rh10 was comparable to AAV8 or AAVrh10 controls.

In some embodiments, the AAV8-8-rh10 haploid was produced using the plasmid of SEQ ID NO: 12 which comprises the construct of SEQ ID NO: 13.

### (ii) AAV8-8-Rh74 Haploid Vectors

In some embodiments, an exemplary AAV haploid vector is AAV8-8-rh74, where VP1 and VP2 are only from the AAV8 serotype and VP3 is only from rhesus monkey AAV74 (AAVrh74) serotype. In some embodiments, the VP3 capsid protein is a chimeric VP3 protein from AAVrh74 serotype. In some embodiments, the AAV8-8-rh74 haploid vector comprises a VP3 capsid protein having an amino acid sequence of SEQ ID NO: 3, or an amino acid sequence at least 85%, or at least 90%, or at least 95% or at least 98% sequence identity to SEQ ID NO:3, where SEQ ID NO: 3 is the amino acid of wild type VP3 capsid protein from AAVrh74.

In some embodiments, the rh74 VP3 is a modified VP3 protein comprising at least 1 or more amino acid modifications. In particular embodiments, the AAVrh74 VP3 capsid protein is a modified VP3 protein comprising W581VV modification, where tryptophan (W or Trp) at amino acid position 581 of SEQ ID NO: 3 is substituted for two consecutive valine (V or val) amino acids (using the nomenclature/numbering from the amino acid sequence of the VP1 capsid protein from AAVrh74). Accordingly, in some embodiments, the AAV haploid vector is a AAV8-8-rh74vv haploid vector which comprises a VP3 capsid protein having an amino acid sequence of SEQ ID NO: 2, or an amino acid sequence at least 85%, or at least 90%, or at least 95% or at least 98% sequence identity to SEQ ID NO:2, where SEQ ID NO: 2 is the amino acid of rh74vv-VP3 capsid protein, which comprises the W581VV modification.

SEQ ID NO: 2 comprising the amino acid of the rh74vv-VP3 capsid protein is encoded by the nucleic acid sequence of SEQ ID NO: 4.

In some embodiments, the AAV haploid vector is a AAV8-8-rh74vv haploid vector which comprises a VP3 capsid protein having an amino acid sequence of SEQ ID NO: 2, or an amino acid sequence at least 85%, or at least 90%, or at least 95% or at least 98% sequence identity to SEQ ID NO:2, where SEQ ID NO: 2 is the amino acid of rh74vv-VP3 capsid protein, which comprises the W581VV modification, and where the rh74vv-VP3 capsid protein is encoded by a nucleic acid sequence comprising SEQ ID NO: 4, or a nucleic acid sequence at least at least 85%, or at least 87%, or at least 88%, or at least 89%, or at least 90%, or at least 95% or at least 98% sequence identity to SEQ ID NO:4.

In one embodiment of any aspect herein, the AAVrh74 VP3 protein in a AAV8-8-rh74vv haploid vector has the amino acid sequence of SEQ ID NO: 2 or a protein having at least 85% sequence identity to SEQ ID NO: 2, or wherein the mutated AAVrh74 VP3 comprises at least one of the following modifications of SEQ ID NO: 2: N263S, G264A, T265S, S266T, G268A, T270del, T274H, E533K, R726H, N736P.

In one embodiment of any aspect herein, the AAVrh10 VP3 protein in a AAV8-8-rh74vv haploid vector is encoded by a nucleic acid of SEQ ID NO: 5 that comprises at least one or more of: Q214N, S462N and D517E mutations as compared to AAVrh10_VP3 nucleic acid of SEQ ID NO: 5, or comprises a nucleic acid sequence at least 85% sequence identity to SEQ ID NO: 5 comprising at least one mutation selected from Q214N, S462N and D517E.

In one embodiment of any aspect herein, the AAVrh74 VP3 protein in a AAV8-8-rh74vv haploid vector comprises the amino acid sequences of SEQ ID NO: 2 or 3 or a protein having at least 85% sequence identity to SEQ ID NO: 2 or SEQ ID NO: 2, or comprises at least one of the following amino acid modifications of N263S, G264A, T265S, S266T, G268A, T270del, T274H, E533K, R726H, N736P of SEQ ID NO: 2.

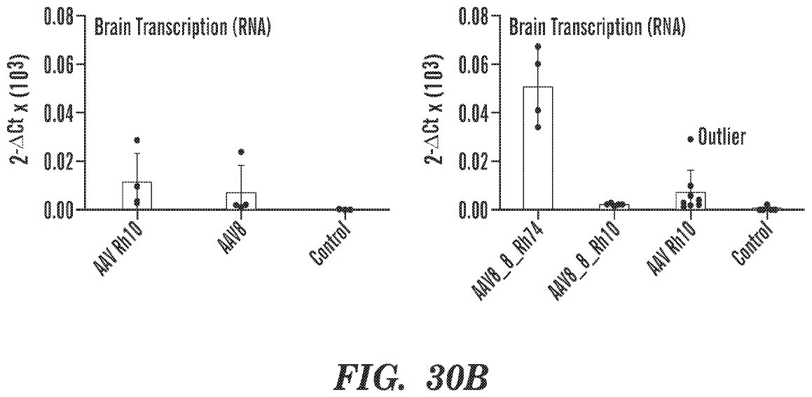

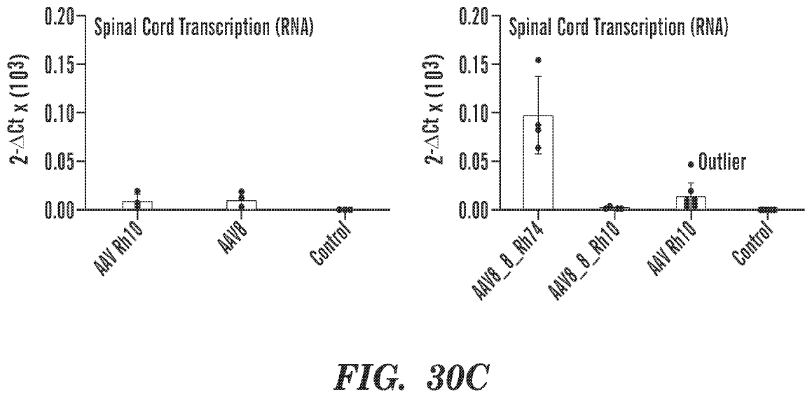

Importantly, as disclosed herein in the Examples and in FIGS. 30B-30C, the haploid AAV8-8-rh74 vector has an improved ability to transduce the brain and spinal cord, and cross the BBB as compared to mice immunized with parental AAV serotypes.

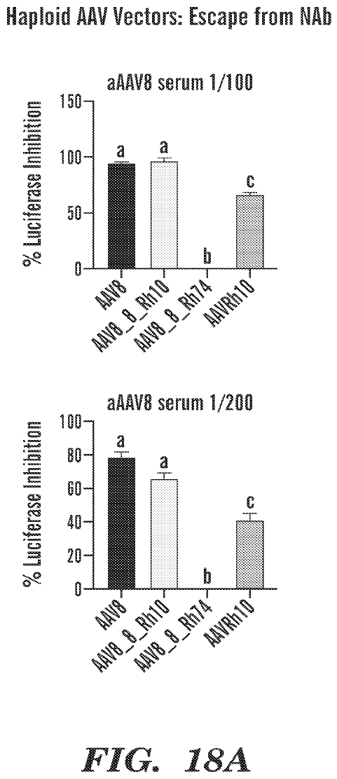

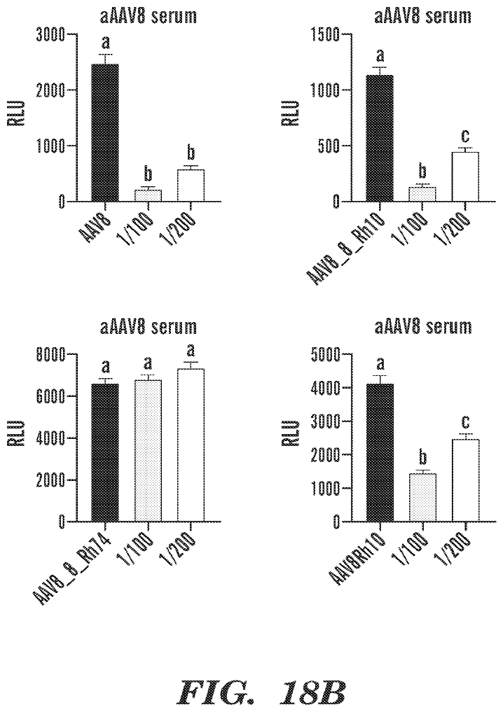

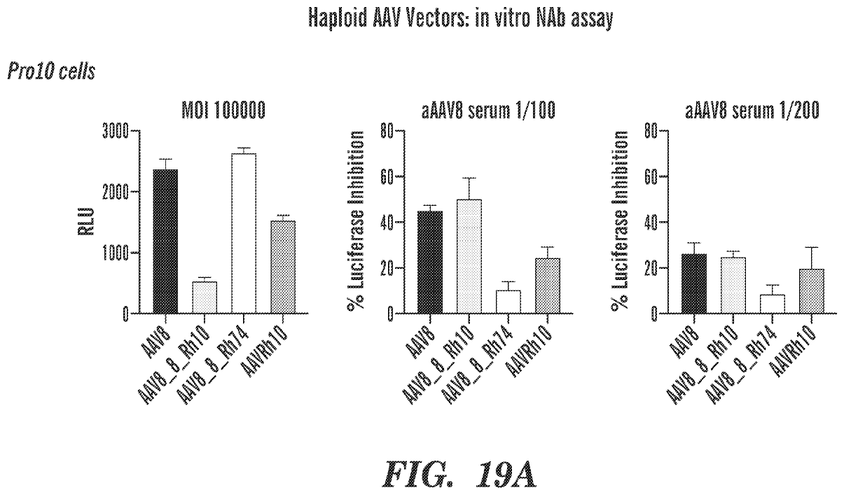

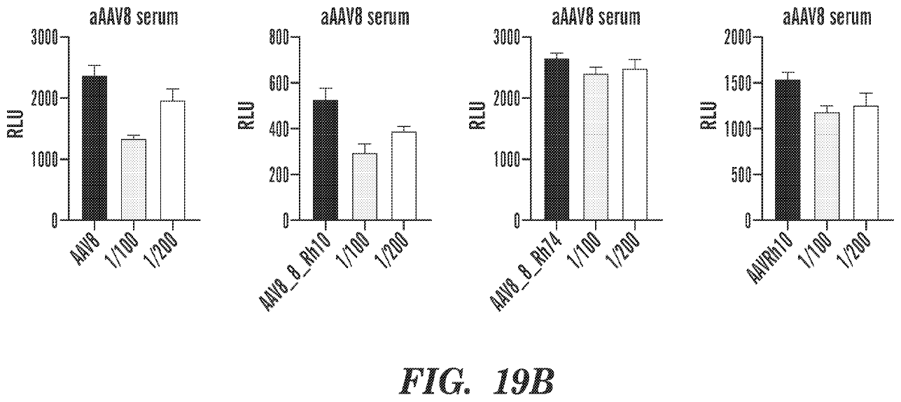

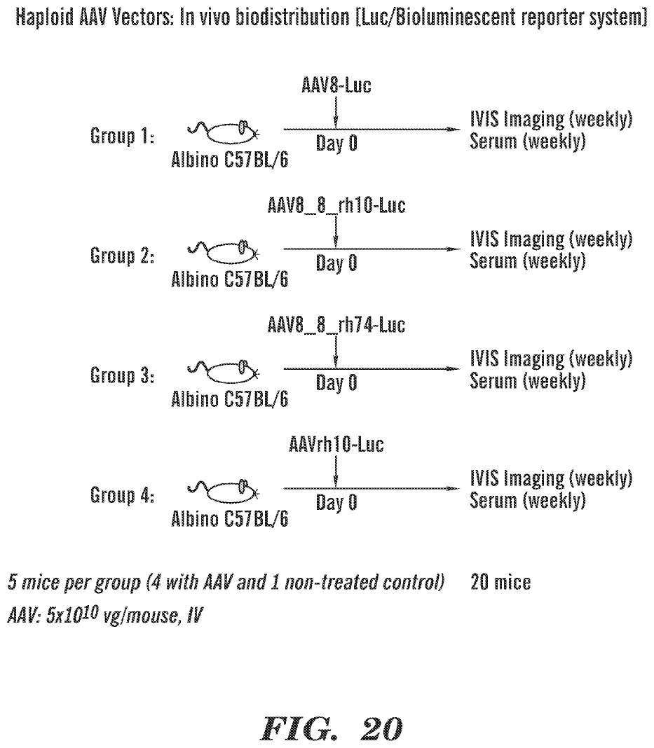

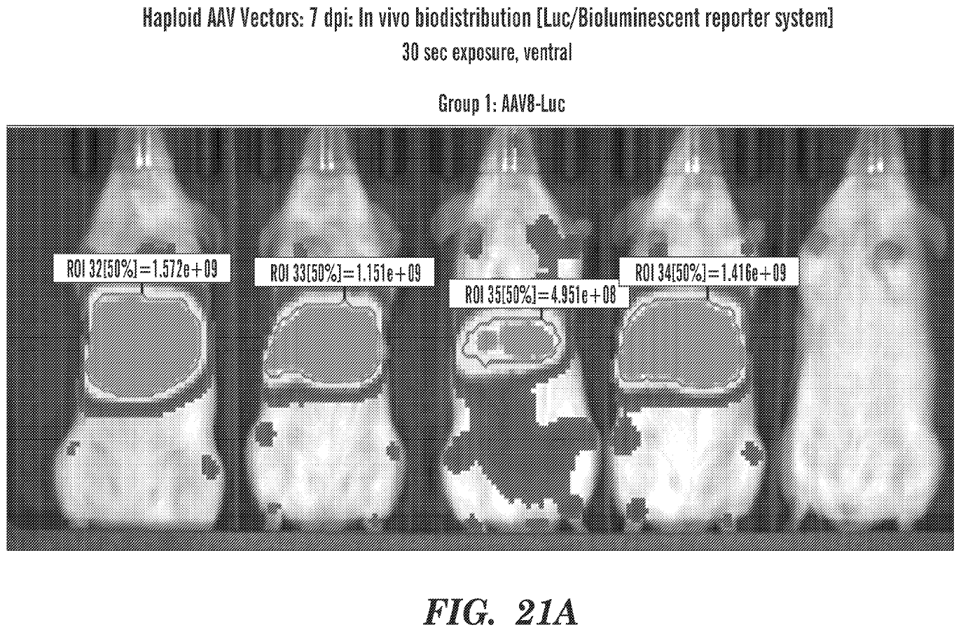

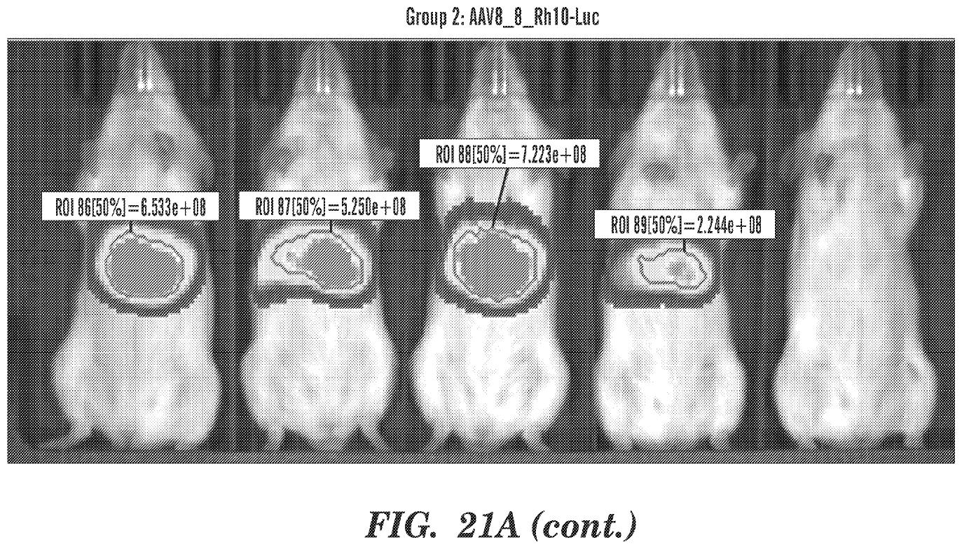

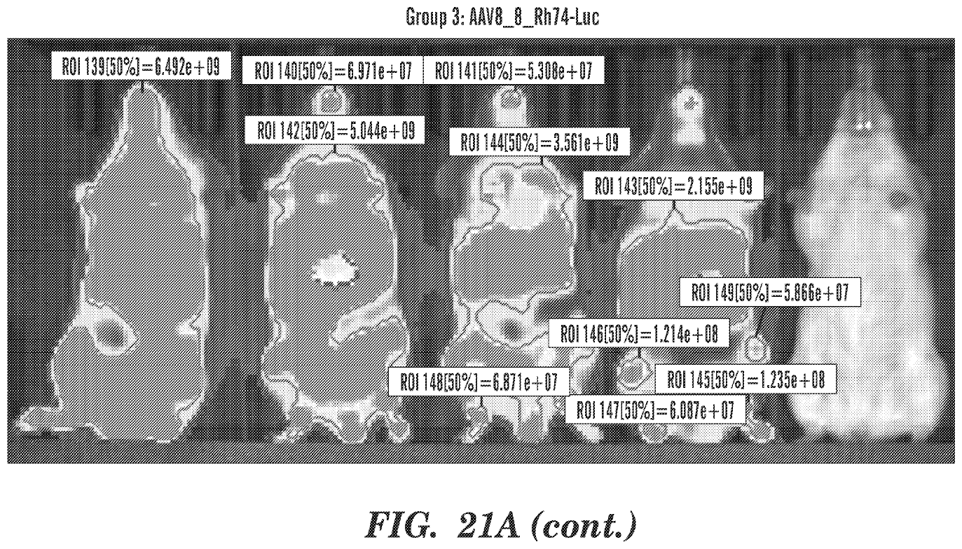

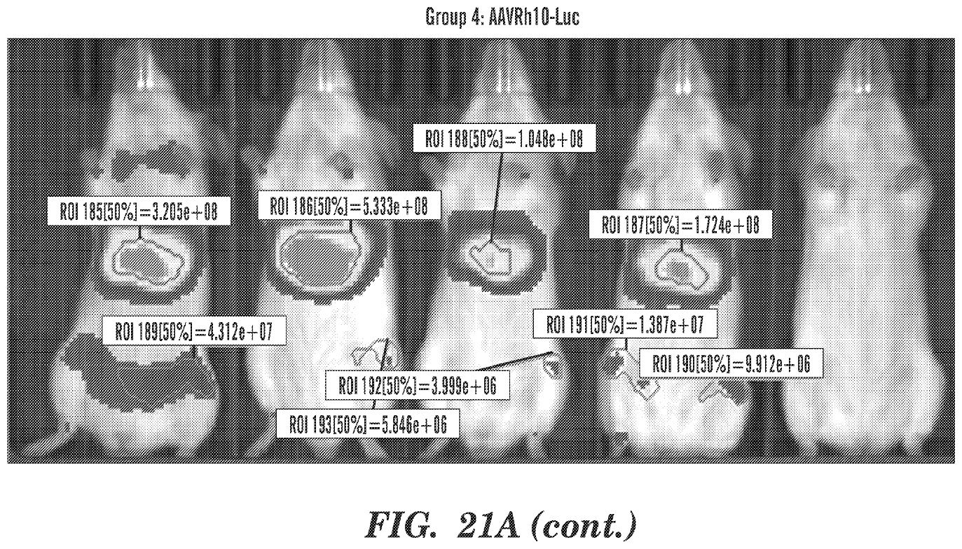

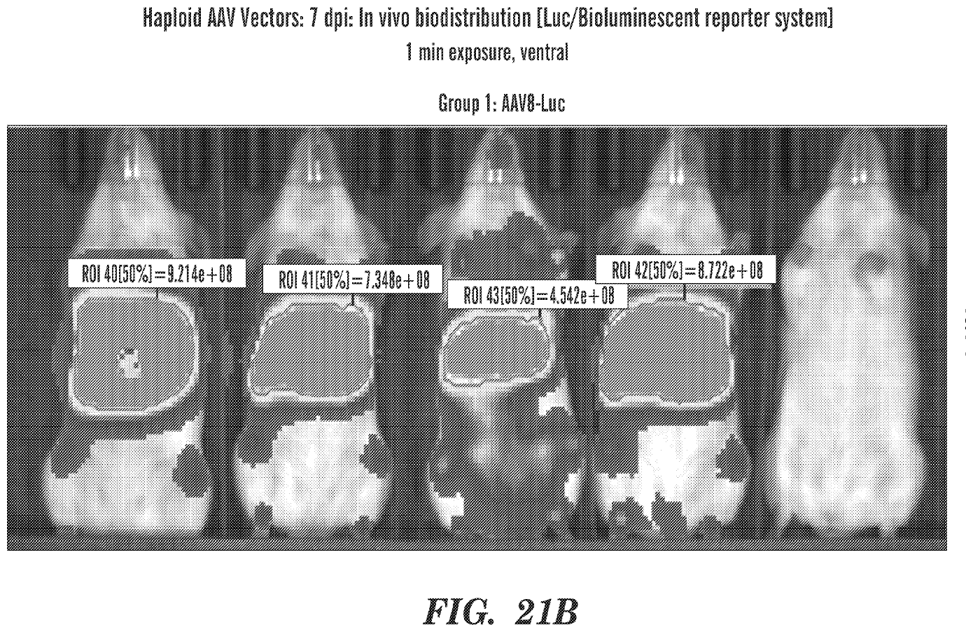

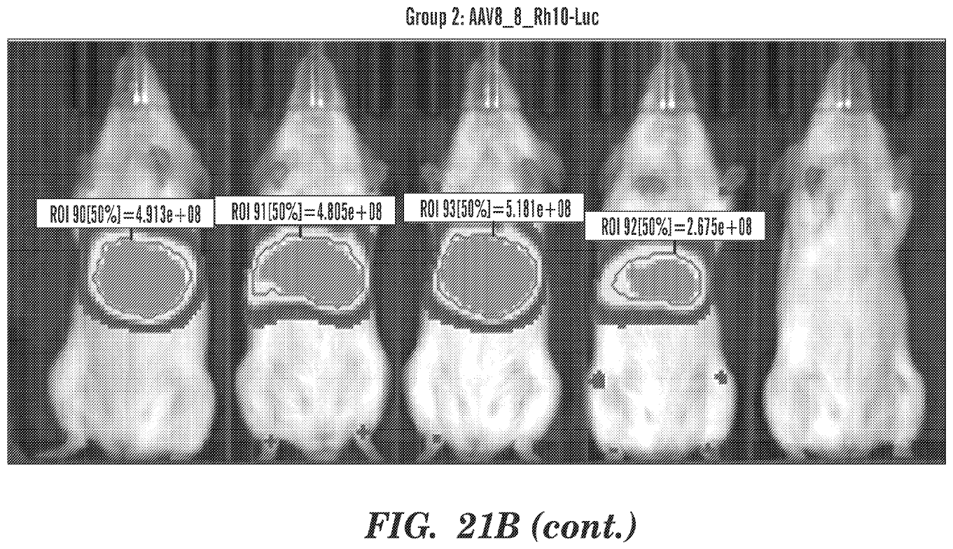

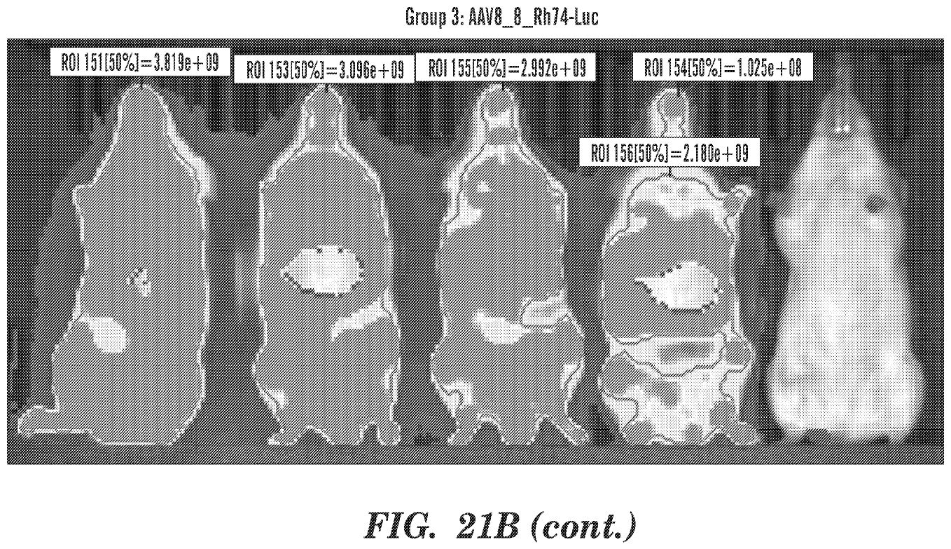

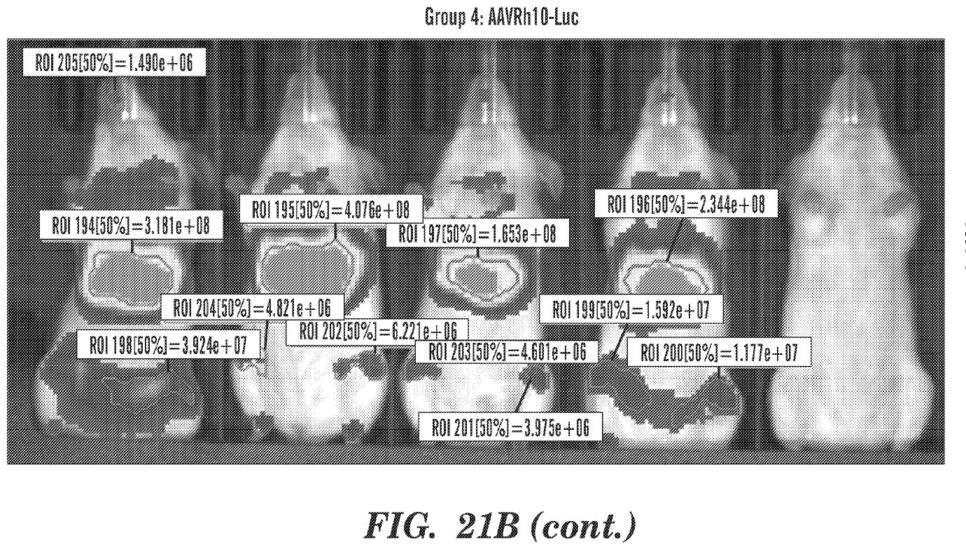

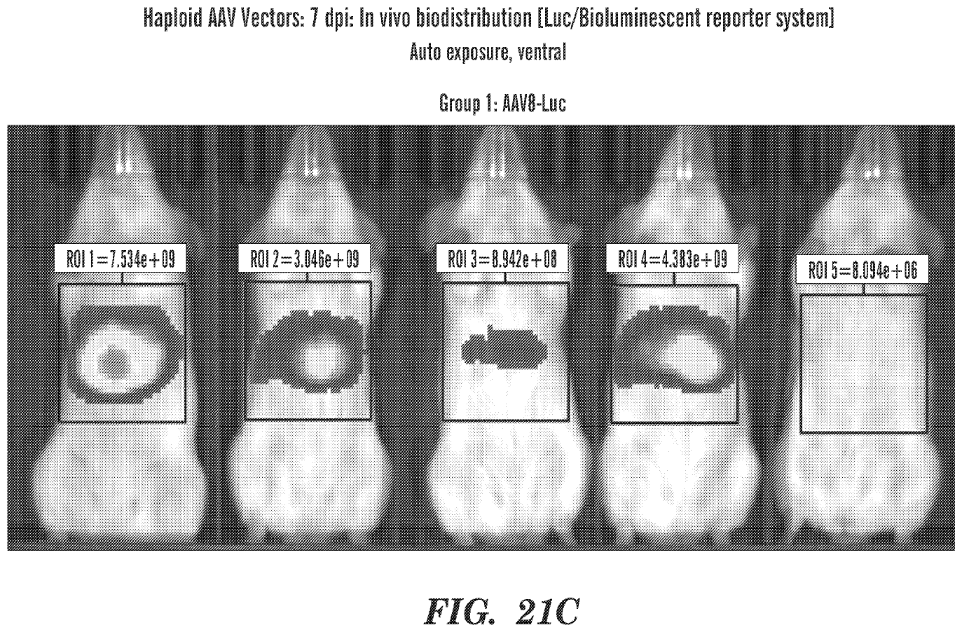

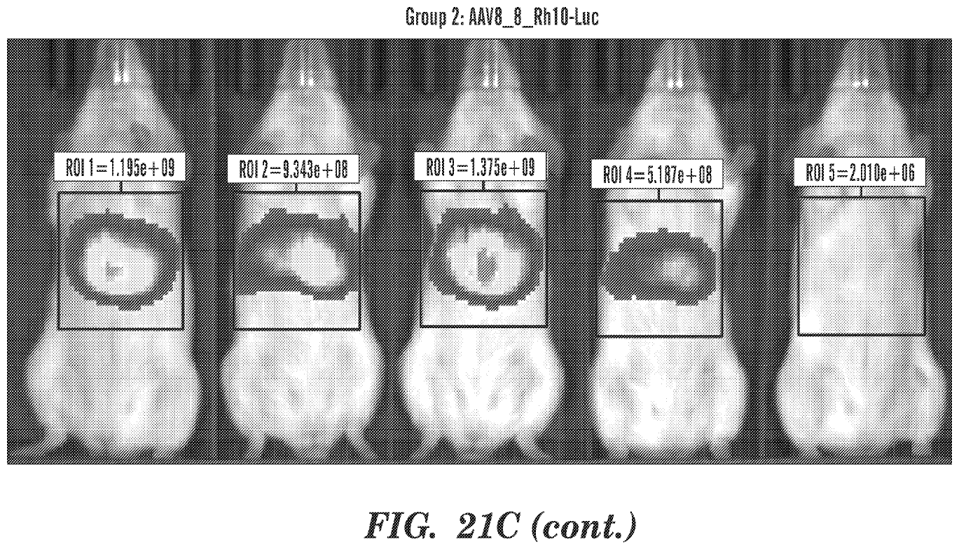

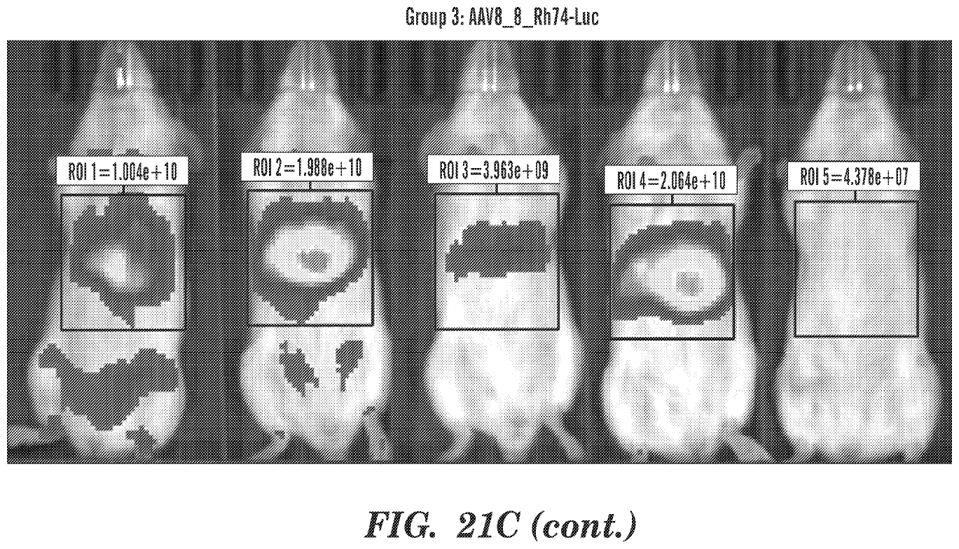

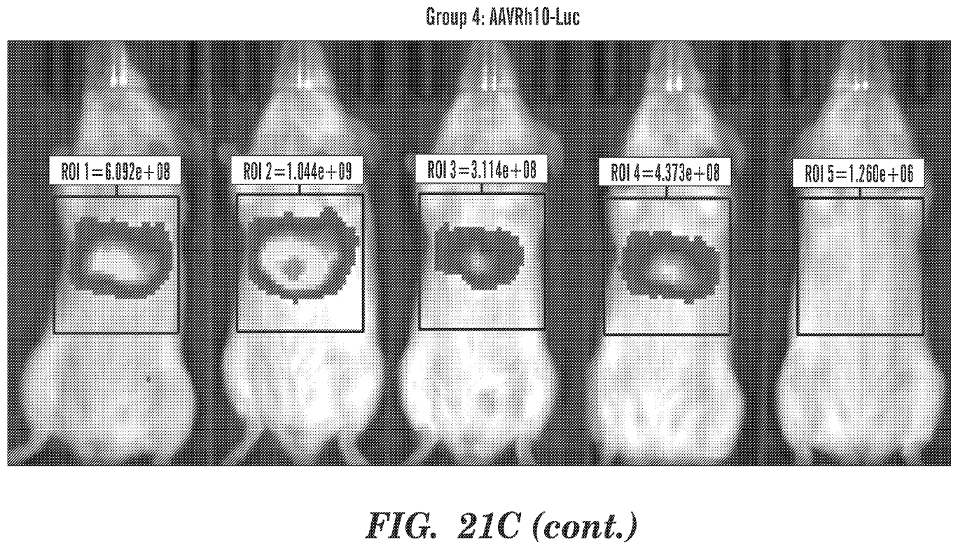

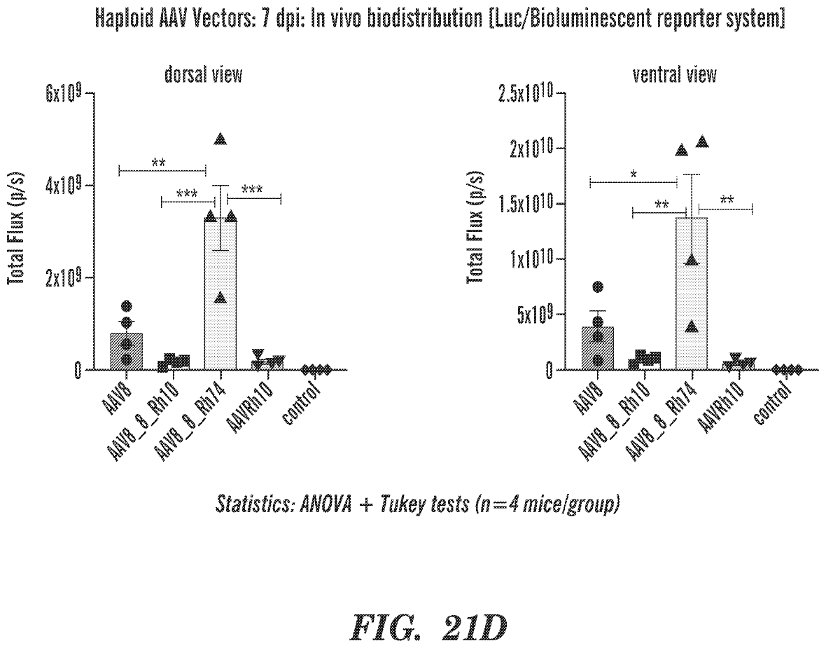

Importantly, as disclosed herein in the Examples, haploid AAV8-8-rh74 vector has an improved ability to escape neutralizing antibodies from sera immunized with parental serotypes. Importantly, AAV8-8-rh74 is surprisingly more efficient in transducing the whole mouse body after systemic administration than parental AAV8 or AAVrh10 serotypes, as well as more efficient than haploid AAV8-8-rh10 (see, e.g., FIG. 21A-21D). Furthermore, AAV8-8-rh74 showed the ability to cross blood brain barrier as opposed to AAV8, AAVrh10 thus showing unexpected result compared to its parent AAV8 and different phenotype shown by AAV8 haploid. Moreover, the haploid vectors AAV8-8-rh74 transduced Pro10 cells similar to AAV8 control, and importantly, transduced GM16095 cells significantly more efficiently than parental vector AAV8. Moreover, the AAV8-8-rh74 haploid was found to escape the anti-AAV8 neutralizing antibodies (see, FIG. 18A-18B; FIG. 19) and produce less humoral response compared to parental AAV8 (FIG. 25A) demonstrating that this AAV8-8-rh74 haploid had a different and less antigenic profile as compared to parental AAV8 vectors.

In some embodiments, the AAV8-8-rh74 haploid was produced using the plasmid of SEQ ID NO: 10 which comprises the construct of SEQ ID NO: 11.

### V. Variants in the AAV Haploid or Polyploid Capsids

In some embodiments the AAV haploid or polyploid virion encompassed herein can be formed by more than the typical 3 viral structural proteins, VP1, VP2, and VP3 (see e.g., Wang, Q. et al., “Syngeneic AAV Pseudo-particles Potentiate Gene Transduction of AAV Vectors,” Molecular Therapy: Methods and Clinical Development, Vol. 4, 149-158 (2017)). Such AAV haploid or polyploid viral capsids also fall within the present invention. For example, an isolated AAV virion having viral capsid structural proteins sufficient to form an AAV haploid or polyploid virion that encapsidates an AAV genome, wherein VP1 is from any AAV serotype listed in Table 1 and VP3 is from any serotype which crosses the BBB. In a further embodiment the isolated AAV haploid or polyploid virion has at least two viral structural proteins from the group consisting of AAV capsid proteins, VP1, VP1.5 and VP3, wherein the two viral proteins are sufficient to form an AAV haploid or polyploid that encapsidates an AAV genome, and wherein VP1 or VP1.5 is from any AAV serotype listed in Table 1 and VP3 is from any serotype which crosses the BBB. For example, the VP1.5 can be from any AAV serotype listed in Table 1 and the VP3 can be from any one or more of the AAV serotypes that cross the BBB, including but not limited to AAV1, AAV6, AAV6.2, AAV7, AAV9, rAAVrh10, rAAVrh74, rAAVrh39, rAAVrh43.

In some embodiments, the capsid of this invention comprises capsid protein VP1.5, wherein said capsid protein VP1.5 is not from the same serotype as VP1 or from the same serotype as the VP3 capsid protein. In some embodiments, the AAV haploid or polyploid capsid protein described herein can comprise capsid protein VP2 as described herein.

In some embodiments, the capsid of this invention comprises capsid protein VP2, wherein said capsid protein VP2 is selected from any of: a AAV8 serotype or any serotype listed in Table 1, or the same serotype as the VP3 protein of the capsid, or a different serotype to that as used for VP3. In some embodiments, the rational polyploid AAV vector described herein can comprise capsid protein VP1.5. VP1.5 is described in U.S. Patent Publication No. 2014/0037585 and the amino acid sequence of VP1.5 is provided herein.

Thus, in certain embodiments the at least one of the viral structural proteins, e.g., VP1, VP2 or VP3 can be a chimeric viral structural protein, i.e., can contain segments from more than one protein. In one embodiment the chimeric viral structural protein is all from the same serotype. In another embodiment, the chimeric viral structural protein is made up of components from a more than one serotype, but these serotypes are different from at least one other serotype. In one embodiment, the viral structural proteins are not chimeric. In one embodiment, the chimeric AAV structural protein does not comprise structural amino acids from canine parvovirus. In one embodiment, the chimeric AAV structural protein does not comprise structural amino acids from b19 parvovirus. In one embodiment, the chimeric AAV structural protein does not comprise structural amino acids from canine parvovirus or b19 parvovirus. In one embodiment, the chimeric AAV structural protein only comprises structural amino acids from AAV. In some embodiments, the rational polyploid AAV vector does not comprise a chimeric VP1 protein. In some embodiments, the rational polyploid AAV vector comprises a chimeric VP1 protein, for example, a chimeric VP1 protein from the AAV8 serotype.

In some embodiments, the rational polyploid AAV virion disclosed herein can be formed by more than the typical 3 viral structural proteins, VP1, VP2, and VP3 (see e.g., Wang, Q. et al., “Syngeneic AAV Pseudo-particles Potentiate Gene Transduction of AAV Vectors,” Molecular Therapy: Methods and Clinical Development, Vol. 4, 149-158 (2017)). Such viral capsids also fall within the present invention. For example, an isolated AAV virion having viral capsid structural proteins sufficient to form an AAV virion that encapsidates an AAV genome, wherein at least one of the viral capsid structural proteins is different from the other viral capsid structural proteins, and wherein each viral capsid structural protein is only of the same type. In a further embodiment the isolated AAV virion has at least two viral structural proteins from the group consisting of AAV capsid proteins, VP1, VP2, VP1.5 and VP3, wherein the two viral proteins are sufficient to form an AAV virion that encapsidates an AAV genome, and wherein at least one of the viral structural proteins present is from a different serotype than the other viral structural protein, and wherein the VP1 is only from one serotype, the VP2 is only from one serotype, and the VP3 is only from one serotype which efficiently crosses the BBB. In one embodiment, the rational polyploid comprises VP1.5. For exemplary purposes only, the VP1.5 can be from AAV serotype 8 and the VP3 can be from a AAV serotype that crosses the BBB and/or alternatively, a AAVrh serotype.

In some embodiments only virions that contain VP1 capsid protein from one serotype (e.g., a serotype listed in Table 1) and VP3 capsid protein from a different serotype to that of VP1 and where the AAV serotype efficiently crosses the BBB are produced. For example, VP1 and VP2 can be from any serotype listed in Table 1, e.g., AAV8 serotype and VP3 is only from an alternative serotype that crosses the BBB. In other embodiments, the VP1 is from only one serotype (e.g., AAV8 serotype) and the VP2 and VP3 are only from another serotype, where the VP2 and VP3 capsid proteins are from a serotype that crosses the BBB. In another embodiment, only particles where VP1 is from a serotype listed in Table 1 (e.g., a AAV8 serotype), VP2 is only from a second serotype, and VP3 only is from yet another serotype which crosses the BBB are produced, and is referred to herein as a polyploid AAV8 virion.

This can be done by, for example, site specific deletions, and/or additions, changing splice donor sites, splice acceptor sites, start codons and combinations thereof.

This permits methods for producing populations of substantially homogenous populations of the polyploid virions-such as the haploid particles.

In some embodiments, the technology herein also provides an AAV capsid wherein the capsid comprises capsid protein VP1, wherein said capsid protein VP1 is from one or more than one first AAV serotype and capsid protein VP2, wherein said capsid protein VP2 is from one or more than one second AAV serotype and wherein at least one of said first AAV serotype is different from at least one of said second AAV serotype, in any combination, and where at least one of VP1, VP2 or VP3 is from a serotype that crosses the BBB, or alternatively from a non-primate AAV serotype, e.g., a rhesus monkey AAV (AAVrh) serotype. In some embodiments no chimeric viral structural protein is present in the virion.

### VI. Modifications to VP1 and VP3 Capsid Proteins

The present invention further provides a composition, which can be a pharmaceutical formulation, comprising the capsid protein, capsid, virus vector, AAV particle composition and/or pharmaceutical formulation of this invention and a pharmaceutically acceptable carrier.

In some non-limiting examples, the present invention provides AAV capsid proteins (VP1, VP1.5, VP2 and/or VP3) comprising a modification in the amino acid sequence in the three-fold axis loop 4 (Opie et al., J. Viral. 77: 6995-7006 (2003)) and virus capsids and virus vectors comprising the modified AAV capsid protein. The rational polyploid or polyploid AAV vectors disclosed herein comprising at least one VP structural protein from a AAV serotype that crosses the BBB, e.g., VP3 from a serotype that crosses the BBB have a different transduction profile after systemic or intrathecal delivery compared to the parental AAV vectors, and surprisingly, have an increased ability to cross the BBB after intrathecal or system delivery and/or transduces an endothelial cell of the BBB, and/or a blood component that crosses the BBB. For example, the AAV8-8-rh74 haploid AAV vector disclosed herein in the Examples shows significant increase in systemic transduction after systematic administration, therefore increasing transduction of each target tissues such as skeletal muscle, cardiac muscle and the like.

In an embodiment, the modified AAV capsid can be comprised of a VP1, a VP2 and/or a VP3 that is created through DNA shuffling to develop cell type specific vectors through directed evolution. DNA shuffling with AAV is generally descried in Li, W. et al., Mol. Ther. 16(7): 1252-12260 (2008), which is incorporated herein by reference. In an embodiment, DNA shuffling can be used to create a VP1, a VP2 and/or a VP3 using the DNA sequence for the capsid genes from two or more different AAV serotypes, AAV chimeras or other AAV. In an embodiment, a haploid AAV can be comprised of a VP1 created by DNA shuffling, a VP2 created by DNA shuffling and/or a VP3 created by DNA shuffling. In some embodiments, the VP1 protein of a rational polyploid virion as disclosed herein is modified, and the VP3 is not a modified protein. In some embodiments, the rational polyploid virion comprises a modified VP3 protein, and where the VP1 is not modified. In some embodiments, a rational polyploid virion disclosed herein comprises a modified VP1 protein and a modified VP3 protein.

Examples of modified VP1 proteins include, but are not limited to, insertion of a peptide in the VP1 protein. Such peptides include, but are not limited to peptides that are targeting peptides, such as peptides targeting cells of the CNS or PNS as disclosed herein. In some embodiments, a targeting peptide is a peptide that penetrates the BBB, for example, a RVG-9R peptide or variant thereof as disclosed in U.S. Pat. No. 8,748,567 or 9,757,470, which are incorporated herein in their entirety by reference. In some embodiments, a peptide can be inserted into, or substituted into at any position selected from between amino acid residues 450-480, amino acid residues 575-600 of the native AAV8 VP1 capsid protein, and/or AAV8 VP2 capsid protein of the polyploid virion (numbering based on AAV8 VP1 numbering) or the corresponding positions of the capsid protein from another AAV.

In another embodiment, the nucleic acid encoding VP1, VP2 and/or VP3 can be created through DNA shuffling. In one embodiment, a first nucleic acid created by DNA shuffling would encode VP1. In this same embodiment, a second nucleic acid created by DNA shuffling would encode VP2 and VP3. In another embodiment, a first nucleic acid created by DNA shuffling would encode VP1. In this same embodiment, a second nucleic acid created by DNA shuffling would encode VP2 and a third nucleic acid would encode VP3. In a further embodiment, a first nucleic acid created by DNA shuffling would encode VP1 and VP2 and a second nucleic acid created by DNA shuffling would encode VP3. In an additional embodiment, a first nucleic acid created by DNA shuffling would encode VP1 and VP3 and a second nucleic acid created by DNA shuffling would encode VP2.

In embodiments of the invention, the rational polyploid vectors disclosed herein have increased transduction of one or more tissues in the CNS and/or peripheral nervous system (PNS). In some embodiments, a rational polyploid vector disclosed herein has enhanced transduction to one or more of: cortex, striatum, thalamus, medulla, hippocampus, cerebellum and spinal cord after systemic or intrathecal administration of a subject in vivo relative to a non-rational polyploid AAV particle that lacks ability to efficiently cross blood brain barrier. In some embodiments, the rational polyploid or polyploid vectors disclosed herein have at least about 50%, 60%, 70%, 80%, 90% or 95%, or 2-fold, five-fold, ten-fold, 50-fold, 100-fold, 1000-fold or higher than 1000-fold transduction levels in the brain and/or spinal cord as compared to a parental AAV vector.

In particular embodiments, the modified AAV capsid protein of the invention comprises one or more modifications in the amino acid sequence of the three-fold axis loop 4 (e.g., amino acid positions 575 to 600 [inclusive] of the native AAV8 VP1 capsid protein or the corresponding region of a capsid protein from another AAV). As used herein, a “modification” in an amino acid sequence includes substitutions, insertions and/or deletions, each of which can involve one, two, three, four, five, six, seven, eight, nine, ten or more amino acids. In particular embodiments, the modification is a substitution. For example, in particular embodiments, 1, 2, 3, 4, 5, 6, 7, 8, 9, 10, 11, 12, 13, 14, 15, 16, 17, 18, 19, 20, 21, 22, 23, 24, 25 or more amino acids from the three-fold axis loop 4 from one AAV can be substituted into at any position selected from between amino acid residues 450-480, amino acid residues 575-600 of the native AAV8 VP1 capsid protein, and/or AAV8 VP2 capsid protein of the polyploid virion (numbering based on AAV8 VP1 numbering) or the corresponding positions of the capsid protein from another AAV. In some embodiments, 1, 2, 3, 4, 5, 6, 7, 8, 9, 10, 11, 12, 13, 14, 15, 16, 17, 18, 19, 20, 21, 22, 23, 24, 25 or more amino acids can be inserted into at any position selected from between amino acid residues 450-480, amino acid residues 575-600 of native AAV8 VP1 and/or, AAV 8 VP2 viral structural protein of rational polyploid virion. In some embodiments, the insertion is a peptide, including but not limited to a targeting peptide, or a peptide that penetrates the BBB. In some embodiments the insertion is a RVG-9R peptides as disclosed in U.S. Pat. No. 8,748,567 or 9,757,470, which are incorporated herein in their entirety by reference. However, the modified virus capsids of the invention are not limited to AAV capsids in which amino acids from one AAV capsid are substituted into another AAV capsid, and the substituted and/or inserted amino acids can be from any source, and can further be naturally occurring or partially or completely synthetic.

As described herein, the nucleic acid and amino acid sequences of the capsid proteins from a number of AAV are known in the art. Thus, the amino acids “corresponding” to amino acid positions 575 to 600 (inclusive) or amino acid positions 585 to 590 (inclusive) of the native AAV8 capsid protein can be readily determined for any other AAV (e.g., by using sequence alignments).

In some embodiments, the invention contemplates that the modified capsid proteins of the invention can be produced by modifying the capsid protein of any AAV now known or later discovered. Further, the AAV capsid protein that is to be modified can be a naturally occurring AAV capsid protein (e.g., an AAV8, AAVrh10, AAVrh74, AAV3a or 3b, AAV9, capsid protein or any of the AAV shown in Table 1) but is not so limited. Those skilled in the art will understand that a variety of manipulations to the AAV capsid proteins are known in the art and the invention is not limited to modifications of naturally occurring AAV capsid proteins. For example, the capsid protein to be modified may already have alterations as compared with naturally occurring AAV (e.g., is derived from a naturally occurring AAV capsid protein, e.g., AAV8, AAVrh10, AAVrh74, AAV3a or 3b, AAV9 or any other AAV now known or later discovered). Such AAV capsid proteins are also within the scope of the present invention.

For example, in some embodiments, the AAV capsid protein to be modified can comprise an amino acid insertion directly following amino acid 264 of the native AAV8 capsid protein sequence (see, e.g., PCT Publication WO 2006/066066) and/or can be an AAV with an altered HI loop as described in PCT Publication WO 2009/108274 and/or can be an AAV that is modified to contain a poly-His sequence to facilitate purification. As another illustrative example, the AAV capsid protein can have a peptide targeting sequence incorporated therein as an insertion or substitution. Further, the AAV capsid protein can comprise a large domain from another AAV that has been substituted and/or inserted into the capsid protein.

Thus, in particular embodiments, a AAV capsid protein, e.g., VP3 protein to be modified can be derived from a naturally occurring AAV but further comprise one or more foreign sequences (e.g., that are exogenous to the native virus) that are inserted and/or substituted into the capsid protein and/or has been altered by deletion of one or more amino acids.

In some embodiments, the rational polyploid virion comprise AAV rhesus monkey modified, or mutated VP3 structural protein wherein VP1 and VP2 are not AAV rhesus monkey serotype. In certain aspects of the embodiment, the mutated VP3 capsid protein is a mutated AAVrh10VP3 or a mutated AAVrh74 VP3 viral structural protein. In certain aspects of the embodiment, the AAV rhesus monkey mutated VP3 comprises at least one mutation at an amino acid that corresponds to an amino acid selected from the group consisting of: N263, G264, T265, S266, G268, T270, T274, E533, R727 and N737 wherein all amino acid positions correspond to native VP3 In some embodiments, the AAV rhesus monkey mutated VP3 viral structural protein comprise at least one, at least two, at least three, at least four, at least five, at least six, at least seven, at least eight, at least nine, or, all mutations at amino acid positions that correspond to amino acid positions N263, G264, T265, S266, G268, T270, T274, E533, R727 and N737 of native VP1 sequence numbering of AAVrh10. In some embodiments, AAV rhesus monkey rh10 mutated VP3 comprise a mutation selected from the group consisting of N263S, G264A, T265S, S266T, G268A, T270del, T274H, E533K, R727H and N737P. In an exemplary embodiment, AAV rhesus monkey rh10 mutated VP3 comprise N263S, G264A, T265S, S266T, G268A, T270del, T274H, E533K, R727H and N737P (AAVrh10LP2, or rh10LP2). In some embodiments, the modified AAVrh10 VP3 structural protein comprises the amino acids of SEQ ID NO: 14 (rh10-LP2 VP3), or a protein that has at least 85%, 90%, 95% or 98% sequence identity to SEQ ID NO: 14. In some embodiments, the modified AAVrh10 VP3 structural protein comprises the amino acids of SEQ ID NO: 14 (rh10-LP2 VP3), or a protein that has at least 85%, 90%, 95% or 98% sequence identity to SEQ ID NO: 14.

In some aspects of the embodiment, the AAV rhesus monkey mutated VP3 comprise a mutation at an amino acid that corresponds to an amino acid selected from the group consisting of N263, G264, T265, S266, G268, T270, T274, E533, R726 and N736 wherein all amino acid positions correspond to native VP1 sequence numbering of AAV rh74. In some embodiments, the AAV rhesus monkey mutated VP3 viral structural protein comprise at least one, at least two, at least three, at least four, at least five, at least six, at least seven, at least eight, at least nine, or, all mutations at amino acid positions that correspond to amino acid positions N263, G264, T265, S266, G268, T270, T274, E533, R726 and N736 of native VP1 sequence numbering of AAVrh74.

In some embodiments, AAV rhesus monkey rh74 mutated VP3 comprise a mutation selected from the group consisting of N263S, G264A, T265S, S266T, G268A, T270del, T274H, E533K, R726H and N736P. In an exemplary embodiment, AAV rhesus monkey rh74 mutated VP3 comprise N263S, G264A, T265S, S266T, G268A, T270del, T274H, E533K, R726H and N736P (AAVrh74LP2, or rh74LP2). Herein the numberings are based on rh74 VP1 numbering. In some embodiments, the modified AAVrh74 VP3 structural protein comprises the amino acids of SEQ ID NO: 15 (rh74vv-LP2 VP3), or a protein that has at least 85%, 90%, 95% or 98% sequence identity to SEQ ID NO: 15. In some aspects of the embodiment, the mutations are located in loop1(VRI) or, in VR-IV or, in extreme C terminal domain of the VP3 viral structural protein. In some aspects of the embodiment, the mutations are located in at least two of these regions.

In yet another aspect of the embodiment, the mutated AAVrh74VP3 viral structural protein further comprise mutation wherein W or, tryptophan at position 581 is replaced by two subsequent Valine (VV) residues. The rh74VP3 mutations comprising of W581VV, N263S, G264A, T265S, S266T, G268A, T270del, T274H, E533K, R726H and N736P is synonymously represented as AAVrh74VVLP2 (or, rh74VVLP2). In some embodiments, the viral structural protein VP3 of rational polyploid virion of the invention comprises rh74VVLP2. Inventors have rationally designed polyploid virions comprising rh10VP3LP2, rh74VP3LP2, and rh74VVP3LP2 and have tested their properties including their antigenicity e.g., evading neutralizing antibody and/or humoral immune response. In some embodiments, the rational AAV polyploid virions for use in the methods and compositions as disclosed herein are selected from any of: AAV8-8-rh10, AAV 8-8-rh74, AAV8-8-rh74vv, AAV 8-8-rh10LP2, AAV 8-8-rh74LP2, AAV 8-8-rh74LP2 vv are interchangeably called as haploid AAV8-8-rh10, AAV 8-8-rh74, AAV8-8-rh74vv, AAV 8-8-rh10LP2, AAV 8-8-rh74LP2, AAV 8-8-rh74LP2 vv virions

In all aspects of the invention, VP1, VP2, VP3 viral structural protein is interchangeably used with VP1, VP2, VP3 capsid protein. Stated another way, the terms “viral capsid protein” and “viral structural protein” are used interchangeably herein, and refer to VP1, VP2, VP3 viral structural proteins (or, structural viral proteins).

Accordingly, when referring herein to a specific AAV capsid protein (e.g., a VP3 protein selected from any of AAV1, AAV6, AAV6.2, AAV7, AAV8, AAV9, rAAVrh10, rAAVrh74, rAAVrh39, rAAVrh4), or VP1 or VP2 capsid protein from any of the AAV shown in Table 1, etc.), it is intended to encompass the native capsid protein as well as capsid proteins that have alterations other than the modifications of the invention. Such alterations include substitutions, insertions and/or deletions. In particular embodiments, the capsid protein comprises 1, 2, 3, 4, 5, 6, 7, 8, 9, 10, 11, 12, 13, 14, 15, 16, 17, 18, 19 or 20, less than 20, less than 30, less than 40 less than 50, less than 60, or less than 70 amino acids inserted therein (other than the insertions of the present invention) as compared with the native AAV capsid protein sequence. In embodiments of the invention, the capsid protein comprises 1, 2, 3, 4, 5, 6, 7, 8, 9, 10, 11, 12, 13, 14, 15, 16, 17, 18, 19 or 20, less than 20, less than 30, less than 40 less than 50, less than 60, or less than 70 amino acid substitutions (other than the amino acid substitutions according to the present invention) as compared with the native AAV capsid protein sequence. In embodiments of the invention, the capsid protein comprises a deletion of 1, 2, 3, 4, 5, 6, 7, 8, 9, 10, 11, 12, 13, 14, 15, 16, 17, 18, 19 or 20, more than 20, more than 30, more than 40, more than 50, more than 60, or more than 70 amino acids (other than the amino acid deletions of the invention) as compared with the native AAV capsid protein sequence.

Using AAV serotype 8 as an exemplary serotype for VP1 and VP2 for a rational polyploid vector disclosed herein, M1 is the VP1 start codon, T138 is the VP2 start codon, and M203 and M211 are VP3 start codons. While deletion of the start codon, typically by a substitution of M1 and T138 will render expression of VP1 and VP2 inoperative, a similar deletion of the VP3 start codon is not sufficient. This is because the viral capsid ORF contains numerous ATG codons with varying strengths as initiation codons. Thus, in designing a construct that will not express VP3 care must be taken to insure that an alternative VP3 species is not produced. With respect to VP3 either elimination of M204 and M212 is necessary or if VP2 is desired, but not VP3, then deletion of M204 and M212 is typically the best approach (Warrington, K. H. Jr., et al., J. of Virol. 78(12): 6595-6609 (June 2004)). This can be done by mutations such as substitution or other means known in the art. The corresponding start codons in other serotypes can readily be determined as well as whether additional ATG sequences such as in VP3 can serve as alternative initiation codons.

Thus, for example, the term “AAV8 capsid protein” includes AAV capsid proteins having the native AAV8 capsid protein sequence (see native AAV8 VP1 capsid protein: GenBank Accession No. AF513852.1, protein ID: AAN03856.1) as well as those comprising substitutions, insertions and/or deletions (as described in the preceding paragraph) in the native AAV8 capsid protein sequence.

In particular embodiments, the AAV capsid protein has the native AAV capsid protein sequence or has an amino acid sequence that is at least about 70%, 75%, 80%, 85%, 90%, 95%, 97%, 98% or 99% similar or identical to a native AAV capsid protein sequence. For example, in particular embodiments, an “AAV8” capsid protein encompasses the native AAV8 capsid protein sequence as well as sequences that are at least about 75%, 80%<85%, 90%, 95%, 97%, 98% or 99% similar or identical to the native AAV8 capsid protein sequence.

Methods of determining sequence similarity or identity between two or more amino acid sequences are known in the art. Sequence similarity or identity may be determined using standard techniques known in the art, including, but not limited to, the local sequence identity algorithm of Smith & Waterman, Adv. Appl. Math. 2,482 (1981), by the sequence identity alignment algorithm of Needleman & Wunsch, J. Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res. 12, 387-395 (1984), or by inspection.

Another suitable algorithm is the BLAST algorithm, described in Altschul et al., J. Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al., (1997) Nucleic Acids Res. 25, 3389-3402.

The invention also provides a virus capsid comprising, consisting essentially of, or consisting of the modified AAV capsid proteins of the invention. In particular embodiments, the virus capsid is a parvovirus capsid, which may further be an autonomous parvovirus capsid or a dependovirus capsid. Optionally, the virus capsid is an AAV capsid. In particular embodiments, the AAV capsid comprises a VP3 protein selected from any of AAV1, AAV6, AAV6.2, AAV7, AAV9, rAAVrh10, rAAVrh74, rAAVrh39, rAAVrh43, and a VP1 and/or VP2 from any other AAV shown in Table 1 or otherwise known or later discovered, and/or is derived from any of the foregoing by one or more insertions, substitutions and/or deletions.

In embodiments of the present invention, the isolated AAV virion or substantially homogenous population of AAV virions is not a product of expression of a mixture of one nucleic acid helper plasmid that express VP1, VP2 and VP3 of one serotype with another nucleic acid helper plasmid that express VP1, VP2 and VP3 of another serotype, such expression being termed “cross-dressing.”

In embodiments of the present invention, the isolated AAV virion does not comprise a mosaic capsid and the substantially homogenous population of AAV virions does not comprise a substantially homogenous population of mosaic capsids.

### VI. Rational Polyploid AAV Vectors for Treating CNS Disorders and Diseases

In some embodiments, the rational polyploid vectors can comprise a capsid comprising a targeting sequence (also referred to as a target peptide) (e.g., substituted or inserted in the viral capsid) that directs the virus capsid to interact with cell-surface molecules present on a desired target tissue(s) (see, e.g., International Patent Publication No. WO 00/28004 and Hauck et al., (2003) J. Virology 77:2768-2774); Shi et al., Human Gene Therapy 17:353-361 (2006) [describing insertion of the integrin receptor binding motif RGD at positions 520 and/or 584 of the AAV capsid subunit]; and U.S. Pat. No. 7,314,912 [describing insertion of the P1 peptide containing an RGD motif following amino acid positions 447, 534, 573 and 587 of the AAV2 capsid subunit]). Other positions within the AAV capsid subunit that tolerate insertions are known in the art (e.g., positions 449 and 588 described by Grifman et al., Molecular Therapy 3:964-975 (2001)).

For example, some of the virus capsids of the invention have relatively inefficient tropism toward most target tissues of interest (e.g., liver, skeletal muscle, heart, diaphragm muscle, kidney, brain, stomach, intestines, skin, endothelial cells, and/or lungs). A targeting sequence can advantageously be incorporated into these low-transduction vectors to thereby confer to the virus capsid a desired tropism and, optionally, selective tropism for particular tissue(s). AAV capsid proteins, capsids and vectors comprising targeting sequences are described, for example in international patent publication WO 00/28004. As another possibility one or more non-naturally occurring amino acids as described by Wang et al., Annu Rev Biophys Biomol Struct. 35:225-49 (2006)) can be incorporated into the AAV capsid subunit at an orthogonal site as a means of redirecting a low-transduction vector to a desired target tissue(s). These unnatural amino acids can advantageously be used to chemically link molecules of interest to the AAV capsid protein including without limitation: glycans (mannose-dendritic cell targeting); RGD, bombesin or a neuropeptide for targeted delivery to specific cancer cell types; RNA aptamers or peptides selected from phage display targeted to specific cell surface receptors such as growth factor receptors, integrins, and the like. Methods of chemically modifying amino acids are known in the art (see, e.g., Greg T. Hermanson, Bioconjugate Techniques, 1′ edition, Academic Press, 1996).

In representative embodiments, the targeting sequence may be a virus capsid sequence (e.g., an autonomous parvovirus capsid sequence, AAV capsid sequence, or any other viral capsid sequence) that directs infection to a particular cell type(s).

B19 infects primary erythroid progenitor cells using globoside as its receptor (Brown et al., (1993) Science 262:114). The structure of B19 has been determined to 8 Å resolutions (Agbandje-McKenna et al., (1994) Virology 203:106). The region of the B19 capsid that binds to globoside has been mapped between amino acids 399-406 (Chapman et al., (1993) Virology 194:419), a looped out region between β-barrel structures E and F. (Chipman et al., (1996) Proc. Nat. Acad. Sci. USA 93:7502). Accordingly, the globoside receptor binding domain of the B19 capsid may be substituted into the AAV capsid protein to target a virus capsid or virus vector comprising the same to erythroid cells.

In representative embodiments, the exogenous targeting sequence may be any amino acid sequence encoding a peptide that alters the tropism of a virus capsid or virus vector comprising the modified AAV capsid protein. In particular embodiments, the targeting peptide or protein may be naturally occurring or, alternately, completely or partially synthetic. Exemplary targeting sequences include ligands and other peptides that bind to cell surface receptors and glycoproteins, such as RGD peptide sequences, bradykinin, hormones, peptide growth factors (e.g., epidermal growth factor, nerve growth factor, fibroblast growth factor, platelet-derived growth factor, insulin-like growth factors I and II, etc.), cytokines, melanocyte stimulating hormone (e.g., α, β or γ), neuropeptides and endorphins, and the like, and fragments thereof that retain the ability to target cells to their cognate receptors. Other illustrative peptides and proteins include substance P, keratinocyte growth factor, neuropeptide Y, gastrin releasing peptide, interleukin 2, hen egg white lysozyme, erythropoietin, gonadoliberin, corticostatin, β-endorphin, leu-enkephalin, rimorphin, α-neo-enkephalin, angiotensin, pneumadin, vasoactive intestinal peptide, neurotensin, motilin, and fragments thereof as described above. As yet a further alternative, the binding domain from a toxin (e.g., tetanus toxin or snake toxins, such as α-bungarotoxin, and the like) can be substituted into the capsid protein as a targeting sequence. In a yet further representative embodiment, the AAV capsid protein can be modified by substitution of a “nonclassical” import/export signal peptide (e.g., fibroblast growth factor-1 and -2, interleukin 1, HIV-1 Tat protein, herpes virus VP22 protein, and the like) as described by Cleves (Current Biology 7:R318 (1997)) into the AAV capsid protein. Also encompassed are peptide motifs that direct uptake by specific cells, e.g., a FVFLP peptide motif (SEQ ID NO: 26) triggers uptake by liver cells.

Phage display techniques, as well as other techniques known in the art, may be used to identify peptides that recognize any cell type of interest.

The targeting sequence may encode any peptide that targets to a cell surface binding site, including receptors (e.g., protein, carbohydrate, glycoprotein or proteoglycan). Examples of cell surface binding sites include, but are not limited to, heparan sulfate, chondroitin sulfate, and other glycosaminoglycans, sialic acid moieties found on mucins, glycoproteins, and gangliosides, MHC I glycoproteins, carbohydrate components found on membrane glycoproteins, including, mannose, N-acetyl-galactosamine, N-acetylglucosamine, fucose, galactose, and the like.

In particular embodiments, a heparan sulfate (HS) or heparin binding domain is substituted into the virus capsid (for example, in an AAV that otherwise does not bind to HS or heparin). It is known in the art that HS/heparin binding is mediated by a “basic patch” that is rich in arginines and/or lysines. In exemplary embodiments, a sequence following the motif BXXB, where “B” is a basic residue and X is neutral and/or hydrophobic. As one nonlimiting example, BXXB is RGNR (SEQ ID NO: 27). In particular embodiments, BXXB is substituted for amino acid positions 262 through 265 in the native AAV2 capsid protein or the corresponding position in the capsid protein of another AAV.

As yet a further alternative, the targeting sequence may be a peptide that can be used for chemical coupling (e.g., can comprise arginine and/or lysine residues that can be chemically coupled through their R groups) to another molecule that targets entry into a cell.

As another option, the AAV capsid protein or virus capsid of the invention can comprise a mutation as described in WO 2006/066066. For example, the capsid protein can comprise a selective amino acid substitution at amino acid position 263, 705, 708 and/or 716 of the native AAV2 capsid protein or a corresponding change(s) in a capsid protein from another AAV. Additionally, or alternatively, in representative embodiments, the capsid protein, virus capsid or vector comprises a selective amino acid insertion directly following amino acid position 264 of the AAV2 capsid protein or a corresponding change in the capsid protein from other AAV. By “directly following amino acid position X” it is intended that the insertion immediately follows the indicated amino acid position (for example, “following amino acid position 264” indicates a point insertion at position 265 or a larger insertion, e.g., from positions 265 to 268, etc.). The foregoing embodiments of the invention can be used to deliver a heterologous nucleic acid to a cell or subject as described herein.

In some embodiments, the rational polyploid or haploid vectors disclosed herein can be used to deliver a heterologous nucleic acid to a cell, including neuronal and non-neuronal cells in the CNS and/or peripheral nervous system. In some embodiments, the rational polyploid or haploid vectors disclosed herein are useful to treat a medical condition or disease associated with aberrant gene expression of a gene in the CNS tissue or cells, and/or in a PNS tissue or cell. The CNS cell may be, for example, a neuron, an astrocyte, an oligodendrocyte, an ependymal cell or a microglial cell. A CNS tissue can be, e.g., more of cortex, striatum, thalamus, medulla, hippocampus, midbrain, purkinji tissue, cerebellum and spinal cord of a subject, including cervical, throratic and lumbar spinal cords, and medulla and choroid plexus of the CNS.

For example, the rational polyploid or haploid vectors disclosed herein can be used to treat brain diseases including cancers in the brain and brain cancers (e.g., glioblastoma), neurodegenerative diseases, including but not limited to, Alzheimer's disease, Huntington's disease, Parkinson's disease, amytrophic lateral sclerosis (ALS), Dopamine transporter deficiency syndrome (a type of childhood parkinsonism, caused by loss-of-function mutation in a single gene, DAT1/SLC6A3). Other medical conditions or diseases of the CNS useful to be treated using the rational polyploid or haploid vectors disclosed herein include for example, a neurological disease and/or disorder. Such neurological diseases and/or disorders include, but are not limited to, for example: dopamine transporter deficiency syndrome, an attention deficit/hyperactivity disorder (ADHD), bipolar disorder, epilepsy, multiple sclerosis, tauopathies, Krabbe's disease, adrenoleukodystrophy, motor neurone disease, cerebral palsy, Batten disease, Gaucher disease, Tay Sachs disease, Rett syndrome, Sandhoff disease, Charcot-Marie-Tooth disease, Angelman syndrome, Canavan disease, Late infantile neuronal ceroid lipofuscinosis, Mucopolysaccharidosis IIIA, Mucopolysaccharidosis IIIB, Metachromatic leukodystrophy, heritable lysosomal storage diseases such as Niemann-Pick disease type C1, and/or neuronal ceroid lipofuscinoses such as Batten disease, progressive supranuclear palsy, corticobasal syndrome, and brain cancer (including astrocytomas and glioblastomas). In some preferred embodiments of the present invention, the gene encodes a therapeutic expression product, preferably a therapeutic polypeptide suitable for use in treating a disease or condition associated with aberrant gene expression, optionally in the CNS.

In some embodiments, a rational polyploid or haploid vector as disclosed herein can be used to express therapeutic expression products useful in the treatment of CNS diseases. The term “CNS disease” is, in principle, understood by the skilled person. The term relates to a disease amenable to treatment and/or prevention by administration of an active compound to the CNS, in particular to a CNS cell. In some embodiments, the CNS disease is a neurological disease and/or disorder.

As a non-limiting example, the CNS disease may be selected from: Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS-Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia-Semantic, Dementia-Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus-Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus-Neurological Sequelae, Lyme Disease-Neurological Complications, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Von Economo's Disease, Von Hippel-Lindau Disease (VHL), Von Recklinghausen's Disease, Wallenberg's Syndrome, Werdnig-Hoffinan Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy.

In some embodiments, the CNS disease is selected from the list consisting of: dopamine transporter deficiency syndrome, an attention deficit/hyperactivity disorder (ADHD), bipolar disorder, epilepsy, multiple sclerosis, tauopathies, Alzheimer's disease, Huntington's disease, Parkinson's disease, Krabbe's disease, adrenoleukodystrophy, motor neurone disease, cerebral palsy, Batten disease, Gaucher disease, Tay Sachs disease, Rett syndrome, Sandhoff disease, Charcot-Marie-Tooth disease, Angelman syndrome, Canavan disease, Late infantile neuronal ceroid lipofuscinosis, Mucopolysaccharidosis IIIA, Mucopolysaccharidosis IIIB, Metachromatic leukodystrophy, heritable lysosomal storage diseases such as Niemann-Pick disease type C1, and/or neuronal ceroid lipofuscinoses such as Batten disease, progressive supranuclear palsy, corticobasal syndrome, and brain cancer (including astrocytomas and glioblastomas).

In some embodiments, a rational polyploid or haploid vector as disclosed herein can be used to express therapeutic expression products useful in the treatment of diseases selected from any of: Methylmalonic acidemia (MMA), alpha 1 anti-trypsin deficiency (AATD), autosomal dominant polycystic kidney disease (ADPKD).

In some embodiments, a rational polyploid or haploid vector as disclosed herein can be used to express therapeutic expression products useful in the treatment of diseases selected from any of: Muscular dystrophies (including myotonic dystrophy (DM1 and DM2), Limb Girdle MD, Duchenne MD, Becker MD, Congenital MD, facioscapulohumeral MD, Emery-Dreifuss MD, Distal MD, Oculopharyngeal MD, Collagen Type VI-related MDs).

In some embodiments, a rational polyploid or haploid vector as disclosed herein can be used to express therapeutic expression products useful in the treatment of diseases of gastrointestinal origin, or a gastrointestinal disorder, for example, any gastrointestinal disorder or disorder of the small intestine selected from: Inflammatory bowel disease (IBD, including ulcerative colitis and Crohn's disease), irritable bowel syndrome (IBS), celiac disease, hereditary hemochromatosis, Lynch syndrome, familial adenomatous polyposis, juvenile polyposis syndrome, Peutz-Jerghers syndrome, eosinophilic gastrointestinal diseases (e.g., eosinophilic gastroenteritis (EGE)), microvillus inclusion disease, megacystis microcolon intestinal hypoperistalsis syndrome, mitochondrial neurogastrointestinal encephalopathy syndrome, intestinal lymphangiectasia, autoimmune gastrointestinal dysmotility, tropical sprue, Whipple's disease, lactose intolerance, and hereditary amyloidosis.

In certain embodiments, the rational polyploid AAV vectors disclosed herein are administered to a subject prophylactically, to prevent on-set of disease. In another embodiment, the AAV particles of the present disclosure are administered to treat (lessen the effects of) a disease or symptoms thereof. In yet another embodiment, the rational polyploid AAV vectors disclosed herein are administered to cure (eliminate) a disease. In another embodiment, the rational polyploid AAV vectors disclosed herein are administered to prevent or slow progression of disease. In yet another embodiment, the AAV particles of the present disclosure are used to reverse the deleterious effects of a disease. Disease status and/or progression may be determined or monitored by standard methods known in the art.

In some embodiments, the rational polyploid AAV vectors disclosed herein are useful in the field of medicine for the treatment, prophylaxis, palliation or amelioration of neurological diseases and/or disorders.

In some embodiments, the rational polyploid AAV vectors disclosed herein are useful in the field of medicine for the treatment, prophylaxis, palliation or amelioration of tauopathy.

In some embodiments, the rational polyploid AAV vectors disclosed herein are useful in the field of medicine for the treatment, prophylaxis, palliation or amelioration of Alzheimer's Disease.

In some embodiments, the rational polyploid AAV vectors disclosed herein are useful in the field of medicine for the treatment, prophylaxis, palliation or amelioration of Friedreich's ataxia, or any disease stemming from a loss or partial loss of frataxin protein.

In some embodiments, the rational polyploid AAV vectors disclosed herein are useful in the field of medicine for the treatment, prophylaxis, palliation or amelioration of Parkinson's Disease.

In some embodiments, the rational polyploid AAV vectors disclosed herein are useful in the field of medicine for the treatment, prophylaxis, palliation or amelioration of Amyotrophic lateral sclerosis.

In some embodiments, the rational polyploid AAV vectors disclosed herein are useful in the field of medicine for the treatment, prophylaxis, palliation or amelioration of Huntington's Disease.

In some embodiments, the rational polyploid AAV vectors disclosed herein are useful in the field of medicine for the treatment, prophylaxis, palliation or amelioration of chronic or neuropathic pain.

In some embodiments, the rational polyploid AAV vectors disclosed herein are useful in the field of medicine for treatment, prophylaxis, palliation or amelioration of a disease associated with the central nervous system (CNS).

In some embodiments, the rational polyploid AAV vectors disclosed herein are useful in the field of medicine for treatment, prophylaxis, palliation or amelioration of a disease associated with the peripheral nervous system (PNS).

In certain embodiments, the AAV particles of the present disclosure are administered to a subject having at least one of the diseases or symptoms described herein.

As used herein, any disease associated with the central or peripheral nervous system and components thereof (e.g., neurons) may be considered a “neurological disease”.

Any neurological disease may be treated with the AAV particles of the disclosure, or pharmaceutical compositions thereof, including but not limited to, Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS-Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Bechet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbar palsy, Bulbospinal Muscular Atrophy, Cerebral Autosomal Dominant Arteriopathy with Sub cortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavernous Malformation, Cerebral Gigantism, Cerebral Hypoxia, Cerebral Palsy, Cerebro-Oculo-Facio-Skeletal Syndrome (COFS), Charcot-Marie-Tooth Disease, Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome Type II, Coffin Lowry Syndrome, Colpocephaly, Coma, Complex Regional Pain Syndrome, Concentric sclerosis (Balo's sclerosis), Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Chronic progressive external ophthalmoplegia, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, De Morsier's Syndrome, Dejerine-Klumpke Palsy, Dementia, Dementia-Multi-Infarct, Dementia-Semantic, Dementia-Subcortical, Dementia With Lewy Bodies, Demyelination diseases, Dentate Cerebellar Ataxia, Dentatorubral Atrophy, Dermatomyositis, Developmental Dyspraxia, Devic's Syndrome, Diabetic Neuropathy, Diffuse Sclerosis, Distal hereditary motor neuronopathies, Dravet Syndrome, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalomyelitis, Encephalopathy, Encephalopathy (familial infantile), Encephalotrigeminal Angiomatosis, Epilepsy, Epileptic Hemiplegia, Episodic ataxia, Erb's Palsy, Erb-Duchenne and Dejerine-Klumpke Palsies, Essential Tremor, Extrapontine Myelinolysis, Faber's disease, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gaucher Disease, Generalized Gangliosidoses (GM1, GM2), Gerstmann's Syndrome, Gerstmann-Straussler-Scheinker Disease, Giant Axonal Neuropathy, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hurler syndrome, Hydranencephaly, Hydrocephalus, Hydrocephalus-Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lichtheim's disease, Lipid Storage Diseases, Lipoid Proteinosis, Lissencephaly, Locked-In Syndrome, Lou Gehrig's Disease, Lupus-Neurological Sequelae, Lyme Disease-Neurological Complications, Lysosomal storage disorders, Machado-Joseph Disease, Macrencephaly, Megalencephaly, Melkersson-Rosenthal Syndrome, Meningitis, Meningitis and Encephalitis, Menkes Disease, Meralgia Paresthetica, Metachromatic Leukodystrophy, Microcephaly, Migraine, Miller Fisher Syndrome, Mini Stroke, Mitochondrial Myopathy, Mitochondrial DNA depletion syndromes, Moebius Syndrome, Monomelic Amyotrophy, Morvan Syndrome, Motor Neuron Diseases, Moyamoya Disease, Mucolipidoses, Mucopolysaccharidoses, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myelitis, Myoclonic Encephalopathy of Infants, Myoclonus, Myoclonus epilepsy, Myopathy, Myopathy-Congenital, Myopathy-Thyrotoxic, Myotonia, Myotonia Congenita, Narcolepsy, NARP (neuropathy, ataxia and retinitis pigmentosa), Neuroacanthocytosis, Neurodegeneration with Brain Iron Accumulation, Neurodegenerative disease, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathic pain, Neuropathy-Hereditary, Neuropathy, Neurosarcoidosis, Neurosyphilis, Neurotoxicity, Nevus Cavemosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Peroneal muscular atrophy, Periodic Paralyses, Peripheral Neuropathy, Periventricular Leukomalacia, Persistent Vegetative State, Pervasive Developmental Disorders, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Primary Dentatum Atrophy, Primary Lateral Sclerosis, Primary Progressive Aphasia, Prion Diseases, Progressive bulbar palsy, Progressive Hemifacial Atrophy, Progressive Locomotor Ataxia, Progressive Multifocal Leukoencephalopathy, Progressive Muscular Atrophy, Progressive Sclerosing Poliodystrophy, Progressive Supranuclear Palsy, Prosopagnosia, Pseudobulbar palsy, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease-Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar Ataxia, Spinocerebellar Atrophy, Spinocerebellar Degeneration, Sporadic ataxia, Steele-Richardson-Olszewski Syndrome, Stiff-Person Syndrome, Striatonigral Degeneration, Stroke, Sturge-Weber Syndrome, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central and Peripheral Nervous Systems, Vitamin B 12 deficiency, Von Economo's Disease, Von Hippel-Lindau Disease (VHL), Von Recklinghausen's Disease, Wallenberg's Syndrome, Werdnig-Hoffman Disease, Wemicke-Korsakoff Syndrome, West Syndrome, Whiplash, Whipple's Disease, Williams Syndrome, Wilson Disease, Wolman's Disease, X-Linked Spinal and Bulbar Muscular Atrophy.

In some embodiments, a therapeutic gene or expression product encoded by a rational polyploid or haploid vector as disclosed herein can be, for example, selected from the group consisting of: NPC1, EAAT2, NPY, CYP46A1, GLB1, APOE (e.g. ApoE2, ApoE3 or ApoE4), HEX, CLN1, CLN2, CLN3, CLN4, CLN5, CLN6, SUMF1, DCTN1, PRPH, SOD1, NEFH, GBA, IDUA, NAGLU, GUSB, ARSA, MANB, AADC, GDNF, SOD1, NTN, ASP, MAPT, APOE, HTT, MECP2, PTCHD1, GIB1, UBE3A, HEXA, MOG.

Additionally, or alternatively, the expression product may be an antibody, antibody fragment or anti-body like scaffold protein. In some embodiments, exemplary polypeptide expression products include neuroprotective polypeptides and anti-angiogenic polypeptides. In one aspect, the rational polyploid vectors disclosed herein comprise a viral genome encoding a polypeptide payload. The polypeptide may be, but is not limited to, an antibody, aromatic L-amino acid decarboxylase (AADC), survival motor neuron 1 (SMN1), frataxin (FXN), ApoE2, GBA1, GRN, ASP A, CLN2, GLB1, SGSH, NAGLU, IDS, NPC1, or GAN.

Additional suitable polypeptides include, but are not limited to, glial derived neurotrophic factor (GDNF), fibroblast growth factor 2 (FGF-2), nurturin, ciliary neurotrophic factor (CNTF), nerve growth factor (NGF; e.g., nerve growth factor-. beta.), brain derived neurotrophic factor (BDNF), neurotrophin-3 (NT-3), neurotrophin-4 (NT-4), neurotrophin-6 (NT-6), epidermal growth factor (EGF), pigment epithelium derived factor (PEDF), a Wnt polypeptide, soluble Fit-1, angiostatin, endostatin, VEGF, an anti-VEGF antibody, a soluble VEGFR, Factor VIII (FVIII), Factor IX (FIX), and a member of the hedgehog family (sonic hedgehog, Indian hedgehog, and desert hedgehog, etc.).

In some embodiments, useful therapeutic expression product include hormones and growth and differentiation factors including, without limitation, insulin, glucagon, growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor alpha superfamily, including TGFa., activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Additionally, or alternatively, a therapeutic gene or expression product encoded by a rational polyploid or haploid vector as disclosed herein can be a gene editing system (such as a CRISPR-Cas9 system, TALEN, ZFN, etc.) directed to the disease allele. Additionally, or alternatively, the expression product may be one or more modulatory polynucleotides, e.g., RNA or DNA molecules as therapeutic agents. For example, the modulatory polynucleotide may be a miRNA or siRNA. Target genes may be any of the genes associated with any neurological disease such as, but not limited to, those listed herein. For example, siRNA duplexes or encoded dsRNA can reduce or silence target gene expression in CNS cells, thereby ameliorating symptoms of neurological disease. In one non-limiting example, the target gene is huntingtin (HTT). In another non-limiting example the target gene is microtubule-associated protein tau (MAPT). In some embodiments, useful expression products include proteins that regulate the immune system including, without limitation, cytokines and lymphokines such as thrombopoietin (TPO), interleukins (IL) IL-1 through IL-25 (including IL-2, IL-4, IL-12 and IL-18), monocyte chemoattractant protein, leukemia inhibitory factor, granulocyte-macrophage colony stimulating factor, Fas ligand, tumor necrosis factors alpha and beta., interferons (alpha, beta, and gamma), stem cell factor, flk-2/flt3 ligand. Gene products produced by the immune system are also useful in the present invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

In some embodiments, useful expression product includes any one of the receptors for the hormones, growth factors, cytokines, lymphokines, regulatory proteins and immune system proteins. Useful heterologous nucleic acid sequences also include receptors for cholesterol regulation and/or lipid modulation, including the low-density lipoprotein (LDL) receptor, high density lipoprotein (HDL) receptor, the very low density lipoprotein (VLDL) receptor, and scavenger receptors. The invention also encompasses the use of gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP-2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

In some embodiments, useful expression products include non-naturally occurring polypeptides, such as chimeric or hybrid polypeptides having a non-naturally occurring amino acid sequence containing insertions, deletions or amino acid substitutions. Further suitable expression products include micro RNA (miRNA), interfering RNA, antisense RNA, ribozymes, and aptamers.

In alternative embodiments, the rational polyploid or haploid vectors disclosed herein can be used to deliver a heterologous nucleic acid to a cell in the subject in vivo. In some embodiments, the rational polyploid or haploid vectors disclosed herein can be used to treat any one or more of the following diseases: a lysosomal storage disorder such as a mucopolysaccharidosis disorder (e.g., Sly syndrome [β-glucuronidase], Hurler Syndrome [α-L-iduronidase], Scheie Syndrome [α-L-iduronidase], Hurler-Scheie Syndrome [α-L-iduronidase], Hunter's Syndrome [iduronate sulfatase], Sanfilippo Syndrome A [heparan sulfamidase], B [N-acetylglucosaminidase], C [acetyl-CoA: α-glucosaminide acetyltransferase], D [N-acetylglucosamine 6-sulfatase], Morquio Syndrome A [galactose-6-sulfate sulfatase], B [β-galactosidase], Maroteaux-Lamy Syndrome [N-acetylgalactosamine-4-sulfatase], etc.), Fabry disease (α-galactosidase), Gaucher's disease (glucocerebrosidase), or a glycogen storage disorder (e.g., Pompe disease; lysosomal acid α-glucosidase) as described herein.

Those skilled in the art will appreciate that for some AAV capsid proteins the corresponding modification will be an insertion and/or a substitution, depending on whether the corresponding amino acid positions are partially or completely present in the virus or, alternatively, are completely absent. Likewise, when modifying AAV other than AAV2, the specific amino acid position(s) may be different than the position in AAV2 (see, e.g., Table 3). As discussed elsewhere herein, the corresponding amino acid position(s) will be readily apparent to those skilled in the art using well-known techniques.

In representative embodiments, the insertion and/or substitution and/or deletion in the capsid protein(s) results in the insertion, substitution and/or repositioning of an amino acid that (i) maintains the hydrophilic loop structure in that region; (ii) an amino acid that alters the configuration of the loop structure; (iii) a charged amino acid; and/or (iv) an amino acid that can be phosphorylated or sulfated or otherwise acquire a charge by post-translational modification (e.g., glycosylation) following 264 in an AAV2 capsid protein or a corresponding change in a capsid protein of another AAV. Suitable amino acids for insertion/substitution include aspartic acid, glutamic acid, valine, leucine, lysine, arginine, threonine, serine, tyrosine, glycine, alanine, proline, asparagine, phenylalanine, tyrosine or glutamine. In particular embodiments, a threonine is inserted or substituted into the capsid subunit. Nonlimiting examples of corresponding positions in a number of other AAV are shown in Table 3 (Position 2). In particular embodiments, the amino acid insertion or substitution is a threonine, aspartic acid, glutamic acid or phenylalanine (excepting AAV that have a threonine, glutamic acid or phenylalanine, respectively, at this position).

In further embodiments, the modified capsid protein or capsid can comprise a mutation as described in WO 2009/108274.

As another, possibility, the AAV capsid protein can comprise a mutation as described by Zhong et al. (Virology 381: 194-202 (2008); Proc. Nat. Acad. Sci. 105: 7827-32 (2008)). For example, the AAV capsid protein can comprise an YF mutation at amino acid position 730.

The modifications described above can be incorporated into the capsid proteins or capsids of the invention in combination with each other and/or with any other modification now known or later discovered.

The invention also encompasses virus vectors comprising the modified capsid proteins and capsids of the invention. In particular embodiments, the virus vector is a parvovirus vector (e.g., comprising a parvovirus capsid and/or vector genome), for example, an AAV vector (e.g., comprising an AAV capsid and/or vector genome). In representative embodiments, the virus vector comprises a modified AAV capsid comprising a modified capsid protein subunit of the invention and a vector genome.

For example, in representative embodiments, the virus vector comprises: (a) a modified virus capsid (e.g., a modified AAV capsid) comprising a modified capsid protein of the invention; and (b) a nucleic acid comprising a terminal repeat sequence (e.g., an AAV TR), wherein the nucleic acid comprising the terminal repeat sequence is encapsidated by the modified virus capsid. The nucleic acid can optionally comprise two terminal repeats (e.g., two AAV TRs).

In representative embodiments, the virus vector is a recombinant virus vector comprising a heterologous nucleic acid encoding a polypeptide or functional RNA of interest. Recombinant virus vectors are described in more detail below.

In some embodiments, the rational polyploid (e.g., rational haploid) AAV vectors disclosed herein have (i) increased ability to cross the BBB after systemic or intrathecal administration as compared to non-polyploid parental AAV vectors, (ii) increased biodistribution in the CNS or PNS or both, and/or ability to transduce brain tissues (e.g., neurons and non-neuronal cells) as compared with the level of transduction by a non-haploid parental AAV vectors, (iii) exhibit enhanced systemic transduction by the virus vector in an animal subject as compared with the level observed by the level of transduction by a non-rational polyploid parental AAV vectors; (iv) decreased humoral immune response as compared to the level of immune response elicited by a non-rational polyploid parental AAV vectors, (v) decreased neutralization by neutralizing antibodies against the parental AAV vector as compared to the level of neutralization of non-polyploid parental AAV vectors to same neutralizing antibodies.

Further, in some embodiments of the invention, the rational polyploid or haploid AAV vectors as disclosed herein demonstrate efficient transduction of CNS and/or PNS target tissues, including but not limited to cortex, striatum, thalamus, medulla, hippocampus, midbrain, purkinji tissue, cerebellum and spinal cord of a subject, including cervical, throratic and lumbar spinal cords, and medulla and choroid plexus of the CNS.

In some embodiments, a population of rational polyploid AAV virions that allow repeat dosing, the population comprising: at least one of AAV VP1, or, VP2 viral structural proteins and a AAV VP3 viral structural protein; wherein, the VP1 and VP2 viral structural proteins are each from AAV 8 viral serotype, and the VP3 viral structural protein is selected from a rhesus monkey AAV rh10 serotype; wherein, the population of rational polyploid AAV 8-8-rh10 virions elicits a reduced humoral response as compared to the humoral response elicited by the parental AAV 8 serotype, and wherein, the repeat dosing comprises a first administration of the population of rational polyploid AAV 8-8-rh10 virions and a second administration of a parental AAV serotype 8 virion. For illustrative purposes, the humoral response as disclosed herein is measured by serum levels of anti AAV8 antibody (e.g IgG) after a subject or animal e.g mice is injected with either the rational polyploid AAV 8-8-rh10 virion or, with AAV8 virion. The reduced humoral response therefore, directs to producing less anti AAV8 IgG when a subject or, animal is administered with rational polyploid (e.g rational haploid) AAV 8-8-rh10 virion as compared to the anti AAV 8 IgG produced when a subject or, animal is administered with AAV 8 virion under similar condition. The less anti AAV8 IgG produced with the rational polyploid (e.g rational haploid) AAV 8-8-rh10 makes it more suitable for using as a gene therapy regimen wherein a second administration is required. For this particular example of an AAV gene therapy regimen, it is suitable to use rational polyploid AAV 8-8-rh10 as a first administration with a subsequent or second administration with AAV8 vector.

In some embodiments, a population of rational polyploid AAV virions that allow repeat dosing, the population comprising: at least one of AAV VP1, or, VP2 viral structural proteins and a AAV VP3 viral structural protein; wherein, the VP1 and VP2 viral structural proteins are each from AAV 8 viral serotype, and the VP3 viral structural protein is selected from a rhesus monkey AAV rh74 serotype; wherein, the population of rational polyploid AAV 8-8-rh74 virions elicits a reduced humoral response as compared to the humoral response elicited by the parental AAV 8 serotype, and wherein, the repeat dosing comprises a first administration of the population of rational polyploid AAV 8-8-rh74 virions and a second administration of a parental AAV serotype 8 virion. For illustrative purposes, the humoral response as disclosed herein is measured by serum levels of anti AAV8 antibody (e.g IgG) after a subject or animal e.g mice is injected with either the rational polyploid AAV 8-8-rh74 virion or, with AAV8 virion.

The reduced humoral response therefore, directs to producing less anti AAV8 IgG when a subject or, animal is administered with rational polyploid (e.g rational haploid) AAV 8-8-rh74 virion as compared to the anti AAV 8 IgG produced when a subject or, animal is administered with AAV 8 virion under similar condition. The less anti AAV8 IgG produced with the rational polyploid (e.g rational haploid) AAV 8-8-rh74 makes it more suitable for using as a gene therapy regimen wherein a second administration is required. For this particular example of an AAV gene therapy regimen, it is suitable to use rational polyploid AAV 8-8-rh74 as a first administration with a subsequent or second administration with AAV8 vector, or vice versa.

Herein, the AAV vector and AAV virion is used interchangeably.

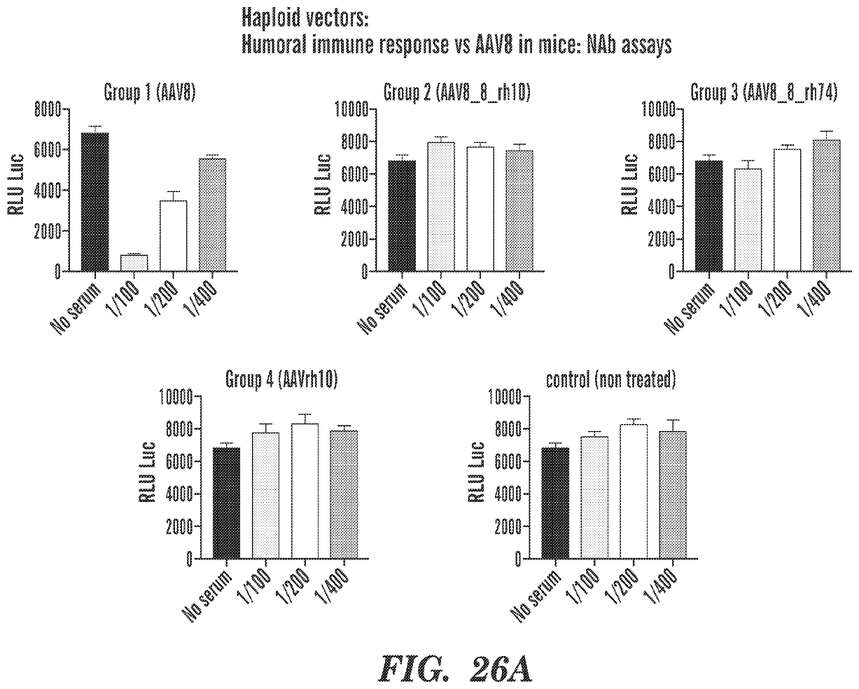

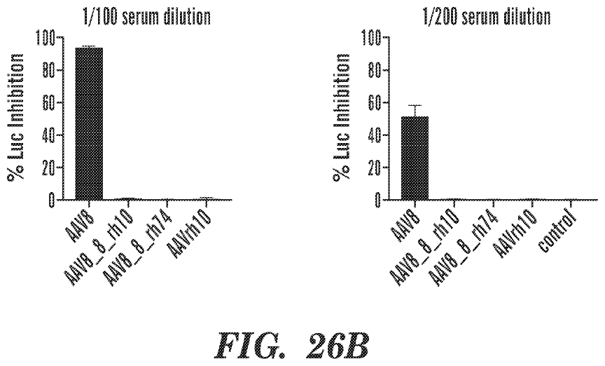

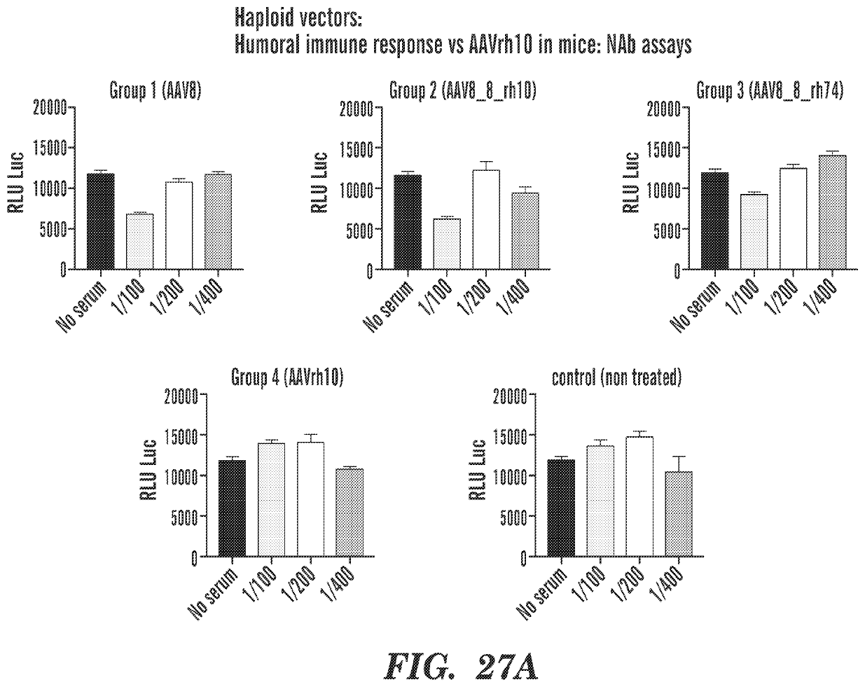

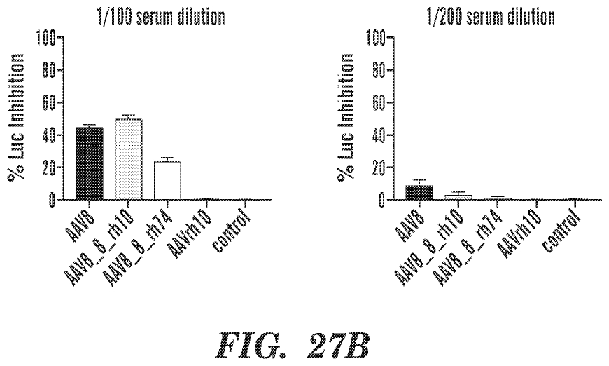

In some embodiments, a population of rational polyploid AAV virions that allow repeat dosing, the population comprising: at least one of AAV VP1, or, VP2 viral structural proteins and a AAV VP3 viral structural protein; wherein, the VP1 and VP2 viral structural proteins are each from AAV 8 viral serotype, and the VP3 viral structural protein is selected from a rhesus monkey AAV rh10 serotype or, AAV rh74 serotype; wherein, the population of rational polyploid AAV 8-8-rh10 virions or, AAV 8-8-rh74 virions elicits a reduced humoral response as compared to the humoral response elicited by the parental AAV 8 serotype, and wherein, the repeat dosing comprises a first administration of a parental AAV serotype 8 virion and a second administration of the population of rational polyploid AAV 8-8-rh10 or, AAV 8-8-rh74 virions. For illustrative purposes, in this particular example, AAV 8-8-rh10 or, AAV 8-8-rh74 elicits reduced humoral response e.g produces less anti AAV8 IgG compared to that produced by AAV8 virion. This AAV gene therapy regimen with first administration with parental AAV serotype, e.g AAV 8 and second administration with rational polyploid (e.g, AAV 8-8-rh10, or, AAV 8-8-rh74) is suitable to have efficient transduction with second administration of rational polyploid (e.g., AAV 8-8-rh10 or, AAV 8-8-rh74). As shown in FIG. 26, first administration with AAV8 produces anti AAV8 IgG in serum that can neutralize or, inhibit the AAV 8 mediated transduction whereas AAV 8-8-rh10 or AAV 8-8-rh74 mediated transduction is not inhibited in presence of the serum containing anti AAV 8 IgG thus confirming the suitable use of AAV 8-8-rh10 or, AAV 8-8-rh74 as a second administration wherein the first administration is with AAV 8.

In some embodiments, a population of rational polyploid AAV virions that allow repeat dosing, the population comprising: at least one of AAV VP1, or, VP2 viral structural proteins and a AAV VP3 viral structural protein; wherein, the VP1 and VP2 viral structural proteins are each from any AAV viral serotype, and the VP3 viral structural protein is selected from a rhesus monkey AAV serotype; wherein, the population of rational polyploid AAV virions elicits a reduced humoral response as compared to the humoral response elicited by the parental AAV serotype of the VP1 or VP2 viral structural proteins, wherein, the VP1 and VP2 are not from a Rhesus AAV serotype, and wherein, the repeat dosing comprises a first administration of a parental AAV serotype of the VP1 structural viral protein or, VP2 structural viral protein and a second administration of the population of rational polyploid AAV virions.

In some embodiments, a method for repeat dosing comprising a first and second administrations, wherein, the repeat dosing comprises the first administration of parental AAV serotypes of VP1 or VP2 viral structural protein, and the second administration of a rational polyploid AAV virion wherein the VP3 viral structural protein of the rational polyploid virion is from an AAV serotype that efficiently crosses blood brain barrier and is different from the serotype of at least one of VP1 or, VP2 viral structural protein, wherein the population of rational polyploid virion elicits a reduced humoral response as compared to the humoral response as elicited by the parental AAV serotypes of VP1 or VP2 viral structural protein, and wherein, VP1 or, VP2 is not from a Rhesus AAV serotype.

It will be understood by those skilled in the art that the modified capsid proteins, virus capsids, virus vectors and AAV particles of the invention exclude those capsid proteins, capsids, virus vectors and AAV particles as they would be present or found in their native state.

### VII. Methods of Producing Virus Vectors

The AAV rational vectors disclosed herein can be produced by any means well known in the art. As an illustrative example only, using AAV haploid virion as an exemplary example, the method comprises (a) transfecting a host cell with one or more plasmids that provide, in combination all functions and genes needed to assemble AAV haploid particles; (b) introducing one or more nucleic acid constructs into a packaging cell line or producer cell line to provide, in combination all functions and genes needed to assemble AAV haploid particles; (c) introducing into a host cell one or more recombinant baculovirus vectors that provide in combination all functions and genes needed to assemble AAV haploid particles; and/or (d) introducing into a host cell one or more minicircle or using closed linear DNA (clDNA) or, barbell shaped DNA that provide in combination all functions and genes needed to assemble AAV haploid particles.

The disclosed herein further provides methods of producing the rational polyploid or haploid AAV vectors as disclosed herein as AAV particles. Thus, the present invention provides a method of making an AAV haploid virion particle comprising the rational polyploid AAV vector of this invention, comprising: (a) transfecting a host cell with one or more plasmids that provide, in combination all functions and genes needed to assemble AAV haploid vector particles; (b) introducing one or more nucleic acid constructs into a packaging cell line or producer cell line to provide, in combination, all functions and genes needed to assemble AAV particles; (c) introducing into a host cell one or more recombinant baculovirus vectors that provide in combination all functions and genes needed to assemble AAV particles; and/or (d) introducing into a host cell one or more recombinant herpesvirus vectors that provide in combination all functions and genes needed to assemble AAV particles. The conditions for formation of an AAV haploid virion are the standard conditions for production of AAV vectors in cells (e.g., mammalian or insect cells), which includes as a nonlimiting example transfection of cells in the presence of an Ad helper plasmid, or other helper virus such as HSV.

Nonlimiting examples of various methods of making the virus vectors of this invention are described in Clement and Grieger (“Manufacturing of recombinant adeno-associated viral vectors for clinical trials” Mol. Ther. Methods Clin Dev. 3:16002 (2016)) and in Grieger et al. (“Production of recombinant adeno-associated virus vectors using suspension HEK293 cells and continuous harvest of vector from the culture media for GMP FIX and FLT1 clinical vector” Mol Ther 24(2):287-297 (2016)), the entire contents of which are incorporated by reference herein.

In one representative embodiment, the technology also provides a method of producing a virus vector, the method comprising providing to a cell: (a) a nucleic acid template comprising at least one TR sequence (e.g., AAV TR sequence), and (b) AAV sequences sufficient for replication of the nucleic acid template and encapsidation into AAV capsids (e.g., AAV rep sequences and AAV cap sequences encoding the AAV capsids of the invention). Optionally, the nucleic acid template further comprises at least one heterologous nucleic acid sequence. In particular embodiments, the nucleic acid template comprises two AAV ITR sequences, which are located 5′ and 3′ to the heterologous nucleic acid sequence (if present), although they need not be directly contiguous thereto.

The nucleic acid template and AAV rep and cap sequences are provided under conditions such that virus vector comprising the nucleic acid template packaged within the AAV capsid is produced in the cell. The method can further comprise the step of collecting the virus vector from the cell. The virus vector can be collected from the medium and/or by lysing the cells.

In one embodiment and as disclosed herein in the Examples, the nucleic acid template is altered so that the capsid (Cap) sequences cannot express all three viral structural proteins, VP1, VP2, and VP3 from a nucleic acid sequence only from one serotype (first nucleic acid sequence). This alteration can be by, for example, eliminating start codons for at least one of the viral structural proteins. The template will also contain at least one additional nucleic acid sequence (second nucleic acid sequence) from a different serotype encoding and capable of expressing the viral structural protein not capable of being expressed by the first nucleic acid sequence, wherein the second nucleic acid sequence is not capable of expressing the viral structural protein capable of expression by the first nucleic acid sequence. In one embodiment, the first nucleic acid sequence is capable of expressing two of the viral structural proteins whereas the second nucleic acid sequence is capable of expressing only the remaining viral sequence. For example, the first nucleic acid sequence is capable of expression of VP1 and VP2 but not VP3 from one serotype and the second nucleic acid sequence is capable of expression of VP3 from an alternative serotype, but not VP1 or VP2. The template is not capable of expressing any other of the three viral structural proteins. In one embodiment the first nucleic acid sequence is only capable of expressing one of the three viral structural proteins, the second nucleic acid sequence is capable of expressing only the other two viral structural proteins, but not the first.

In another embodiment there is a third nucleic acid sequence from a third serotype. In this embodiment each of the three nucleic acid sequences is only capable of expressing one of the three capsid viral structural proteins, VP1, VP2, and VP3, and each does not express a viral structural protein expressed by another of the sequences so that collectively a capsid is produced containing VP1, VP2, and VP3, wherein each of the viral structural proteins in the capsid are all from the same serotype only and in this embodiment VP1, VP2, and VP3 are all from different serotypes.

The alteration to prevent expression can be by any means known in the art. For example, eliminating start codons, splice acceptors, splice donors, and combinations thereof. Deletions and additions can be use as well as site specific changes to change reading frames. Nucleic acid sequences can also be synthetically produced. These helper templates typically do not contain ITRs.

The cell can be a cell that is permissive for AAV viral replication. Any suitable cell known in the art may be employed. In particular embodiments, the cell is a mammalian cell. As another option, the cell can be a trans-complementing packaging cell line that provides functions deleted from a replication-defective helper virus, e.g., 293 cells or other Ela trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the Ela or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67).

As a further alternative, the rep/cap sequences may be stably incorporated into a cell. Typically, the AAV rep/cap sequences will not be flanked by the terminal repeats (TRs), to prevent rescue and/or packaging of these sequences.

The nucleic acid template can be provided to the cell using any method known in the art. For example, the template can be supplied by a non-viral (e.g., plasmid) or viral vector. In particular embodiments, the nucleic acid template is supplied by a herpesvirus or adenovirus vector (e.g., inserted into the E1a or E3 regions of a deleted adenovirus). As another illustration, Palombo et al., (1998) J Virology 72:5025, describes a baculovirus vector carrying a reporter gene flanked by the AAV TRs. EBV vectors may also be employed to deliver the template, as described above with respect to the rep/cap genes.

In another representative embodiment, the nucleic acid template is provided by a replicating rAAV virus. In still other embodiments, an AAV provirus comprising the nucleic acid template is stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a non-infectious adenovirus miniplasmid that carries all of the helper genes that promote efficient AAV production as described by Ferrari et al., (1997) Nature Med. 3:1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper viruses sequences cannot be packaged into AAV virions, e.g., are not flanked by TRs.

Those skilled in the art will appreciate that it may be advantageous to provide the AAV replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In one particular embodiment, the AAV rep/cap sequences and the adenovirus helper sequences are supplied by a single adenovirus helper vector. This vector further can further comprise the nucleic acid template. The AAV rep/cap sequences and/or the rAAV template can be inserted into a deleted region (e.g., the E1a or E3 regions) of the adenovirus.

In a further embodiment, the AAV rep/cap sequences and the adenovirus helper sequences are supplied by a single adenovirus helper vector. According to this embodiment, the rAAV template can be provided as a plasmid template.

In another illustrative embodiment, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as an EBV based nuclear episome).

In a further exemplary embodiment, the AAV rep/cap sequences and adenovirus helper sequences are provided by a single adenovirus helper. The rAAV template can be provided as a separate replicating viral vector. For example, the rAAV template can be provided by a rAAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and, if present, the rAAV template are embedded in the adenovirus backbone and are flanked by the 5′ and 3′ cis sequences, so that these sequences may be packaged into adenovirus capsids. As described above, the adenovirus helper sequences and the AAV rep/cap sequences are generally not flanked by TRs so that these sequences are not packaged into the AAV virions. Zhang et al., ((2001) Gene Ther. 18:704-12) describe a chimeric helper comprising both adenovirus and the AAV rep and cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., (1999) Gene Therapy 6:986 and WO 00/17377.

As a further alternative, the AAV haploid vector of the invention can be produced in insect cells using baculovirus vectors to deliver the rep/cap genes and rAAV template as described, for example, by Urabe et al., (2002) Human Gene Therapy 13:1935-43.

AAV haploid vector stocks free of contaminating helper virus may be obtained by any method known in the art. For example, AAV and helper virus may be readily differentiated based on size. AAV haploid vectors may also be separated away from helper virus based on affinity for a heparin substrate (Zolotukhin et al. (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of AAV virus. Adenovirus mutants defective for late gene expression are known in the art (e.g., ts100K and ts149 adenovirus mutants).

In some embodiments, methods to generate rAVV haploid vectors as disclosed herein can use a rAAV producing cell line, according to the methods as described in U.S. Pat. No. 9,441,206, which is incorporated herein in its entirety by reference. In particular, AAV haploid vector or rAAV virions are produced using a method comprising: (a) providing a rAAV producing cell line an AAV haploid vector expression system; (b) culturing the cells under conditions in which AAV haploid particles are produced; and (c) optionally isolating the AAV haploid vector particles. Ratios of triple transfection of the plasmid and transfection cocktail volumes can be optimized, with varying plasmid ratios of XX680, AAV rep/cap helper and TR plasmid to determine the optimal plasmid ratio for rAAV vector production.

In some instances, the cells are cultured in suspension under conditions in which AAV8 haploid particles are produced. In another embodiment, the cells are cultured in animal component-free conditions. The animal component-free medium can be any animal component-free medium (e.g., serum-free medium) compatible with the rAAV producer cell line. Examples include, without limitation, SFM4Transfx-293 (Hyclone), Ex-Cell 293 (JRH Biosciences), LC-SFM (Invitrogen), and Pro10 cells, or Pro293-S (Lonza). Conditions sufficient for the replication and packaging of the AAV particles can be, e.g., the presence of AAV sequences sufficient for replication of an rAAV genome described herein and encapsidation into AAV capsids (e.g., AAV rep sequences and AAV cap sequences) and helper sequences from adenovirus and/or herpesvirus.

Bacterial DNA sequences from the plasmid backbone can be inadvertently packaged into AAV8 haploid capsids during manufacturing of the recombinant AAV vectors leading to activations of the innate immune system through its interaction with TLR9 (Akira, 2006; Chadeuf, 2005; Wright, 2014). Accordingly, in some embodiments, various technologies can be used to eliminate plasmid backbone sequences in recombinant AAV haploid preparations, for example minicircles which have limited scalability (Schnodt, 2016). Another method to avoid bacterial DNA sequence in the plasmid backbone is to use closed ended linear duplex DNA, which includes a range of DNA replication technology, including but not limited to doggy bone DNA (dbDNA™) for specifically manufacturing of recombinant AAV vectors. Using closed ended linear duplex DNA, such as dbDNA™ eliminates the bacterial backbone and has been used to produce vaccines and lentivirus (Walters et al, 2014; Scott et al, 2015; Karda et al, 2019) and was shown to be unable to trigger TLR9 responses by DNA vaccine developers.

Accordingly, in alternative embodiments, generation of rational polyploid or haploid AAV vectors as disclosed herein, exemplified by the production of AAV8-8-rh10 or AAV8-8-rh74 haploids for example, can be performed using closed ended linear duplex DNA, including but not limited to barbell shaped DNA, as disclosed in US Application 2018/0037943 and Karbowniczek et al., Bioinsights, 2017, both of which are incorporated herein in its entirety by reference. In brief, a plasmid for AAV production using a closed ended linear duplex DNA technology can comprise the ITRs, promoter and gene of interest is flanked by a 56 bp palindromic protelomerase recognition sequence. The plasmid is denatured, and in the presence of a Phi29 DNA polymerase, and appropriate primers, Phi29 initiates rolling circle amplification (RCA), creating a double stranded cancatameric repeats of the original construct. When protelomerase is added, binding of the palindromic protelomerase recognition sequences occurs and cleavage-joining reaction occurs to result in a monomeric double stranded (ds) linear covalently closed DNA construct. Addition of common restriction enzymes remove the undesired DNA plasmid backbone sequence and digestion with exonuclease activity, resulting in dbDNA which can be size fractionated to isolate the dbDNA sequence encoding the ITRs, promoter and gene of interest. An exemplary plasmid for generation of rAAV vectors using closed ended linear duplex DNA such as dbDNA™ technology, comprises in the following 5′ to 3′ direction: 5′-protelomerase RS, 5′ITR, LSP promoter, hGAA, 3′UTR, hGH poly(A), 3′ ITR, 3′-protelomerase RS (sense strand), where the sense strand is linked to the complementary antisense strand for a stranded (ds) linear covalently closed DNA construct. The use of closed ended linear duplex DNA, e.g., doggy bone DNA (dbDNA™) as a starting material for the manufacturing of an AAV vector for use in the methods and composition as disclosed herein eliminates the bacterial backbone used to propagate the plasmid containing AAV vector with an inability for the product to trigger Toll-like receptor 9 (TLR9) responses.

Additional methods of making AAV particles are well known in the art and are described in e.g., U.S. Pat. Nos. U.S. Pat. Nos. 6,204,059, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508, 5,064,764, 6,194,191, 6,566,118, 8,137,948; or International Publication Nos. WO1996039530, WO1998010088, WO1999014354, WO1999015685, WO1999047691, WO2000055342, WO2000075353 and WO2001023597; Methods In Molecular Biology, ed. Richard, Humana Press, N J (1995); O'Reilly et ah, Baculovirus Expression Vectors, A Laboratory Manual, Oxford Univ. Press (1994); Samulski et al.,/. Vir.63:3822-8 (1989); Kajigaya et al., Proc. Nat′l. Acad. Sci. USA 88: 4646-50 (1991); Ruffing et al., J. Vir. 66:6922-30 (1992); Kimbauer et al., Vir., 219:37-44 (1996); Zhao et al., Vir. 272: 382-93 (2000); the contents of each of which are herein incorporated by reference in their entirety. In certain embodiments, the AAV particles are made using the methods described in International Patent Publication WO2015191508, the contents of which are herein incorporated by reference in their entirety.

The viral replication cell may be selected from any biological organism, including prokaryotic (e.g, bacterial) cells, and eukaryotic cells, including, insect cells, yeast cells and mammalian cells. Viral replication cells commonly used for production of recombinant AAV viral particles include, but are not limited to, HEK293 cells, COS cells, HeLa cells, KB cells, and other mammalian cell lines as described in U.S. Pat. Nos. 6,156,303, 5,387,484, 5,741,683, 5,691,176, and 5,688,676; U.S. Patent Application Publication No. 2002/0081721, and International Patent Publication Nos. WO 2000047757, WO 2000024916, and WO 1996017947, the contents of each of which are herein incorporated by reference in their entirety. Viral replication cells may comprise other mammalian cells such as A549, WEH1, 3T3, 10T1/2, BHK, MDCK, COS 1, COS 7, BSC 1, BSC 40, BMT 10, VERO, W138, Saos, C2C12, L cells, HT1080, HepG2 and primary fibroblast, hepatocyte and myoblast cells derived from mammals. Viral replication cells may comprise cells derived from mammalian species including, but not limited to, human, monkey, mouse, rat, rabbit, and hamster. Viral replication cells may comprise cells derived from a cell type, including but not limited to fibroblast, hepatocyte, tumor cell, cell line transformed cell, etc.

### VIII. Recombinant AAV Polyploid Virus Vectors

The present invention provides a method of administering a nucleic acid molecule to a cell, the method comprising contacting the cell with the rAVV haploid vectors and/or the composition or pharmaceutical formulation of this invention.

The present invention further provides a method of delivering a nucleic acid to a subject, the method comprising administering to the subject the AAV haploid virus vector, the AAV particle and/or the composition or pharmaceutical formulation of this invention.

In particular embodiments, the subject is human, and in some embodiments, the subject has or is at risk for a disorder that can be treated by gene therapy protocols. Nonlimiting examples of such disorders include neurological disorders including, but not limited to: epilepsy, depression, Huntington's disease, Parkinson's disease or Alzheimer's disease, ADHD, ASD, an autoimmune disease, cystic fibrosis, thalassemia, Hurler's Syndrome, Sly syndrome, Scheie Syndrome, Hurler-Scheie Syndrome, Hunter's Syndrome, Sanfilippo Syndrome A, B, C, D, Morquio Syndrome, Maroteaux-Lamy Syndrome, Krabbe's disease, phenylketonuria, Batten's disease, spinal cerebral ataxia, LDL receptor deficiency, hyperammonemia, anemia, arthritis, a retinal degenerative disorder including macular degeneration, adenosine deaminase deficiency, a metabolic disorder, and cancer including tumor-forming cancers.

In some embodiments of the methods of this invention, the rAVV haploid vectors and/or the composition or pharmaceutical formulation of this invention can be administered to skeletal muscle, cardiac muscle and/or diaphragm muscle.

In the methods described herein, the rAVV haploid vector and/or the composition or pharmaceutical formulation of this invention can be administered/delivered to a subject of this invention via a systemic route (e.g., intravenously, intraarterially, intraperitoneally, etc.). In some embodiments, the virus vector and/or composition can be administered to the subject via an intracerebroventrical, intracisternal, intraparenchymal, intracranial and/or intrathecal route. In particular embodiments, the rational polyploid or haploid AAV vectors as disclosed herein and/or pharmaceutical formulation of this invention are administered intrathecally or intravenously.

The rAVV haploid vectors as disclosed herein are useful for the delivery of nucleic acid molecules to cells in vitro, ex vivo, and in vivo. In particular, the rAVV haploid vectors can be advantageously employed to deliver or transfer nucleic acid molecules to animal cells, including mammalian cells.

Any heterologous nucleic acid sequence(s) of interest may be delivered in the rAVV haploid vectors of the present invention. Nucleic acid molecules of interest include nucleic acid molecules encoding polypeptides, including therapeutic (e.g., for medical or veterinary uses) and/or immunogenic (e.g., for vaccines) polypeptides.

Therapeutic polypeptides include, but are not limited to, cystic fibrosis transmembrane regulator protein (CFTR), dystrophin (including mini- and micro-dystrophins, see, e.g., Vincent et al., (1993) Nature Genetics 5:130; U.S. Patent Publication No. 2003/017131; International Patent Publication No. WO/2008/088895, Wang et al., Proc. Natl. Acad. Sci. USA 97:13714-13719 (2000); and Gregorevic et al., Mol. Ther. 16:657-64 (2008)), myostatin propeptide, follistatin, activin type II soluble receptor, IGF-1, anti-inflammatory polypeptides such as the Ikappa B dominant mutant, sarcospan, utrophin (Tinsley et al., (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL areceptor, lipoprotein lipase, ornithine transcarbamylase, β-globin, α-globin, spectrin, α₁-antitrypsin, adenosine deaminase, hypoxanthine guanine phosphoribosyl transferase, glucocerebrosidase, sphingomyelinase, lysosomal hexosaminidase A, branched-chain keto acid dehydrogenase, RP65 protein, cytokines (e.g., α-interferon, β-interferon, interferon-γ, interleukin-2, interleukin-4, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-α and -β, and the like), lysosomal acid α-glucosidase, α-galactosidase A, receptors (e.g., the tumor necrosis growth factor-α soluble receptor), S100A1, parvalbumin, adenylyl cyclase type 6, a molecule that modulates calcium handling (e.g., SERCA_{2A}, Inhibitor 1 of PP1 and fragments thereof [e.g., WO 2006/029319 and WO 2007/100465]), a molecule that effects G-protein coupled receptor kinase type 2 knockdown such as a truncated constitutively active bARKct, anti-inflammatory factors such as IRAP, anti-myostatin proteins, aspartoacylase, monoclonal antibodies (including single chain monoclonal antibodies; an exemplary Mab is the Herceptin® Mab), neuropeptides and fragments thereof (e.g., galanin, Neuropeptide Y (see, U.S. Pat. No. 7,071,172), angiogenesis inhibitors such as Vasohibins and other VEGF inhibitors (e.g., Vasohibin 2 [see, WO JP2006/073052]). Other illustrative heterologous nucleic acid sequences encode suicide gene products (e.g., thymidine kinase, cytosine deaminase, diphtheria toxin, and tumor necrosis factor), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. AAV vectors can also be used to deliver monoclonal antibodies and antibody fragments, for example, an antibody or antibody fragment directed against myostatin (see, e.g., Fang et al., Nature Biotechnology 23:584-590 (2005)).

Heterologous nucleic acid sequences encoding polypeptides include those encoding reporter polypeptides (e.g., an enzyme). Reporter polypeptides are known in the art and include, but are not limited to, Green Fluorescent Protein (GFP), luciferase, 0-galactosidase, alkaline phosphatase, luciferase, and chloramphenicol acetyltransferase gene.

Optionally, the heterologous nucleic acid molecule encodes a secreted polypeptide (e.g., a polypeptide that is a secreted polypeptide in its native state or that has been engineered to be secreted, for example, by operable association with a secretory signal sequence as is known in the art).

Alternatively, in particular embodiments of this invention, the heterologous nucleic acid molecule may encode an antisense nucleic acid molecule, a ribozyme (e.g., as described in U.S. Pat. No. 5,877,022), RNAs that effect spliceosome-mediated trans-splicing (see, Puttaraju et al., (1999) Nature Biotech. 17:246; U.S. Pat. Nos. 6,013,487; 6,083,702), interfering RNAs (RNAi) including siRNA, shRNA or miRNA that mediate gene silencing (see, Sharp et al., (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like.

In one aspect, the rational polyploid vectors comprise a viral genome encoding an RNAi agent payload, where, for example, the RNAi agent may be, but is not limited to, a dsRNA, siRNA, shRNA, pre-miRNA, pri-miRNA, miRNA, stRNA, lncRNA, piRNA, or snoRNA. When the RNAi agent is expressed, it inhibits or suppresses the expression of a gene of interest in a cell, wherein the gene of interest may be, but is not limited to, SOD1, MAPT, APOE, HTT, C90RF72, TDP-43, APP, BACE, SNCA, ATXN1, ATXN2, ATXN3, ATXN7, SCN1A-SCN5A, or SCN8A-SCN11 A.

In one aspect, the rational polyploid vectors disclosed herein comprise a viral genome encoding a polypeptide payload. The polypeptide may be, but is not limited to, an antibody, aromatic L-amino acid decarboxylase (AADC), survival motor neuron 1 (SMN1), frataxin (FXN), APOE (APOE2, APOE3, or APOE4), GBA1, GRN, ASP A, CLN2, GLB1, SGSH, NAGLU, IDS, NPC1, or GAN.

Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10:132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.).

Further, a nucleic acid sequence that directs alternative splicing can be delivered. To illustrate, an antisense sequence (or other inhibitory sequence) complementary to the 5′ and/or 3′ splice site of dystrophin exon 51 can be delivered in conjunction with a U1 or U7 small nuclear (sn) RNA promoter to induce skipping of this exon. For example, a DNA sequence comprising a U1 or U7 snRNA promoter located 5′ to the antisense/inhibitory sequence(s) can be packaged and delivered in a modified capsid of the invention.

The virus vector may also comprise a heterologous nucleic acid molecule that shares homology with and recombines with a locus on a host cell chromosome. This approach can be utilized, for example, to correct a genetic defect in the host cell.

The present invention also provides rAVV haploid vectors that express an immunogenic polypeptide, peptide and/or epitope, e.g., for vaccination. The nucleic acid molecule may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura et al., (1994) Proc. Nat. Acad. Sci USA 91:8507; U.S. Pat. No. 5,916,563 to Young et al., U.S. Pat. No. 5,905,040 to Mazzara et al., U.S. Pat. Nos. 5,882,652, and 5,863,541 to Samulski et al.). The antigen may be presented in the parvovirus capsid. Alternatively, the immunogen or antigen may be expressed from a heterologous nucleic acid molecule introduced into a recombinant vector genome. Any immunogen or antigen of interest as described herein and/or as is known in the art can be provided by the virus vector of the present invention.

An immunogenic polypeptide for delivery by a rAAV haploid vector for use as a vaccine as disclosed herein can be any polypeptide, peptide, and/or epitope suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP160 protein, the HIV or SW matrix/capsid proteins, and the HIV or SIV gag, pol and env gene products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia L1 or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens) a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell. Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, PRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124), MART-1, gp100 MAGE-1, MAGE-2, MAGE-3, CEA, TRP-1, TRP-2, P-15, tyrosinase (Brichard et al., (1993) J. Exp. Med. 178:489); HER-2/neu gene product (U.S. Pat. No. 4,968,603), CA 125, LK26, FB5 (endosialin), TAG 72, AFP, CA19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified (see, e.g., Rosenberg, (1996) Ann. Rev. Med. 47:481-91).

As a further alternative, the heterologous nucleic acid molecule can encode any polypeptide, peptide and/or epitope that is desirably produced in a cell in vitro, ex vivo, or in vivo. For example, the AAV haploid vector may be introduced into cultured cells and the expressed gene product isolated therefrom.

It will be understood by those skilled in the art that the heterologous nucleic acid molecule(s) of interest can be operably associated with appropriate control sequences. For example, the heterologous nucleic acid molecule can be operably associated with expression control elements, such as transcription/translation control signals, origins of replication, polyadenylation signals, internal ribosome entry sites (IRES), promoters, and/or enhancers, and the like.

Further, regulated expression of the heterologous nucleic acid molecule(s) of interest can be achieved at the post-transcriptional level, e.g., by regulating selective splicing of different introns by the presence or absence of an oligonucleotide, small molecule and/or other compound that selectively blocks splicing activity at specific sites (e.g., as described in WO 2006/119137, which is incorporated herein in by reference).

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In particular embodiments, the promoter/enhancer elements can be native to the target cell or subject to be treated. In representative embodiments, the promoters/enhancer element can be native to the heterologous nucleic acid sequence. In some embodiments, the promoter sequence or regulatory sequence is a CNS specific promoter. In some embodiments, the CNS specific promoter is disclosed in UK Patent application GB 2007539.6, which is incorporated herein in its entirety by reference. The promoter/enhancer element is generally chosen so that it functions in the target cell(s) of interest, for example CNS tissues, including neuronal and non-neuronal cells in the CNS or PNS. Further, in particular embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In embodiments wherein the heterologous nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The AAV haploid vector according to the present invention provide a means for delivering heterologous nucleic acid molecules into a broad range of cells, including dividing and non-dividing cells. The AAV haploid vector can be employed to deliver a nucleic acid molecule of interest to a cell in vitro, e.g., to produce a polypeptide in vitro or for ex vivo or in vivo gene therapy. The virus vectors are additionally useful in a method of delivering a nucleic acid to a subject in need thereof, e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide.

Further, the method can be practiced because the production of the polypeptide or functional RNA in the subject may impart some beneficial effect.

The virus vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

In general, the virus vectors of the present invention can be employed to deliver a heterologous nucleic acid molecule encoding a polypeptide or functional RNA to treat and/or prevent any disorder or disease state for which it is beneficial to deliver a therapeutic polypeptide or functional RNA. Illustrative disease states include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the Ikappa B dominant mutant, sarcospan, utrophin, mini-utrophin, antisense or RNAi against splice junctions in the dystrophin gene to induce exon skipping [see, e.g., WO 2003/095647], antisense against U7 snRNAs to induce exon skipping [see, e.g., WO 2006/021724], and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [α-galactosidase] and Pompe disease [lysosomal acid α-glucosidase]) and other metabolic disorders, congenital emphysema (al-antitrypsin), Lesch-Nyhan Syndrome (hypoxanthine guanine phosphoribosyl transferase), Niemann-Pick disease (sphingomyelinase), Tay-Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor 1(1-1) and fragments thereof (e.g., I1C), serca2a, zinc finger proteins that regulate the phospholamban gene, Barkct, β2-adrenergic receptor, β2-adrenergic receptor kinase (BARK), phosphoinositide-3 kinase (PI3 kinase), S100A1S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFα soluble receptor), hepatitis (α-interferon), LDL receptor deficiency (LDL receptor), hyperammonemia (omithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The invention can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

The invention can also be used to produce induced pluripotent stem cells (iPS). For example, a AVV haploid vector of the invention can be used to deliver stem cell associated nucleic acid(s) into a non-pluripotent cell, such as adult fibroblasts, skin cells, liver cells, renal cells, adipose cells, cardiac cells, neural cells, epithelial cells, endothelial cells, and the like. Nucleic acids encoding factors associated with stem cells are known in the art. Nonlimiting examples of such factors associated with stem cells and pluripotency include Oct-3/4, the SOX family (e.g., SOX1, SOX2, SOX3 and/or SOX15), the Klf family (e.g., Klf1, Klf2, Klf4 and/or Klf5), the Myc family (e.g., C-myc, L-myc and/or N-myc), NANOG and/or LIN28.

The invention can also be practiced to treat and/or prevent a metabolic disorder such as diabetes (e.g., insulin), hemophilia (e.g., Factor IX or Factor VIII), a lysosomal storage disorder such as a mucopolysaccharidosis disorder (e.g., Sly syndrome [β-glucuronidase], Hurler Syndrome [α-L-iduronidase], Scheie Syndrome [α-L-iduronidase], Hurler-Scheie Syndrome [α-L-iduronidase], Hunter's Syndrome [iduronate sulfatase], Sanfilippo Syndrome A [heparan sulfamidase], B [N-acetylglucosaminidase], C [acetyl-CoA:α-glucosaminide acetyltransferase], D [N-acetylglucosamine 6-sulfatase], Morquio Syndrome A [galactose-6-sulfate sulfatase], B [β-galactosidase], Maroteaux-Lamy Syndrome [N-acetylgalactosamine-4-sulfatase], etc.), Fabry disease (α-galactosidase), Gaucher's disease (glucocerebrosidase), or a glycogen storage disorder (e.g., Pompe disease; lysosomal acid α-glucosidase).

Gene transfer has substantial potential use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, AAV haploid vectors according to the present invention permit the treatment and/or prevention of genetic diseases.

The AVV haploid vectors according to the present invention may also be employed to provide a functional RNA to a cell in vitro or in vivo. Expression of the functional RNA in the cell, for example, can diminish expression of a particular target protein by the cell. Accordingly, functional RNA can be administered to decrease expression of a particular protein in a subject in need thereof. Functional RNA can also be administered to cells in vitro to regulate gene expression and/or cell physiology, e.g., to optimize cell or tissue culture systems or in screening methods.

In addition, AAV haploid vectors according to the instant invention find use in diagnostic and screening methods, whereby a nucleic acid of interest is transiently or stably expressed in a cell culture system, or alternatively, a transgenic animal model.

The AVV haploid vectors of the present invention can also be used for various non-therapeutic purposes, including but not limited to use in protocols to assess gene targeting, clearance, transcription, translation, etc., as would be apparent to one skilled in the art. The AAV haploid vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

As a further aspect, the AVV haploid vectors of the present invention may be used to produce an immune response in a subject. According to this embodiment, a AAV haploid vector comprising a heterologous nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

Alternatively, the AVV haploid vector may be administered to a cell ex vivo and the altered cell is administered to the subject. The AVV haploid vector comprising the heterologous nucleic acid is introduced into the cell, and the cell is administered to the subject, where the heterologous nucleic acid encoding the immunogen can be expressed and induce an immune response in the subject against the immunogen. In particular embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the “transfer of preformed substances (antibody, transfer factor, thymic graft, and interleukin-2) from an actively immunized host to a non-immune host.” Id.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof.

In particular embodiments, the AAV haploid vector or cell comprising the heterologous nucleic acid molecule can be administered in an immunogenically effective amount, as described below.

The AAV haploid vectors of the present invention can also be administered for cancer immunotherapy by administration of a AAV haploid vector expressing one or more cancer cell antigens (or an immunologically similar molecule) or any other immunogen that produces an immune response against a cancer cell. To illustrate, an immune response can be produced against a cancer cell antigen in a subject by administering a AAV haploid vector comprising a heterologous nucleic acid encoding the cancer cell antigen, for example to treat a patient with cancer and/or to prevent cancer from developing in the subject. The AAV haploid vector may be administered to a subject in vivo or by using ex vivo methods, as described herein. Alternatively, the cancer antigen can be expressed as part of the virus capsid or be otherwise associated with the virus capsid (e.g., as described above).

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers.

Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In representative embodiments, the invention provides a method of treating and/or preventing tumor-forming cancers.

The term “tumor” is also understood in the art, for example, as an abnormal mass of undifferentiated cells within a multicellular organism. Tumors can be malignant or benign. In representative embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In particular embodiments; these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

In particular embodiments, cells may be removed from a subject with cancer and contacted with a AAV haploid vector expressing a cancer cell antigen according to the instant invention. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., α-interferon, β-interferon, γ-interferon, ω-interferon, rτ-interferon, interleukin-1α, interleukin-1β, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-18, B cell Growth factor, CD40 Ligand, tumor necrosis factor-α, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the AAV haploid vector.

Cytokines may be administered by any method known in the art. Exogenous cytokines may be administered to the subject, or alternatively, a nucleic acid encoding a cytokine may be delivered to the subject using a suitable vector, and the cytokine produced in vivo.

### XI. Subjects, Pharmaceutical Formulations, and Modes of Administration

The AAV haploid virus vector and/or rational polyploid AAV vector as disclosed herein for use in the methods of administration as disclosed herein can be formulated in a pharmaceutical composition with a pharmaceutically acceptable excipient, i.e., one or more pharmaceutically acceptable carrier substances and/or additives, e.g., buffers, carriers, excipients, stabilizers, etc. The pharmaceutical composition may be provided in the form of a kit. Pharmaceutical compositions comprising the AAV haploid virus vector and/or rational polyploid AAV vector as disclosed herein for use in the methods of administration as disclosed herein and uses thereof are known in the art.

Accordingly, a further aspect of the invention provides a pharmaceutical composition comprising a AAV haploid vector as disclosed herein for use in the methods of administration as disclosed herein. Relative amounts of the active ingredient (e.g. a AAV haploid virus vector and/or rational polyploid AAV vector aa disclosed herein), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1 percent and 99 percent (w/w) of the active ingredient. By way of example, the composition may comprise between 0.1 percent and 100 percent, e.g., between. 5 and 50 percent, between 1-30 percent, between 5-80 percent, at least 80 percent (w/w) active ingredient.

The pharmaceutical compositions can be formulated using one or more excipients or diluents to (1) increase stability; (2) increase cell transfection or transduction; (3) permit the sustained or delayed release of the payload; (4) alter the biodistribution (e.g., target the viral particle to specific tissues or cell types); (5) increase the translation of encoded protein; (6) alter the release profile of encoded protein and/or (7) allow for regulatable expression of the payload of the invention. In some embodiments, a pharmaceutically acceptable excipient may be at least 95 percent, at least 96 percent, at least 97 percent, at least 98 percent, at least 99 percent, or 100 percent pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia. Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21 st Edition, A. R. Gennaro, Lippincott, Williams and Wilkins, Baltimore, M D, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

The AAV haploid virus vector and/or rational polyploid AAV vector as disclosed herein for use in the methods of administration as disclosed herein may be used in combination with one or more other therapeutic, prophylactic, research or diagnostic agents. By “in combination with,” it is not intended to imply that the agents must be administered at the same time and/or formulated for delivery together, although these methods of delivery are within the scope of the present invention. Compositions can be administered concurrently with, prior to, or subsequent to, one or more other desired therapeutics or medical procedures. In some embodiments, the delivery of one treatment (e.g., gene therapy vectors) is still occurring when the delivery of the second (e.g., one or more therapeutic) begins, so that there is overlap in terms of administration. This is sometimes referred to herein as “simultaneous” or “concurrent delivery.” In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered. The composition described herein and the at least one additional therapy can be administered simultaneously, in the same or in separate compositions, or sequentially. For sequential administration, the gene therapy vectors described herein can be administered first, and the one or more therapeutic can be administered second, or the order of administration can be reversed. The gene therapy vectors and the one or more therapeutic can be administered during periods of active disorder, or during a period of remission or less active disease. The gene therapy vectors can be administered before another treatment, concurrently with the treatment, post-treatment, or during remission of the disorder.

When administered in combination, the AAV haploid virus vector and/or rational polyploid AAV vector as disclosed herein for use in the methods of administration as disclosed herein and the one or more therapeutic (e.g., second or third therapeutic), or all, can be administered in an amount or dose that is higher, lower or the same as the amount or dosage of each used individually, e.g., as a monotherapy. In certain embodiments, the administered amount or dosage of a AAV haploid vector as disclosed herein for use in the methods of administration as disclosed herein and the one or more therapeutic (e.g., second or third agent), or all, is lower (e.g., at least 20%, at least 30%, at least 40%, or at least 50%) than the amount or dosage of each used individually. In other embodiments, the amount or dosage of the AAV haploid vector as disclosed herein for use in the methods of administration as disclosed herein and the one or more therapeutic (e.g., second or third agent), or all, that results in a desired effect (e.g., treatment of a cardiovascular disease or heart disease) is lower (e.g., at least 20%, at least 30%, at least 40%, or at least 50% lower) than the amount or dosage of each individually required to achieve the same therapeutic effect.

In some embodiments, the methods of administration of a AAV haploid vector as disclosed herein can deliver a rAVV vector disclosed herein alone, or in combination with an additional agent, for example, an immune modulator as disclosed herein.

AAV haploid vectors, AAV particles and capsids according to the present invention find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammal” as used herein includes, but is not limited to, humans, non-human primates, bovines, ovines, caprines, equines, felines, canines, lagomorphs, etc.

Human subjects include neonates, infants, juveniles, adults and geriatric subjects.

In representative embodiments, the subject is “in need of” the methods of the invention.

In particular embodiments, the present invention provides a pharmaceutical composition comprising a AAV haploid virus vector and/or rational polyploid AAV vector and/or AAV haploid particle of the invention in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form. For administration to a subject or for other pharmaceutical uses, the carrier will be sterile and/or physiologically compatible.

By “pharmaceutically acceptable” it is meant a material that is not toxic or otherwise undesirable, i.e., the material may be administered to a subject without causing any undesirable biological effects.

One aspect of the present invention is a method of transferring a nucleic acid molecule to a cell in vitro. The AAV haploid vector may be introduced into the cells at the appropriate multiplicity of infection according to standard transduction methods suitable for the particular target cells. Titers of AAV haploid vector to administer can vary, depending upon the target cell type and number, and the particular AAV haploid vector, and can be determined by those of skill in the art without undue experimentation. In representative embodiments, at least about 10³ infectious units, optionally at least about 10⁵ infectious units are introduced to the cell.

The cell(s) into which the AAV haploid vector is introduced can be of any type, including but not limited to neural cells (including cells of the peripheral and central nervous systems, in particular, brain cells such as neurons and oligodendrocytes), lung cells, cells of the eye (including retinal cells, retinal pigment epithelium, and corneal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. In representative embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above.

The AAV haploid vector can be introduced into cells in vitro for the purpose of administering the modified cell to a subject. In particular embodiments, the cells have been removed from a subject, the AAV haploid vector is introduced therein, and the cells are then administered back into the subject. Methods of removing cells from subject for manipulation ex vivo, followed by introduction back into the subject are known in the art (see, e.g., U.S. Pat. No. 5,399,346). Alternatively, the recombinant AAV haploid vector can be introduced into cells from a donor subject, into cultured cells, or into cells from any other suitable source, and the cells are administered to a subject in need thereof (i.e., a “recipient” subject).

Suitable cells for ex vivo nucleic acid delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In particular embodiments, the cells transduced with the AAV haploid vector are administered to the subject in a treatment effective or prevention effective amount in combination with a pharmaceutical carrier.

In some embodiments, the AAV haploid vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In particular embodiments, the dosage is sufficient to produce a protective immune response (as defined above).

The degree of protection conferred need not be complete or permanent, as long as the benefits of administering the immunogenic polypeptide outweigh any disadvantages thereof.

A further aspect of the invention is a method of administering the AAV haploid vector and/or haploid virus capsid to subjects. Administration of the AAV haploid vectors and/or capsids according to the present invention to a human subject or an animal in need thereof can be by any means known in the art. Optionally, the AAV haploid vector and/or haploid capsid is delivered in a treatment effective or prevention effective dose in a pharmaceutically acceptable carrier.

The AAV haploid vectors and/or haploid capsids of the invention can further be administered to elicit an immunogenic response (e.g., as a vaccine). Typically, immunogenic compositions of the present invention comprise an immunogenically effective amount of AAV haploid vector and/or capsid in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the AAV rational polyploid e.g., haploid vector and/or capsid to be administered to a subject depend upon the mode of administration, the disease or condition to be treated and/or prevented, the individual subject's condition, the particular AAV haploid vector or haploid capsid, and the nucleic acid to be delivered, and the like, and can be determined in a routine manner. Exemplary doses for achieving therapeutic effects are titers of at least about 10⁵, 10⁶, 10⁷, 10⁸, 10⁹, 10¹⁰, 10¹¹, 10¹², 10¹³, 10¹⁴, 10¹⁵ transducing units, optionally about 10⁸ to about 10¹³ transducing units.

In one embodiment, the population is at least 1×10⁴ viral genomes (vg)/ml, is at least 1×10⁵ viral genomes (vg)/ml, is at least 1×10⁶ viral genomes (vg)/ml, at least 1×10⁷ viral genomes (vg)/ml, at least 1×10⁸ viral genomes (vg)/ml, at least 1×10⁹ viral genomes (vg)/ml, at least 1×10¹⁰ vg/per ml, at least 1×10¹¹ vg/per ml, at least 1×10¹² vg/per ml. In one embodiment, the population ranges from about 1×10⁵ vg/ml to about 1×10¹³ vg/ml.

In some embodiments, at least about 1.6×10¹² to about 4.0×10¹² vg/kg will be administered per dose in a pharmaceutically acceptable carrier. In a further embodiment, dosages of the haploid AAV vector as disclosed herein to be administered to a subject depend upon the mode of administration, the severity and type of disease to be treated and/or prevented, the individual subject's condition, age and gender, and the particular VP3 structural protein, and VP1 and/or VP2 structural proteins present in the polyploid AAV vector, the transgene being delivered, and the promoter controlling transgene expression, and the like, and can be determined in a routine manner. Exemplary doses for achieving therapeutic effects are titers of at least about 1.5×10¹¹ vg/kg, or at least about 1.5×10¹² vg/kg, or at least about 4.0×10¹² vg/kg. It is encompassed that the dose for achieving therapeutic effects as disclosed herein may also be determined by the strength of the specific promoter, including brain and neuronal promoters operatively linked to the nucleic acid encoding the transgene, as well as the presence of any signal sequences, and ability of the cell to cleave the signal sequence when secreted from the cell.

In some embodiments, as the polyploid AAV vectors disclosed herein elicits less of a humoral immune response as compared to the humoral response as elicited by the parental AAV subtypes of the VP1 or, AAV VP2 structural proteins, the dose of the polyploid AAV vectors is higher than 1.6×10¹², or higher than about 4.0×10¹² vg/kg.

In some embodiments, exemplary doses for achieving therapeutic effects of a polyploid AAV vector as disclosed herein is within the range of 1.0E⁹ vg/kg to 5.0E¹¹ vg/kg. In some embodiments, the dose administered to a subject is at least about 1.0E⁹ vg/kg, at least about 1.0E¹⁰ vg/kg, at least about 1.0E11 vg/kg, at least about 1.0E12 vg/kg, about 1.1E12 vg/kg, about 1.2E12 vg/kg, about 1.3E12 vg/kg, about 1.4E12 vg/kg, about 1.5E12 vg/kg, about 1.6E12 vg/kg, about 1.7E12 vg/kg, about 1.8E12 vg/kg, about 1.9E12 vg/kg, about 2.0E12 vg/kg, about 3.0E12 vg/kg, about 4.0E12 vg/kg, about 5.0E12 vg/kg, about 6.0E12 vg/kg, about 7.0E12 vg/kg, about 8.0E12 vg/kg, about 9.0E12 vg/kg, about 1.0E13 vg/kg, about 1.2E13 vg/kg, about 1.2E13 vg/kg, about 1.2E13 vg/kg, about 1.3E13 vg/kg, about 1.4E13 vg/kg, about 1.5E13 vg/kg, about 1.6E13 vg/kg, about 1.7E13 vg/kg, about 1.8E13 vg/kg, about 1.9E13 vg/kg, about 2.0E13 vg/kg, about 3.0E13 vg/kg, about 4.0E13 vg/kg, about 5.0E13 vg/kg.

In some embodiments, exemplary doses for achieving therapeutic effects according to the methods as disclosed herein are titers of at between 1.2E12 and 4.0E12 vg/kg, for example, least about 1.0E12 vg/kg, about 1.1E12 vg/kg, about 1.2E12 vg/kg, about 1.3E12 vg/kg, about 1.4E12 vg/kg, about 1.5E12 vg/kg, about 1.6E12 vg/kg, about 1.7E12 vg/kg, about 1.8E12 vg/kg, about 1.9E12 vg/kg, about 2.0E12 vg/kg, about 2.1E12 vg/kg, about 2.2E12 vg/kg, about 2.3E12 vg/kg, about 2.4E12 vg/kg, about 2.5E12 vg/kg, about 2.6E12 vg/kg, about 2.7E12 vg/kg, about 2.8E12 vg/kg, about 2.9E12 vg/kg, about 3.0E12 vg/kg, about 3.1E12 vg/kg, about 3.2E12 vg/kg, about 3.3E12 vg/kg, about 3.4E12 vg/kg, about 3.5E12 vg/kg, about 3.6E12 vg/kg, about 3.7E12 vg/kg, about 3.8E12 vg/kg, about 3.9E12 vg/kg, about 4.0E12 vg/kg.

In some embodiments, a polyploid AAV vector as disclosed herein useful for the methods to treat a disease or disorder of the brain or spinal cord, or a neuronal or neurodegenerative disease, exemplary doses for achieving therapeutic effects are titers of at least about 1.0E12 to 4.0E12 vg/kg, or about 1.2E12 to 3.0E12 vg/kg, or about 1.2E12 to 2.5E12 vg/kg, or about 2.5E12 to 4.0E12 vg/kg.

In particular embodiments, more than one administration (e.g., two, three, four, five, six, seven, eight, nine, ten, etc., or more administrations) may be employed to achieve the desired level of gene expression over a period of various intervals, e.g., hourly, daily, weekly, monthly, yearly, etc. Dosing can be single dosage or cumulative (serial dosing), and can be readily determined by one skilled in the art. For instance, treatment of a disease or disorder may comprise a one-time administration of an effective dose of a pharmaceutical composition AAV haploid vector disclosed herein. Alternatively, treatment of a disease or disorder may comprise multiple administrations of an effective dose of a AAV haploid vector carried out over a range of time periods, such as, e.g., once daily, twice daily, trice daily, once every few days, or once weekly. The timing of administration can vary from individual to individual, depending upon such factors as the severity of an individual's symptoms. For example, an effective dose of a AAV haploid vector disclosed herein can be administered to an individual once every six months for an indefinite period of time, or until the individual no longer requires therapy. A person of ordinary skill in the art will recognize that the condition of the individual can be monitored throughout the course of treatment and that the effective amount of a AAV haploid vector disclosed herein that is administered can be adjusted accordingly.

In an embodiment, the period of administration of a AAV haploid vector is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). Administration can also be to a tumor (e.g., in or near a tumor or a lymph node). The most suitable route in any given case will depend on the nature and severity of the condition being treated and/or prevented and on the nature of the particular vector that is being used.

Injectables can be prepared in conventional forms, either as liquid solutions or suspensions, solid forms suitable for solution or suspension in liquid prior to injection, or as emulsions. Alternatively, one may administer the AAV haploid vector and/or virus capsids of the invention in a local rather than systemic manner, for example, in a depot or sustained-release formulation. Further, the AAV haploid vector and/or virus capsid can be delivered adhered to a surgically implantable matrix (e.g., as described in U.S. Patent Publication No. US2004/0013645. The AAV haploid vectors and/or virus capsids disclosed herein can be administered to the lungs of a subject by any suitable means, optionally by administering an aerosol suspension of respirable particles comprised of the AAV haploid vectors and/or virus capsids, which the subject inhales. The respirable particles can be liquid or solid. Aerosols of liquid particles comprising the AAV haploid vectors and/or virus capsids may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the AAV haploid vectors and/or capsids may likewise be produced with any solid particulate medicament aerosol generator, by techniques known in the pharmaceutical art.

The AAV haploid vectors and virus capsids can be administered to tissues of the CNS (e.g., brain, eye) and may advantageously result in broader distribution of the AAV haploid vector or capsid than would be observed in the absence of the present invention.

In particular embodiments, the delivery vectors of the invention may be administered to treat diseases of the CNS, including genetic disorders, neurodegenerative disorders, psychiatric disorders and tumors. Illustrative diseases of the CNS include, but are not limited to Alzheimer's disease, Parkinson's disease, Huntington's disease, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay-Sachs disease, Lesch-Nyan disease, epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The delivery vectors of the present invention can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector of the invention.

Retinitis pigmentosa, by comparison, is characterized by retinal degeneration. In representative embodiments, retinitis pigmentosa can be treated by intraocular (e.g., vitreal administration) of a delivery vector encoding one or more neurotrophic factors.

Age-related macular degeneration involves both angiogenesis and retinal degeneration. This disorder can be treated by administering the inventive deliver vectors encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the inventive delivery vectors. Such agents include N-methyl-D-aspartate (NMDA) antagonists, cytokines, and neurotrophic factors, delivered intraocularly, optionally intravitreally.

In other embodiments, the present invention may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, the invention can also be used to treat epilepsy, which is marked by multiple seizures overtime.

In one representative embodiment, somatostatin (or an active fragment thereof) is administered to the brain using a delivery vector of the invention to treat a pituitary tumor. According to this embodiment, the delivery vector encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins are known in the art.

In particular embodiments, the vector can comprise a secretory signal as described in U.S. Pat. No. 7,071,172.

In representative embodiments of the invention, the AAV haploid vector and/or virus capsid is administered to the CNS (e.g., to the brain or to the eye). The AAV haploid vector and/or capsid may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes. cortex, basal ganglia, hippocampus and portaamygdala), limbic system, neocortex, corpus striatum, cerebrum, and inferior colliculus. The AAV haploid vector and/or capsid may also be administered to different regions of the eye such as the retina, cornea and/or optic nerve.

The AAV haploid vector and/or capsid may be delivered into the cerebrospinal fluid (e.g., by lumbar puncture) for more disperse administration of the delivery vector.

The AAV haploid vector and/or capsid may further be administered intravascularly to the CNS in situations in which the blood-brain barrier has been perturbed (e.g., brain tumor or cerebral infarct).

The AAV haploid vector and/or capsid can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons.

In particular embodiments, the AAV haploid vector and/or capsid is administered in a liquid formulation by direct injection (e.g., stereotactic injection) to the desired region or compartment in the CNS. In other embodiments, the AAV haploid vector and/or capsid may be provided by topical application to the desired region or by intra-nasal administration of an aerosol formulation. Administration to the eye may be by topical application of liquid droplets. As a further alternative, the AAV haploid vector and/or capsid may be administered as a solid, slow-release formulation (see, e.g., U.S. Pat. No. 7,201,898).

In yet additional embodiments, the AAV haploid vector can used for retrograde transport to treat and/or prevent diseases and disorders involving motor neurons (e.g., amyotrophic lateral sclerosis (ALS); spinal muscular atrophy (SMA), etc.). For example, the AAV haploid vector can be delivered to muscle tissue from which it can migrate into neurons.

Aspects of the present specification disclose, in part, treating an individual suffering from a disease or disorder. As used herein, the term “treating,” refers to reducing or eliminating in an individual a clinical symptom of the disease or disorder; or delaying or preventing in an individual the onset of a clinical symptom of a disease or disorder. For example, the term “treating” can mean reducing a symptom of a condition characterized by a disease or disorder, by, e.g., at least 20%, at least 25%, at least 30%, at least 35%, at least 40%, at least 45%, at least 50%, at least 55%, at least 60%, at least 65%, at least 70%, at least 75%, at least 80%, at least 85%, at least 90% at least 95%, or at least 100%. The actual symptoms associated with a specific disease or disorder are well known and can be determined by a person of ordinary skill in the art by taking into account factors, including, without limitation, the location of the disease or disorder, the cause of the disease or disorder, the severity of the disease or disorder, and/or the tissue or organ affected by the disease or disorder. Those of skill in the art will know the appropriate symptoms or indicators associated with a specific type of disease or disorder and will know how to determine if an individual is a candidate for treatment as disclosed herein.

In aspects of this embodiment, a therapeutically effective amount of a AAV haploid vector disclosed herein reduces a symptom associated with a disease or disorder by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100%. In other aspects of this embodiment, a therapeutically effective amount of a AAV haploid vector disclosed herein reduces a symptom associated with a disease or disorder by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a AAV haploid vector disclosed herein reduces a symptom associated with disease or disorder by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

In one embodiment, a AAV haploid vector disclosed herein is capable of increasing the level and/or amount of a protein encoded in the AAV haploid vector that is administered to a patient by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment. In other aspects of this embodiment, AAV haploid vector is capable of reducing the severity of a disease or disorder in an individual suffering from the disease or disorder by, e.g., about 10% to about 100%, about 20% to about 100%, about 30% to about 100%, about 40% to about 100%, about 50% to about 100%, about 60% to about 100%, about 70% to about 100%, about 80% to about 100%, about 10% to about 90%, about 20% to about 90%, about 30% to about 90%, about 40% to about 90%, about 50% to about 90%, about 60% to about 90%, about 70% to about 90%, about 10% to about 80%, about 20% to about 80%, about 30% to about 80%, about 40% to about 80%, about 50% to about 80%, or about 60% to about 80%, about 10% to about 70%, about 20% to about 70%, about 30% to about 70%, about 40% to about 70%, or about 50% to about 70% as compared to a patient not receiving the same treatment.

In aspects of this embodiment, a therapeutically effective amount of a AAV haploid vector disclosed herein increases the amount of protein that is encoded within the AAV haploid vector in an individual by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 100% as compared to an individual not receiving the same treatment. In other aspects of this embodiment, a therapeutically effective amount of a AAV haploid vector disclosed herein reduces the severity of a disease or disorder or maintains the severity of a disease or disorder in an individual by, e.g., at most 10%, at most 15%, at most 20%, at most 25%, at most 30%, at most 35%, at most 40%, at most 45%, at most 50%, at most 55%, at most 60%, at most 65%, at most 70%, at most 75%, at most 80%, at most 85%, at most 90%, at most 95% or at most 100%. In yet other aspects of this embodiment, a therapeutically effective amount of a AAV haploid vector disclosed herein reduces or maintains the severity of a disease or disorder in an individual by, e.g., about 10% to about 100%, about 10% to about 90%, about 10% to about 80%, about 10% to about 70%, about 10% to about 60%, about 10% to about 50%, about 10% to about 40%, about 20% to about 100%, about 20% to about 90%, about 20% to about 80%, about 20% to about 20%, about 20% to about 60%, about 20% to about 50%, about 20% to about 40%, about 30% to about 100%, about 30% to about 90%, about 30% to about 80%, about 30% to about 70%, about 30% to about 60%, or about 30% to about 50%.

A AAV haploid vector is administered to an individual or a patient. An individual or a patient is typically a human being, but can be an animal, including, but not limited to, dogs, cats, birds, cattle, horses, sheep, goats, reptiles and other animals, whether domesticated or not.

In an embodiment, a AAV haploid vector of the present invention can be used to create an AAV that targets a specific tissue including, but not limited to, the central nervous system, retina, heart, lung, skeletal muscle and liver. These targeted AAV haploid vectors can be used to treat diseases that are tissue specific, or for the production of proteins that are endogenously produced in a specific normal tissue, such as a Factor IX (FIX), Factor VIII, FVIII and other proteins known in the art.

### X. Immune Modulation

In any embodiment of the methods and compositions as disclosed herein, a subject being administered a rAAV vector or rAAV genome as disclosed herein is also administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In another embodiment, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors β1 and β2, TNF and others that are publicly known).

In some embodiments, the methods and compositions using the AAV haploid vectors and AAV genomes as described herein, further comprises administering an immune modulator. In some embodiments, the immune modulator can be administered at the time of AAV haploid vector administration, before rAAV haploid vector administration or, after the rAAV haploid vector administration.

In some embodiments, the immune modulator is an immunoglobulin degrading enzyme such as IdeS, IdeZ, IdeS/Z, Endo S, or, their functional variant. Non-limiting examples of references of such immunoglobulin degrading enzymes and their uses as described in U.S. Pat. Nos. 7,666,582, 8,133,483, US 20180037962, US 20180023070, US 20170209550, U.S. Pat. No. 8,889,128, WO2010/057626, U.S. Pat. Nos. 9,707,279, 8,323,908, US 20190345533, US 20190262434, and WO2020/016318, each of which are incorporated in their entirety by reference.

In some embodiments, the immune modulator is Proteasome inhibitor. In certain aspects, the proteasome inhibitor is Bortezomib. In some aspects of the embodiment, the immune modulator comprises bortezomib and anti CD20 antibody, Rituximab. In other aspects of the embodiment, the immune modulator comprises bortezomib, Rituximab, methotrexate, and intravenous gamma globulin. Non-limiting examples of such references, disclosing proteasome inhibitors and their combination with Rituximab, methotrexate and intravenous gamma globulin, as described in U.S. Pat. Nos. 10,028,993, 9,592,247, and, U.S. Pat. No. 8,809,282, each of which are incorporated in their entirety by reference.

In alternative embodiments, the immune modulator is an inhibitor of the NF-kB pathway. In certain aspects of the embodiment, the immune modulator is Rapamycin or, a functional variant. Non-limiting examples of references disclosing rapamycin and its use described in U.S. Pat. No. 10,071,114, US 20160067228, US 20160074531, US 20160074532, US 20190076458, U.S. Pat. No. 10,046,064, are incorporated in their entirety. In other aspects of the embodiment, the immune modulator is synthetic nanocarriers comprising an immunosuppressant. Non limiting examples of references of immunosuppressants, immunosuppressants coupled to synthetic nanocarriers, synthetic nanocarriers comprising rapamycin, and/or, tolerogenic synthetic nanocarriers, their doses, administration and use as described in US20150320728, US 20180193482, US 20190142974, US 20150328333, US20160243253, U.S. Pat. No. 10,039,822, US 20190076522, US 20160022650, U.S. Pat. Nos. 10,441,651, 10,420,835, US 20150320870, US 2014035636, U.S. Pat. Nos. 10,434,088, 10,335,395, US 20200069659, U.S. Pat. No. 10,357,483, US 20140335186, U.S. Pat. Nos. 10,668,053, 10,357,482, US 20160128986, US 20160128987, US 20200038462, US 20200038463, each of which are incorporated in their entirety by reference.

In some embodiments, the immune modulator is synthetic nanocarriers comprising rapamycin (ImmTOR™ nanoparticles) (Kishimoto, et al., 2016, Nat Nanotechnol, 11(10): 890-899; Maldonado, et al., 2015, PNAS, 112(2): E156-165), as disclosed in US20200038463, U.S. Pat. No. 9,006,254 each of which is incorporated herein in its entirety. In some embodiments, the immune modulator is an engineered cell, e.g., an immune cell that has been modified using SQZ technology as disclosed in WO2017192786, which is incorporated herein in its entirety by reference.

In some embodiments, the immune modulator is selected from the group consisting of poly-ICLC, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, Juvlmmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PEPTEL, vector system, PLGA microparticles, resiquimod, SRL172, Virosomes and other Virus-like particles, YF-17D, VEGF trap, R848, beta-glucan, Pam3Cys, and Aquila's QS21 stimulon. In another further embodiment, the immunomodulator or adjuvant is poly-ICLC

In some embodiments, the immune modulator is a small molecule that inhibit the innate immune response in cells, such as chloroquine (a TLR signaling inhibitor) and 2-aminopurine (a PKR inhibitor), can also be administered in combination with the composition comprising at least one rAAV as disclosed herein. Some non-limiting examples of commercially available TLR-signaling inhibitors include BX795, chloroquine, CLI-095, OxPAPC, polymyxin B, and rapamycin (all available for purchase from INVIVOGEN™). In addition, inhibitors of pattern recognition receptors (PRR) (which are involved in innate immunity signaling) such as 2-aminopurine, BX795, chloroquine, and H-89, can also be used in the compositions and methods comprising at least one rAAV vector as disclosed herein for in vivo protein expression as disclosed herein.

In some embodiments, a AAV haploid vector can also encode a negative regulators of innate immunity such as NLRX1. Accordingly, in some embodiments, a AAV haploid vector can also optionally encode one or more, or any combination of NLRX1, NS1, NS3/4A, or A46R. Additionally, in some embodiments, a composition comprising at least one AAV haploid vector as disclosed herein can also comprise a synthetic, modified-RNA encoding inhibitors of the innate immune system to avoid the innate immune response generated by the tissue or the subject.

In some embodiments, an immune modulator for use in the administration methods as disclosed herein is an immunosuppressive agent. As used herein, the term “immunosuppressive drug or agent” is intended to include pharmaceutical agents which inhibit or interfere with normal immune function. Examples of immunosuppressive agents suitable with the methods disclosed herein include agents that inhibit T-cell/B-cell co-stimulation pathways, such as agents that interfere with the coupling of T-cells and B-cells via the CTLA4 and B7 pathways, as disclosed in U.S. Patent Pub. No 2002/0182211. In one embodiment, an immunosuppressive agent is cyclosporine A. Other examples include myophenylate mofetil, rapamicin, and anti-thymocyte globulin. In one embodiment, the immunosuppressive drug is administered in a composition comprising at least one rAAV vector as disclosed herein, or can be administered in a separate composition but simultaneously with, or before or after administration of a composition comprising at least one AAV haploid vector according to the methods of administration as disclosed herein. An immunosuppressive drug is administered in a formulation which is compatible with the route of administration and is administered to a subject at a dosage sufficient to achieve the desired therapeutic effect. In some embodiments, the immunosuppressive drug is administered transiently for a sufficient time to induce tolerance to the rAAV vector as disclosed herein.

In any embodiment of the methods and compositions as disclosed herein, a subject being administered a AAV haploid vector or rAAV genome as disclosed herein is also administered an immunosuppressive agent. Various methods are known to result in the immunosuppression of an immune response of a patient being administered AAV. Methods known in the art include administering to the patient an immunosuppressive agent, such as a proteasome inhibitor. One such proteasome inhibitor known in the art, for instance as disclosed in U.S. Pat. No. 9,169,492 and U.S. patent application Ser. No. 15/796,137, both of which are incorporated herein by reference, is bortezomib. In some embodiments, an immunosuppressive agent can be an antibody, including polyclonal, monoclonal, scfv or other antibody derived molecule that is capable of suppressing the immune response, for instance, through the elimination or suppression of antibody producing cells. In a further embodiment, the immunosuppressive element can be a short hairpin RNA (shRNA). In such an embodiment, the coding region of the shRNA is included in the rAAV cassette and is generally located downstream, 3′ of the poly-A tail. The shRNA can be targeted to reduce or eliminate expression of immunostimulatory agents, such as cytokines, growth factors (including transforming growth factors f31 and 02, TNF and others that are publicly known).

The use of such immune modulating agents facilitates the ability to for one to use multiple dosing (e.g., multiple administration) over numerous months and/or years. This permits using multiple agents as discussed below, e.g., a AAV haploid vector encoding multiple genes, or multiple administrations to the subject.

In some embodiments, the present application may be defined in any of the following paragraphs:

1. A population of rational polyploid AAV virions suitable for use in crossing the blood brain barrier, the rational polyploid AAV virions comprising at least one of AAV VP1 or VP2 viral structural proteins and an AAV VP3 viral structural protein;
wherein the at least one of VP1 or VP2 viral structural proteins are each from any AAV serotype, and the VP3 viral structural protein is from an AAV serotype that efficiently crosses the blood brain barrier and is different from the serotype of at least one of VP1 or VP2, and
wherein the population of rational polyploid AAV virions is capable of crossing the blood brain barrier (BBB) and/or transducing an endothelial cell of the BBB and/or a blood component that crosses the BBB upon systemic or intrathecal administration.

2. The population of paragraph 1, wherein the population exhibits enhanced transduction activity across the blood brain barrier (BBB) relative to a non-rational polyploid AAV particle that lacks ability to cross the blood brain barrier.

3. The population of any of paragraphs 1-2, wherein the VP3 viral structural protein is an AAV rhesus monkey serotype.

4. The population of any of paragraphs 1-3, wherein the VP3 viral structural protein is from a serotype that efficiently crosses the blood brain barrier selected from the group consisting of AAV1, AAV6, AAV6.2, AAV7, AAV9, AAVrh10, AAVrh74, AAVrh39, and AAVrh43.

5. The population of any of paragraphs 1-4, wherein the population has enhanced transduction to one or more of cortex, striatum, thalamus, medulla, hippocampus, cerebellum and spinal cord of a subject relative to a non-rational polyploid AAV particle that lacks ability to efficiently cross the blood brain barrier.

6. The population of any of paragraphs 1-5, wherein, the population has enhanced transduction relative to AAV2 in one or more of CNS regions selected from the group consisting of medulla, cervical, thoracic, lumbar, and choroid plexus.

7. The population of any of paragraphs 1-6, wherein the population has enhanced binding to brain microvascular endothelial cell (BMVEC) relative to AAV8.

8. The population of any of paragraphs 1-7, wherein the population has biodistribution in the CNS.

9. The population of any of paragraphs 1-8, wherein the population has CNS biodistribution of at least 0.05 vg/cell, 0.1 vg/cell, at least 0.2 vg/cell, at least 0.4 vg/cell, at least 0.6 vg/cell, at least 0.8 vg/cell, at least 1 vg/cell, at least 5 vg/cell, at least 10 vg/cell, at least 20 vg/cell, at least 25 vg/cell, or preferably more.

10. The population of any of paragraphs 1-9, wherein the at least one of VP1 or VP2 is selected from an AAV serotype that crosses blood brain barrier.

11. The population of any of paragraphs 1-9, wherein the at least one of VP1 or VP2 is selected from an AAV serotype that do not cross blood brain barrier.

12. The population of any of paragraphs 1-11, wherein that least one of VP1 or VP2 is not selected from AAV rhesus monkey serotype.

13. The population of any of paragraphs 1-11, wherein the at least one of VP1 or VP2 is selected from an AAV rhesus monkey serotype.

14. The population of any of paragraphs 1-13, wherein the population elicits a lower humoral immune response when administered to a subject as compared to a humoral response as elicited by a parental AAV vector of the subtype of the VP1 or VP2 structural protein.

15. The population of any of paragraphs 1-14, wherein the population evades neutralizing antibodies against the parental serotypes of AAV VP1, VP2, or VP3 viral structural proteins.

16. A method for delivering a transgene across the blood brain barrier of a subject, the method comprising administering to the subject a population of rational polyploid AAV virions of any of paragraphs 1-15.

17. A method for repeat dosing of AAV to a subject, the method comprising a first administration performed by administering to the subject the population of rational polyploid AAV virions from any of paragraphs 1-16, and a second administration performed by administering to the subject parental AAV serotypes of the at least one of VP1 or VP2 viral structural protein, wherein the population of rational polyploid AAV virions elicits a reduced humoral response in the subject as compared to a humoral response as elicited by the parental AAV serotypes of the VP1 or VP2 viral structural protein, and, wherein the at least one of the VP1 or VP2 is not from a Rhesus AAV serotype.

18. A population of rational polyploid AAV virions that allows repeat dosing, the population comprising: a rational polyploid AAV virion comprising at least one of AAV VP1 or VP2 viral structural proteins and a AAV VP3 viral structural protein; wherein the at least one of VP1 or VP2 viral structural proteins are each from any AAV viral serotype, and the VP3 viral structural protein is selected from a rhesus monkey AAV serotype; wherein the population of rational polyploid AAV virions elicits a reduced humoral response as compared to a humoral response elicited by the parental AAV serotype of the VP1 or VP2 viral structural proteins; wherein the at least one of VP1 or VP2 are not from a Rhesus AAV serotype, and wherein the repeat dosing comprises a first administration of the population of rational polyploid AAV virions and a second administration of a parental AAV serotype of the VP1 structural viral protein or VP2 structural viral protein.

19. A population of rational polyploid AAV virions, the population comprising: (a) VP1 and VP2 AAV viral structural proteins selected from an AAV8 viral serotype, and (b) VP3 selected from an AAV rhesus monkey serotype AAV rh10 or AAVrh74, wherein the population of rational polyploid AAV virions elicits a reduced humoral response when administered to a subject relative to a corresponding humoral response elicited by a parental AAV8 serotype.

20. A method for repeat dosing comprising first and second AAV administrations to a subject, the method comprising: the first administration performed by administering to the subject a population of rational polyploid AAV virions from any of paragraphs 1-16 or 18-19, and the second administration performed by administering the parental AAV serotype of VP1 or VP2 viral structural proteins, wherein the first administration elicits a reduced humoral response in the subject as compared to a corresponding humoral response as elicited by the parental AAV serotypes of VP1 or VP2 viral structural protein, and wherein VP1 or VP2 are not from a Rhesus AAV serotype.

21. The population of any of paragraphs 18-20, wherein the population evades neutralizing antibodies against the parental serotypes of AAV VP1, VP2, or VP3 viral structural proteins.

22. A method for delivering a transgene across the blood brain barrier of a subject, the method comprising administering to the subject the population of rational polyploid AAV virions of any of paragraphs 18-21.

23. The population of any of the preceding paragraphs, wherein the VP3 protein is a mutated VP3 protein from AAVrh10 or AAVrh74 serotype.

24. The population of paragraph 23, wherein the mutated AAVrh74 VP3 protein has the amino acid sequence of SEQ ID NO: 2 or a protein having at least 85% sequence identity to SEQ ID NO: 2, or wherein the mutated AAVrh74 VP3 comprises at least one of the following modifications of SEQ ID NO: 2: N263S, G264A, T265S, S266T, G268A, T270del, T274H, E533K, R726H, N736P.

25. The population of paragraph 24, wherein the mutated AAVrh10 VP3 protein is encoded by a nucleic acid of SEQ ID NO: 5 that comprises at least one or more of: Q214N, S462N and D517E mutations as compared to AAVrh10_VP3 nucleic acid of SEQ ID NO: 5, or comprises a nucleic acid sequence having at least 85% sequence identity to SEQ ID NO: 5 comprising at least one mutation selected from Q214N, S462N and D517E.

26. The population of paragraph 25, wherein the VP3 protein is a AAVrh74 VP3 protein comprising the amino acid sequence of SEQ ID NO: 2 or 3 or a protein having at least 85% sequence identity to SEQ ID NO: 2 or SEQ ID NO: 3, or comprises at least one of the following amino acid modifications of N263S, G264A, T265S, S266T, G268A, T270del, T274H, E533K, R726H, N736P of SEQ ID NO: 2.

27. A substantially homogenous population of virions of any of paragraphs 1-26, wherein the population is at least 10¹ virions.

28. A nucleic acid comprising, in a 5′ to 3′ direction: a. a first nucleic acid encoding an AAVrh10 VP3 capsid protein operatively linked to a first promoter; b. a first poly A sequence; c. a second nucleic acid encoding a rep protein; d. a third nucleic acid encoding AAV8 VP1 and VP2 viral structural proteins, the third nucleic acid sequence not being capable of expressing an AAV8 VP3 viral structural protein; and e.a second poly A sequence.

29. A nucleic acid comprising, in a 5′ to 3′ direction: a. a first nucleic acid encoding a AAVrh74 VP3 capsid protein operatively linked to a first promoter; b. a first poly A sequence; c. a second nucleic acid encoding a rep protein; d. a third nucleic acid encoding AAV8 VP1 and VP2 viral structural proteins, the third nucleic acid sequence not being capable of expressing a AAV8 VP3 viral structural protein; and e. a second poly A sequence.

30. A viral vector comprising: a. an AAV virion from the population of any of the proceeding paragraphs; and b. a nucleic acid comprising at least one terminal repeat sequence and a heterologous gene, wherein the nucleic acid is encapsulated by the AAV virion.

31. The population of any of the preceding paragraphs comprising a chimeric or modified viral structural protein, wherein the modified viral structural protein comprises insertion, deletion or, substitution of one or more amino acids.

32. The substantially homogenous population of paragraph 27, wherein the substantially homogenous population elicits significantly fewer anti-AAV IgG antibodies against parental AAV serotypes of VP1 or VP2 structural proteins in serum in vivo as compared to a substantially homogenous population of virions comprising parental AAV serotype.

33. The substantially homogeneous population of paragraph 32, wherein the parental AAV serotype is AAV8.

34. A population of rational polyploid AAV virions that allow repeat dosing, the population comprising:
at least one of AAV VP1 or VP2 viral structural proteins and a AAV VP3 viral structural protein;
wherein the VP1 and VP2 viral structural proteins are each from any AAV viral serotype except for a Rhesus AAV serotype, and the VP3 viral structural protein is selected from a rhesus monkey AAV serotype;
wherein the population of rational polyploid AAV virions evade neutralizing antibodies against a parental AAV rhesus monkey serotype of the VP3 viral structural protein, wherein the repeat dosing comprises a first administration of the parental AAV rhesus monkey serotype of the VP3 structural protein and a second administration of the population of rational polyploid AAV virions, and wherein the VP3 structural protein of the rational polyploid virions is a AAV rhesus monkey mutated viral structural protein VP3.

35. The population of rational polyploid AAV virions of paragraph 34, wherein the AAV rhesus monkey mutated viral structural protein VP3 is from a mutated AAV rh10 VP3 viral structural protein or from a mutated AAV rh74 VP3 viral structural protein.

36. The population of rational polyploid AAV virions of any of paragraphs 34-35, wherein the mutated viral structural protein VP3 comprises a mutation at an amino acid that corresponds to an amino acid selected from the group consisting of N263, G264, T265, S266T, G268, T270, T274, and E533, wherein all the amino acid positions correspond to a native VP1 sequence numbering of AAV rh10 or AAVrh74.

37. The population of paragraph 36, wherein the mutation is selected from the group consisting of N263S, G264A, T265S, S266T, G268A, T270del, T274H, and E533K.

38. The population of rational polyploid AAV virions of any of paragraphs 34-37, wherein the mutated viral structural protein VP3 further comprises a mutation at an amino acid that corresponds to an amino acid selected from the group consisting of R727 and N737, wherein all the amino acid positions correspond to a native VP1 sequence numbering of AAVrh10.

39. The population of rational polyploid AAV virions of paragraph 38, wherein the mutation is selected from the group consisting of R727H and N737P.

40. The population of rational polyploid AAV virions of any of paragraphs 34-37, wherein the mutated viral structural protein VP3 further comprises a mutation at an amino acid that corresponds to an amino acid selected from the group consisting of R726 and N736, wherein all the amino acid positions correspond to a native VP1 sequence numbering of AAV rh74.

41. The population of rational polyploid AAV virions of paragraph 40, wherein the mutation is selected from the group consisting of R726H and N736P.

42. The population of rational polyploid AAV virions of paragraph 41, wherein the mutated viral structural protein VP3 further comprises a mutation at an amino acid that corresponds to W at 581, wherein the W is replaced by two subsequent V residues (VV) and wherein all amino acid positions correspond to a native VP1 sequence numbering of AAV rh74.

43. The population of rational polyploid AAV virions of any of paragraphs 34-42, wherein the AAV VP1 or VP2 viral structural protein is any AAV serotype selected from Table 1.

44. The population of rational polyploid AAV virions of any of paragraphs 34-43, wherein the AAV VP1 or VP2 structural protein is AAV8.

45. Use of a population of rational polyploid AAV virions in the manufacturer of a medicament for use for delivering a transgene across a blood brain barrier, the medicament comprising a population of rational polyploid AAV virions of any of paragraphs 1-15 or 18-19, 21, 23-27, 31-44.

46. The use of paragraph 45, wherein the population of rational polyploid AAV virions comprises VP1 and VP2 AAV viral structural proteins selected from an AAV8 viral serotype, and VP3 viral structural protein selected from an AAV rhesus monkey serotype AAV rh10 or AAVrh74.

47. The use of paragraph 45, wherein the population of rational polyploid AAV virions comprises VP1 and VP2 AAV viral structural proteins from an AAV8 viral serotype, and a VP3 structural protein from an AAV rhesus monkey serotype AAVrh74.

48. The use of paragraph 45, wherein the medicament is useful to treat a brain disease or brain disorder or a neurodegenerative disease, or a neurological disease.

49. The use of paragraph 45, wherein the medicament is useful to treat diseases of the central nervous system (CNS) or peripheral nervous system (PNS).

50. The use of paragraph 45, wherein the medicament is useful to treat a subject with a brain cancer or cancer in the brain.

51. The use of paragraph 45, wherein the medicament is useful to treat a subject with a disease or disorder selected from: Alzheimer's disease, Huntington's disease, Parkinson's disease, Amyotrophic Lateral sclerosis (ALS), and Dopamine transporter deficiency syndrome.

52. Use of a nucleic acid in the manufacturer of a medicament comprising a population of rational polyploid AAV virions for use for delivering a transgene across a blood brain barrier, the nucleic acid comprising any of paragraphs 28 or 29.

53. Use of a population of rational polyploid AAV virions in the preparation of a first medicament and a second medicament for use in a method for repeat dosing of a first administration of the first medicament and second administration of the second medicament, wherein the repeat dosing comprises the first administration of the first medicament comprising a rational polyploid AAV virion from any of paragraphs 1-15 or 18-19, and the second administration of the second medicament comprising a parental AAV serotypes of the at least one of VP1 or VP2 viral structural protein, wherein the population of rational polyploid AAV virion elicits a reduced humoral response as compared to a humoral response as elicited by the parental AAV serotypes of the at least one of the VP1 or VP2 viral structural protein, and wherein the VP1 or VP2 is not from a Rhesus AAV serotype.

54. Use of a population of rational polyploid AAV virions in the preparation of a medicament for evading neutralizing antibodies against parental serotypes of AAV VP1, VP2, or VP3 the medicament comprising a population of rational polyploid AAV virions of any of paragraphs 1-15 or 18-19, 21, 23-27 and 31-44.

55. Use of a population of rational polyploid AAV virions in the preparation of a medicament for delivering a transgene to the small intestine, the medicament comprising the population of rational polyploid AAV virions of any of paragraphs 1-15 or 18-19, 21, 23-27 and 31-44.

56. Use of a population of rational polyploid AAV virions in the preparation of a medicament for the treatment of a gastrointestinal disease or disorder, the medicament comprising the population of rational polyploid AAV virions of any of paragraphs 1-15 or 18-19, 21, 23-27 and 31-44.

For example, and without limitation, Applicants reserve the right to disclaim any one or more of the following subject-matters from any claim of the present application, now or as amended in the future, or any patent derived therefrom:

The modified virus capsids can be used as “capsid vehicles,” as has been described, for example, in U.S. Pat. No. 5,863,541. Molecules that can be packaged by the modified virus capsid and transferred into a cell include heterologous DNA, RNA, polypeptides, small organic molecules, metals, or combinations of the same.

### EXAMPLES

### Materials and Method

### Cell Lines.

Pro10 cells, HEK293 cells, Huh7 cells and C2C12 cells were maintained at 37° C. in 5% CO2 in Dulbecco's Modified Eagle's Medium with 10% fetal bovine serum and 10% penicillin-streptomycin.

Recombinant haploid AAV8 Virus Production.

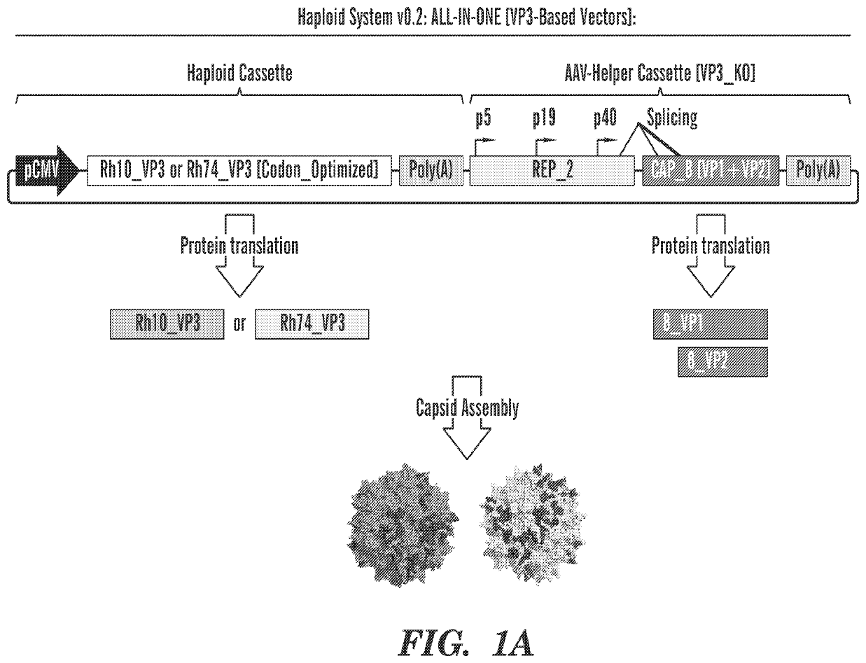

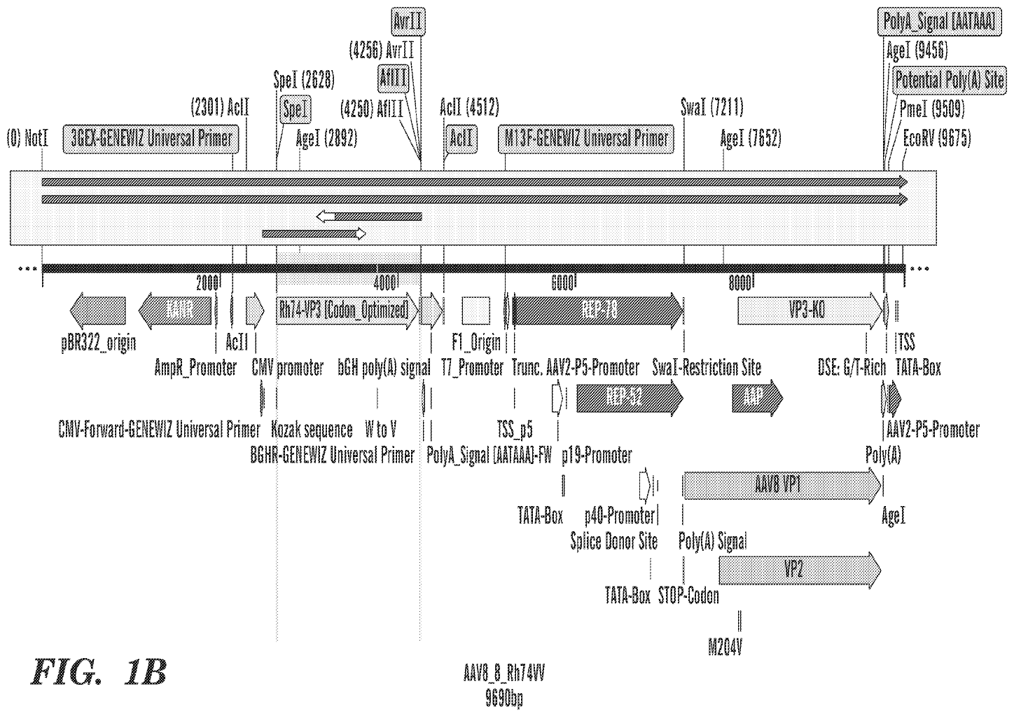

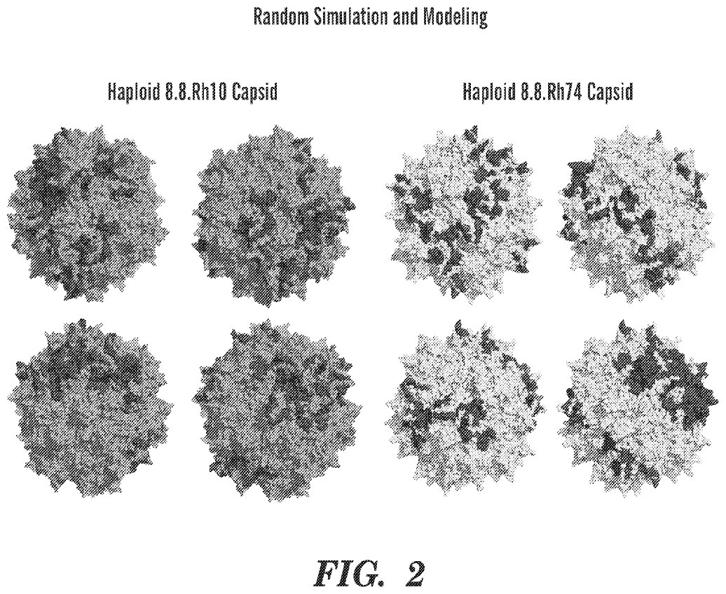

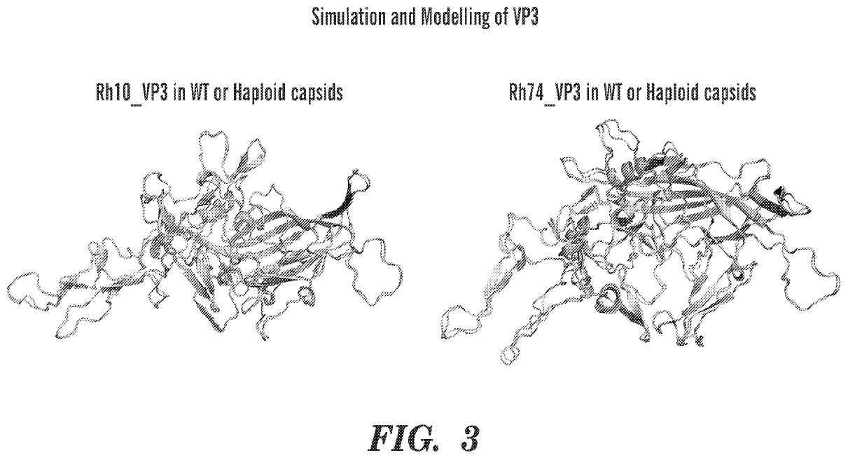

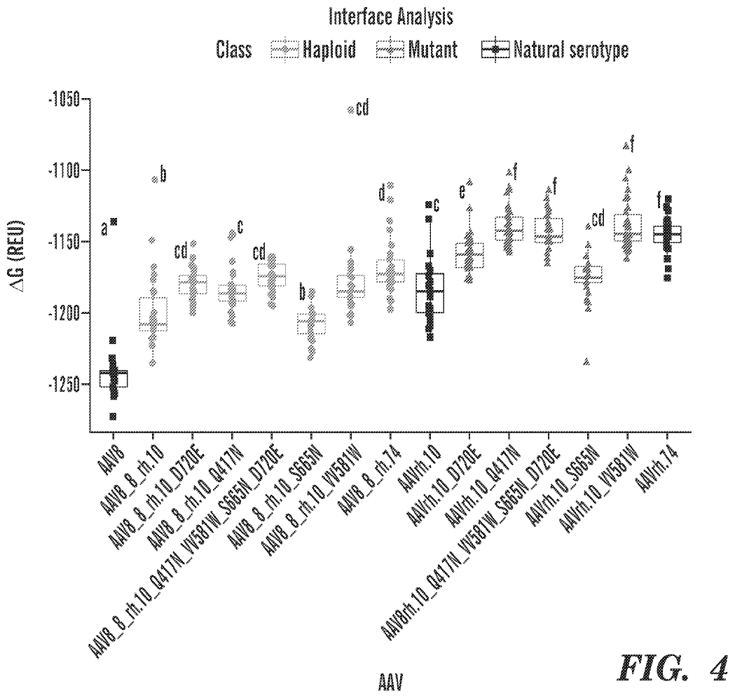

Recombinant AAV was produced by a triple-plasmid transfection system. A 15-cm dish of HEK293 cells was transfected with 9 μg of AAV transgene plasmid pTR/CBA-Luc, 12 μg of AAV helper plasmid, and 15 μg of Ad helper plasmid XX680. To generate the AAV8-8-rh10 and AAV8-8-rh74 haploid virions, the plasmid shown in FIG. 1, comprising either the rh10 VP3 nucleic acid for AAV8-8-rh10 production or the rh74 VP3 nucleic acid for AAV8-8-rh74 haploid production was co-transfected. Sixty hours post-transfection, HEK293 cells were collected and lysed. Supernatant was subjected to CsCl gradient ultra-centrifugation. Virus titer was determined by quantitative PCR.

In some embodiments, the rAAV genomes were packed into haploid AAV capsids to generate haploid rAAV vectors using a rAAV producing cell line. Solely for proof of principal of rAAV vector construction, the capsids used were AAV8 haploid capsids.

Making rAAV in the rAAV producing cell line: triple transfection technique was used to make rAAV in a suspension rAAV producer cell line, which can be scaled up for making clinical grade vector. Alternatively, different plasmids can be used, e.g., 1) pXX680-ad helper and 2) pXR3 the Rep and Cap 3) and the Transgene plasmid (ITR-transgene-ITR).

Methods to generate rAVV polyploid e.g., rational polyploid vectors using a rAAV producing cell line, can be performed according to the methods as described in U.S. Pat. No. 9,441,206, which is incorporated herein in its entirety by reference. In particular, rAAV vectors or rAAV virions are produced using a method comprising: (a) providing a rAAV producing cell line an AAV expression system; (b) culturing the cells under conditions in which AAV particles are produced; and (c) optionally isolating the AAV particles. Ratios of triple transfection of the plasmid and transfection cocktail volumes can be optimized, with varying plasmid ratios of XX680, AAV rep/cap helper and TR plasmid to determine the optimal plasmid ratio for rAAV vector production.

In some instances, the cells are cultured in suspension under conditions in which AAV8 haploid particles are produced. In another embodiment, the cells are cultured in animal component-free conditions. The animal component-free medium can be any animal component-free medium (e.g., serum-free medium) compatible with the rAAV producer cell line. Examples include, without limitation, SFM4Transfx-293 (Hyclone), Ex-Cell 293 (JRH Biosciences), LC-SFM (Invitrogen), and Pro10 cells, or Pro293-S (Lonza). Conditions sufficient for the replication and packaging of the AAV particles can be, e.g., the presence of AAV sequences sufficient for replication of an rAAV genome described herein and encapsidation into AAV capsids (e.g., AAV rep sequences and AAV cap sequences) and helper sequences from adenovirus and/or herpesvirus.

Bacterial DNA sequences from the plasmid backbone can be packaged into AAV8 haploid capsids during manufacturing of the recombinant AAV vectors leading to activations of the innate immune system through its interaction with TLR9 (Akira, 2006; Chadeuf, 2005; Wright, 2014).

In some embodiments, various technologies can be used to eliminate plasmid backbone sequences in recombinant AAV haploid preparations, for example minicircles which have limited scalability (Schnodt, 2016). Another method to avoid bacterial DNA sequence in the plasmid backbone is to use closed ended linear duplex DNA, which includes a range of DNA replication technology, including but not limited to doggy bone DNA (dbDNA™) for specifically manufacturing of recombinant AAV vectors. Using closed ended linear duplex DNA, such as dbDNA™ eliminates the bacterial backbone and has been used to produce vaccines and lentivirus (Walters et al, 2014; Scott et al, 2015; Karda et al, 2019) and was shown to be unable to trigger TLR9 responses by DNA vaccine developers.

Accordingly, in alternative embodiments, generation of rAAV rational polyploid vectors disclosed herein, e.g., AAV8-8-rh10 or AAV8-8-rh10 haploids for example, for use in the methods and compositions as disclosed herein can be performed using closed ended linear duplex DNA, including but not limited to Doggybone technology (dbDNA™), as disclosed in US Application 2018/0037943 and Karbowniczek et al., Bioinsights, 2017, both of which are incorporated herein in its entirety by reference. In brief, a plasmid for AAV production using a closed ended linear duplex DNA technology can comprise the ITRs, promoter and gene of interest is flanked by a 56 bp palindromic protelomerase recognition sequence. In some aspects of the embodiment, the ITR is 145 bp or less. In certain aspects of the embodiment, the ITR is 130 bp. The plasmid is denatured, and in the presence of a Phi29 DNA polymerase, and appropriate primers, Phi29 initiates rolling circle amplification (RCA), creating a double stranded cancatameric repeats of the original construct. When protelomerase is added, binding of the palindromic protelomerase recognition sequences occurs and cleavage-joining reaction occurs to result in a monomeric double stranded (ds) linear covalently closed DNA construct. Addition of common restriction enzymes remove the undesired DNA plasmid backbone sequence and digestion with exonuclease activity, resulting in barbell shaped DNA which can be size fractionated to isolate the barbell shaped DNA sequence encoding the ITRs, promoter and gene of interest. An exemplary plasmid for generation of rAAV vectors using closed ended linear duplex DNA including barbell shaped DNA, comprises in the following 5′ to 3′ direction: 5′-protelomerase RS, 5′ITR, LSP promoter, hGAA, 3′UTR, hGH poly(A), 3′ ITR, 3′-protelomerase RS (sense strand), where the sense strand is linked to the complementary antisense strand for a stranded (ds) linear covalently closed DNA construct. The use of closed ended linear duplex DNA, e.g., barbell shaped DNA as a starting material for the manufacturing of an AAV vector for use in the methods and composition as disclosed herein eliminates the bacterial backbone used to propagate the plasmid containing AAV vector with an inability for the product to trigger Toll-like receptor 9 (TLR9) responses.

### Western and Immune-Blot.

According to the virus titer, the same amount of virions were loaded in each lane, followed by electrophoresis on a NuPage 4-10% polyacrylamide Bis-Tris gel (Invitrogen, Carlsbad, Calif.) and then transferred to PVDF membrane via iBlot® 2 Dry Blotting System (Invitrogen, Carlsbad, Calif.). The membrane was incubated with the anti-Cap antibody specific to AAV capsid proteins.

A native immunoblot assay was carried out as previously described. Briefly, purified capsids were transferred to a Hybond-ECL membrane (Amersham, Piscataway, N.J.) by using vacuum dot-blotter. The membranes were blocked for 1 h in 10% milk PBS and then incubated with monoclonal antibody ADK8. The membranes were incubated with a peroxidase-coupled goat anti-mouse antibody for 1 hr. The proteins were visualized by Amersham Imager 600 (GE Healthcare Biosciences, Pittsburgh, Pa.).

### In Vitro Transduction Assay.

Huh7, C2C12 cells and GM16095 cells were transduced by recombinant viruses with 1×10⁴ vg/cell in a flat-bottom, 24-well plate. Forty-eight hours later, cells were harvested and evaluated by a luciferase assay system (Promega, Madison, Wis.).

### Animal Study.

Animal experiments performed in this study were conducted with C57BL/6 mice. The mice were maintained in accordance to NIH guidelines, as approved by the UNC Institutional Animal Care and Use Committee (IACUC). Six- or seven-week-old female C57BL/6 mice were injected intravenously (iv) with either 5×10¹⁰ vg/mouse or 2.5×10¹² vg/mouse of rational haploid vectors AAV8-Luc (parental control), AAV8-8-rh10-Luc, AAV8-8-rh74-Luc and AAVrh10-Luc (parental control) viruses via the tail vein (n=4). One additional saline-injected mouse was used as negative control in each group (n=7). Vectors were diluted in the formulation buffer (FB; 10 mM phosphate, 2.7 mM KCl, 350 mM NaCl, 5% sorbitol, 0.001% pluronic F68, pH 7.4). One additional mouse receiving vehicle (FB) was included in each group as negative control.

In vivo phase handling: The general condition of the animal, the body position, the ability to interact and the response to stimuli were observed. Any abnormality was recorded. The body weight of mice was monitored weekly during the study (data not shown). Prior to luciferase measurement, animals were anesthetized with an intraperitoneal injection of ketamine (Ketamidor® 100 mg/mL, Richter Pharma AG) mixed with xylacine (Rompun® 20 mg/mL, Bayer Animal Health GmbH) at a dose of ketamine 75 mg/kg and xylacine 15 mg/kg.

Luciferase expression was imaged one week post-injection using a Xenogen IVIS Lumina (Caliper Lifesciences, Waltham, Mass.) following i.p. injection of D-luciferin substrate (Nanolight Pinetop, Ariz.). Bioluminescent images were analyzed using Living Image (PerkinElmer, Waltham, Mass.). Mice were imaged at the indicated time points.

Next, the transduction efficiency of AAV8-8-rh10 and AAV8-8-rh74 haploid viruses in the mouse brain, spinal cord and small intestine was evaluated. AAV8 and AAVrh10 viruses were also injected as controls. A dose of C57BL/6 mice were injected with 3×10¹⁰ vg of recombinant viruses via the tail vein and the imaging was carried out at day 3 post-AAV injection.

### Transgene Expression

To determine the transgene expression, total RNA from tissues was extracted with the Maxwell® 16 LEV simplyRNA Cells/Tissue Kit (Promega) following the manufacturer's instructions. RNA was then treated with DNase I and retro-transcribed into cDNA using M-MLV retro-transcriptase enzyme and random primers. Procedures were performed in a C1000 Touch ThermalCycler (BioRad).

Quantitative analysis was performed by reverse transcription (RT)-qPCR using TaqMan Fast Advanced Master Mix in a CFX Connect Real-time System. cDNA was quantified by real-time qPCR using specific assays for the detection of luciferase (Mr03987587_mr) or mouse gapdh housekeeping gene (Mm99999915_g1; both selected by Askbio and purchased from Thermo Fisher Scientific), and used as a reference gene for normalization of the luciferase data. The relative quantification was carried out using the 2-ΔCt method.

### Statistical Analysis.

The data were presented as mean±SD. The Student t test was used to carry out all statistical analyses. P values <0.05 were considered a statistically significant difference.

### Example 1

Generation of Haploid AAV8 Virions Using Rational Design Methodology—EnhancedAAV Transduction from Haploid AAV8 Vectors by Assembly of AAV8 Haploid Virions with VP1/VP2 from AAV8 Vector and VP3 from Only One Rhesus Monkey AAV (AAVrh) Serotype by Application of Rational Polyploid Methodology

Example 1 is an illustrative example that discloses exemplary combinations of VP1 and VP3 capsid proteins from AAV8 and any serotype that crosses the BBB, respectively, for example a rhesus monkey AAV (AAVrh) serotype, in any order, and optionally a VP2 capsid protein from AAV8 or any serotype that crosses the BBB, including, but not limited to a rhesus monkey AAV (AAVrh) serotype. While AAVrh10 and AAVrh74 capsid proteins are shown as exemplary serotypes that cross the BBB (and which are also AAVrh serotypes), these can be readily replaced or substituted with a VP3 protein from any other serotype that crosses the BBB (including but not limited to, e.g., AAV1, AAV6, AAV6.2, AAV7, AAV9, rAAVrh10, rAAVrh74, rAAVrh39, rAAVrh43 or other AAVrh serotypes). Similarity, AAV8 is shown as an exemplary serotype for the VP1 and/or VP2 protein, but it is encompassed herein that the VP1 and/or VP2 protein from AAV8 can be replaced or substituted for any serotype, e.g., AAV2 or any other serotype disclosed in Table 1 herein.

Herein, the inventors demonstrate enhanced transduction in CNS (and/or PNS) can be achieved from haploid vectors with VP1/VP2 from AAV8 vector capsid and VP3 from an alternative one that crosses BBB, such as rhAAV10 or rhAAV74, or modified VP3 proteins from rhAAV10 or rhAAV74.

The generation of VP1, VP2 and VP3 by different AAV serotypes offers two different strategies for producing these different proteins. Interestingly, the VP proteins are translated from a single CAP nucleotide sequence with overlapping sequences for VP1, VP2 and VP3.

The Cap gene encodes for 3 proteins-VP1, VP2 and VP3. VP1 gene contains the VP1, VP2 and VP3 proteins, and VP2 contains the VP2 and VP3 protein. Therefore, the Cap gene has 3 segments, start of VP1—start of VP2—start of VP3—end of all 3 VP proteins.

In embodiments of rational haploids, the sourcing of the Cap genes can come from two different AAV serotypes (designated as serotypes X or Y (e.g., for any serotype selected from Table 1, e.g., AAV8) and serotype Z (for a AAV serotype that crosses the BBB, including but not limited to AAVrh)), there are 6 possible combinations of the three Cap proteins. In one case, the VP1 identified as serotype AAV8, (or chimeric or other nonnaturally occurring AAV8) is only from AAV8 and the VP2/VP3 identified as serotype Y, is only from serotype Y, and is a serotype that is different from the serotype (or chimeric or other nonnaturally occurring AAV) of VP1. In one case, both VP1 and VP2 are only from AAV8, and VP3 is only from serotype Z, where serotype Z is an AAV serotype that crosses the BBB and/or is a non-human primate AAV serotype. Methods to create a VP1 of AAV8 and VP2/VP3 of a serotype Z; or VP1/VP2 from a AAV8 serotype and VP3 form a serotype Z, are disclosed in the Examples set forth herein. In one case, VP1 and VP3 are only from a first serotype and VP2 is only from a second serotype.

TABLE 5
Exemplary combinations of AAV8 haploid or AAV8 polyploid vectors,
where Serotype Z is any AAV serotype that crosses the BBB,
or alternatively, is from a non-human primate, including a
rhesus monkey AAV serotype (AAVrh), and serotype X is a third
AAV serotype that is not AAV8 or serotype Z, and where serotype
X can be any AAV serotype that is a rhesus monkey AAV serotype
(AAVrh), or can be any serotype or chimeric or nonnaturally
occurring serotype that is not AAV8 or the serotype Z.

| VP1 | VP2 | VP3 |
|---|---|---|
| AAV8 | AAV8 | Serotype Z |
| AAV8 | Serotype Z | Serotype Z |
| AAV8 | Serotype X | Serotype Z |
| AAV8 | Serotype Z | Serotype X |
| AAV8 | Serotype X | Serotype X |

In some embodiments, VP1 is not AAV8, providing the AAV8 haploid comprises at least one of VP1, VP2 or VP3 from AAV8, the following combinations are shown in Table 6:

TABLE 6
Exemplary AAV8 haploid vectors, where VP1 is not AAV8
but comprises at least one of VP1, VP2 or VP3 from
AAV8, and where Serotype Z is from any serotype that
crosses the BBB, or alternatively, from any a rhesus
monkey AAV serotype (AAVrh), and is not AAV8,

| VP1 | VP2 | VP3 |
|---|---|---|
| AAV8 | Serotype Z | Serotype Z |
| AAV8 | Serotype Z | AAV8 |
| AAV8 | AAV8 | Serotype Z |
| Serotype Z | Serotype Z | AAV8 |
| Serotype Z | AAV8 | Serotype Z |
| Serotype Z | AAV8 | AAV8 |

In some embodiments, the sourcing the Cap genes from three different AAV serotypes (designated as AAV8, X and Z), where there are 6 possible combination of the three Cap proteins. In this case, the VP1 identified from AAV8, (or chimeric or other nonnaturally occurring AAV of AAV8) that is different from the serotype of VP2 and VP3; the VP2 identified as serotype X, which is a serotype that is different from the serotype of VP1 and VP3 and is from a second serotype; and, the serotype of VP3 identified as serotype Z, which is a serotype that is different from the serotype of VP1 and the serotype of VP2, is from a third serotype. Methods to create a VP1 of a first serotype, a VP2 of a second serotype and a VP3 of a third serotype are disclosed in the Examples set forth herein.

TABLE 7
Exemplary AAV8 haploid vectors comprising at least one VP
protein from AAV8, where VP1, VP2 or VP3 are each from different
serotypes, and where Serotype Z is any AAV serotype that
crosses the BBB and/or is a non-human primate AAV serotype
and is not AAV8, and serotype X can be any AAV serotype that
crosses the BBB and/or is a non-human primate AAV serotype,
or can be any serotype or chimeric or nonnaturally occurring
serotype that is not AAV8 or the serotype Y.

| VP1 | VP2 | VP3 |
|---|---|---|
| AAV8 | Serotype X | Serotype Z |
| AAV8 | Serotype Z | Serotype X |
| Serotype X | AAV8 | Serotype Z |
| Serotype X | Serotype Z | AAV8 |
| Serotype Z | AAV8 | Serotype X |
| Serotype Z | Serotype X | AAV8 |

In an embodiment, when VP1 is AAV8 and VP2 and VP3 are identified as a second serotype Z, it is understood that in one embodiment, this would mean that VP1 is only from AAV8 and that VP2 and VP3 is only from serotype Z, where serotype Z is from any serotype that crosses the BBB. In another embodiment, when VP1 is identified AAV8, VP2 as a second serotype X and VP3 as a third serotype Z, it is understood that in one embodiment, this would mean that VP1 is only from AAV8; that VP2 is only from serotype X; and VP3 is only from serotype Z. As described in more detail in the Examples below, in one embodiment, to create a haploid vector using two different serotypes you could include a nucleotide sequence for VP1 from AAV8 (or chimeric or other non-naturally occurring AAV) that expresses only VP1 from AAV8 and a second nucleotide sequence for VP2 and/or VP3 only from a second serotype, or alternatively VP2 only from a second serotype, and VP3 only from a third serotype. In one embodiment, VP1/VP2 are only from AAV8 serotype and VP3 is only from a second serotype, e.g., a serotype that crosses the BBB and/or is a non-human primate AAV serotype and is not AAV8.

In the case of 3 different Cap genes, the helper plasmid can be generated with a full copy of the nucleotide sequence for the particular VP protein from the three AAV serotypes. The individual Cap genes will generate the VP proteins associated with that particular AAV serotype (designated as AAV8, X and Z). These nucleotide sequences can be modified with non-functional or inactivated start sites to allow only the expression of the preferred VP protein, as disclosed herein in Examples 3-4 herein.

TABLE 8
a single construct with nucleotide sequences for VP proteins.

| VP1 | VP2 | VP3 |
|---|---|---|
| AAV8 | Serotype X | Serotype Z |
| AAV8 | Serotype Z | Serotype X |
| Serotype X | AAV8 | Serotype Z |
| Serotype X | Serotype Z | AAV8 |
| Serotype Z | AAV8 | Serotype X |
| Serotype Z | Serotype X | AAV8 |

In an embodiment, when VP1 is identified as AAV8 and VP2 is identified as a second serotype X and VP3 is identified as a third serotype Z, it is understood that in one embodiment, this would mean that VP1 is only from AAV8; that VP2 is only from serotype X and VP3 is only from serotype Z. As described in more detail in the Examples below, to create such a haploid vector would include a nucleotide sequence for VP1 from AAV8 that expresses only VP1 from AAV8 and not VP2 or VP3 from AAV8; a second nucleotide sequence that expresses VP2 of serotype X and not VP3 of serotype X; and a third nucleotide sequence that expresses VP3 of serotype Z.

In certain embodiments, the haploid virions comprise only VP1 and VP3 capsid proteins. In some embodiments, the haploid comprises VP1 from AAV8 and VP3 from any serotype that crosses the BBB and/or is a non-human primate AAV serotype and is not AAV8. In certain embodiments, the haploid virions comprise VP1, VP2, and VP3 capsid proteins. In some embodiments, the haploid comprises VP1 from AAV8, VP2 and/or VP3 from any serotype that crosses the BBB and/or is a non-human primate AAV serotype that is not AAV8.

It should be noted that in each of these embodiments of various combinations of VP1 with VP3 to form a haploid AAV8 virion; or various serotype combinations of VP1/VP2/VP3 to from a haploid AAV8 virion, the nucleotide sequences that express the capsid proteins can be expressed from one or more vector, e.g., plasmid. In one embodiment, the nucleic acid sequences that express VP1, or VP2, or VP3, are codon optimized so that recombination between the nucleotide sequences is significantly reduced, particularly when expressed from one vector, e.g., plasmid etc.

### Example 2

Rational Polyploid e.g., Rational Polyploid Vector with VP1/VP2 from AAV8 and VP3 from a Serotype which Crosses the BBB and/or a Non-Human Primate AAV Serotype Enhances AAV Transduction.

Example 2 is an illustrative example that discloses exemplary combinations of VP1 and VP3 capsid proteins from AAV8 and any serotype that crosses the BBB, respectively, for example a rhesus monkey AAV (AAVrh) serotype, in any order, and optionally a VP2 capsid protein from AAV8 or any serotype that crosses the BBB, including, but not limited to a rhesus monkey AAV (AAVrh) serotype. While AAVrh10 and AAVrh74 capsid proteins are shown as exemplary serotypes that cross the BBB (and which are also AAVrh serotypes), these can be readily replaced or substituted with a VP3 protein from any other serotype that crosses the BBB (including but not limited to, e.g., AAV1, AAV6, AAV6.2, AAV7, AAV9, rAAVrh10, rAAVrh74, rAAVrh39, rAAVrh43 or other AAVrh serotypes). Similarity, AAV8 is shown as an exemplary serotype for the VP1 and/or VP2 protein, the VP1 and/or VP2 protein from AAV8 can be readily replaced or substituted by one of ordinary skill in the art for any serotype disclosed in Table 1 herein.

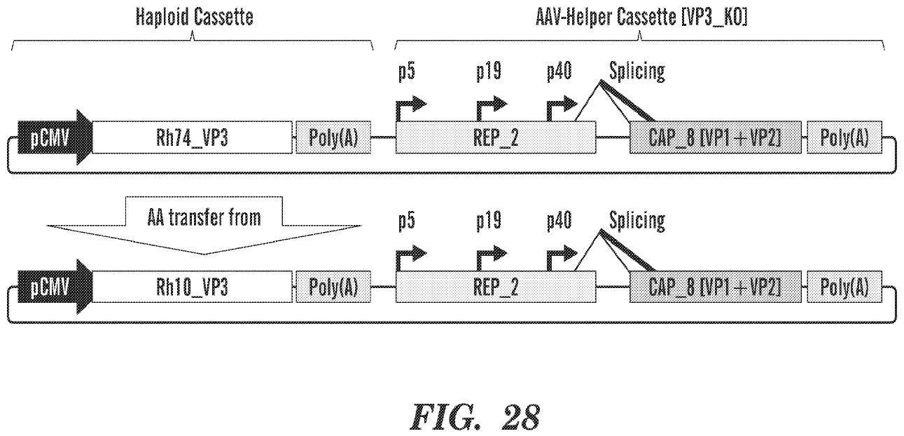

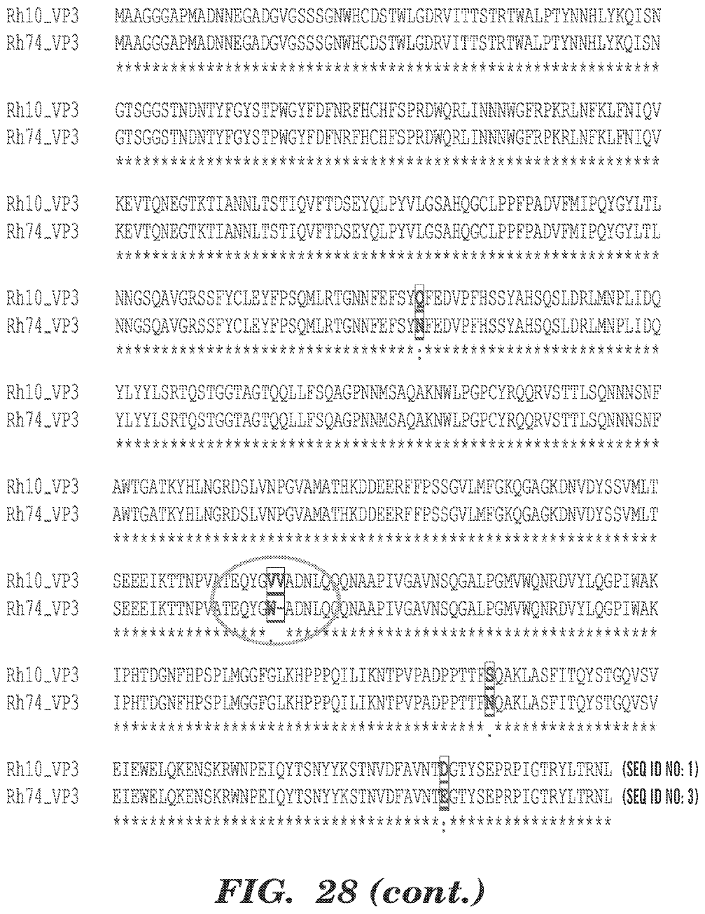

To elucidate which AAV subunits in individual rational polyploid e.g., haploid AAV8 vector contributes to higher transduction than AAV8, different constructs were made as follows: AAV8-8-rh10 and AAV8-8-rh74, which expressed AAV8 VP1/VP2 only, and VP3 only from AAVrh10 or AAVrh74. These plasmids were used to produce haploid AAV8 vectors. Exemplary plasmid constructs are shown in FIG. 1 and FIG. 28.

After injection of 5×10¹⁰ vg of these haploid AAV8 vectors per mice via intravenous administration via tail vein injection, biodistribution was evaluated by weekly IVIS imaging. Haploid AAV8-8-rh74 vector induced a significantly higher systemic transduction than AAV8 or AAVrh10 (see FIG. 21A-21D) showing unexpected ability to cross BBB compared to AAV8, AAVrh10.

### Example 3

Creation of AAV8-8-Z Rational Polyploid Capsids from AAV8 and a Second Serotype (e.g., AAVrh10 or AAVrh74) and Mutation of Start Codons

Example 3 is an illustrative example that discloses exemplary combinations of VP1 and VP3 capsid proteins from AAV8 and any serotype that crosses the BBB, respectively, for example a rhesus monkey AAV (AAVrh) serotype, in any order, and optionally a VP2 capsid protein from AAV8 or any serotype that crosses the BBB, including, but not limited to a rhesus monkey AAV (AAVrh) serotype. While AAVrh10 and AAVrh74 capsid proteins are shown as exemplary serotypes that cross the BBB (and which are also AAVrh serotypes), these can be readily replaced or substituted with a VP3 protein from any other serotype that crosses the BBB (including but not limited to, e.g., AAV1, AAV6, AAV6.2, AAV7, AAV9, rAAVrh10, rAAVrh74, rAAVrh39, rAAVrh43 or other AAVrh serotypes). Similarity, AAV8 is shown as an exemplary serotype for the VP1 and/or VP2 protein, the VP1 and/or VP2 protein from AAV8 can be readily replaced or substituted by one of ordinary skill in the art for any serotype disclosed in Table 1 herein.

In this example, rational polyploid AAV8 virions e.g., haploid AAV8 virions are assembled from capsids of two different serotypes, as shown in FIG. 1 and FIG. 28. This example discusses production of exemplary AAV8-8-rh10 or AAV8-8-rh74 haploid virions. However, one of ordinary skill in the art would readily be able to use a VP3 from any AAV serotype which crosses the BBB and/or is a non-human primate AAV serotype. In addition, one could readily substitute the AAV8 VP2 protein for any AAV serotype which crosses the BBB and/or is a non-human primate AAV serotype, which can be the same serotype, or a different serotype to that used for the VP3 capsid protein.

In this experiment, a nucleotide sequence for VP1, VP2 and VP3 from AAV8 serotype only are ligated into a helper plasmid and the VP3 from a second AAV serotype (e.g., an AAV serotype that crosses the BBB and/or is from a non-human primate AAV serotype) only is ligated into the same or different helper plasmid, such that the helper plasmid/s include the VP1, VP2 and VP3 capsid proteins from two different serotypes. Either prior to ligation or following ligation of the first and second serotype nucleotide sequences coding for VP1, VP2 and VP3 capsid proteins into the helper plasmid, the capsid nucleotide sequences are altered to provide a VP1 and VP2 from the AAV8 serotype only and a VP3 from a second serotype only. In this example, two ACG start sites of VP3 of AAV8 is mutated such that these start codons cannot initiate the translation of the RNA transcribed from the nucleotide sequence of the VP3 capsid protein from the AAV8 serotype. In particular, of the AAV8 nucleotide sequence, two ATG initiation codons are changed to GTG to result in amino acid substitutions M203V and M211V, which prevent translation and expression of the VP3 AAV8 capsid protein. Similarly, in some embodiments, the helper plasmid comprises only the VP3 gene of the other serotype (e.g., AAVrh10 or AAVrh74). In alternative embodiments, the ATG start site of VP1 and VP2 can be mutated in the nucleotide sequence coding for the capsid proteins of the second serotype (e.g., AAVrh10 or AAVrh74), such that these codons cannot initiate the translation of the RNA coding for VP1 and VP2, but translation can be initiated for both VP3. Thus, in this example, a haploid AAV8 virion is created that includes VP1 and VP2, but not VP3 from AAV8 serotype only and a VP3, but not VP1 and VP2 from a second serotype only (e.g., AAVrh10 or AAVrh74).

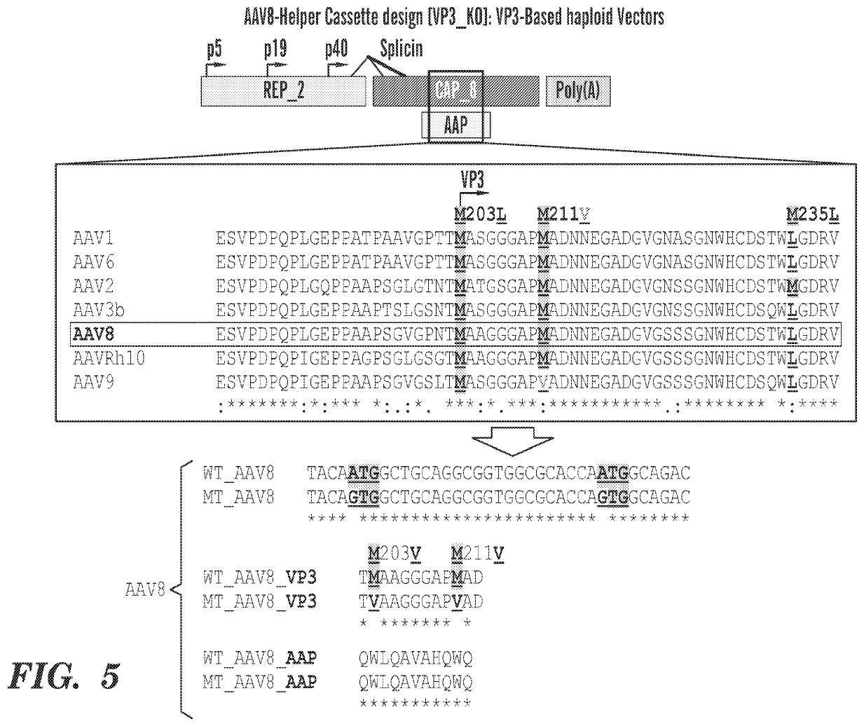

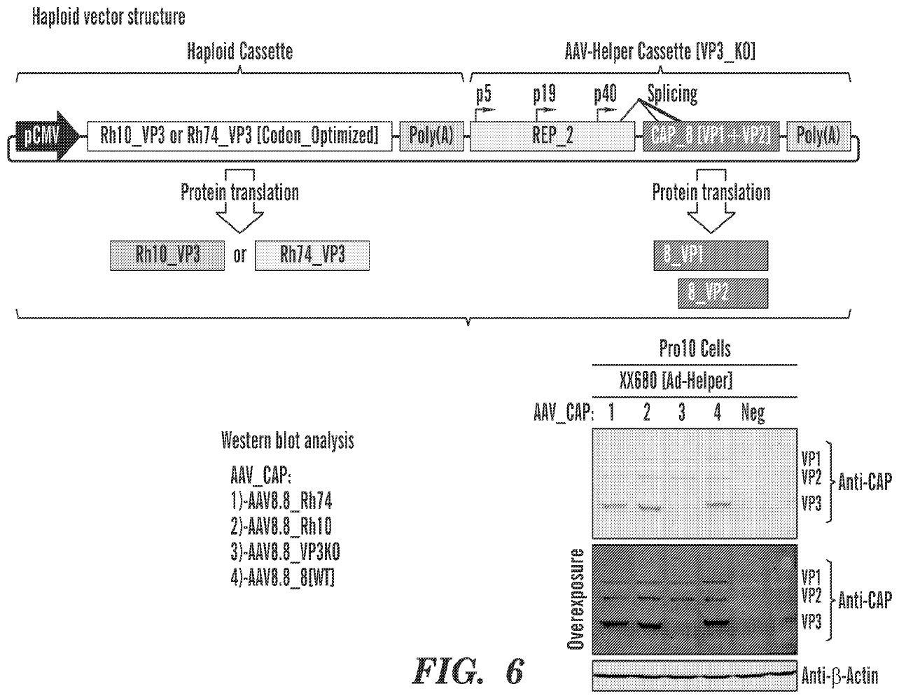

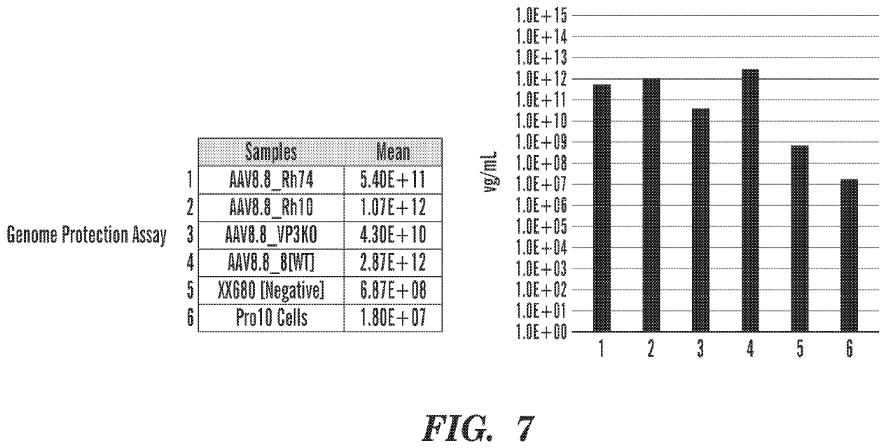

In applying this rational methodology technique of creating a haploid AAV8 virions through mutation of start codons, the start codon of VP3 of AAV8 was mutated as shown with highlights in FIG. 5, such that VP1 and VP2 are translated from an RNA transcribed from the plasmid set forth in FIG. 1 and FIG. 6. Thus, mutation of the start codons provides a method of knocking out the expression of one or more of VP1, VP2 and VP3. Thus, in this example, a haploid AAV8 virion is created that includes a VP1 and VP2, but not VP3 from AAV8 serotype only and a VP3, but not a VP1 or VP2 from a second AAV serotype only (e.g., AAVrh10 or AAVrh74). Representative haploid AAV8 vectors can be, e.g., AAV8-8-rh10 and AAV8-8-rh74.

Creation of AAV8-Y-Z Haploid Capsids from Two Different Serotypes (AAV8 and AAVrh10 or AAVrh74) and Mutation of Splice Acceptor Sites

In this example, polyploid AAV virions are assembled from capsids of two different serotypes. The nucleotide sequence for VP1, VP2 and VP3 from AAV8 serotype only are ligated into a helper plasmid and the VP1, VP2 and VP3 from a second AAV serotype (e.g., AAVrh10 or AAVrh74) only is ligated into the same or different helper plasmid, such that the helper plasmid/s include the VP1, VP2 and VP3 capsid proteins from two different serotypes. Either prior to ligation or following ligation of the first and second serotype nucleotide sequences coding for VP1, VP2 and VP3 capsid proteins into the helper plasmid/s, the capsid nucleotide sequences are altered to provide a VP1 from AAV8 serotype only and a VP2 and VP3 from a second AAV serotype (e.g., AAVrh10 or AAVrh74) only. In this example, the nucleotide sequence of the first serotype has been altered by mutating the A2 Splice Acceptor Site as shown in FIG. 1. In this example, by mutating the A2 Splice Acceptor Site, the VP2 and VP3 capsid proteins from AAV8 are not produced. Similarly, by mutating the A1 Splice Acceptor Site, the VP1 capsid protein from the second AAV serotype is not produced, while VP2 and VP3 capsid proteins are produced. Thus, in this example, a haploid AAV8 virion is created that includes a VP1, but not VP2 or VP3 from AAV8 serotype only and a VP2 and VP3, but not a VP1 from a second AAV serotype only (e.g., AAVrh10 or AAVrh74). Representative haploid AAV8 vectors can be, e.g., AAV8-rh10-rh10 and AAV8-rh74-rh74.

Creation of AAV8-X-Y Polyploid Capsids from Three Different Serotypes (AAV8, Serotypes Represented by an X and 1) and Mutation of Start Codons and Splice Acceptor Sites

In this example, polyploid AAV virions are assembled from capsids of three different serotypes. A helper plasmid is constructed so that the nucleotide sequence for VP1, VP2 and VP3 from the AAV8 serotype only, the VP1, VP2 and VP3 from a second AAV serotype (referred to as “X” AAV serotype) only and the VP1, VP2 and VP3 from a third AAV serotype only (referred to as “Z” AAV serotype, e.g., a serotype that crosses the BBB, herein exemplified by AAVrh10 or AAVrh10) are ligated into a helper plasmid/s, such that the helper plasmid/s include/s the nucleic acid sequences for VP1, VP2 and VP3 capsid proteins from three different serotypes. Either prior to ligation or following ligation of the nucleotide sequences coding for VP1, VP2 and VP3 capsid proteins from each of the three different serotypes into the helper plasmid, the capsid nucleotide sequences are altered to provide VP1 from the AAV8 serotype only, VP2 from the X AAV serotype only, and VP3 from the Z serotype only (e.g., a BBB serotype, AAVrh10 or AAVrh74). In this example, the VP1 nucleotide sequence of the AAV8 serotype has been altered by mutating the start codons for the VP2 and VP3 capsid proteins. In this example, the ACG start codon of VP2 and the two ATG start codons of VP3 are mutated such that these codons cannot initiate the translation of the RNA transcribed from the nucleotide sequence of the VP2 and VP3 capsid proteins from the first serotype. Similarly, the VP1 and VP3 nucleotide sequence of the second X serotype have been altered by mutating the start codons for the VP1 and VP3 capsid proteins. In this example, the ATG start site of VP1 and the two or three ATG start codons of VP3 are mutated such that these codons cannot initiate the translation of the RNA transcribed from the nucleotide sequence of the X serotype VP1 and VP3 capsid proteins. Further, the VP1 and VP2 nucleotide sequence of the third Z serotype (e.g., AAVrh10 or AAVrh74) have been altered by mutating the start codons for the VP1 and VP2 capsid proteins. In this example, the ATG start codon of VP1 and the ACG start codon of VP2 are mutated such that these codons cannot initiate the translation of the RNA transcribed from the nucleotide sequence of the VP1 and VP2 capsid proteins from the Z serotype (e.g., a serotype that crosses the BBB, e.g., exemplary serotypes AAVrh10 or AAVrh74). Alternatively, the helper plasmid comprises only the nucleic acid encoding the VP3 from the third Z serotype (e.g., AAVrh10 or AAVrh74). Thus, in this example, a polyploid AAV virion is created that includes a VP1, but not VP2, nor VP3 from the AAV8 serotype only; a VP2, but not a VP1, nor VP3 from a second Z serotype only; and, VP3, but not VP1, nor VP2 from a third Z serotype only (e.g., AAVrh10 or AAVrh74). Representative haploid AAV8 vectors produced by this methodology can be, e.g., AAV8-X-rh10 and AAV8-X-rh74, where X is a VP2 protein from any AAV serotype, but in particular, an AAV serotype which crosses the BBB and/or is a non-human primate AAV serotype.

Creation of AAV8 Rational Polyploid e.g., AAV8 Haploid Capsids from Two Different Serotypes Using Two Plasmids

In this example, a haploid AAV8 virion comprising VP1/VP2 from AAV8 and VP3 from AAVRh10 or AAVrh74 is created using two plasmids. A helper plasmid is created that includes a plasmid backbone along with Ad Early Genes and Rep (e.g., from AAV2). This helper plasmid has ligated into it the nucleotide sequence coding for the capsid proteins from AAV8 only and a separate nucleotide sequence coding for the capsid proteins of AAVrh10 or AAVrh74 only. With regard to the nucleotide sequence coding for the capsid proteins of AAV8, this nucleotide sequence has had either the start codons for VP3 mutated to prevent translation of VP3 and/or the A2 Splice Acceptor Site has been mutated to prevent splicing. With regard to the nucleotide sequence coding for the capsid proteins of AAVrh10 orAAVrh74, this nucleotide sequence has had either the start codon for VP1 and VP2 mutated to prevent translation and/or the A1 Splice Acceptor Site has been mutated to prevent splicing, or comprises only the portion of the nucleic acid sequence encoding the VP3 capsid protein. The helper plasmid, along with a plasmid encoding the transgene with two ITRs are transfected into HEK293 cell line with ATCC No. PTA 13274 (see e.g., U.S. Pat. No. 9,441,206). The virus is purified from the supernatant and characterized.

Creation of Haploid Capsids from Two Different Serotypes Using Three Plasmids

In this example, a haploid AAV8 virion comprising VP1/VP2 from AAV8 and VP3 from AAVrh10 or AAVth74 is created using three plasmids. A first helper plasmid is created that includes the Ad Early Genes. A second helper plasmid is created that includes a plasmid backbone along with Rep (e.g., AAV2). This second helper plasmid has ligated into it the nucleotide sequence coding for the capsid proteins from AAV8 only and a separate nucleotide sequence coding for the capsid proteins of AAVrh10 or AAVrh74 only. With regard to the nucleotide sequence coding for the capsid proteins of AAV8, this nucleotide sequence has had either the start codons for VP3 mutated to prevent translation and/or the A2 Splice Acceptor Site has been mutated to prevent splicing. With regard to the nucleotide sequence coding for the capsid proteins of AAVrh10 or AAVrh74, this nucleotide sequence has had either the start codon for VP1 and VP2 mutated to prevent translation and/or the A1 Splice Acceptor Site has been mutated to prevent splicing, or the nucleotide sequence encodes just the VP3 capsid protein of AAVrh10 or AAVrh74. The helper plasmids, along with a plasmid encoding the transgene with two ITRs are transfected into HEK293 cell line with ATCC No. PTA 13274 (see e.g., U.S. Pat. No. 9,441,206).

Other methods to generate the AAV8 haploids, e.g., AAV-8-X-Y or AAV8-Y-Y as disclosed herein, with exemplary haploids AAV8-8-rh10 or AAV8-8-rh74 discussed herein, using one, two, three or four plasmids with mutagenesis of ATG start codons of any one or more of VP1, VP2 or VP3, or using DNA shuffling can be used as disclosed in U.S. Pat. No. 10,550,405, which is incorporated herein in its entirety by reference.

### Example 4

### Production, Purification and Analysis of AAV8-8-Rh10 and AAV8-8-Rh74 Rational Polyploids.

Example 4 is an illustrative example that discloses exemplary combinations of VP1 and VP3 capsid proteins from AAV8 and any serotype that crosses the BBB, respectively, for example a rhesus monkey AAV (AAVrh) serotype, and optionally a VP2 capsid protein from AAV8 or any serotype that crosses the BBB, including, but not limited to a rhesus monkey AAV (AAVrh) serotype. While AAVrh10 and AAVrh74 capsid proteins are shown as exemplary serotypes that cross the BBB (and which are also AAVrh serotypes), these can be readily replaced or substituted with a VP3 protein from any other serotype that crosses the BBB (including but not limited to, e.g., AAV1, AAV6, AAV6.2, AAV7, AAV9, rAAVrh10, rAAVrh74, rAAVrh39, rAAVrh43 or other AAVrh serotypes). Similarly, AAV8 is shown as an exemplary serotype for the VP1 and/or VP2 protein, although the VP1 and/or VP2 protein from AAV8 can be readily replaced or substituted by one of ordinary skill in the art for any serotype disclosed in Table 1 herein.

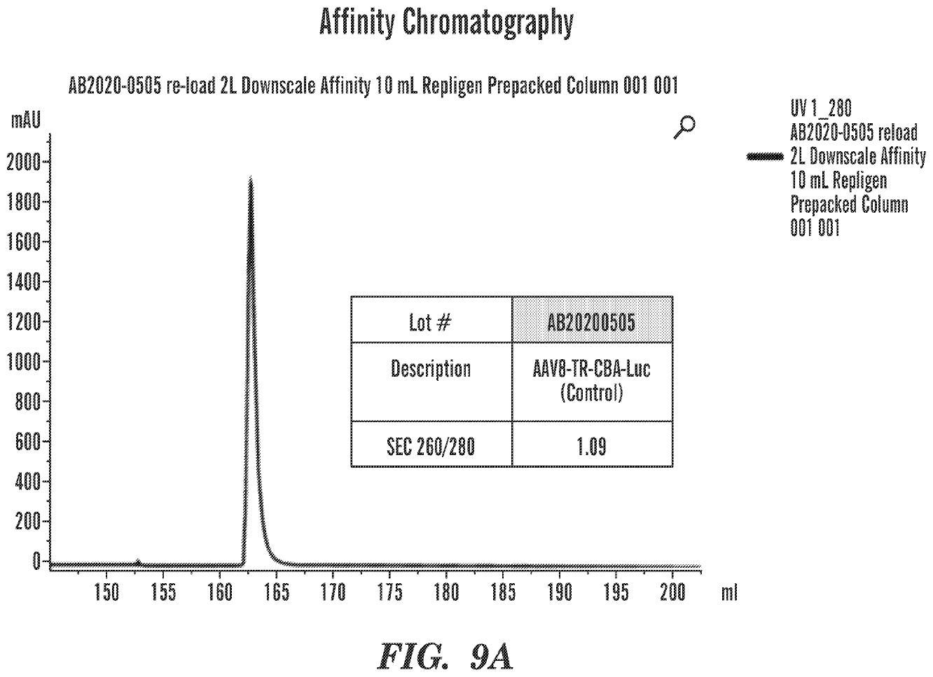

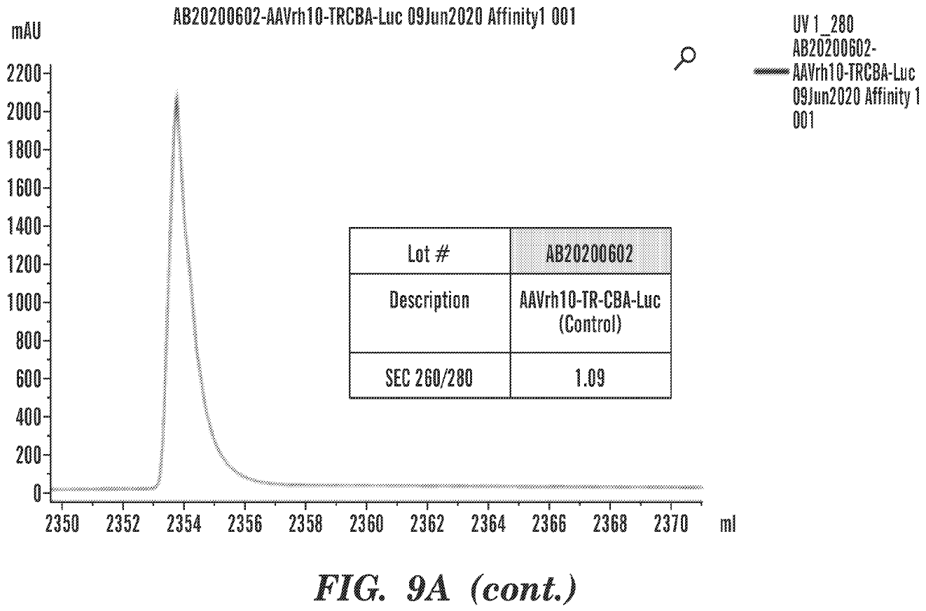

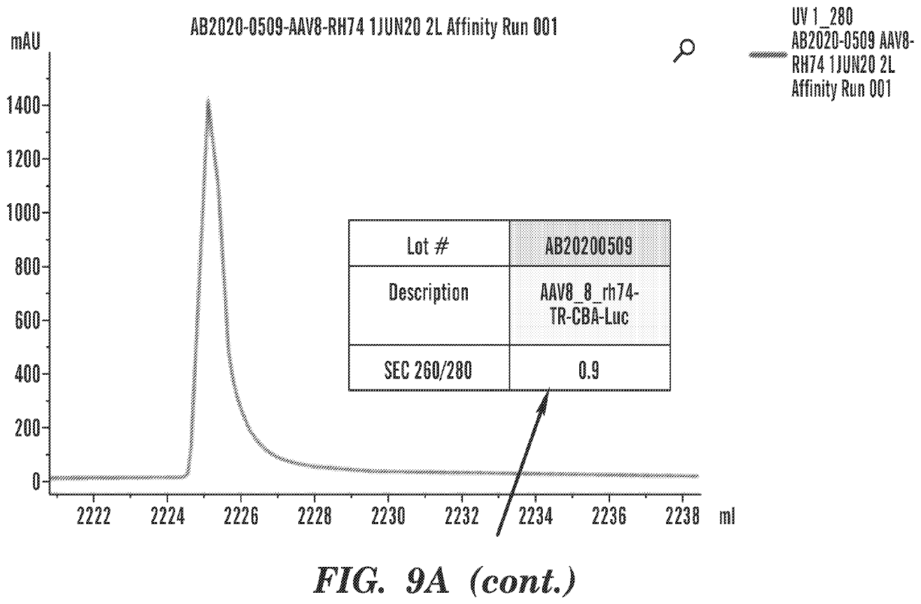

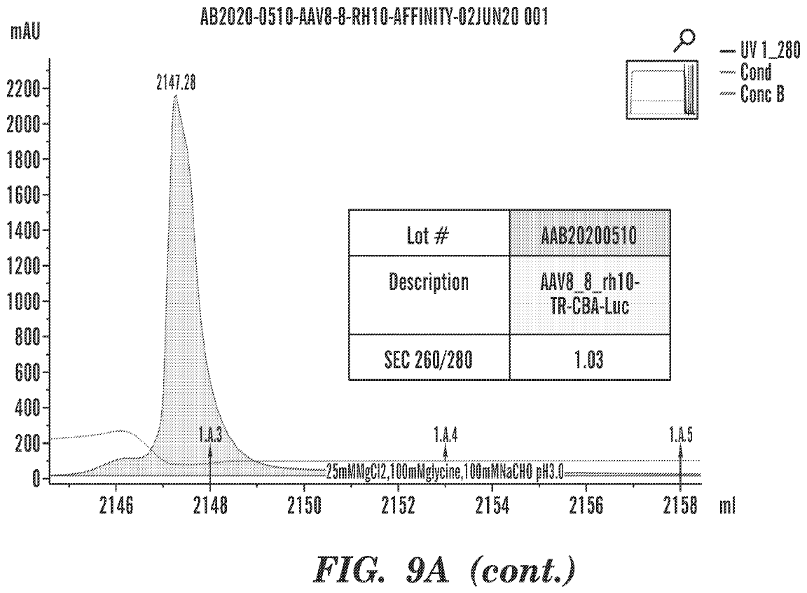

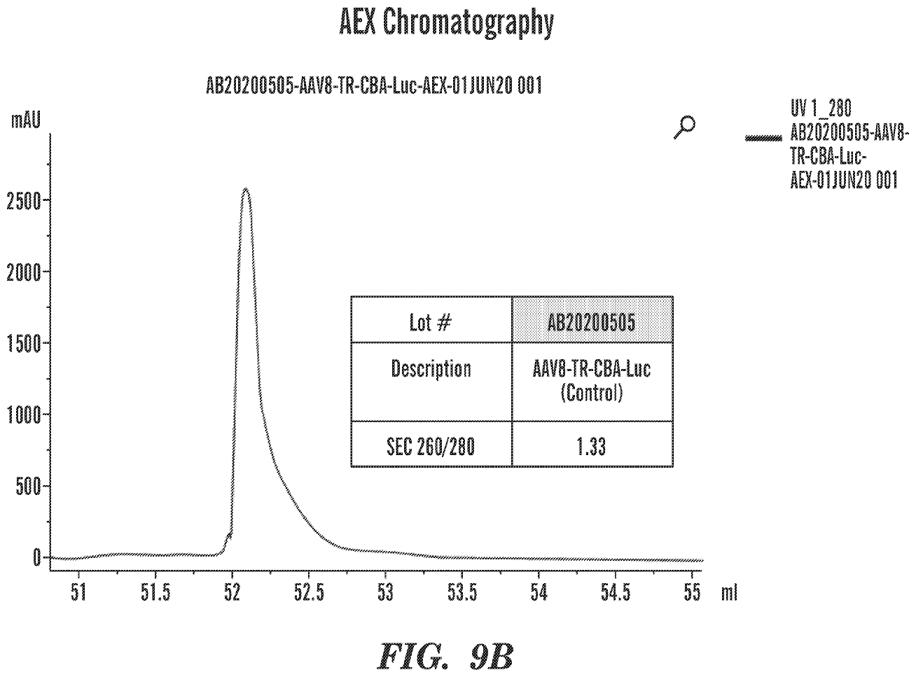

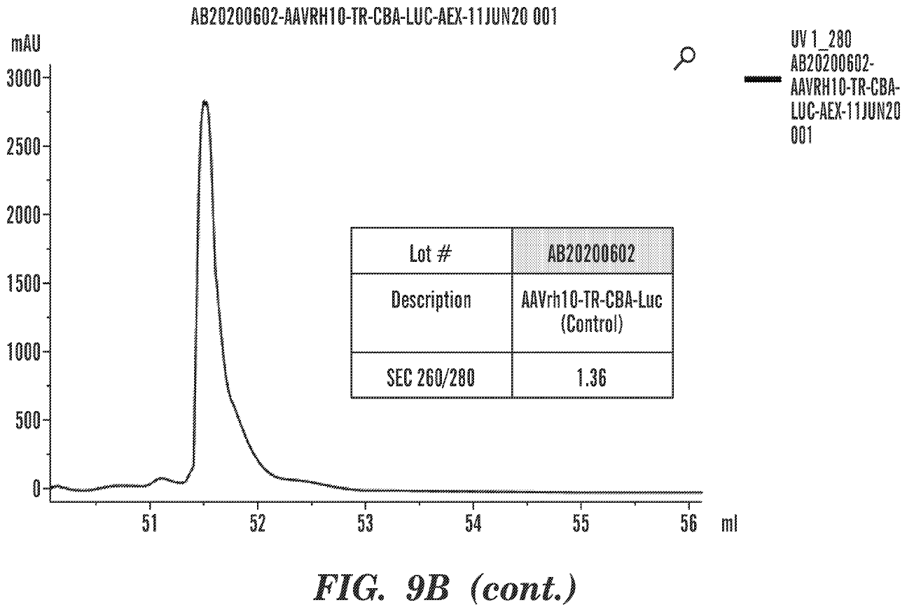

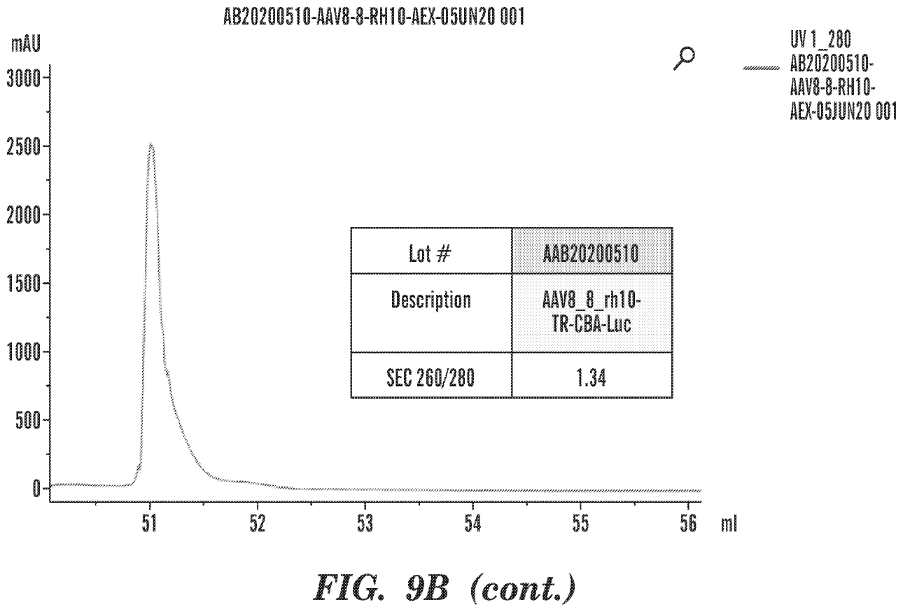

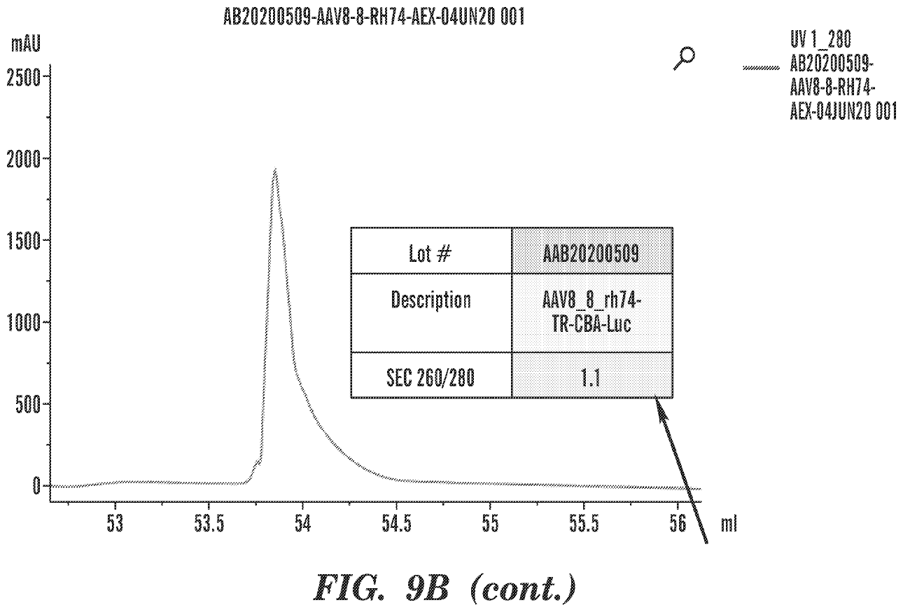

The inventors assessed the yield of the AAV8-8-rh10 or AAV8-8-rh74 haploids, including variants thereof, e.g., AAV8-8-rh74vv, AAV 8-8-rh10LP2, AAV 8-8-rh74LP2, AAV 8-8-rh74vvLP2, as compared to the yield and production of AAV8 and AAVrh10 virions. FIG. 6 shows expression of VP1, VP2 and VP3 proteins as detected by western blot from AAV8-8-rh10 or AAV8-8-rh74 haploids. Production was assessed by cell specific productivity, as determined by qPCR and ELISA, and demonstrated that AAV8-8-rh10 productivity was comparable to AAV8 and AAVrh10 control, while AAV8-8-rh74 was at a lower productivity level as compared to AAV8-8-rh10 and controls. Comparison of means statistically insignificant (see FIG. 8). Affinity chromatography and Anion exchange chromatography (AEX) results were also shown (see FIG. 9A-9B).

Production and purification of AAV8-8-rh10 was similar to AAV8 and AAVrh10 controls with comparable recovery profiles (FIG. 10C). AAV8-8-rh74 production and purification was marginally lower in upstream production compared to AAV8 and AAVrh10 controls with poor recovery in downstream unit operations (see FIG. 10D). AAV8 and AAVrh10 controls performed as expected (FIG. 10A-10B). AAV 8-8-rh74LP2 had significantly less production yield or production titer (5.02 E+09 vg/ml) than AAV 8-8-rh10 LP2 (6.11E+12 vg/ml).

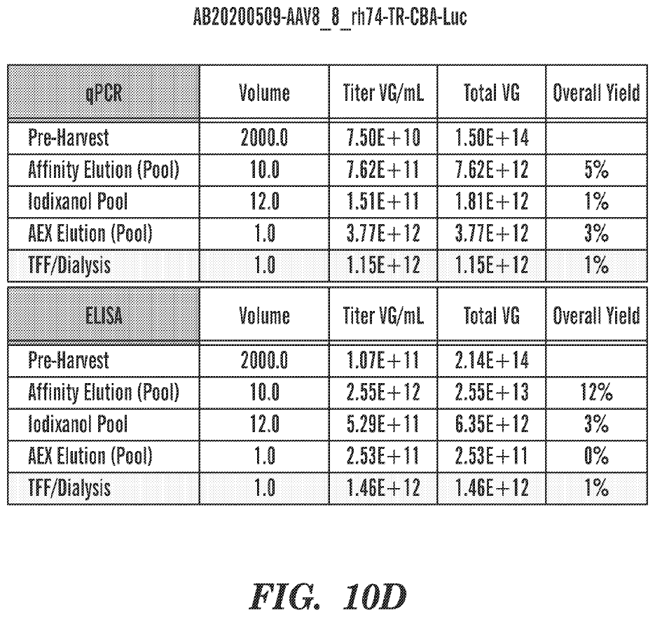

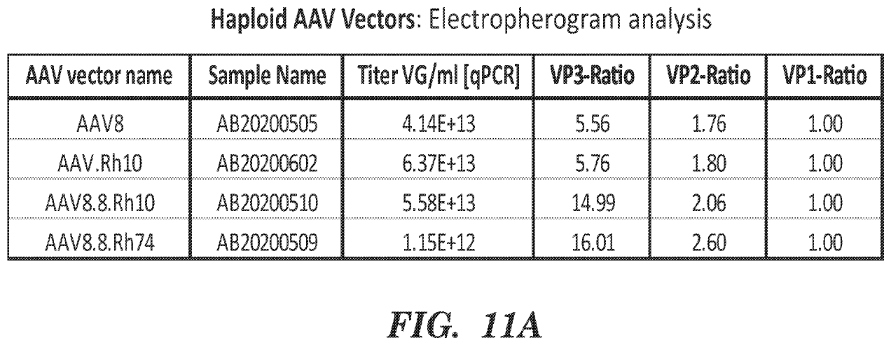

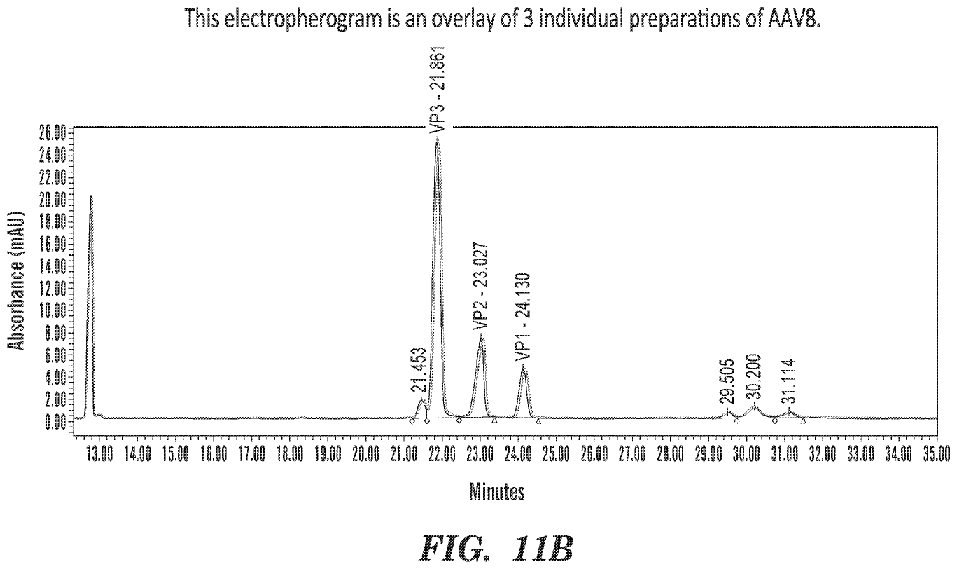

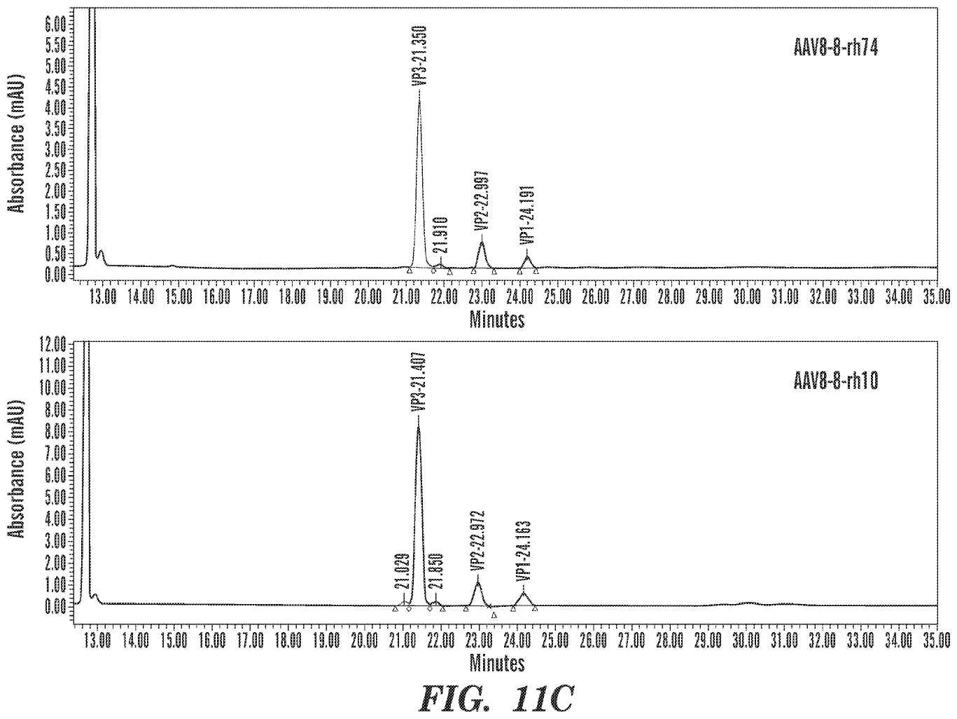

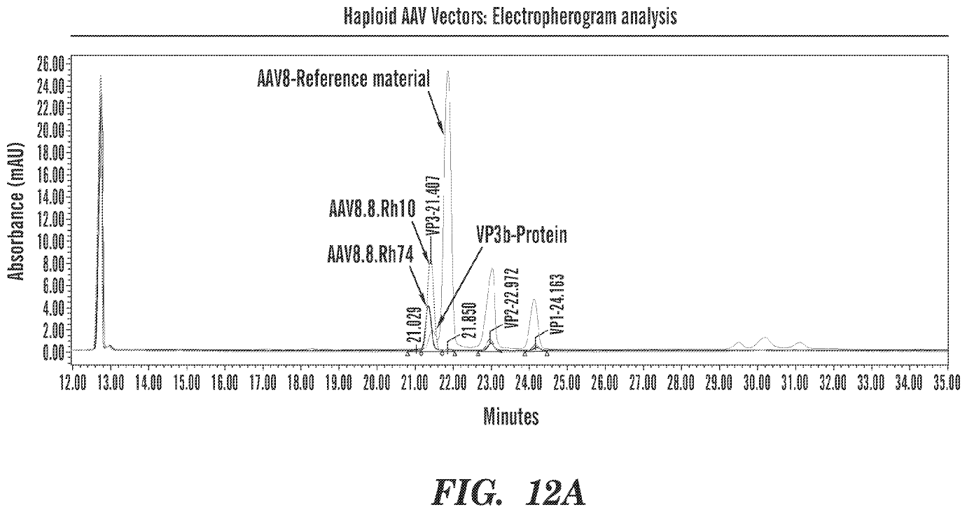

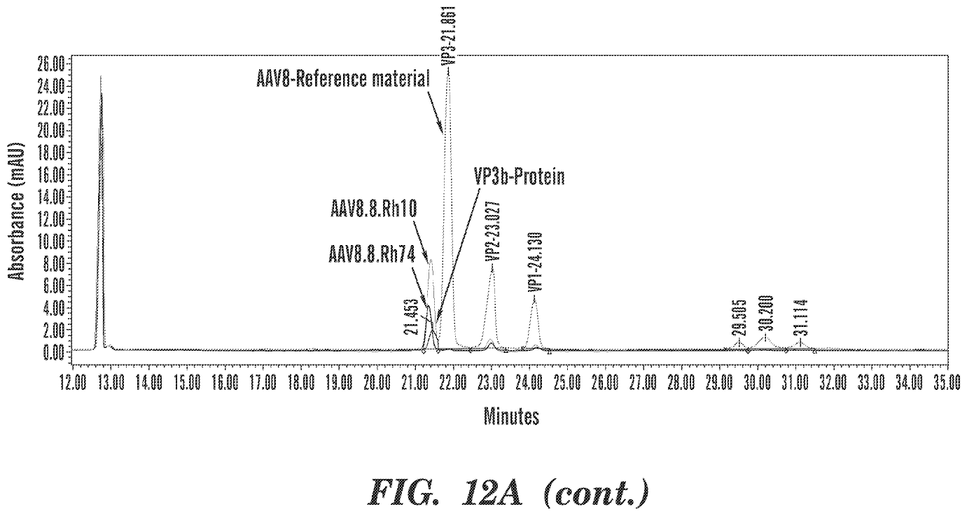

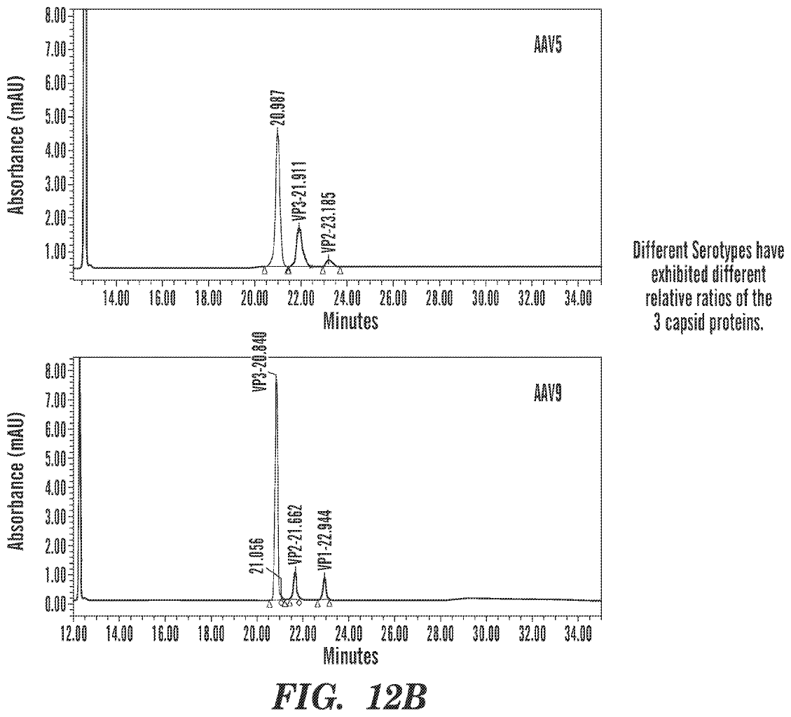

Results from CE-SDS method was developed using AAV8 final vector material (see FIG. 11B). The ratios of 6:2:1 have been very consistent with vector across hundreds of preparations/analyses. However, these ratios have changed slightly for other serotypes (AAV9 is closer to 9:1:1) (see FIG. 12B). These ratios are used as acceptance criteria but is restricted to AAV8 haploid serotypes. Interestingly, the inventors discovered that the VP3 protein in these AAV8-8-rh74 and AAV8-8-rh10 samples is migrating to overlay with the minor front peak (which is called out as VP3b), whereas VP2 and VP1 align perfectly with the AAV8 standard (see FIG. 12A).

### Example 5

Rh74 VP3 Optimization-Production of AAV8-8-rh74vv

Example 5 is an illustrative example that discloses exemplary combinations of VP1 and VP3 capsid proteins from AAV8 and any serotype that crosses the BBB, respectively, for example a rhesus monkey AAV (AAVrh) serotype, in any order, and optionally a VP2 capsid protein from AAV8 or any serotype that crosses the BBB, including, but not limited to a rhesus monkey AAV (AAVrh) serotype. While AAVrh10 and AAVrh74 capsid proteins are shown as exemplary serotypes that cross the BBB (and which are also AAVrh serotypes), these can be readily replaced or substituted with a VP3 protein from any other serotype that crosses the BBB (including but not limited to, e.g., AAV1, AAV6, AAV6.2, AAV7, AAV9, rAAVrh10, rAAVrh74, rAAVrh39, rAAVrh43 or other AAVrh serotypes). Similarly, AAV8 is shown as an exemplary serotype for the VP1 and/or VP2 protein, but the VP1 and/or VP2 protein from AAV8 can be readily replaced or substituted by one of ordinary skill in the art for any serotype disclosed in Table 1 herein.

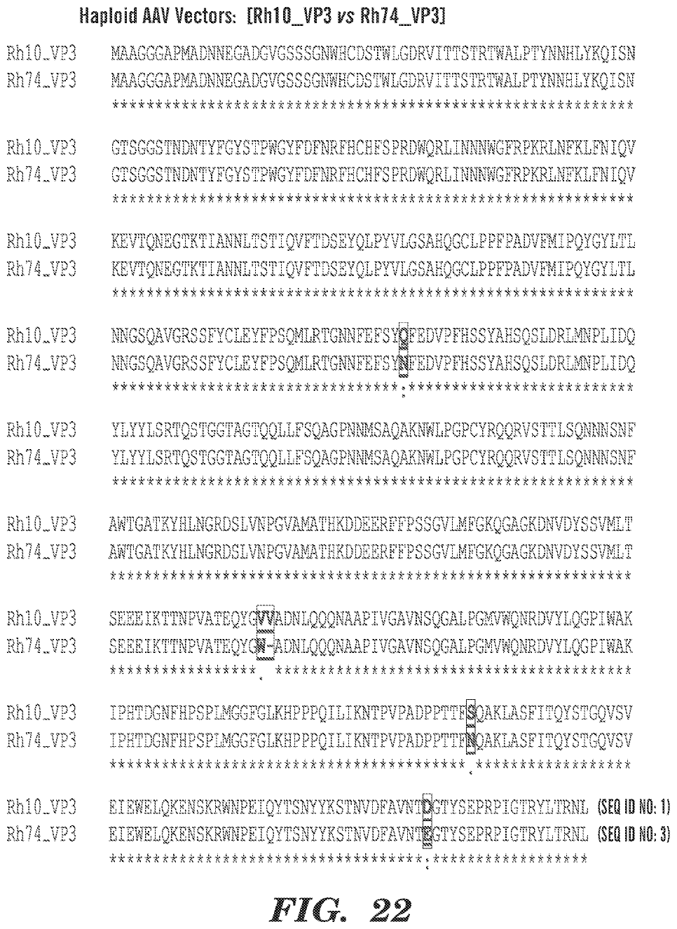

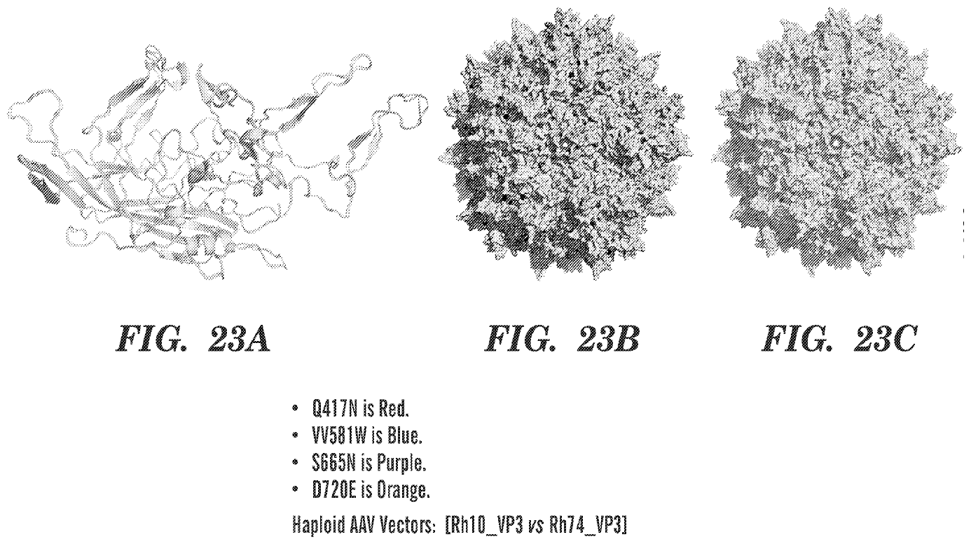

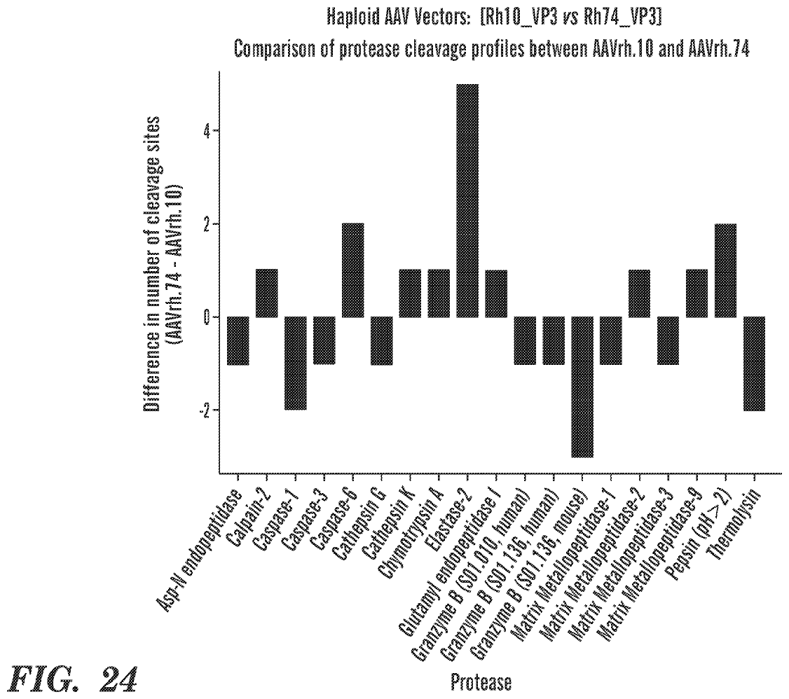

As previous Examples 1-4 demonstrated that AAV8-8-rh74 has a lower yield of production as compared to parental AAV8, or compared to the AAV8-8-rh10 haploid, the inventors compared the sequences of the VP3 capsid protein of AAVrh74 and AAVrh10. As shown in FIG. 22, there are 4 amino acids differences between the VP3 protein of AAVrh10 (SEQ ID NO: 1) and AAVrh74 (SEQ ID NO: 3). In particular, using SEQ ID NO: 1 (AAVrh10 VP3) as the reference sequence, there are the following amino acid changes: Q417N, VV581W, S665N and D720E to change SEQ ID NO: 1 (AAVrh10-VP3) to SEQ ID NO: 3 which is the amino acid sequence for the VP3 capsid protein for AAVrh74 (i.e., AAVrh74-VP3) (see., FIG. 23A-23C and FIG. 28).

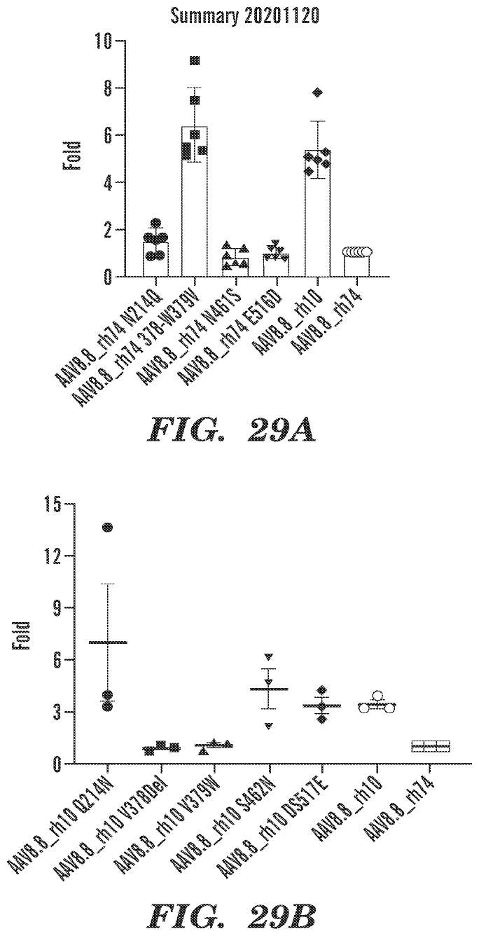

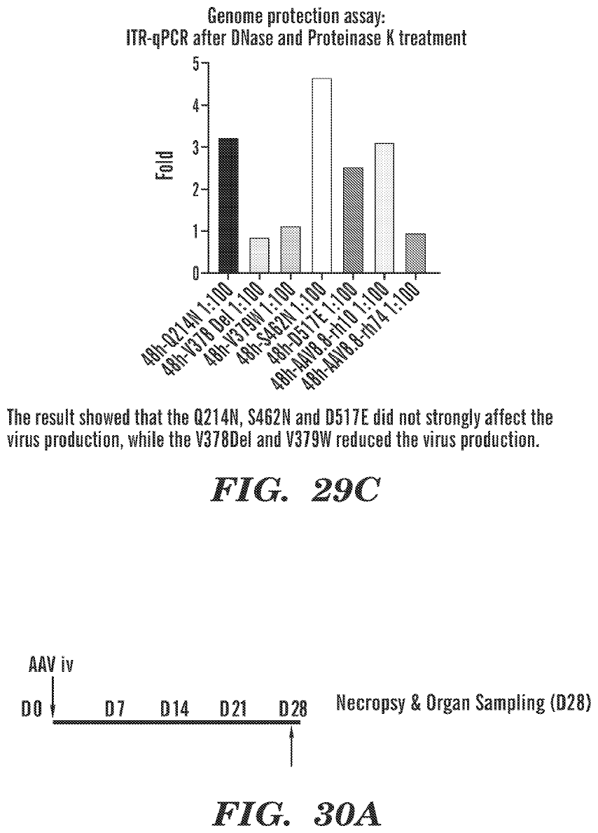

As the AAV8-8-rh74 haploid vector had significantly less production yield than AAV 8-8-rh10, the inventors assessed each mutation individually. In particular, the inventors made individual rh74 to rh10 (rh74>rh10) amino acid modifications, and changed the AAV8-8-rh74 vector to comprise one modification selected from: N417Q modification, W581VV modification, N664S or E719D modification in SEQ ID NO: 3, (the nomenclature/numbering is used from the amino acid sequence of the VP1 capsid protein from AAVrh74) and compared the fold of production to the unmodified AAV8-8-rh10 and AAV8-8-rh74 haploid capsids (see FIG. 29A). As shown in FIG. 29A, the change of the amino acid of the VP3 AAVrh74 capsid protein to have the -W581VV mutation significantly increased the yield of production by 4-fold, similar to that of the unmodified AAV8-8-rh10 yield. That is, substituting W at position 581 of SEQ ID NO: 3 to VV increases the yield of production significantly, whereas the other mutations did not have a significant effect on increasing the production yield of AAV8-8-rh74 haploid capsids. To confirm this, the inventors did the corresponding amino acid substitutions in the VP3 capsid protein for AAVrh10 (SEQ ID NO: 1), (i.e. individual modifications for VP3rh10>VP3rh74), where the differences in amino acids of VP3 AAVrh74 are introduced into the VP3 capsid protein of AAVrh10 (SEQ ID NO: 1)—in particular, Q417N, V581del, V582W, S665N and D720E substitutions are introduced into VP3 capsid of AAVrh10 (SEQ ID NO: 1). As shown in FIG. 29B, only the V581del and V582W modifications significantly reduced the production yield of the AAV8-8-rh10 vector. This is confirmed by qPCR analysis after DNase and proteinase K treatment (see FIG. 29C). In some alternative embodiments, the rational polyploid population, as disclosed herein, comprises mutated AAVrh10 VP3 protein, wherein the mutated AAVrh10VP3 comprises VP3 mutation, wherein the VP3 mutation is selected from the group consisting of Q214N, S462N, D517E, V378del, V379W (numberings are based on AAVrh10VP3 numbering). In yet another alternative embodiment, the rational polyploid population comprises mutated AAVrh10 VP3 protein, wherein the mutated AAVrh10VP3 comprises VP3 mutation, wherein the VP3 mutation is essentially consisting of all of Q214N, S462N, D517E, V378del, V379W (numberings are based on AAVrh10VP3 numbering). In some embodiments, the rational polyploid population comprises mutated AAVrh10 VP3 protein, wherein the mutated AAVrh10VP3 comprises VP3 mutation, wherein the VP3 mutation is selected from the group consisting of Q417N, S665N, D720E, V581del, V582W (numberings are based on AAVrh10VP1 numbering). In some embodiments, the rational polyploid population comprises mutated AAVrh10 VP3 protein, wherein the mutated AAVrh10VP3 comprises VP3 mutation, wherein the VP3 mutation is essentially consisting of all of Q417N, S665N, D720E, V581del, V582W (numberings are based on AAVrh10VP1 numbering).

Alternatively, the inventors made individual rh74 to rh10 (rh74>rh10) amino acid modifications, and changed the AAV8-8-rh74 vector to comprise one modification selected from: N214Q modification, 378-W379V (or 378del-W379VV) modification, N461S or E516D modification in SEQ ID NO: 3, (so that particular amino acids are changed to those similar to the VP3 AAVrh10 capsid protein) and compared the fold of production to the unmodified AAV8-8-rh10 and AAV8-8-rh74 haploid capsids (see FIG. 29A). That is, substituting W at position 378 of SEQ ID NO: 3 to VV increases the yield of production significantly, whereas the other mutations did not have a significant effect on increasing the production yield of AAV8-8-rh74 haploid capsids. To confirm this, the inventors did the corresponding amino acid substitutions in the VP3 capsid protein for AAVrh10 (SEQ ID NO: 1), (i.e. individual modifications for VP3rh10>VP3rh74), where the differences in amino acids of VP3 AAVrh74 are introduced into the VP3 capsid protein of AAVrh10 (SEQ ID NO: 1)—in particular, Q214N, V378del, V379W, S462N and D517E substitutions are introduced into VP3 capsid of AAVrh10 (SEQ ID NO: 1); herein numberings are based on AAVrh10 VP3 numbering. Only the V378del and V379W modifications significantly reduce the production yield of the AAV8-8-rh10 vector. This is confirmed by qPCR analysis after DNase and proteinase K treatment (see FIG. 29C). In some alternative embodiments, the rational polyploid population, as disclosed herein, comprises mutated AAVrh74 VP3 protein, wherein the mutated AAVrh74VP3 comprises VP3 mutation, wherein the VP3 mutation is selected from the group consisting of N214Q, N461S, E516D, W378VV (numberings are based on AAVrh74VP3 numbering). In yet another alternative embodiment, the rational polyploid population comprises mutated AAVrh74 VP3 protein, wherein the mutated AAVrh74VP3 comprises VP3 mutation, wherein the VP3 mutation is essentially consisting of all of N214Q, N461S, E516D, W378VV (numberings are based on AAVrh74VP3 numbering). In some embodiments, the rational polyploid population comprises mutated AAVrh74 VP3 protein, wherein the mutated AAVrh74VP3 comprises VP3 mutation, wherein the VP3 mutation is selected from the group consisting of N417Q, N664S, E719D, W581VV (numberings are based on AAVrh74VP1 numbering). In some embodiments, the rational polyploid population comprises mutated AAVrh74VP3 protein, wherein the mutated AAVrh74VP3 comprises VP3 mutation, wherein the VP3 mutation is essentially consisting of all of N417Q, N664S, E719D, W581VV (numberings are based on AAVrh74VP1 numbering). Accordingly, the inventors discovered that a simple modification of amino acid W at amino acid position 581 to VV of SEQ ID NO: 3 (VP3 capsid protein for AAVrh74) significantly increased the production yield, yet maintained the increased systemic bioavailability and reduced humoral response and/or antigenicity and/or ability to evade AAV8 neutralizing antibodies in vivo. Accordingly, in some embodiments, the AAV8 haploid is a AAV8-8-rh74vv haploid, where the VP3 protein comprises the VP3-AAVrh74 capsid protein corresponding to SEQ ID NO: 2 (where W581 is replaced with VV). In some embodiments, the AAV8 haploid is a AAV 8-8-rh74 vv LP2 haploid, where the VP3 protein comprises the VP3-AAVrh74 capsid protein corresponding to SEQ ID NO: 2 (wherein VP3 of AAV rh74 comprise the following mutation-W581 is replaced with VV and all of the following mutation-N263S, G264A, T265S, S266T, G268A, T270del, T274H, E533K, R726H, N736P (numberings are based on AAV rh74 VP1 numbering). In some embodiments, the AAV8 haploid is a AAV 8-8-rh10 LP2 haploid, where the VP3 protein comprises the VP3-AAVrh10 capsid protein corresponding to SEQ ID NO: 5 (wherein VP3 of rh10 comprises the following mutation N263S, G264A, T265S, S266T, G268A, T270del, T274H, E533K, R727H, N737P; numberings are based on AAV rh10 VP1 numbering. The production yield of AAV8-8-rh74 vv LP2 is improved over AAV 8-8-rh74 LP2 and is comparable to that of AAV 8-8-rh10LP2 or, AAV 8-8-rh10, as shown in table 9.

TABLE 9
Production yield of AAV 8-8-rh10LP2 and AAV 8-8-rh74LP2
and AAV 8-8-rh74vv rational polyploid vectors.

| Rational polyploid vector | Titre (vg/ml) |
|---|---|
| AAV8-8-Rh10LP2 | 6.11 × 10¹² |
| AAV8-8-Rh74LP2 | 5.02 × 10⁹ |
| AAV8-8-Rh74VV | 1.25 × 10¹³ |
| AAV8-8-8 | 1.107 × 10¹³ |

Accordingly, in some embodiments a AAV haploid disclosed herein comprises a rh74 VP3 capsid protein which is a modified VP3 protein comprising at least 1 or more amino acid modifications, for example, the AAVrh74 VP3 capsid protein is a modified VP3 protein comprising W581VV modification, where tryptophan (W or Trp) at amino acid position 581 of SEQ ID NO: 3 is substituted for two valine (V or val) amino acids. Accordingly, in some embodiments, the AAV haploid vector is a AAV8-8-rh74vv haploid vector which comprises a VP3 capsid protein having an amino acid sequence of SEQ ID NO: 2, or an amino acid sequence at least 85%, or at least 90%, or at least 95% or at least 98% sequence identity to SEQ ID NO:2, where SEQ ID NO: 2 is the amino acid of rh74vv-VP3 capsid protein, which comprises the W581VV modification

SEQ ID NO: 2 comprising the amino acid of the rh74vv-VP3 capsid protein is encoded by the nucleic acid sequence of SEQ ID NO: 4.

### Example 6

### Characterization of AAV8 Haploid Viruses In Vitro.

Example 6 is an illustrative example that discloses exemplary combinations of VP1 and VP3 capsid proteins from AAV8 and any serotype that crosses the BBB, respectively, for example a rhesus monkey AAV (AAVrh) serotype, in any order, and optionally a VP2 capsid protein from AAV8 or any serotype that crosses the BBB, including, but not limited to a rhesus monkey AAV (AAVrh) serotype. While AAVrh10 and AAVrh74 capsid proteins are shown as exemplary serotypes that cross the BBB (and which are also AAVrh serotypes), these can be readily replaced or substituted with a VP3 protein from any other serotype that crosses the BBB (including but not limited to, e.g., AAV1, AAV6, AAV6.2, AAV7, AAV9, rAAVrh10, rAAVrh74, rAAVrh39, rAAVrh43 or other AAVrh serotypes). Similarity, AAV8 is shown as an exemplary serotype for the VP1 and/or VP2 protein, although the VP1 and/or VP2 protein from AAV8 can be readily replaced or substituted by one of ordinary skill in the art for any serotype disclosed in Table 1 herein.

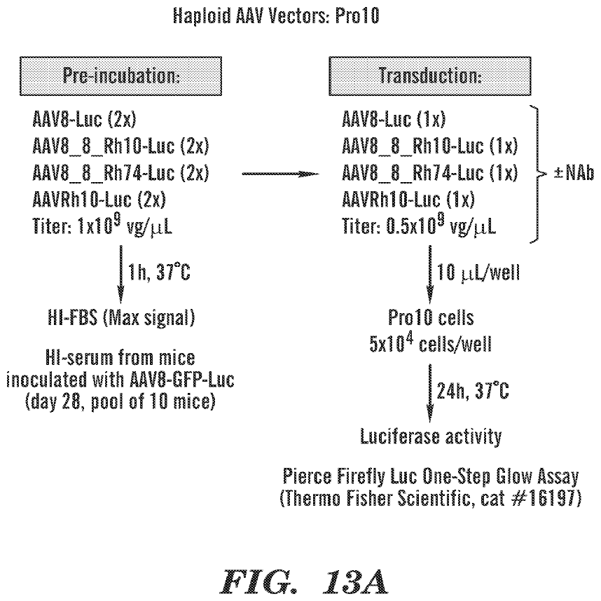

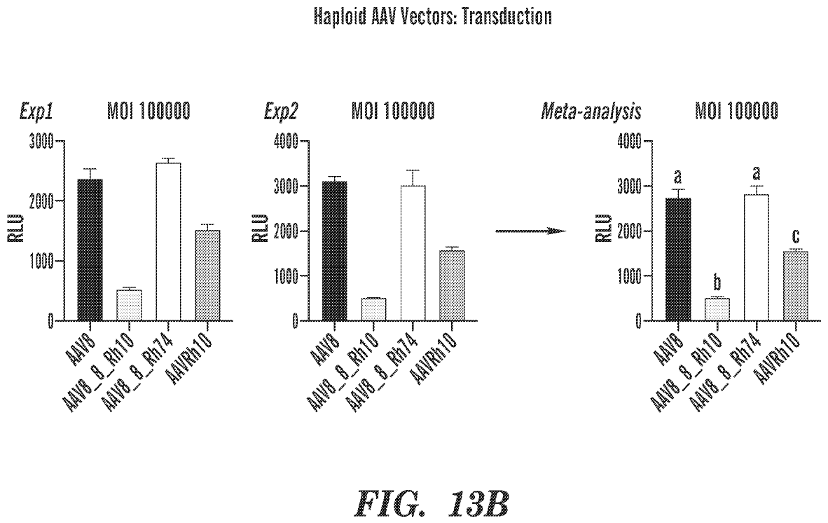

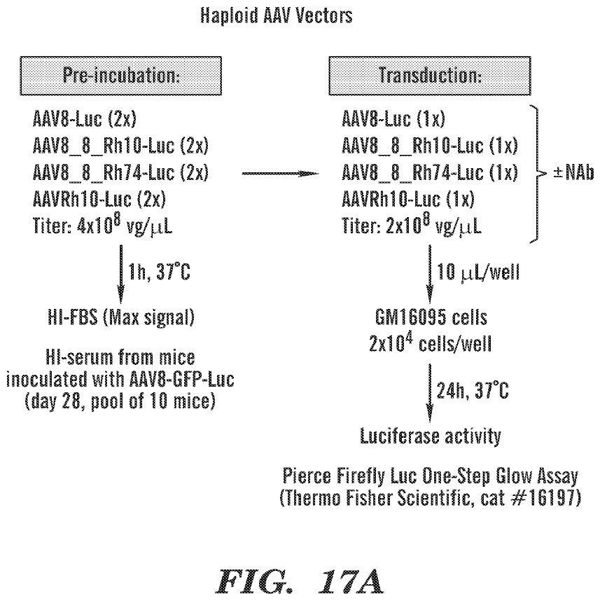

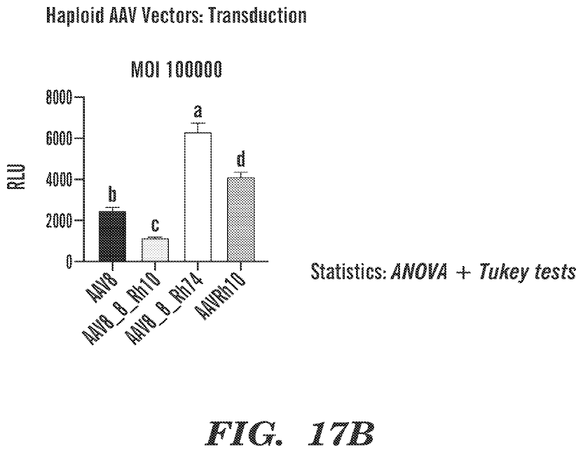

The transduction efficiency of the AAV8-8-rh10 or AAV8-8-rh74 haploids was assessed in Pro10 cells, which showed that there were some differences in the ability of the haploid vectors to transduce Pro10 cells: AAV8-8-Rh74 haploid transduced Pro10 cells similar to AAV8 control, whereas AAVrh10 and AAV8-8-Rh10 vectors were less efficient than AAV8 in this cell line, with AAV8-8-Rh10 haploid was significantly less efficient than either AAV8 and AAVRh10 (see FIG. 13B). In addition, the efficiency of AAV8-8-rh10 or AAV8-8-rh74 haploids to transduce GM16095 cells (FIG. 17B) was assessed and demonstrated that AAV8-8-Rh74 haploid transduced GM16095 cells significantly more efficiently than AAV8 or AAVrh10 control, whereas AAVrh10 is more efficient than AAV8 in this GM16095 cell line, and AAV8-8-Rh10 haploid was significantly less efficient than either AAV8 and AAVRh10.

### Example 7

### Characterization of AAV8 Haploid Viruses In Vivo Shows Increased CNS Transduction by the AAV8 Haploid Viruses.

Example 7 is an illustrative example that discloses exemplary combinations of VP1 and VP3 capsid proteins from AAV8 and any serotype that crosses the BBB, respectively, for example a rhesus monkey AAV (AAVrh) serotype, in any order, and optionally a VP2 capsid protein from AAV8 or any serotype that crosses the BBB, including, but not limited to a rhesus monkey AAV (AAVrh) serotype. While AAVrh10 and AAVrh74 capsid proteins are shown as exemplary serotypes that cross the BBB (and which are also AAVrh serotypes), these can be readily replaced or substituted with a VP3 protein from any other serotype that crosses the BBB (including but not limited to, e.g., AAV1, AAV6, AAV6.2, AAV7, AAV9, rAAVrh10, rAAVrh74, rAAVrh39, rAAVrh43 or other AAVrh serotypes). Similarity, AAV8 is shown as an exemplary serotype for the VP1 and/or VP2 protein, although the VP1 and/or VP2 protein from AAV8 can be readily replaced or substituted by one of ordinary skill in the art for any serotype disclosed in Table 1 herein.

As described above, the transduction efficiency of haploid virus AAV8-8-rh74 haploids is the higher than that of the AAV parental serotypes in Pro10 cells or GM16095 cell line. Next was studied whether the high transduction of AAV8-8-rh74 in vitro was translated into mouse in vivo. AAV8-8-rh10 or AAV8-8-rh74 haploids and parental vectors (AAV8 and AAVrh10) were intravenously injected (via tail vein injection) into C57BL/6 mouse. A total vector of 5×10¹⁰ vg for each virus was administered per mouse. Compared to AAV8, significant distribution of AAV8-8-Rh74 was determined in vivo, which was greater than AAV8 or AAVrh10 (FIG. 21A-21D).

Furthermore, AAV 8-8-rh10, AAV 8-8-rh74, or, AAV8 genome copy numbers (measured by qPCR) are measured in mouse brain wherein, all virions are administered systemically and the result shows that 8-8-rh10 and 8-8-rh74 both have significant brain transduction whereas that of AAV 8 in brain is minimal.

In an in vitro endothelial cell permeability analysis in a well-defined system using BBB hCEMC/D3 endothelial cells, a significant increase in the endothelial cell permeability is observed with AAV 8-8-rh10 or, with AAV 8-8-rh74 as compared to that of AAV8. These observations indicate that rational polyploid (e.g rational haploid) population of AAV 8-8-rh10 or, AAV 8-8-rh74 have increased ability to cross BBB than that of AAV8. Further to assess the transduction efficiency of the vectors to the CNS, GFP immunohistochemistry (IHC) and/or native eGFP fluorescence of several brain regions, the spinal cord and retina are performed. AAV 8-8-rh74 or, AAV 8-8-rh10 show transduction in the entire adult CNS with high efficiency.

In another example, the rational polyploid AAV 2-2-9 is generated following the methodology as described in international patent application PCT/US2018/022725 and U.S. Pat. No. 10,550,405 both of which are incorporated by reference in its entirety. In rational polyploid AAV 2-2-9, the VP1 and VP2 are from AAV2 serotype that can't cross blood brain barrier and VP3 is from AAV9 serotype that can efficiently cross blood brain barrier. The resultant rational polyploid AAV 2-2-9 virion can cross Blood brain barrier as shown by the results. Enhanced luciferase transduction (measured by imaging analysis) in CNS region e.g., in brain regions is obtained with AAV 2-2-9 when compared to that of AAV2 where both virions are administered systemically. Furthermore, the AAV 2-2-9 genome copy numbers in brain are significantly high than that of AAV2 confirming that AAV 2-2-9 has significant high transduction in brain than that of AAV2 when both virions are administered systemically. In an in vitro endothelial cell permeability analysis in a well-defined system using BBB hCEMC/D3 endothelial cells, a significant increase in the endothelial cell permeability is observed with AAV 2-2-9 as compared to that of AAV2. These observations indicate that rational polyploid (e.g., rational haploid) population of AAV 2-2-9 have increased ability to cross BBB than that of AAV2. Further to assess the transduction efficiency of the vectors to the CNS, GFP immunohistochemistry (IHC) and/or native eGFP fluorescence of several brain regions, the spinal cord and retina are performed. AAV 2-2-9 show transduction in the entire adult CNS with high efficiency.

Notably, the significant distribution of AAV8-8-Rh74 was identified in the CNS regions e.g., brain and spinal cord, as demonstrated by distribution assessed by dorsal and ventral view (FIG. 21D; ventral view for FIG. 21A-21C).

To further evaluate the in vivo biodistribution of the rational haploid AAV vector disclosed herein, mice (n=4/group) were injected with a dose of 2.5E¹² vg/kg via the tail vein (FIG. 30A). Transgene expression was evaluated on transcriptional level in various organs on D28 post vector injection (FIG. 30A). After RNA extraction, levels of transgene expression were quantified by RT-qPCR.

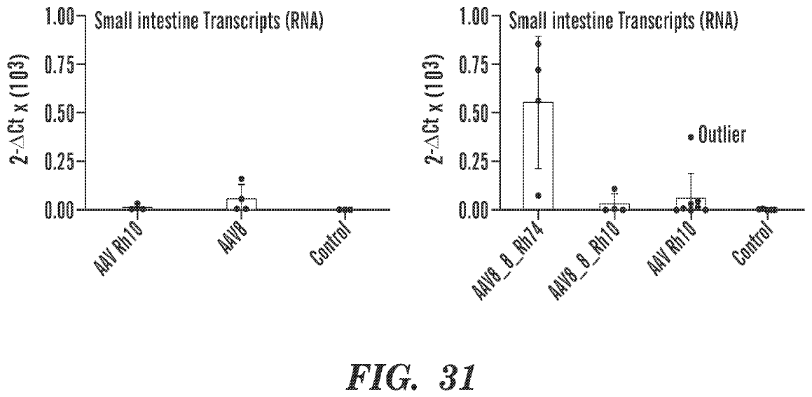

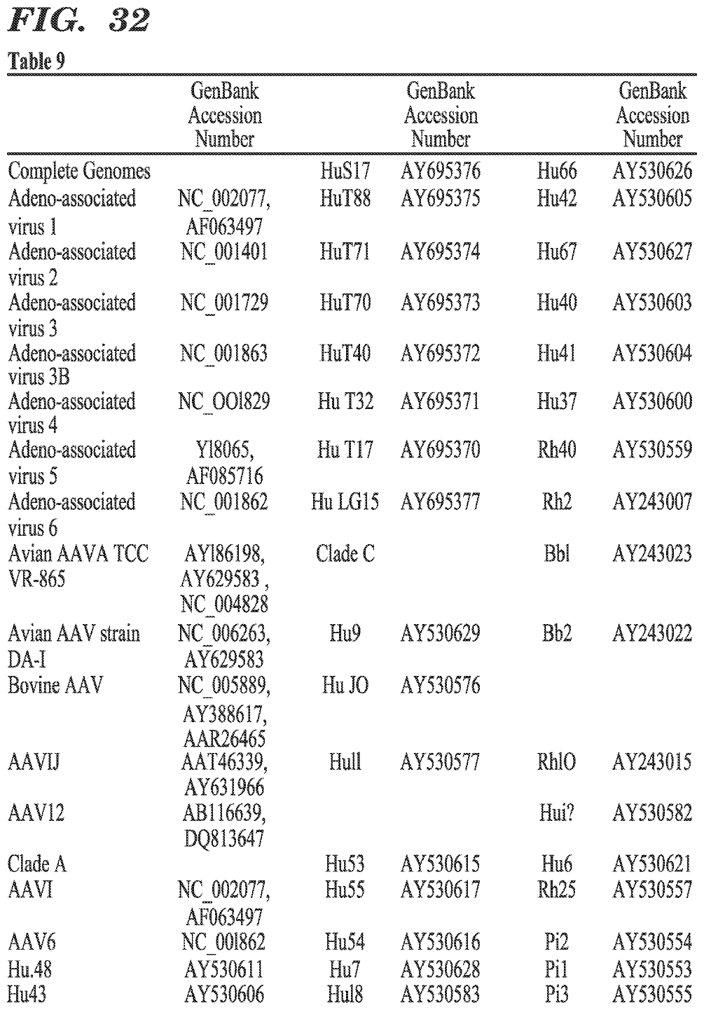

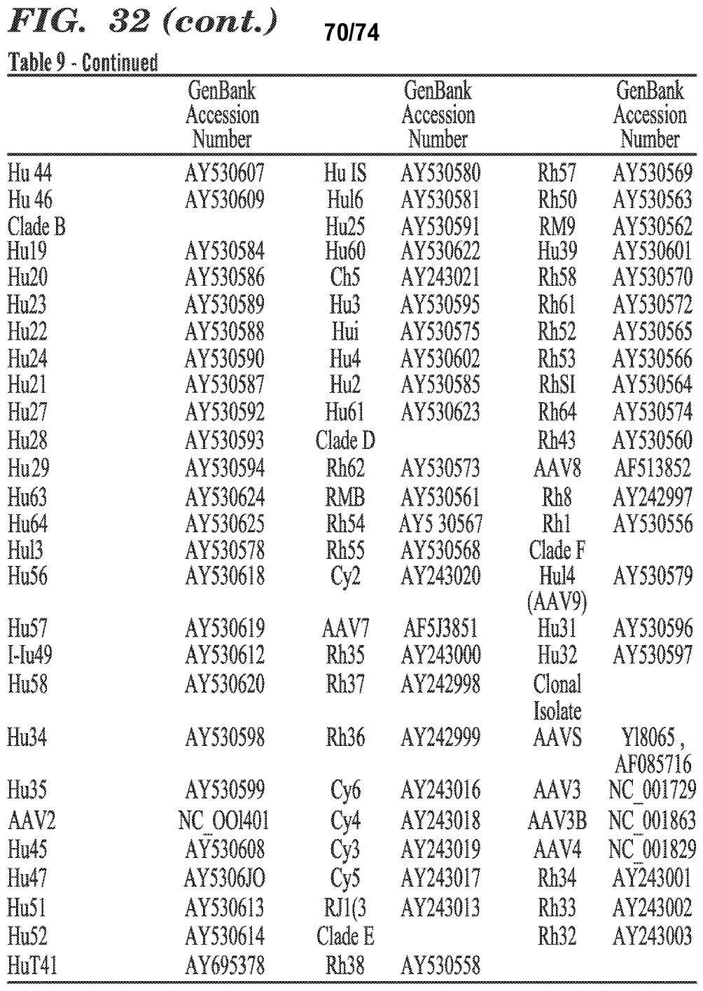

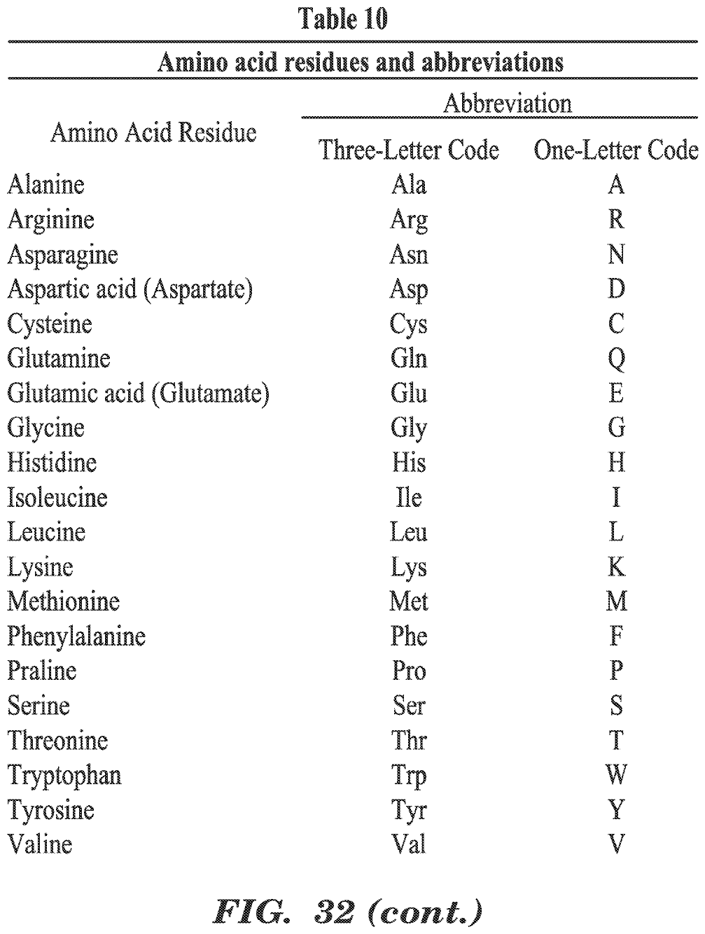

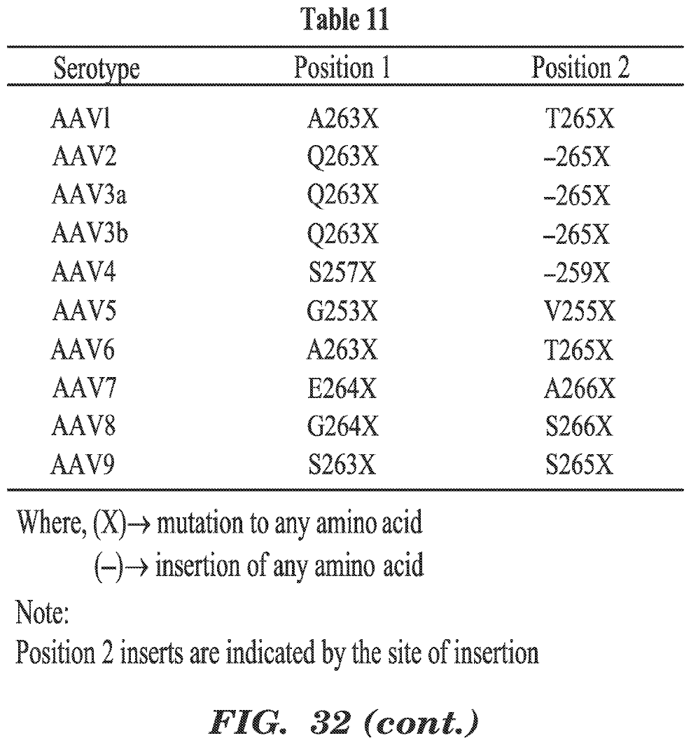

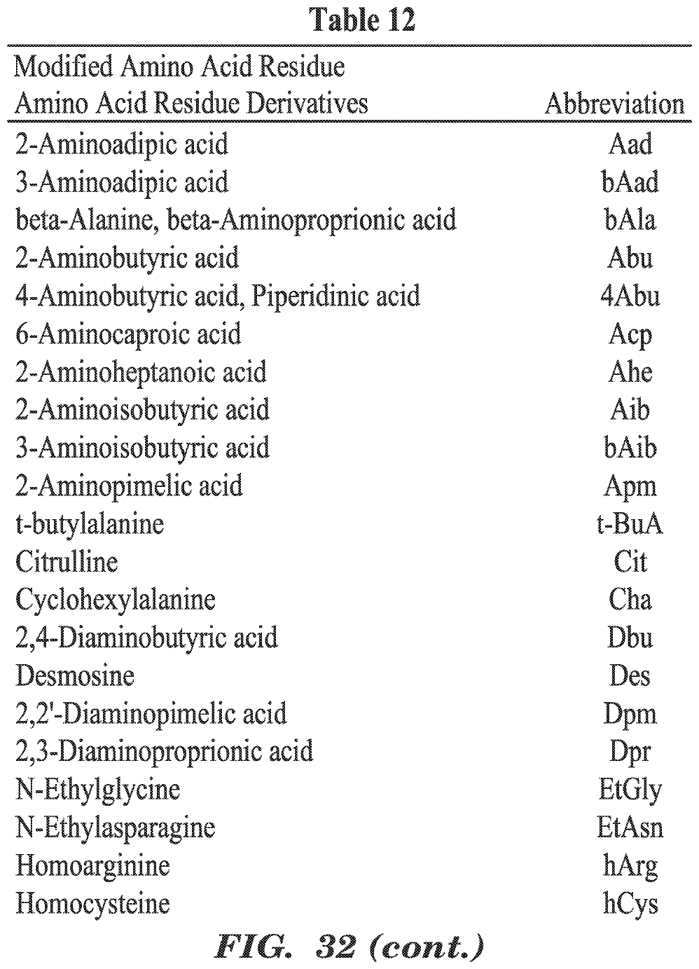

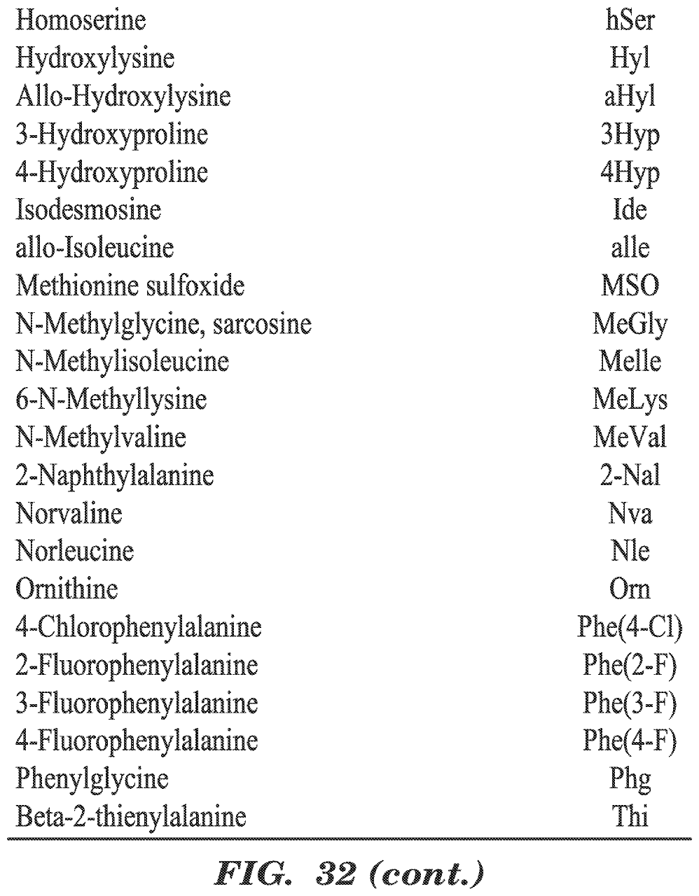

FIG. 30B shows that mice treated with AAV8 had few transcripts in the brain, indicating that AAV8 does not cross the BBB. In contrast, AAV8-8-Rh74 resulted in many transcripts detected in the brain. Because the administration was intravenous, the data demonstrates that replacing the AAV8 VP3 with AAVRh74 VP3 results in a haploid vector that crosses the BBB and targets the brain and is useful for the treatment of diseases or disorders of the brain or central nervous system (CNS) disorders, or neurological diseases, as disclosed herein. Rh74 also crosses the BBB (data not shown). Similar results were detected in the spinal cord, where FIG. 30C shows that AAV8 does not target the spinal cord. However, AAV8-8-Rh74 does target the spinal cord. Therefore, FIG. 30C shows that replacing the AAV8 VP3 with AAVRh74 VP3 results in a haploid vector having tropism to the spinal cord, and is useful for the treatment of diseases or disorders of the spinal cord, or peripheral nervous system (PNS) disorders. In addition, FIG. 31 shows that AAV8 does not target the small intestine, however, AAV8-8-Rh74 does target the small intestine. Therefore, FIG. 31 shows that replacing the AAV8 VP3 with AAVRh74 VP3 results in a haploid vector having tropism to the small intestine and is useful for the treatment of gastrointestinal disorders or disorders of the small intestine.

In brain (FIG. 30B), spinal cord (FIG. 30C), and small intestine (FIG. 31), significance was achieved with novel vector AAV8-8-Rh74, as compared to the AAV8 or AAVRh10 parental vectors or saline control. AAV8-8-Rh74 was able to cross the BBB more effectively than AAV8-8-Rh10, and was more effective at transducing the small intestine as compared to AAV8-8-Rh10, and parental vectors AAV8 or AAVRh10.

These results demonstrate that AAV8-8-rh74 haploid virus is able to cross the BBB upon intravenous (or, systemic) administration and further supports that polyploid or, haploid virions produced from the rational design were one construct comprises the VP1/VP2 from AAV8 and VP3 is either from AAVrh10 or from AAVrh74.

### Example 8

The Ability of Haploid Viruses AAV8-8-rh10 and AAV8-8-rh74 to Escape Neutralizing Antibody.

Example 8 is an illustrative example that discloses exemplary combinations of VP1 and VP3 capsid proteins from AAV8 and any serotype that crosses the BBB, respectively, for example a rhesus monkey AAV (AAVrh) serotype, in any order, and optionally a VP2 capsid protein from AAV8 or any serotype that crosses the BBB, including, but not limited to a rhesus monkey AAV (AAVrh) serotype. While AAVrh10 and AAVrh74 capsid proteins are shown as exemplary serotypes that cross the BBB (and which are also AAVrh serotypes), these can be readily replaced or substituted with a VP3 protein from any other serotype that crosses the BBB (including but not limited to, e.g., AAV1, AAV6, AAV6.2, AAV7, AAV9, rAAVrh10, rAAVrh74, rAAVrh39, rAAVrh43 or other AAVrh serotypes). Similarity, AAV8 is shown as an exemplary serotype for the VP1 and/or VP2 protein, although the VP1 and/or VP2 protein from AAV8 can be readily replaced or substituted by one of ordinary skill in the art for any serotype disclosed in Table 1 herein.

Therapeutic effect has been achieved in clinical trials in patients with blood diseases and blind disorders using adeno-associated virus (AAV) vector. However, two concerns restrict broadening AAV vector application: AAV capsid specific cytotoxic T cell (CTL) and neutralizing antibodies (Nabs). Enhancing AAV transduction with low dose of AAV vector will potentially decrease capsid antigen load and hopefully ablate capsid CTL mediated clearance of AAV transduced target cells without compromise of transgene expression. Currently, 12 serotypes and over 100 variants or mutants have been explored for gene delivery due to their different tissue tropism and transduction efficiency. It has been demonstrated that there is compatibility of capsid among AAV serotypes, and integration of specific amino acids from one serotype into another AAV capsid enhances AAV transduction. By taking advantage of different mechanisms for effective AAV transduction from different serotypes, enhanced AAV transduction was achieved using mosaic virus in which AAV capsid subunits are derived from different serotypes in vitro and in vivo. The recent structural studies on interaction of AAV vectors with monoclonal neutralizing antibodies demonstrated that Nab binds to residues on several different subunits of one virion surface, which suggests that change of subunit assembly of AAV virion may ablate the AAV Nab binding site and then escape Nab activity. We have demonstrated that the mosaic AAV vector is able to evade Nab activity. These results indicate that substitution of AAV capsid subunits has the potential to enhance AAV transduction and the ability of neutralizing antibody evasion.

Herein, the inventors demonstrate that these AAV8-8-rh10 and AAV8-8-rh74vv haploid viruses enhance the transduction efficiency in vitro and in vivo, and even escape neutralization from parental vector immunized sera. Each individual haploid virus virion is composed of 60 subunits from different AAV serotype capsids. Insertion of some capsid subunits from one serotype into other capsid subunits from a different serotype may change the virion surface structure. It is well known that most AAV monoclonal antibodies recognize residues on the different subunits of one single virion.

Ability of the AAV8-8-rh74 haploids to Escape Nab.

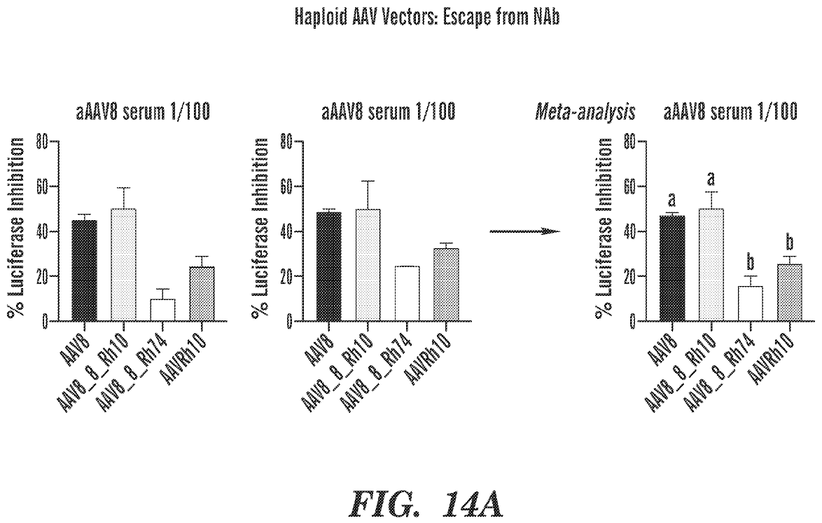

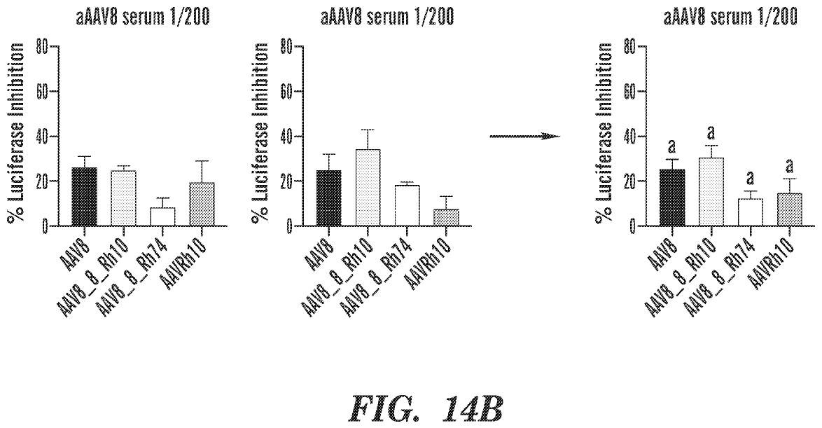

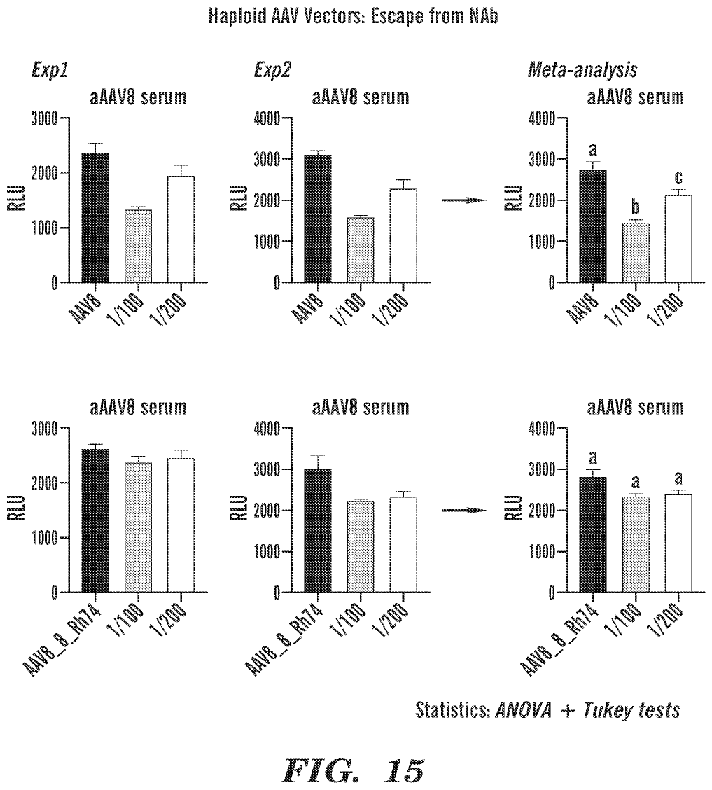

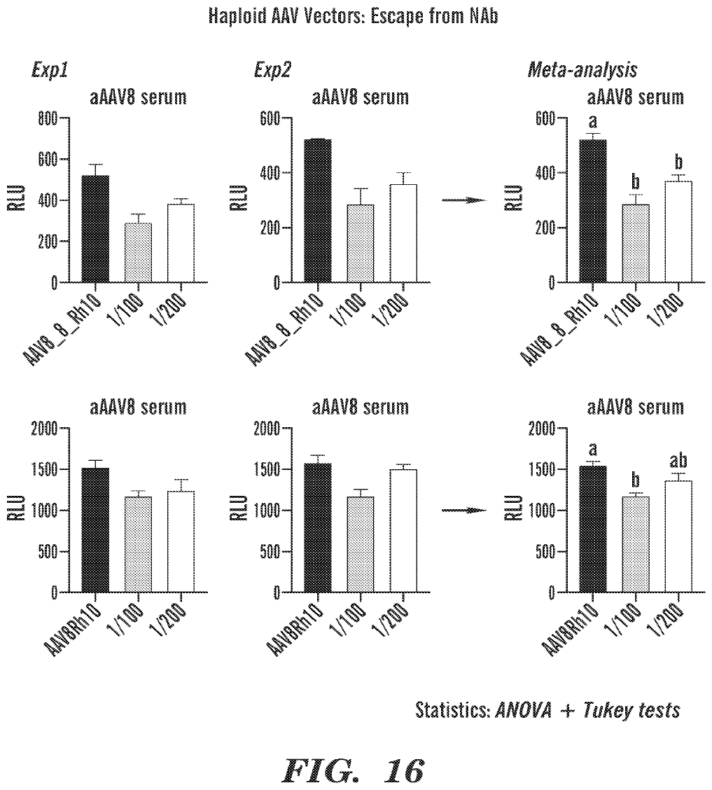

Adeno-associated virus (AAV) vectors have been successfully used in clinical trials in patients with hemophilia and blindness. Exploration of effective strategies to enhance AAV transduction and escape neutralizing antibody activity is still imperative. Previous studies have shown the compatibility of capsids from AAV serotypes and recognition sites of AAV Nab located on different capsid subunits of one virion. In this study, the inventors assessed the AAV8-8-rh10 and AAV8-8-rh74 haploid capsids Nab escape activity followed by transduction (see, e.g., FIGS. 14A-FIGS. 16 and 18A-18B, 19A-19B). To determine whether the tropism of these haploid vectors was changed in the rational polyploid vectors, the transduction efficacy of the haploid viruses was analyzed by transducing human Pro10 cells and GM16095 cell lines (FIGS. 13A-13B and 17A-17B).

Accordingly, the inventors analyzed the immunological profile of haploid AAV8-8-rh10 or AAV8-8-rh74 haploid viruses against sera from AAV-immunized mice. Nab titers were used to evaluate the ability of serum to inhibit vector transduction. Sera were collected from mice treated with parental viruses at week 4 post-injection.

FIG. 18A-18B demonstrates that AAV8-8-Rh74 haploid vector, but not the AAV8-8-rh10 haploid vector was able to escape from anti-AAV8 NAb, and no differences were found in the luciferase transgene expression in the presence and absence of Nab. Additionally, from transfection of Pro10 cells in vitro was assessed in the presence of antiserum AAV8 (aAAV8) at 1/100 and 1/200 concentration, and demonstrated that AAV8-8-Rh74 haploid vector, but not the AAV8-8-rh10 haploid vector, was able to escape from anti-AAV8 NAb, and no differences were found in the luciferase transgene expression in the presence and absence of Nab (FIG. 19A-19B).

It is interesting to note that AAV8-immunized mouse sera had similar neutralizing activity against AAV8 and AAVrh10 virus, regardless of the amount of AAV8 capsid incorporation.

After intravenous injection, all of the haploid viruses induced higher transduction than parental AAV vectors (2- to 9-fold over AAV8 or AAVrh10) with the highest of these being the haploid vector AAV8-8rh74 (see FIG. 21A-21D). Notably, the systemic transduction of the haploid vector AAV8-8-rh74 was over 4-fold higher than that of AAV8. Additionally, haploid virus AAV8-8rh74 was able to escape AAV8 neutralization and had very low Nab cross-reactivity with AAV8. Neutralizing antibody analysis demonstrated that AAV8-8rh74 haploid vector was able to escape neutralizing antibody activity from mouse sera immunized with parental serotypes. These results indicate that AAV8 haploid virus comprising rhesus monkey AAV serotypes (AAVrh) might potentially acquire advantage from parental serotypes for enhancement of transduction and evasion of Nab recognition. This strategy should be explored in future clinical trials in patients with positive neutralizing antibodies.

Ability of AAV8-8-rh10LP2 and AAV8-8-rh74LP2 to escape NAb It is expected that the AAV8-8-rh10LP2 and AAV8-8-rh74LP2 or, AAV 8-8-rh74vvLP2 can efficiently escape Neutralizing antibody recognition of AAV rh10 or, AAV rh74 serotype. Mice are intravenously injected with AAVrh10 or AAVrh74 comprising luciferase transgene that leads to the transgene expression in CNS. Mice are then injected with either AAV 8-8-rh10 LP2, AAV 8-8-rh74 LP2, AAV 8-8-rh74vvLP2, AAVrh10, or, AAVrh74 each comprising the luciferase transgene. In this repeat administration, only AAV8-8-rh10LP2, AAV 8-8-rh74 LP2, or AAV 8-8-rh74vvLP2 leads to successful transduction and luciferase expression in CNS supporting the fact that only AAV8-8-rh10LP2, AAV 8-8-rh74LP2, or, AAV 8-8-rh74vvLP2 can escape the Neutralizing Antibodies against rh10 or, rh74 AAV serotype and not other groups.

Ability of AAV8-8-Rh10 or, AAV8-8-Rh74 Haploids with Reduced Humoral Response

The antigenicity or the ability of the AAV8 haploids e.g., AAV8-8-rh10 or AAV8-8-rh74 was evaluated by measuring the IgG levels in mice when inoculated with haploid vectors as shown in FIG. 25. FIG. 25A shows anti-AAV8 IgG levels (1/1000 serum dilution) and FIG. 25B shows anti-AAV8 IgG levels (1/5000 serum dilution), showing significantly reduced humoral response e.g., as shown by reduced anti-AAV8 IgG levels detected in the serum from mice inoculated with both haploid vectors, in comparison to the mice injected with AAV8. No cross-reactivity against AAV8 was found with serum from the mice inoculated with AAVrh10 at the serum dilutions tested. FIG. 25C shows anti-AAVrh10 IgG levels (1/1000 serum dilution) and FIG. 25D shows anti-AAVrh10 IgG levels (1/5000 serum dilution), and shows that AAVrh10 was significantly less immunogenic than AAV8, and no significant differences were observed in the anti-AAVrh10 IgG levels between the mice inoculated with AAVrh10 and the rest of the experimental groups at the serum dilution tested.

The results demonstrate that these AAV8 haploid vectors with VP3 from Rh serotype (e.g., AAV8-8-rh10 or AAV8-8-rh74) can be used for in vivo or, clinical application as they exhibit lower HUMORAL RESPONSE.

### Example 9

### Treatment of Diseases

In each of the following Example 9 for treatment of diseases: e.g., of the central nervous system, heart, lung, skeletal muscle, and liver; including e.g., Parkinson's disease, Alzheimer's disease, ALS, the AAV8 haploid capsid virion described therein that is generated using the specified AAV serotypes and is generated using the rational polyploid method of Example 1, to generate a haploid capsid where VP1 and VP2 is from AAV8 and VP3 only is from any serotype that crosses the BBB, for example, AAV1, AAV6, AAV6.2, AAV7, AAV9, rAAVrh10, rAAVrh74, rAAVrh39, rAAVrh43, or other rhesus monkey AAV (AAVrh) serotypes. Without wishing to be limited to theory, Example 9 is an illustrative example that discloses exemplary combinations of VP1 and VP3 capsid proteins from AAV8 any serotype that crosses the BBB, respectively, for example a rhesus monkey AAV (AAVrh) serotype, in any order, and optionally a VP2 capsid protein from AAV8 or any serotype that crosses the BBB, including, but not limited to a rhesus monkey AAV (AAVrh) serotype. While AAVrh10 and AAVrh74 capsid proteins are shown as exemplary serotypes that cross the BBB (and which are also AAVrh serotypes), these can be readily replaced or substituted with a VP3 protein from any other serotype that crosses the BBB (including but not limited to, e.g., AAV1, AAV6, AAV6.2, AAV7, AAV9, rAAVrh10, rAAVrh74, rAAVrh39, rAAVrh43 or other AAVrh serotypes). Similarity, AAV8 is shown as an exemplary serotype for the VP1 and/or VP2 protein, but it is envisioned that the VP1 and/or VP2 protein from AAV8 can be replaced or substituted for any serotype disclosed in Table 1 herein.

Systemic Transduction of Haploid Vectors AAV8-8Rh10 or AAV8-8-rh74

The haploid AAVs from Example 1 were next injected intravenously at a dose of 5×10¹⁰ vg of AAV/luc per mouse. At week 3 post injection, imaging was conducted for a period of 30 seconds as seen in FIG. 21A, 1-minute exposure as seen in FIG. 21B, or auto exposure as shown in FIG. 21C. FIG. 21D provides the data from 4 mice after the IV injection with the fold increase of transduction calculated by transduction from compared to the parental AAV8 or AAVrh10.

### Liver Transduction of Haploid Vectors

In this experiment a haploid AAV8-8Rh10 or AAV8-8-rh74 vectors were injected into C57BL6 mice via the retro-orbital vein at a dose of 3×10¹⁰ particles. Imaging was performed one week later. Liver transduction was quantitated based on data that represented the average of 5 mice and standard deviations.

### Chimeric Capsid Proteins and AAV Haploid Virus Vector Transduction

As explained above, a series of constructs for AAV helper plasmids are made with mutants in start codes of capsid ORFs, in which only one or two viral VP proteins would be expressed. Chimeric AAV helper constructs in which VP1/2 protein was driven from two different serotypes (AAV8 and AAV9) can also made. These constructs can be to produce a bunch of haploid virus vectors and evaluate their transduction efficacy in mice. It is found that enhanced transduction is achieved from haploid vectors with VP1/VP2 from serotypes 8, and VP3 from AAVrh10 or rh74 when compared to AAV8-only and AAVrh10-only vectors. It is further assessed if AAV vectors made from the chimeric VP1/VP2 capsid with N-terminus from AAV8 and C-terminus from AAV9 and VP3 from AAVrh10 or rh74 induce much higher transduction. This demonstrated that there is a simple and effective method that enhances AAV transduction and further AAV haploid vectors.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present invention, which is defined solely by the claims. Accordingly, the present invention is not limited to that precisely as shown and described.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present invention so claimed are inherently or expressly described and enabled herein.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

TABLE 1
AAV Serotypes and exemplary Published corresponding capsid sequence

| Serotype and where capsid sequence is published | Serotype and where capsid sequence is published |
|---|---|
| AAV3.3b (See SEQ ID NO:72 in US20030138772) | AAV3-3 (See SEQ ID NO: 200 US20150315612) |
| AAV3-3 (See SEQ ID NO:217 US20150315612) | AAV3a ((See SEQ ID NO: 5 in U.S. Pat. No. 6,156,303) |
| AAV3a (See SEQ ID NO: 9 in U.S. Pat. No. 6,156,303) | AAV3b (See SEQ ID NO: 6 in U.S. Pat. No. 6,156,303) |
| AAV3b (See SEQ ID NO:10 in U.S. Pat. No. 6,156,303) | AAV3b (See SEQ ID NO: 1 in U.S. Pat. No. 6,156,303) |
| AAV4 (See SEQ ID NO: 17 US20140348794) | AAV4 ((See SEQ ID NO:5 in US20140348794) |
| AAV4 (See SEQ ID NO: 3 in US20140348794) | AAV4 (See SEQ ID NO:14 in US20140348794) |
| AAV4 (See SEQ ID NO: 15 in US20140348794) | AAV4 (See SEQ ID NO: 19 in US20140348794) |
| AAV4 (See SEQ ID NO: 12 in US20140348794) | AAV4 (See SEQ ID NO: 13 in US20140348794) |
| AAV4 (See SEQ ID NO: 7 in US20140348794) | AAV4 (See SEQ ID NO: 8 in US20140348794) |
| AAV4 (See SEQ ID NO: 9 in US20140348794) | AAV4 (See SEQ ID NO: 2 in US20140348794) |
| AAV4 (See SEQ ID NO: 10 in US20140348794) | AAV4 (See SEQ ID NO: 11 in US20140348794) |
| AAV4 (See SEQ ID NO: 18 in US20140348794) | AAV4 (See SEQ ID NO:63 in US20030138772) and |
| | US20160017295 SEQ |
| ID NO: (See SEQ ID NO: 4 in US20140348794) | AAV4 (See SEQ ID NO: 16 in US20140348794) |
| AAV4 (See SEQ ID NO: 20 in US20140348794) | AAV4 (See SEQ ID NO: 6 in US20140348794) |
| AAV4 (See SEQ ID NO: 1 in US20140348794) | AAV42.2 (See SEQ ID NO: 9 in US20030138772) |
| AAV42.2 (See SEQ ID NO: 102 in | AAV42.3b (See SEQ ID NO: 36 in US20030138772) |
| US20030138772) | |
| AAV42.3B (See SEQ ID NO: 107 in | AAV42.4 (See SEQ ID NO: 33 in US20030138772) |
| US20030138772) | |
| AAV42.4 (See SEQ ID NO: 88 in | AAV42.8 (See SEQ ID NO: 27 in US20030138772) |
| US20030138772) | |
| AAV42.8 (See SEQ ID NO: 85 in | AAV43.1 (See SEQ ID NO: 39 in US20030138772) |
| US20030138772) | |
| AAV43.1 (See SEQ ID NO: 92 in | AAV43.12 (See SEQ ID NO: 41 in US20030138772) |
| US20030138772) | |
| AAV43.12 (See SEQ ID NO: 93 in | AAV8 (See SEQ ID NO: 15 in US20150159173) |
| US20030138772) | |
| AAV8 (See SEQ ID NO: 7 in US20150376240) | AAV8 (See SEQ ID NO:4 in |
| | US20030138772; US20150315612 SEQ |
| ID NO: 182 | AAV8 (See SEQ ID NO: 95 in US20030138772), |
| | US20140359799 SEQ |
| AAV8 (See SEQ ID NO: 31 in US20150159173) | AAV8 (See, e.g., SEQ ID NO: 8 in US20160017295, or |
| | SEQ ID NO:7 in U.S. Pat. No. 7198951, or SEQ ID NO: 223 in |
| | US20150315612) |
| AAV8 (See SEQ ID NO: 8 in US20150376240) | AAV8 (See SEQ ID NO: 214 in US20150315612) |
| AAV-8b (See SEQ ID NO: 5 in US20150376240) | AAV-8b (See SEQ ID NO: 3 in US20150376240) |
| AAV-8h (See SEQ ID NO: 6 in US20150376240) | AAV-8h (See SEQ ID NO: 4 in US20150376240) |
| AAV9 (See SEQ ID NO: 5 in US20030138772) | AAV9 (See SEQ ID NO: 1 in U.S. Pat. No. 7,198,951) |
| AAV9 (See SEQ ID NO: 9 in US20160017295) | AAV9 (See SEQ ID NO: 100 in US20030138772), |
| | U.S. Pat. No. 7198951 SEQ ID NO: 2 |
| | AAV9 (See SEQ ID NO: 3 in U.S. Pat. No. 7,198,951) |
| AAV9 (AA Vhu. 14) (See SEQ ID NO: 3 in | AAV9 (AAVhu. 14) (See SEQ ID NO: 123 in |
| US20150315612) | US20150315612) |
| AAVA3.1 (See SEQ ID NO: 120 in | AAVA3.3 (See SEQ ID NO: 57 in US20030138772) |
| US20030138772) | |
| AAVA3.3 (See SEQ ID NO: 66 in | AAVA3.4 (See SEQ ID NO: 54 in US20030138772) |
| US20030138772) | |
| AAVA3.4 (See SEQ ID NO: 68 in | AAVA3.5 (See SEQ ID NO: 55 in US20030138772) |
| US20030138772) | |
| AAVA3.5 (See SEQ ID NO: 69 in | AAVA3.7 (See SEQ ID NO: 56 in US20030138772) |
| US20030138772) | |
| AAVA3.7 (See SEQ ID NO: 67 in | AAV29. (See SEQ ID NO: 11 in (AAVbb.1) 161 |
| US20030138772) | US20030138772) |
| AAVC2 (See SEQ ID NO: 61 in US20030138772) | AAVCh.5 (See SEQ ID NO:46 in US20150159173); |
| | US20150315612 SEQ |
| ID NO: 234 | AAVcy.2 (AAV13.3) (See SEQ ID NO: 15 in |
| | US20030138772) |
| AAV24.1 (See SEQ ID NO: 101 in | AAVcy.3 (AAV24.1) (See SEQ ID NO: 16 in |
| US20030138772) | US20030138772) |
| AAV27.3 (See SEQ ID NO: 104 in | AAVcy.4 (AAV27.3) (See SEQ ID NO: 17 in |
| US20030138772) | US20030138772) |
| AAVcy.5 (See SEQ ID NO: 227 in | AAV7.2 (See SEQ ID NO: 103 in US20030138772) |
| US20150315612) | |
| AAVcy.5 (AAV7.2) (See SEQ ID NO: 18 in | AAV16.3 (See SEQ ID NO: 105 in US20030138772) |
| US20030138772) | |
| AAVcy.6 (AAV16.3) (See SEQ ID NO: 10 in | AAVcy.5 (See SEQ ID NO: 8 in US20150159173) |
| US20030138772) | |
| AAVcy.5 (See SEQ ID NO: 24 in | AAVCy.5Rl (See SEQ ID NO: in US20150159173 |
| US20150159173) | |
| AAVCy.5R2 (See SEQ ID NO: in | AAVCy.5R3 (See SEQ ID NO: in US20150159173 |
| US20150159173) | |
| AAVCy.5R4 (See SEQ ID NO: in | AAVDJ (See SEQ ID NO: 3 in US20140359799) and |
| US20150159173) | SEQ ID NO: 2 in U.S. Pat. No. 7,588,772) |
| | AAVDJ (See SEQ ID NO: 2 in US20140359799; and |
| | SEQ ID NO: 1 in U.S. Pat. No. 7,588,772) |
| | AAVDJ-8 (See SEQ ID NO: in US7588772; Grimm et |
| | al 2008 |
| AAVDJ-8 (See SEQ ID NO: in US7588772; | AAVF5 (See SEQ ID NO: 110 in US20030138772) |
| Grimm et al 2008 | |
| AAVH2 (See SEQ ID NO: 26 in US20030138772) | AAVH6 (See SEQ ID NO: 25 in US20030138772) |
| AAVhEl. 1 (See SEQ ID NO: 44 in US9233131) | AAVhErl.14 (See SEQ ID NO: 46 in U.S. Pat. No. 9,233,131) |
| AAVhErl. 16 (See SEQ ID NO: 48 in US9233131) | AAVhErl.18 (See SEQ ID NO: 49 in U.S. Pat. No. 9,233,131) |
| AAVhErl.23 (AAVhEr2.29) (See SEQ ID NO: 53 | AAVhErl.35 (See SEQ ID NO: 50 in U.S. Pat. No. 9,233,131) |
| in US9233131) | |
| AAVhErl.36 (See SEQ ID NO: 52 in US9233131) | AAVhErl.5 (See SEQ ID NO: 45 in U.S. Pat. No. 9,233,131) |
| AAVhErl.7 (See SEQ ID NO: 51 in US9233131) | AAVhErl.8 (See SEQ ID NO: 47 in U.S. Pat. No. 9,233,131) |
| AAVhEr2.16 (See SEQ ID NO: 55 in US9233131) | AAVhEr2.30 (See SEQ ID NO: 56 in U.S. Pat. No. 9,233,131) |
| AAVhEr2.31 (See SEQ ID NO: 58 in US9233131) | AAVhEr2.36 (See SEQ ID NO: 57 in U.S. Pat. No. 9,233,131) |
| AAVhEr2.4 (See SEQ ID NO: 54 in US9233131) | AAVhEr3.1 (See SEQ ID NO: 59 in U.S. Pat. No. Pat. No. 9,233,131) |
| AAVhu.l (See SEQ ID NO: 46 in US20150315612) | AAVhu.l (See SEQ ID NO: 144 in US20150315612) |
| AAVhu.10 (AAV16.8) (See SEQ ID NO: 56 in | AAVhu.10 (AAV16.8) (See SEQ ID NO: 156 in |
| US20150315612) | US20150315612) |
| AAVhu.ll (AAV16.12) (See SEQ ID NO: 57 in | AAVhu.11 (AAV16.12) (See SEQ ID NO: 153 in |
| US20150315612) | US20150315612) |
| AAVhu. 12 (See SEQ ID NO: 59 in | AAVhu. 12 (See SEQ ID NO: 154 in US20150315612) |
| US20150315612) | |
| AAVhu. 13 (See SEQ ID NO: 16 in | |
| US2015015917 and ID NO: 71 in US20150315612) | |
| AAVhu.13 (See SEQ ID NO: 32 in | |
| US20150159173 and ID NO: 129 US20150315612) | |
| AAVhu. 136.1 (See SEQ ID NO: 165 in | AAVhu. 140.1 (See SEQ ID NO: 166 in |
| US20150315612) | US20150315612) |
| AAVhu.140.2 (See SEQ ID NO: 167 in | AAVhu. 145.6 (See SEQ ID NO: 178 in |
| US20150315612) | US20150315612) |
| AAVhu.15 (See SEQ ID NO: 147 in | AAVhu. 15 (AAV33.4) (See SEQ ID NO: 50 in |
| US20150315612) | US20150315612) |
| AAVhu. 156.1 (See SEQ ID NO: 179 in | AAVhu. 16 (See SEQ ID NO: 148 in US20150315612) |
| US20150315612) | |
| AAVhu.16 (AAV33.8) (See SEQ ID NO: 51 in | AAVhu. 17 (See SEQ ID NO: 83 in US20150315612) |
| US20150315612) | |
| AAVhu.17 (AAV33.12) (See SEQ ID NO: 4 in | AAVhu. 172.1 (See SEQ ID NO: 171 in |
| US20150315612) | US20150315612) |
| AAVhu.172.2 (See SEQ ID NO: 172 in | AAVhu. 173.4 (See SEQ ID NO: 173 in |
| US20150315612) | US20150315612) |
| AAVhu.173.8 (See SEQ ID NO: 175 in | AAVhu.18 (See SEQ ID NO: 52 in US20150315612) |
| US20150315612) | |
| AAVhu. 18 (See SEQ ID NO: 149 in | AAVhu. 19 (See SEQ ID NO: 62 in US20150315612) |
| US20150315612) | |
| AAVhu. 19 (See SEQ ID NO: 133 in | AAVhu.2 (See SEQ ID NO: 48 in US20150315612) |
| US20150315612) | |
| AAVhu.2 (See SEQ ID NO: 143 in | AAVhu.20 (See SEQ ID NO: 63 in US20150315612) |
| US20150315612) | |
| AAVhu.20 (See SEQ ID NO: 134 in | AAVhu.21 (See SEQ ID NO: 65 in US20150315612) |
| US20150315612) | |
| AAVhu.21 (See SEQ ID NO: 135 in | AAVhu.22 (See SEQ ID NO: 67 in US20150315612) |
| US20150315612) | |
| AAVhu.22 239 (See SEQ ID NO: 138 in | AAVhu.23 (See SEQ ID NO: 60 in US20150315612) |
| US20150315612) | |
| AAVhu.23.2 (See SEQ ID NO: 137 in | AAVhu.24 (See SEQ ID NO: 66 in US20150315612) |
| US20150315612) | |
| AAVhu.24 (See SEQ ID NO: 136 in | AAVhu.25 (See SEQ ID NO: 49 in US20150315612) |
| US20150315612) | |
| AAVhu.25 (See SEQ ID NO: 146 in | AAVhu.26 (See SEQ ID NO: 17 in US20150159173 and |
| US20150315612) | SEQ ID NO: 61 in US20150315612) |
| | AAVhu.26 (See SEQ ID NO: 33 in US20150159173), |
| | US20150315612 SEQ |
| | AAVhu.27 (See SEQ ID NO: 64 in US20150315612) |
| AAVhu.27 (See SEQ ID NO: 140 in | AAVhu.28 (See SEQ ID NO: 68 in US20150315612) |
| US20150315612) | |
| AAVhu.28 (See SEQ ID NO: 130 in | AAVhu.29 (See SEQ ID NO: 69 in US20150315612) |
| US20150315612) | |
| AAVhu.29 (See SEQ ID NO: 42 in | |
| US20150159173 and SEQ ID NO: 132 in | |
| US20150315612) | |
| AAVhu.29 (See SEQ ID NO: 225 in | AAVhu.29R (See SEQ ID NO: in US20150159173 |
| US20150315612) | |
| AAVhu.3 (See SEQ ID NO: 44 in | AAVhu.3 (See SEQ ID NO: 145 in US20150315612) |
| US20150315612) | |
| AAVhu.30 (See SEQ ID NO: 70 in | AAVhu.30 (See SEQ ID NO: 131 in US20150315612) |
| US20150315612) | |
| AAVhu.31 (See SEQ ID NO: 1 in | AAVhu.31 (See SEQ ID NO: 121 in US20150315612) |
| US20150315612) | |
| AAVhu.32 (See SEQ ID NO: 2 in | AAVhu.32 (See SEQ ID NO: 122 in US20150315612) |
| US20150315612) | |
| AAVhu.33 (See SEQ ID NO: 75 in | AAVhu.33 (See SEQ ID NO: 124 in US20150315612) |
| US20150315612) | |
| AAVhu.34 (See SEQ ID NO: 72 in | AAVhu.34 (See SEQ ID NO: 125 in US20150315612) |
| US20150315612) | |
| AAVhu.35 (See SEQ ID NO: 73 in | AAVhu.35 (See SEQ ID NO: 164 in US20150315612) |
| US20150315612) | |
| AAVhu.36 (See SEQ ID NO: 74 in | AAVhu.36 (See SEQ ID NO: 126 in US20150315612) |
| US20150315612) | |
| AAVhu.37 (See SEQ ID NO: 34 in | |
| US20150159173 and SEQ ID NO: 88 in | |
| US20150315612) | |
| AAVhu.37 (AAV106.1) (See SEQ ID NO: 10 in | |
| US20150315612 and SEQ ID NO: 18 in | |
| US20150159173) | |
| AAVhu.38 (See SEQ ID NO: 161 in | AAVhu.39 (See SEQ ID NO: 102 in US20150315612) |
| US20150315612) | |
| AAVhu.39 (AAVLG-9) (See SEQ ID NO: 24 in | AAVhu.4 (See SEQ ID NO: 47 in US20150315612) |
| US20150315612) | |
| AAVhu.4 (See SEQ ID NO: 141 in | AAVhu.40 (See SEQ ID NO: 87 in US20150315612) |
| US20150315612) | |
| AAVhu.40 (AAV114.3) (See SEQ ID NO: 11 in | AAVhu.41 (See SEQ ID NO: 91 in US20150315612) |
| US20150315612) | |
| AAVhu.41 (AAV127.2) (See SEQ ID NO: 6 in | AAVhu.42 (See SEQ ID NO: 85 in US20150315612) |
| US20150315612) | |
| AAVhu.42 (AAV127.5) (See SEQ ID NO:8 in | AAVhu.43 (See SEQ ID NO: 160 in US20150315612) |
| US20150315612) | |
| AAVhu.43 (See SEQ ID NO: 236 in | AAVhu.43 (AAV128.1) (See SEQ ID NO: 80 in |
| US20150315612) | US20150315612) |
| AAVhu.44 (See SEQ ID NO: 45 in | |
| US20150159173 and SEQ ID NO: 158 in | |
| US20150315612) | |
| AAVhu.44 (AAV128.3) (See SEQ ID NO: 81 in | AAVhu.44Rl (See SEQ ID NO: in US20150159173 |
| US20150315612) | |
| AAVhu.44R2 (See SEQ ID NO: in | AAVhu.44R3 (See SEQ ID NO: in US20150159173 |
| US20150159173 | |
| AAVhu.45 (See SEQ ID NO: 76 in | AAVhu.45 (See SEQ ID NO: 127 in US20150315612) |
| US20150315612) | |
| AAVhu.46 (See SEQ ID NO: 82 in | AAVhu.46 (See SEQ ID NO: 159 in US20150315612) |
| US20150315612) | |
| AAVhu.46 (See SEQ ID NO: 224 in | AAVhu.47 (See SEQ ID NO: 77 in US20150315612) |
| US20150315612) | |
| AAVhu.47 (See SEQ ID NO: 128 in | AAVhu.48 (See SEQ ID NO: 38 in US20150159173) |
| US20150315612) | |
| AAVhu.48 (See SEQ ID NO: 157 in | AAVhu.48 (AAV130.4) (See SEQ ID NO: 78 in |
| US20150315612) | US20150315612) |
| AAVhu.48Rl (See SEQ ID NO: in | AAVhu.48R2 (See SEQ ID NO: in US20150159173 |
| US20150159173 | |
| AAVhu.48R3 (See SEQ ID NO: in | AAVhu.49 (See SEQ ID NO: 209 in US20150315612) |
| US20150159173 | |
| AAVhu.49 (See SEQ ID NO: 189 in | AAVhu.5 (See SEQ ID NO: 45 in US20150315612) |
| US20150315612) | |
| AAVhu.5 (See SEQ ID NO: 142 in | AAVhu.51 (See SEQ ID NO: 208 in US20150315612) |
| US20150315612) | |
| AAVhu.51 (See SEQ ID NO: 190 in | AAVhu.52 (See SEQ ID NO: 210 in US20150315612) |
| US20150315612) | |
| AAVhu.52 (See SEQ ID NO: 191 in | AAVhu.53 (See SEQ ID NO: 19 in US20150159173) |
| US20150315612) | |
| AAVhu.53 (See SEQ ID NO: 35 in | AAVhu.53 (AAV145.1) (See SEQ ID NO: 176 in |
| US20150159173) | US20150315612) |
| AAVhu.54 (See SEQ ID NO: 188 in | AAVhu.54 (AAV145.5) (See SEQ ID NO: 177 in |
| US20150315612) | US20150315612) |
| AAVhu.55 (See SEQ ID NO: 187 in | AAVhu.56 (See SEQ ID NO: 205 in US20150315612) |
| US20150315612) | |
| AAVhu.56 (AAV145.6) (See SEQ ID NO: 168 in | AAVhu.56 (AAV145.6) (See SEQ ID NO: 192 in |
| US20150315612) | US20150315612) |
| AAVhu.57 (See SEQ ID NO: 206 in | AAVhu.57 (See SEQ ID NO: 169 in US20150315612) |
| US20150315612) | |
| AAVhu.57 (See SEQ ID NO: 193 in | AAVhu.58 (See SEQ ID NO: 207 in US20150315612) |
| US20150315612) | |
| AAVhu.58 (See SEQ ID NO: 194 in | AAVhu.6 (AAV3.1) (See SEQ ID NO: 5 in |
| US20150315612) | US20150315612) |
| AAVhu.6 (AAV3.1) (See SEQ ID NO: 84 in | AAVhu.60 (See SEQ ID NO: 184 in US20150315612) |
| US20150315612) | |
| AAVhu.60 (AAV161.10) (See SEQ ID NO: 170 in | AAVhu.61 (See SEQ ID NO: 185 in US20150315612) |
| US20150315612) | |
| AAVhu.61 (AAV161.6) (See SEQ ID NO: 174 in | AAVhu.63 (See SEQ ID NO: 204 in US20150315612) |
| US20150315612) | |
| AAVhu.63 (See SEQ ID NO: 195 in | AAVhu.64 (See SEQ ID NO: 212 in US20150315612) |
| US20150315612) | |
| AAVhu.64 (See SEQ ID NO: 196 in | AAVhu.66 (See SEQ ID NO: 197 in US20150315612) |
| US20150315612) | |
| AAVhu.67 (See SEQ ID NO: 215 in | AAVhu.67 (See SEQ ID NO: 198 in US20150315612) |
| US20150315612) | |
| AAVhu.7 (See SEQ ID NO: 226 in | AAVhu.7 (See SEQ ID NO: 150 in US20150315612) |
| US20150315612) | |
| AAVhu.7 (AAV7.3) (See SEQ ID NO: 55 in | AAVhu.71 (See SEQ ID NO: 79 in US20150315612) |
| US20150315612) | |
| AAVhu.8 (See SEQ ID NO: 53 in | AAVhu.8 (See SEQ ID NO: 12 in US20150315612) |
| US20150315612) | |
| AAVhu.8 (See SEQ ID NO: 151 in | AAVhu.9 (AAV3.1) (See SEQ ID NO: 58 in |
| US20150315612) | US20150315612) |
| AAVhu.9 (AAV3.1) (See SEQ ID NO: 155 in | AAV-LK01 (See SEQ ID NO: 2 in US20150376607) |
| US20150315612) | |
| AAV-LK01 (See SEQ ID NO: 29 in | AAV-LK02 (See SEQ ID NO: 3 in US20150376607) |
| US20150376607) | |
| AAV-LK02 (See SEQ ID NO: 30 in | AAV-LK03 (See SEQ ID NO: 4 in US20150376607) |
| US20150376607) | |
| AAV-LK03 (See SEQ ID NO: 12 in | |
| WO2015121501 and SEQ ID NO: 31 in | |
| US20150376607) | |
| AAV-LK04 (See SEQ ID NO: 5 in | AAV-LK04 (See SEQ ID NO: 32 in US20150376607) |
| US20150376607) | |
| AAV-LK05 (See SEQ ID NO: 6 in | AAV-LK05 (See SEQ ID NO: 33 in US20150376607) |
| US20150376607) | |
| AAV-LK06 (See SEQ ID NO: 7 in | AAV-LK06 (See SEQ ID NO: 34 in US20150376607) |
| US20150376607) | |
| AAV-LK07 (See SEQ ID NO: 8 in | AAV-LK07 (See SEQ ID NO: 35 in US20150376607) |
| US20150376607) | |
| AAV-LK08 (See SEQ ID NO: 9 in | AAV-LK08 (See SEQ ID NO: 36 in US20150376607) |
| US20150376607) | |
| AAV-LK09 (See SEQ ID NO: 10 in | AAV-LK09 (See SEQ ID NO: 37 in US20150376607) |
| US20150376607) | |
| AAV-LK10 (See SEQ ID NO: 11 in | AAV-LK10 (See SEQ ID NO: 38 in US20150376607) |
| US20150376607) | |
| AAV-LK11 (See SEQ ID NO: 12 in | AAV-LK11 (See SEQ ID NO: 39 in US20150376607) |
| US20150376607) | |
| AAV-LK12 (See SEQ ID NO: 13 in | AAV-LK12 (See SEQ ID NO: 40 in US20150376607) |
| US20150376607) | |
| AAV-LK13 (See SEQ ID NO: 14 in | AAV-LK13 (See SEQ ID NO: 41 in US20150376607) |
| US20150376607) | |
| AAV-LK14 (See SEQ ID NO: 15 in | AAV-LK14 (See SEQ ID NO: 42 in US20150376607) |
| US20150376607) | |
| AAV-LK15 (See SEQ ID NO: 16 in | AAV-LK15 (See SEQ ID NO: 43 in US20150376607) |
| US20150376607) | |
| AAV-LK16 (See SEQ ID NO: 17 in | AAV-LK16 (See SEQ ID NO: 44 in US20150376607) |
| US20150376607) | |
| AAV-LK17 (See SEQ ID NO: 18 in | AAV-LK17 (See SEQ ID NO: 45 in US20150376607) |
| US20150376607) | |
| AAV-LK18 (See SEQ ID NO: 19 in | AAV-LK18 (See SEQ ID NO: 46 in US20150376607) |
| US20150376607) | |
| AAV-LK19 (See SEQ ID NO: 20 in | AAV-LK19 (See SEQ ID NO: 47 in US20150376607) |
| US20150376607) | |
| AAV-PAEC (See SEQ ID NO: 1 in | AAV-PAEC (See SEQ ID NO: 48 in US20150376607) |
| US20150376607) | |
| AAV-PAEC11 (See SEQ ID NO: 26 in | AAV-PAEC11 (See SEQ ID NO: 54 in |
| US20150376607) | US20150376607) |
| AAV-PAEC 12 (See SEQ ID NO: 27 in | AAV-PAEC 12 (See SEQ ID NO: 51 in |
| US20150376607) | US20150376607) |
| AAV-PAEC 13 (See SEQ ID NO: 28 in | AAV-PAEC 13 (See SEQ ID NO: 49 in |
| US20150376607) | US20150376607) |
| AAV-PAEC2 (See SEQ ID NO: 21 in | AAV-PAEC2 (See SEQ ID NO: 56 in US20150376607) |
| US20150376607) | |
| AAV-PAEC4 (See SEQ ID NO: 22 in | AAV-PAEC4 (See SEQ ID NO: 55 in US20150376607) |
| US20150376607) | |
| AAV-PAEC6 (See SEQ ID NO: 23 in | AAV-PAEC6 (See SEQ ID NO: 52 in US20150376607) |
| US20150376607) | |
| AAV-PAEC7 (See SEQ ID NO: 24 in | AAV-PAEC7 (See SEQ ID NO: 53 in US20150376607) |
| US20150376607) | |
| AAV-PAEC8 (See SEQ ID NO: 25 in | AAV-PAEC8 (See SEQ ID NO: 50 in US20150376607) |
| US20150376607) | |
| AAVpi.1 (See SEQ ID NO: 28 in US20150315612) | AAVpi.l (See SEQ ID NO: 93 in US20150315612; |
| | AAVpi.2 408, see SEQ ID NO: 30 in US20150315612) |
| AAVpi.2 (See SEQ ID NO: 95 in | AAVpi.3 (See SEQ ID NO: 29 in US20150315612) |
| US20150315612) | |
| AAVpi.3 (See SEQ ID NO: 94 in | AAVrh.10 (See SEQ ID NO: 9 in US20150159173) |
| US20150315612) | |
| AAVrh. 10 (See SEQ ID NO: 25 in | AAV44.2 (See SEQ ID NO: 59 in US20030138772) |
| US20150159173) | |
| AAVrh.10 (AAV44.2) (See SEQ ID NO: 81 in | AAV42.1B (See SEQ ID NO: 90 in US20030138772) |
| US20030138772) | |
| AAVrh.12 (AAV42.1b) (See SEQ ID NO: 30 in | AAVrh.13 (See SEQ ID NO: 10 in US20150159173) |
| US20030138772) | |
| AAVrh.13 (See SEQ ID NO: 26 in | AAVrh. 13 (See SEQ ID NO: 228 in US20150315612) |
| US20150159173) | |
| AAVrh.13R (See SEQ ID NO: in US20150159173 | AAV42.3A (See SEQ ID NO: 87 in US20030138772) |
| AAVrh.14 (AAV42.3a) (See SEQ ID NO: 32 in | AAV42.5A (See SEQ ID NO: 89 in US20030138772) |
| US20030138772) | |
| AAVrh.17 (AAV42.5a) (See SEQ ID NO: 34 in | AAV42.5B (See SEQ ID NO: 91 in US20030138772) |
| US20030138772) | |
| AAVrh.18 (AAV42.5b) (See SEQ ID NO: 29 in | AAV42.6B (See SEQ ID NO: 112 in US20030138772) |
| US20030138772) | |
| AAVrh.19 (AAV42.6b) (See SEQ ID NO: 38 in | AAVrh.2 (See SEQ ID NO: 39 in US20150159173) |
| US20030138772) | |
| AAVrh.2 (See SEQ ID NO: 231 in | AAVrh.20 (See SEQ ID NO: 1 in US20150159173) |
| US20150315612) | |
| AAV42.10 (See SEQ ID NO: 106 in | AAVrh.21 (AAV42.10) (See SEQ ID NO: 35 in |
| US20030138772) | US20030138772) |
| AAV42.11 (See SEQ ID NO: 108 in | AAVrh.22 (AAV42.11) (See SEQ ID NO: 37 in |
| US20030138772) | US20030138772) |
| AAV42.12 (See SEQ ID NO: 113 in | AAVrh.23 (AAV42.12) (See SEQ ID NO: 58 in |
| US20030138772) | US20030138772) |
| AAV42.13 (See SEQ ID NO: 86 in | AAVrh.24 (AAV42.13) (See SEQ ID NO: 31 in |
| US20030138772) | US20030138772) |
| AAV42.15 (See SEQ ID NO: 84 in | AAVrh.25 (AAV42.15) (See SEQ ID NO: 28 in |
| US20030138772) | US20030138772) |
| AAVrh.2R (See SEQ ID NO: in US20150159173 | AAVrh.31 (AAV223.1) (See SEQ ID NO: 48 in |
| | US20030138772) |
| AAVCI (See SEQ ID NO: 60 in US20030138772) | AAVrh.32 (AAVC1) (See SEQ ID NO: 19 in 446 |
| | US20030138772) |
| AAVrh.32/33 (See SEQ ID NO: 2 in | AAVrh.51 (AAV2-5) (See SEQ ID NO: 104 in |
| US20150159173) | US20150315612) |
| AAVrh.52 (AAV3-9) (See SEQ ID NO: 18 in | AAVrh.52 (AAV3-9) (See SEQ ID NO: 96 in |
| US20150315612) | US20150315612) |
| AAVrh.53 (See SEQ ID NO: in US20150315612) | AAVrh.53 (AAV3-11) (See SEQ ID NO: 17 in |
| | US20150315612) |
| AAVrh.53 (AAV3-11) (See SEQ ID NO: 186 in | AAVrh.54 (See SEQ ID NO: 40 in US20150315612) |
| US20150315612) | |
| AAVrh.54 (See SEQ ID NO: 49 in | |
| US20150159173 and SEQ ID NO: 116 in | |
| US20150315612) | |
| AAVrh.55 (See SEQ ID NO: 37 in | AAVrh.55 (AAV4-19) (See SEQ ID NO: 117 in |
| US20150315612) | US20150315612) |
| AAVrh.56 (See SEQ ID NO: 54 in | AAVrh.56 (See SEQ ID NO: 152 in US20150315612) |
| US20150315612) | |
| AAVrh.57 (See SEQ ID NO: in 497 | AAVrh.57 (See SEQ ID NO: 105 in US20150315612) |
| US20150315612 SEQ ID NO: 26 | |
| AAVrh.58 (See SEQ ID NO: 27 in | AAVrh.58 (See SEQ ID NO: 48 in US20150159173 and |
| US20150315612) | SEQ ID NO: 106 in US20150315612) |
| | AAVrh.58 (See SEQ ID NO: 232 in US20150315612) |
| AAVrh.59 (See SEQ ID NO: 42 in | AAVrh.59 (See SEQ ID NO: 110 in US20150315612) |
| US20150315612) | |
| AAVrh.60 (See SEQ ID NO: 31 in | AAVrh.60 (See SEQ ID NO: 120 in US20150315612) |
| US20150315612) | |
| AAVrh.61 (See SEQ ID NO: 107 in | AAVrh.61 (AAV2-3) (See SEQ ID NO: 21 in |
| US20150315612) | US20150315612) |
| AAVrh.62 (AAV2-15) (See SEQ ID NO: 33 in | AAVrh.62 (AAV2-15) (See SEQ ID NO: 114 in |
| US20150315612) | US20150315612) |
| AAVrh.64 (See SEQ ID NO: 15 in | AAVrh.64 (See SEQ ID NO: 43 in US20150159173 and |
| US20150315612) | SEQ ID NO: 99 in US20150315612) |
| | AAVrh.64 (See SEQ ID NO: 233 in US20150315612) |
| AAVRh.64Rl (See SEQ ID NO: in | AAVRh.64R2 (See SEQ ID NO: in US20150159173 |
| US20150159173 | |
| AAVrh.65 (See SEQ ID NO: 35 in | AAVrh.65 (See SEQ ID NO: 112 in US20150315612) |
| US20150315612) | |
| AAVrh.67 (See SEQ ID NO: 36 in | AAVrh.67 (See SEQ ID NO: 230 in US20150315612) |
| US20150315612) | |
| AAVrh.67 (See SEQ ID NO: 47 in | |
| US20150159173 and SEQ ID NO: 47 in | |
| US20150315612) | |
| AAVrh.68 (See SEQ ID NO: 16 in | AAVrh.68 (See SEQ ID NO: 100 in US20150315612) |
| US20150315612) | |
| AAVrh.69 (See SEQ ID NO: 39 in | AAVrh.69 (See SEQ ID NO: 119 in US20150315612) |
| US20150315612) | |
| AAVrh.70 (See SEQ ID NO: 20 in | AAVrh.70 (See SEQ ID NO: 98 in US20150315612) |
| US20150315612) | |
| AAVrh.71 (See SEQ ID NO: 162 in | AAVrh.72 (See SEQ ID NO: 9 in US20150315612) |
| US20150315612) | |
| AAVrh.73 (See SEQ ID NO: 5 in | AAVrh.74 (See SEQ ID NO: 6 in US20150159173) |
| US20150159173) | |
| AAVrh.8 (See SEQ ID NO: 41 in | AAVrh.8 (See SEQ ID NO: 235 in US20150315612) |
| US20150159173) | |
| AAVrh.8R (See SEQ ID NO: 9 in | AAVrh.8R A586R mutant (See SEQ ID NO: 10 in |
| US20150159173, WO2015168666) | WO2015168666) |
| AAVrh.8R R533A mutant (See SEQ ID NO: 11 in | BAAV (bovine AAV) (See SEQ ID NO: 8 in |
| WO2015168666) | U.S. Pat. No. 9,193,769) |
| BAAV (bovine AAV) (See SEQ ID NO: 10 in | BAAV (bovine AAV) (See SEQ ID NO: 4 in |
| U.S. Pat. No. 9,193,769) | U.S. Pat. No. 9,193,769) |
| BAAV (bovine AAV) (See SEQ ID NO: 2 in | BAAV (bovine AAV) (See SEQ ID NO: 6 in |
| U.S. Pat. No. 9,193,769) | U.S. Pat. No. 9,193,769) |
| BAAV (bovine AAV) (See SEQ ID NO: 1 in | BAAV (bovine AAV) (See SEQ ID NO: 5 in |
| U.S. Pat. No. 9,193,769) | U.S. Pat. No. 9,193,769) |
| BAAV (bovine AAV) (See SEQ ID NO: 3 in | BAAV (bovine AAV) (See SEQ ID NO: 11 in |
| U.S. Pat. No. 9,193,769) | U.S. Pat. No. 9,193,769) |
| BAAV (bovine AAV) (See SEQ ID NO: 5 in | BAAV (bovine AAV) (See SEQ ID NO: 6 in |
| U.S. Pat. No. 7,427,396) | U.S. Pat. No. 7,427,396) |
| BAAV (bovine AAV) (See SEQ ID NO: 7 in | BAAV (bovine AAV) (See SEQ ID NO: 9 in |
| U.S. Pat. No. 9,193,769) | U.S. Pat. No. 9,193,769) |
| BNP61 AAV (See SEQ ID NO: 1 in | BNP61 AAV (See SEQ ID NO: 2 in US20150238550) |
| US20150238550) | |
| BNP62 AAV (See SEQ ID NO: 3 in | BNP63 AAV (See SEQ ID NO: 4 in US20150238550) |
| US20150238550) | |
| caprine AAV (See SEQ ID NO: 3 in U.S. Pat. No. 7,427,396) | caprine AAV (See SEQ ID NO: 4 in U.S. Pat. No. 7,427,396) |
| true type AAV (ttAAV) (See SEQ ID NO: 2 in | AAAV (Avian AAV) (See SEQ ID NO: 12 in |
| WO2015121501) | U.S. Pat. No. 9,238,800) |
| AAAV (Avian AAV) (See SEQ ID NO: 2 in | AAAV (Avian AAV) (See SEQ ID NO: 6 in |
| U.S. Pat. No. 9,238,800) | U.S. Pat. No. 9,238,800) |
| AAAV (Avian AAV) (See SEQ ID NO: 4 in | AAAV (Avian AAV) (See SEQ ID NO: 8 in |
| U.S. Pat. No. 9,238,800) | U.S. Pat. No. 9,238,800) |
| AAAV (Avian AAV) (See SEQ ID NO: 14 in | AAAV (Avian AAV) (See SEQ ID NO: 10 in |
| U.S. Pat. No. 9,238,800) | U.S. Pat. No. 9,238,800) |
| AAAV (Avian AAV) (See SEQ ID NO: 15 in | AAAV (Avian AAV) (See SEQ ID NO: 5 in |
| U.S. Pat. No. 9,238,800) | U.S. Pat. No. 9,238,800) |
| AAAV (Avian AAV) (See SEQ ID NO: 9 in | AAAV (Avian AAV) (See SEQ ID NO: 3 in |
| U.S. Pat. No. 9,238,800) | U.S. Pat. No. 9,238,800) |
| AAAV (Avian AAV) (See SEQ ID NO: 7 in | AAAV (Avian AAV) (See SEQ ID NO: 11 in |
| U.S. Pat. No. 9,238,800) | U.S. Pat. No. 9,238,800) |
| AAAV (Avian AAV) (See SEQ ID NO: in | AAAV (Avian AAV) (See SEQ ID NO: 1 in |
| U.S. Pat. No. 9,238,800) | U.S. Pat. No. 9,238,800) |
| AAV Shuffle 100-1 (See SEQ ID NO: 23 in | AAV Shuffle 100-1 (See SEQ ID NO: 11 in |
| US20160017295) | US20160017295) |
| AAV Shuffle 100-2 (See SEQ ID NO: 37 in | AAV Shuffle 100-2 (See SEQ ID NO: 29 in |
| US20160017295) | US20160017295) |
| AAV Shuffle 100-3 (See SEQ ID NO: 24 in | AAV Shuffle 100-3 (See SEQ ID NO: 12 in |
| US20160017295) | US20160017295) |
| AAV Shuffle 100-7 (See SEQ ID NO: 25 in | AAV Shuffle 100-7 (See SEQ ID NO: 13 in |
| US20160017295) | US20160017295) |
| AAV Shuffle 10-2 (See SEQ ID NO: 34 in | AAV Shuffle 10-2 (See SEQ ID NO: 26 in |
| US20160017295) | US20160017295) |
| AAV Shuffle 10-6 (See SEQ ID NO: 35 in | AAV Shuffle 10-6 (See SEQ ID NO: 27 in |
| US20160017295) | US20160017295) |
| AAV Shuffle 10-8 (See SEQ ID NO: 36 in | AAV Shuffle 10-8 (See SEQ ID NO: 28 in |
| US20160017295) | US20160017295) |
| AAV SM 100-10 (See SEQ ID NO: 41 in | AAV SM 100-10 (See SEQ ID NO: 33 in |
| US20160017295) | US20160017295) |
| AAV SM 100-3 (See SEQ ID NO: 40 in | AAV SM 100-3 (See SEQ ID NO: 32 in |
| US20160017295) | US20160017295) |
| AAV SM 10-1 (See SEQ ID NO: 38 in | AAV SM 10-1 (See SEQ ID NO: 30 in US20160017295) |
| US20160017295) | |
| AAV SM 10-2 (See SEQ ID NO: 10 in | AAV SM 10-2 (See SEQ ID NO: 22 in US20160017295) |
| US20160017295) | |
| AAV SM 10-8 (See SEQ ID NO: 39 in | AAV SM 10-8 (See SEQ ID NO: 31 in US20160017295) |
| US20160017295) | |
| AAV CBr-7.1 (See SEQ ID NO: 4 in | AAV CBr-7.1 (See SEQ ID NO: 54 in WO2016065001) |
| WO2016065001) | |
| AAV CBr-7.10 (See SEQ ID NO: 11 in | AAV CBr-7.10 (See SEQ ID NO: 61 in |
| WO2016065001) | WO2016065001) |
| AAV CBr-7.2 (See SEQ ID NO: 5 in | AAV CBr-7.2 (See SEQ ID NO: 55 in WO2016065001) |
| WO2016065001) | |
| AAV CBr-7.3 (See SEQ ID NO: 6 in | AAV CBr-7.3 (See SEQ ID NO: 56 in WO2016065001) |
| WO2016065001) | |
| AAV CBr-7.4 (See SEQ ID NO: 7 in | AAV CBr-7.4 (See SEQ ID NO: 57 in WO2016065001) |
| WO2016065001) | |
| AAV CBr-7.5 (See SEQ ID NO: 8 in | AAV CHt-6.6 (See SEQ ID NO: 35 in WO2016065001) |
| WO2016065001) | |
| AAV CHt-6.6 (See SEQ ID NO: 85 in | AAV CHt-6.7 (See SEQ ID NO: 36 in WO2016065001) |
| WO2016065001) | |
| AAV CHt-6.7 (See SEQ ID NO: 86 in | AAV CHt-6.8 (See SEQ ID NO: 37 in WO2016065001) |
| WO2016065001) | |
| AAV CHt-6.8 (See SEQ ID NO: 87 in | AAV CHt-Pl (See SEQ ID NO: 29 in WO2016065001) |
| WO2016065001) | |
| AAV CHt-P1 (See SEQ ID NO: 79 in | AAV CHt-P2 (See SEQ ID NO: 1 in WO2016065001) |
| WO2016065001) | |
| AAV CHt-P2 (See SEQ ID NO: 51 in | AAV CHt-P5 (See SEQ ID NO: 2 in WO2016065001) |
| WO2016065001) | |
| AAV CHt-P5 (See SEQ ID NO: 52 in | AAV CHt-P6 (See SEQ ID NO: 30 in WO2016065001) |
| WO2016065001) | |
| AAV CHt-P6 (See SEQ ID NO: 80 in | AAV CHt-P8 (See SEQ ID NO: 31 in WO2016065001) |
| WO2016065001) | |
| AAV CHt-P8 (See SEQ ID NO: 81 in | AAV CHt-P9 (See SEQ ID NO: 3 in WO2016065001) |
| WO2016065001) | |
| AAV CHt-P9 (See SEQ ID NO: 53 in | AAV CKd-1 (See SEQ ID NO: 57 in U.S. Pat. No. 8,734,809) |
| WO2016065001) | |
| AAV CKd-1 (See SEQ ID NO: 131 in | AAV CKd-10 (See SEQ ID NO: 58 in U.S. Pat. No. 8,734,809) |
| U.S. Pat. No. 8,734,809) | |
| AAV CKd-10 (See SEQ ID NO: 132 in | AAV CKd-2 (See SEQ ID NO: 59 in U.S. Pat. No. 8,734,809) |
| U.S. Pat. No. 8,734,809) | |
| AAV CKd-2 (See SEQ ID NO: 133 in | AAV CKd-3 (See SEQ ID NO: 60 in U.S. Pat. No. 8,734,809) |
| U.S. Pat. No. 8,734,809) | |
| AAV CKd-3 (See SEQ ID NO: 134 in | AAV CKd-4 (See SEQ ID NO: 61 in U.S. Pat. No. 8,734,809) |
| U.S. Pat. No. 8,734,809) | |
| AAV CKd-4 (See SEQ ID NO: 135 in | AAV CKd-6 (See SEQ ID NO: 62 in U.S. Pat. No. 8,734,809) |
| U.S. Pat. No. 8,734,809) | |
| AAV CKd-6 (See SEQ ID NO: 136 in | AAV CKd-7 (See SEQ ID NO: 63 in U.S. Pat. No. 8,734,809) |
| U.S. Pat. No. 8,734,809) | |
| AAV CKd-7 (See SEQ ID NO: 137 in | AAV CKd-8 (See SEQ ID NO: 64 in U.S. Pat. No. 8,734,809) |
| U.S. Pat. No. 8,734,809) | |
| AAV CKd-8 (See SEQ ID NO: 138 in | AAV CKd-B 1 (See SEQ ID NO: 73 in U.S. Pat. No. 8,734,809) |
| U.S. Pat. No. 8,734,809) | |
| AAV CKd-B 1 (See SEQ ID NO: 147 in | AAV CKd-B2 (See SEQ ID NO: 74 in U.S. Pat. No. 8,734,809) |
| U.S. Pat. No. 8,734,809) | |
| AAV CKd-B2 (See SEQ ID NO: 148 in | AAV CKd-B3 (See SEQ ID NO: 75 in U.S. Pat. No. 8,734,809) |
| U.S. Pat. No. 8,734,809) | |
| AAV CKd-B3 (See SEQ ID NO: in U.S. Pat. No. 8,734,809 | AAV CKd-B3 (See SEQ ID NO: 149 in U.S. Pat. No. 8,734,809) |
| AAV CLv-1 (See SEQ ID NO: 65 in U.S. Pat. No. 8,734,809) | AAV CLv-1 (See SEQ ID NO: 139 in U.S. Pat. No. 8,734,809) |
| AAV CLvl-1 (See SEQ ID NO: 171 in | AAV Civ 1-10 (See SEQ ID NO: 178 in U.S. Pat. No. 8,734,809) |
| U.S. Pat. No. 8,734,809) | |
| AAV CLvl-2 (See SEQ ID NO: 172 in | AAV CLv-12 (See SEQ ID NO: 66 in U.S. Pat. No. 8,734,809) |
| U.S. Pat. No. 8,734,809) | |
| AAV CLv-12 (See SEQ ID NO: 140 in | AAV CLvl-3 (See SEQ ID NO: 173 in U.S. Pat. No. 8,734,809) |
| U.S. Pat. No. 8,734,809) | |
| AAV CLv-13 (See SEQ ID NO: 67 in | AAV CLv-13 (See SEQ ID NO: 141 in U.S. Pat. No. 8,734,809) |
| U.S. Pat. No. 8,734,809) | |
| AAV CLvl-4 (See SEQ ID NO: 174 in | AAV Civ 1-7 (See SEQ ID NO: 175 in U.S. Pat. No. 8,734,809) |
| U.S. Pat. No. 8,734,809) | |
| AAV Civ 1-8 (See SEQ ID NO: 176 in | AAV Civ 1-9 (See SEQ ID NO: 177 in U.S. Pat. No. 8,734,809) |
| U.S. Pat. No. 8,734,809) | |
| AAV CLv-2 (See SEQ ID NO: 68 in U.S. Pat. No. 8,734,809) | AAV CLv-2 (See SEQ ID NO: 142 in U.S. Pat. No. 8,734,809) |
| AAV CLv-3 (See SEQ ID NO: 69 in U.S. Pat. No. 8,734,809) | AAV CLv-3 (See SEQ ID NO: 143 in U.S. Pat. No. 8,734,809) |
| AAV CLv-4 (See SEQ ID NO: 70 in U.S. Pat. No. 8,734,809) | AAV CLv-4 (See SEQ ID NO: 144 in U.S. Pat. No. 8,734,809) |
| AAV CLv-6 (See SEQ ID NO: 71 in U.S. Pat. No. 8,734,809) | AAV CLv-6 (See SEQ ID NO: 145 in U.S. Pat. No. 8,734,809) |
| AAV CLv-8 (See SEQ ID NO: 72 in U.S. Pat. No. 8,734,809) | AAV CLv-8 (See SEQ ID NO: 146 in U.S. Pat. No. 8,734,809) |
| AAV CLv-DI (See SEQ ID NO: 22 in | AAV CLv-DI (See SEQ ID NO: 96 in U.S. Pat. No. 8,734,809) |
| U.S. Pat. No. 8,734,809) | |
| AAV CLv-D2 (See SEQ ID NO: 23 in | AAV CLv-D2 (See SEQ ID NO: 97 in U.S. Pat. No. 8,734,809) |
| U.S. Pat. No. 8,734,809) | |
| AAV CLv-D3 (See SEQ ID NO: 24 in | AAV CLv-D3 (See SEQ ID NO: 98 in U.S. Pat. No. 8,734,809) |
| U.S. Pat. No. 8,734,809) | |
| AAV CLv-D4 (See SEQ ID NO: 25 in | AAV CLv-D4 (See SEQ ID NO: 99 in U.S. Pat. No. 8,734,809) |
| U.S. Pat. No. 8,734,809) | |
| AAV CLv-D5 (See SEQ ID NO: 26 in | AAV CLv-D5 (See SEQ ID NO: 100 in U.S. Pat. No. 8,734,809) |
| U.S. Pat. No. 8,734,809) | |
| AAV CLv-D6 (See SEQ ID NO: 27 in | AAV CLv-D6 (See SEQ ID NO: 101 in U.S. Pat. No. 8,734,809) |
| U.S. Pat. No. 8,734,809) | |
| AAV CLv-D7 (See SEQ ID NO: 28 in | AAV CLv-D7 (See SEQ ID NO: 102 in U.S. Pat. No. 8,734,809) |
| U.S. Pat. No. 8,734,809) | |
| AAV CLv-D8 (See SEQ ID NO: 29 in | AAV CLv-D8 (See SEQ ID NO: 103 in U.S. Pat. No. 8,734,809); |
| U.S. Pat. No. 8,734,809) | AAV CLv-K1 762, see SEQ ID NO: 18 in |
| | WO2016065001) |
| AAV CLv-Kl (See SEQ ID NO: 68 in | AAV CLv-K3 (See SEQ ID NO: 19 in WO2016065001) |
| WO2016065001) | |
| AAV CLv-K3 (See SEQ ID NO: 69 in | AAV CLv-K6 (See SEQ ID NO: 20 in WO2016065001) |
| WO2016065001) | |
| AAV CLv-K6 (See SEQ ID NO: 70 in | AAV CLv-L4 (See SEQ ID NO: 15 in WO2016065001) |
| WO2016065001) | |
| AAV CLv-L4 (See SEQ ID NO: 65 in | AAV CLv-L5 (See SEQ ID NO: 16 in WO2016065001) |
| WO2016065001) | |
| AAV CLv-L5 (See SEQ ID NO: 66 in | AAV CLv-L6 (See SEQ ID NO: 17 in WO2016065001) |
| WO2016065001) | |
| AAV CLv-L6 (See SEQ ID NO: 67 in | AAV CLv-MI (See SEQ ID NO: 21 in WO2016065001) |
| WO2016065001) | |
| AAV CLv-MI (See SEQ ID NO: 71 in | AAV CLv-Mll (See SEQ ID NO: 22 in WO2016065001) |
| WO2016065001) | |
| AAV CLv-MI 1 (See SEQ ID NO: 72 in | AAV CLv-M2 (See SEQ ID NO: 23 in WO2016065001) |
| WO2016065001) | |
| AAV CLv-M2 (See SEQ ID NO: 73 in | AAV CLv-M5 (See SEQ ID NO: 24 in WO2016065001) |
| WO2016065001) | |
| AAV CLv-M5 (See SEQ ID NO: 74 in | AAV CLv-M6 (See SEQ ID NO: 25 in WO2016065001) |
| WO2016065001) | |
| AAV CLv-M6 (See SEQ ID NO: 75 in | AAV CLv-M7 (See SEQ ID NO: 26 in WO2016065001) |
| WO2016065001) | |
| AAV CLv-M7 (See SEQ ID NO: 76 in | AAV CLv-M8 (See SEQ ID NO: 27 in WO2016065001) |
| WO2016065001) | |
| AAV CLv-M8 (See SEQ ID NO: 77 in | AAV CLv-M9 (See SEQ ID NO: 28 in WO2016065001) |
| WO2016065001) | |
| AAV CLv-M9 (See SEQ ID NO: 78 in | AAV CLv-RI (See SEQ ID NO: 30 in U.S. Pat. No. 8,734,809) |
| WO2016065001) | |
| AAV CLv-RI (See SEQ ID NO: 104 in | AAV CLv-R2 (See SEQ ID NO: 31 in U.S. Pat. No. 8,734,809) |
| U.S. Pat. No. 8,734,809) | |
| AAV CLv-R2 (See SEQ ID NO: 105 in | AAV CLv-R3 (See SEQ ID NO: 32 in U.S. Pat. No. 8,734,809) |
| U.S. Pat. No. 8,734,809) | |
| AAV CLv-R3 (See SEQ ID NO: 106 in | AAV CLv-R4 (See SEQ ID NO: 33 in U.S. Pat. No. 8,734,809) |
| U.S. Pat. No. 8,734,809) | |
| AAV CLv-R4 (See SEQ ID NO: 107 in | AAV CLv-R5 (See SEQ ID NO: 34 in U.S. Pat. No. 8,734,809) |
| U.S. Pat. No. 8,734,809) | |
| AAV CLv-R5 (See SEQ ID NO: 108 in | AAV CLv-R6 (See SEQ ID NO: 35 in U.S. Pat. No. 8,734,809) |
| U.S. Pat. No. 8,734,809) | |
| AAV CLv-R6 (See SEQ ID NO: 109 in | AAV CLv-R7 (See SEQ ID NO: 110 in U.S. Pat. No. 8,734,809) |
| U.S. Pat. No. 8734809); AAV CLv-R7 802 (see SEQ ID NO: | |
| 36 in U.S. Pat. No. 8,734,809) | |
| AAV CLv-R8 (See SEQ ID NO: 37 in | AAV CLv-R8 (See SEQ ID NO: 111 in U.S. Pat. No. 8,734,809) |
| U.S. Pat. No. 8,734,809) | |
| AAV CLv-R9 (See SEQ ID NO: 38 in | AAV CLv-R9 (See SEQ ID NO: 112 in U.S. Pat. No. 8,734,809) |
| U.S. Pat. No. 8,734,809) | |
| AAV CSp-1 (See SEQ ID NO: 45 in U.S. Pat. No. 8,734,809) | AAV CSp-1 (See SEQ ID NO: 119 in U.S. Pat. No. 8,734,809) |
| AAV CSp-10 (See SEQ ID NO: 46 in U.S. Pat. No. 8,734,809) | AAV CSp-10 (See SEQ ID NO: 120 in U.S. Pat. No. 8,734,809) |
| AAV CSp-11 (See SEQ ID NO: 47 in U.S. Pat. No. 8,734,809) | AAV CSp-11 (See SEQ ID NO: 121 in U.S. Pat. No. 8,734,809) |
| AAV CSp-2 (See SEQ ID NO: 48 in U.S. Pat. No. 8,734,809) | AAV CSp-2 (See SEQ ID NO: 122 in U.S. Pat. No. 8,734,809) |
| AAV CSp-3 (See SEQ ID NO: 49 in U.S. Pat. No. 8,734,809) | AAV CSp-3 (See SEQ ID NO: 123 in U.S. Pat. No. 8,734,809) |
| AAV CSp-4 (See SEQ ID NO: 50 in U.S. Pat. No. 8,734,809) | AAV CSp-4 (See SEQ ID NO: 124 in U.S. Pat. No. 8,734,809) |
| AAV CSp-6 (See SEQ ID NO: 51 in U.S. Pat. No. 8,734,809) | AAV CSp-6 (See SEQ ID NO: 125 in U.S. Pat. No. 8,734,809) |
| AAV CSp-7 (See SEQ ID NO: 52 in U.S. Pat. No. 8,734,809) | AAV CSp-7 (See SEQ ID NO: 126 in U.S. Pat. No. 8,734,809) |
| AAV CSp-8 (See SEQ ID NO: 53 in US8734809) | AAV CSp-8 (See SEQ ID NO: 127 in U.S. Pat. No. 8,734,809) |
| AAV CSp-8.10 (See SEQ ID NO: 38 in | AAV CSp-8.10 (See SEQ ID NO: 88 in |
| WO2016065001) | WO2016065001) |
| AAV CSp-8.2 (See SEQ ID NO: 39 in | AAV CSp-8.2 (See SEQ ID NO: 89 in WO2016065001) |
| WO2016065001) | |
| AAV CSp-8.4 (See SEQ ID NO: 40 in | AAV CSp-8.4 (See SEQ ID NO: 90 in WO2016065001) |
| WO2016065001) | |
| AAV CSp-8.5 (See SEQ ID NO: 41 in | AAV CSp-8.5 (See SEQ ID NO: 91 in WO2016065001) |
| WO2016065001) | |
| AAV CSp-8.6 (See SEQ ID NO: 42 in | AAV CSp-8.6 (See SEQ ID NO: 92 in WO2016065001) |
| WO2016065001) | |
| AAV CSp-8.7 (See SEQ ID NO: 43 in | AAV CSp-8.7 (See SEQ ID NO: 93 in WO2016065001) |
| WO2016065001) | |
| AAV CSp-8.8 (See SEQ ID NO: 44 in | AAV CSp-8.8 (See SEQ ID NO: 94 in WO2016065001) |
| WO2016065001) | |
| AAV CSp-8.9 (See SEQ ID NO: 45 in | AAV CSp-8.9 (See SEQ ID NO: 95 in WO2016065001) |
| WO2016065001) | |
| AAV CSp-9 842 (See SEQ ID NO: 54 in | AAV CSp-9 (See SEQ ID NO: 128 in U.S. Pat. No. 8,734,809) |
| U.S. Pat. No. 8,734,809) | |
| AAV.hu.48R3 (See SEQ ID NO: 183 in | AAV.VR-355 (See SEQ ID NO: 181 in U.S. Pat. No. 8,734,809) |
| U.S. Pat. No. 8,734,809) | |
| AAV3B (See SEQ ID NO: 48 in WO2016065001) | AAV3B (See SEQ ID NO: 98 in WO2016065001) |
| AAV4 (See SEQ ID NO: 49 in WO2016065001) | AAV4 (See SEQ ID NO: 99 in WO2016065001) |
| AAV5 (See SEQ ID NO: 50 in WO2016065001) | AAV5 (See SEQ ID NO: 100 in WO2016065001) |
| AAVF1/HSC1 (See SEQ ID NO: 20 in | AAVF1/HSC1 (See SEQ ID NO: 2 in WO2016049230) |
| WO2016049230) | |
| AAVF11/HSC11 (See SEQ ID NO: 26 in | AAVF11/HSC11 (See SEQ ID NO: 4 in |
| WO2016049230) | WO2016049230) |
| AAVF12/HSC12 (See SEQ ID NO: 30 in | AAVF12/HSC12 (See SEQ ID NO: 12 in |
| WO2016049230) | WO2016049230) |
| AAVF13/HSC13 (See SEQ ID NO: 31 in | AAVF13/HSC13 (See SEQ ID NO: 14 in |
| WO2016049230) | WO2016049230) |
| AAVF14/HSC14 (See SEQ ID NO: 32 in | AAVF14/HSC14 (See SEQ ID NO: 15 in |
| WO2016049230) | WO2016049230) |
| AAVF15/HSC15 (See SEQ ID NO: 33 in | AAVF15/HSC15 (See SEQ ID NO: 16 in |
| WO2016049230) | WO2016049230) |
| AAVF16/HSC16 (See SEQ ID NO: 34 in | AAVF16/HSC16 (See SEQ ID NO: 17 in |
| WO2016049230) | WO2016049230) |
| AAVF17/HSC17 (See SEQ ID NO: 35 in | AAVF17/HSC17 (See SEQ ID NO: 13 in |
| WO2016049230) | WO2016049230) |
| AAVF2/HSC2 (See SEQ ID NO: 21 in | AAVF2/HSC2 (See SEQ ID NO: 3 in WO2016049230) |
| WO2016049230) | |
| AAVF3/HSC3 (See SEQ ID NO: 22 in | AAVF3/HSC3 (See SEQ ID NO: 5 in WO2016049230) |
| WO2016049230) | |
| AAVF4/HSC4 (See SEQ ID NO: 23 in | AAVF4/HSC4 (See SEQ ID NO: 6 in WO2016049230) |
| WO2016049230) | |
| AAVF5/HSC5 (See SEQ ID NO: 25 in | AAVF5/HSC5 (See SEQ ID NO: 11 in WO2016049230) |
| WO2016049230) | |
| AAVF6/HSC6 (See SEQ ID NO: 24 in | AAVF6/HSC6 (See SEQ ID NO: 7 in WO2016049230) |
| WO2016049230) | |
| AAVF7/HSC7 (See SEQ ID NO: 27 in | AAVF7/HSC7 (See SEQ ID NO: 8 in WO2016049230) |
| WO2016049230) | |
| AAVF8/HSC8 (See SEQ ID NO: 28 in | AAVF8/HSC8 (See SEQ ID NO:9 in WO2016049230) |
| WO2016049230) | |
| AAVF9/HSC9 (See SEQ ID NO: 10 in | AAVF9/HSC9 882 (see SEQ ID NO: 29 in |
| WO2016049230) | WO2016049230) |

1. A population of rational polyploid AAV virions suitable for use in crossing the blood brain barrier, the rational polyploid AAV virions comprising at least one of AAV VP1 or VP2 viral structural proteins and an AAV VP3 viral structural protein;
wherein the at least one of VP1 or VP2 viral structural proteins are each from any AAV serotype, and the VP3 viral structural protein is from an AAV serotype that efficiently crosses the blood brain barrier and is different from the serotype of at least one of VP1 or VP2, and
wherein the population of rational polyploid AAV virions is capable of crossing the blood brain barrier (BBB) and/or transducing an endothelial cell of the BBB and/or a blood component that crosses the BBB upon systemic or intrathecal administration.

2. The population of claim 1, wherein the population exhibits enhanced transduction activity across the blood brain barrier (BBB) relative to a non-rational polyploid AAV particle that lacks ability to cross the blood brain barrier.

3. The population of any of claims 1-2, wherein the VP3 viral structural protein is an AAV rhesus monkey serotype.

4. The population of any of claims 1-3, wherein the VP3 viral structural protein is from a serotype that efficiently crosses the blood brain barrier selected from the group consisting of AAV1, AAV6, AAV6.2, AAV7, AAV9, AAVrh10, AAVrh74, AAVrh39, and AAVrh43.

5. The population of any of claims 1-4, wherein the population has enhanced transduction to one or more of cortex, striatum, thalamus, medulla, hippocampus, cerebellum and spinal cord of a subject relative to a non-rational polyploid AAV particle that lacks ability to efficiently cross the blood brain barrier.

6. The population of any of claims 1-5, wherein, the population has enhanced transduction relative to AAV2 in one or more of CNS regions selected from the group consisting of medulla, cervical, thoracic, lumbar, and choroid plexus.

7. The population of any of claims 1-6, wherein the population has enhanced binding to brain microvascular endothelial cell (BMVEC) relative to AAV8.

8. The population of any of claims 1-7, wherein the population has biodistribution in the CNS.

9. The population of any of claims 1-8, wherein the population has CNS biodistribution of at least 0.05 vg/cell, 0.1 vg/cell, at least 0.2 vg/cell, at least 0.4 vg/cell, at least 0.6 vg/cell, at least 0.8 vg/cell, at least 1 vg/cell, at least 5 vg/cell, at least 10 vg/cell, at least 20 vg/cell, at least 25 vg/cell, or preferably more.

10. The population of any of claims 1-9, wherein the at least one of VP1 or VP2 is selected from an AAV serotype that crosses blood brain barrier.

11. The population of any of claims 1-9, wherein the at least one of VP1 or VP2 is selected from an AAV serotype that do not cross blood brain barrier.

12. The population of any of claims 1-11, wherein that least one of VP1 or VP2 is not selected from AAV rhesus monkey serotype.

13. The population of any of claims 1-11, wherein the at least one of VP1 or VP2 is selected from an AAV rhesus monkey serotype.

14. The population of any of claims 1-13, wherein the population elicits a lower humoral immune response when administered to a subject as compared to a humoral response as elicited by a parental AAV vector of the subtype of the VP1 or VP2 structural protein.

15. The population of any of claims 1-14, wherein the population evades neutralizing antibodies against the parental serotypes of AAV VP1, VP2, or VP3 viral structural proteins.

16. A method for delivering a transgene across the blood brain barrier of a subject, the method comprising administering to the subject a population of rational polyploid AAV virions of any of claims 1-15.

17. A method for repeat dosing of AAV to a subject, the method comprising a first administration performed by administering to the subject the population of rational polyploid AAV virions from any of claims 1-16, and a second administration performed by administering to the subject parental AAV serotypes of the at least one of VP1 or VP2 viral structural protein,
wherein the population of rational polyploid AAV virions elicits a reduced humoral response in the subject as compared to a humoral response as elicited by the parental AAV serotypes of the VP1 or VP2 viral structural protein, and
wherein the at least one of the VP1 or VP2 is not from a Rhesus AAV serotype.

18. A population of rational polyploid AAV virions that allows repeat dosing, the population comprising:
a rational polyploid AAV virion comprising at least one of AAV VP1 or VP2 viral structural proteins and a AAV VP3 viral structural protein;
wherein the at least one of VP1 or VP2 viral structural proteins are each from any AAV viral serotype, and the VP3 viral structural protein is selected from a rhesus monkey AAV serotype;
wherein the population of rational polyploid AAV virions elicits a reduced humoral response as compared to a humoral response elicited by the parental AAV serotype of the VP1 or VP2 viral structural proteins;
wherein the at least one of VP1 or VP2 are not from a Rhesus AAV serotype, and
wherein the repeat dosing comprises a first administration of the population of rational polyploid AAV virions and a second administration of a parental AAV serotype of the VP1 structural viral protein or VP2 structural viral protein.

19. A population of rational polyploid AAV virions, the population comprising:
a. VP1 and VP2 AAV viral structural proteins selected from an AAV8 viral serotype, and
b. VP3 selected from an AAV rhesus monkey serotype AAV rh10 or AAVrh74,
wherein the population of rational polyploid AAV virions elicits a reduced humoral response when administered to a subject relative to a corresponding humoral response elicited by a parental AAV8 serotype.

20. A method for repeat dosing comprising first and second AAV administrations to a subject, the method comprising:
the first administration performed by administering to the subject a population of rational polyploid AAV virions from any of claims 1-16 or 18-19, and
the second administration performed by administering the parental AAV serotype of VP1 or VP2 viral structural proteins,
wherein the first administration elicits a reduced humoral response in the subject as compared to a corresponding humoral response as elicited by the parental AAV serotypes of VP1 or VP2 viral structural protein, and
wherein VP1 or VP2 are not from a Rhesus AAV serotype.

21. The population of any of claims 18-20, wherein the population evades neutralizing antibodies against the parental serotypes of AAV VP1, VP2, or VP3 viral structural proteins.

22. A method for delivering a transgene across the blood brain barrier of a subject, the method comprising administering to the subject the population of rational polyploid AAV virions of any of claims 18-21.

23. The population of any of the preceding claims, wherein the VP3 protein is a mutated VP3 protein from AAVrh10 or AAVrh74 serotype.

24. The population of claim 23, wherein the mutated AAVrh74 VP3 protein has the amino acid sequence of SEQ ID NO: 2 or a protein having at least 85% sequence identity to SEQ ID NO: 2, or wherein the mutated AAVrh74 VP3 comprises at least one of the following modifications of SEQ ID NO: 2: N263S, G264A, T265S, S266T, G268A, T270del, T274H, E533K, R726H, N736P.

25. The population of claim 24, wherein the mutated AAVrh10 VP3 protein is encoded by a nucleic acid of SEQ ID NO: 5 that comprises at least one or more of: Q214N, S462N and D517E mutations as compared to AAVrh10_VP3 nucleic acid of SEQ ID NO: 5, or comprises a nucleic acid sequence having at least 85% sequence identity to SEQ ID NO: 5 comprising at least one mutation selected from Q214N, S462N and D517E.

26. The population of claim 25, wherein the VP3 protein is a AAVrh74 VP3 protein comprising the amino acid sequence of SEQ ID NO: 2 or 3 or a protein having at least 85% sequence identity to SEQ ID NO: 2 or SEQ ID NO: 3, or comprises at least one of the following amino acid modifications of N263S, G264A, T265S, S266T, G268A, T270del, T274H, E533K, R726H, N736P of SEQ ID NO: 2.

27. A substantially homogenous population of virions of any of claims 1-26, wherein the population is at least 10¹ virions.

28. A nucleic acid comprising, in a 5′ to 3′ direction:
a. a first nucleic acid encoding an AAVrh10 VP3 capsid protein operatively linked to a first promoter;
b. a first poly A sequence;
c. a second nucleic acid encoding a rep protein;
d. a third nucleic acid encoding AAV8 VP1 and VP2 viral structural proteins, the third nucleic acid sequence not being capable of expressing an AAV8 VP3 viral structural protein; and
e. a second poly A sequence.

29. A nucleic acid comprising, in a 5′ to 3′ direction:
a. a first nucleic acid encoding a AAVrh74 VP3 capsid protein operatively linked to a first promoter;
b. a first poly A sequence;
c. a second nucleic acid encoding a rep protein;
d. a third nucleic acid encoding AAV8 VP1 and VP2 viral structural proteins, the third nucleic acid sequence not being capable of expressing a AAV8 VP3 viral structural protein; and
e. a second poly A sequence.

30. A viral vector comprising:
a. an AAV virion from the population of any of the proceeding claims; and
b. a nucleic acid comprising at least one terminal repeat sequence and a heterologous gene, wherein the nucleic acid is encapsulated by the AAV virion.

31. The population of any of the preceding claims comprising a chimeric or modified viral structural protein, wherein the modified viral structural protein comprises insertion, deletion or, substitution of one or more amino acids.

32. The substantially homogenous population of claim 27, wherein the substantially homogenous population elicits significantly fewer anti-AAV IgG antibodies against parental AAV serotypes of VP1 or VP2 structural proteins in serum in vivo as compared to a substantially homogenous population of virions comprising parental AAV serotype.

33. The substantially homogeneous population of claim 32, wherein the parental AAV serotype is AAV8.

34. A population of rational polyploid AAV virions that allow repeat dosing, the population comprising:
at least one of AAV VP1 or VP2 viral structural proteins and a AAV VP3 viral structural protein;
wherein the VP1 and VP2 viral structural proteins are each from any AAV viral serotype except for a Rhesus AAV serotype, and the VP3 viral structural protein is selected from a rhesus monkey AAV serotype;
wherein the population of rational polyploid AAV virions evade neutralizing antibodies against a parental AAV rhesus monkey serotype of the VP3 viral structural protein,
wherein the repeat dosing comprises a first administration of the parental AAV rhesus monkey serotype of the VP3 structural protein and a second administration of the population of rational polyploid AAV virions, and
wherein the VP3 structural protein of the rational polyploid virions is a AAV rhesus monkey mutated viral structural protein VP3.

35. The population of rational polyploid AAV virions of claim 34, wherein the AAV rhesus monkey mutated viral structural protein VP3 is from a mutated AAV rh10 VP3 viral structural protein or from a mutated AAV rh74 VP3 viral structural protein.

36. The population of rational polyploid AAV virions of any of claims 34-35, wherein the mutated viral structural protein VP3 comprises a mutation at an amino acid that corresponds to an amino acid selected from the group consisting of N263, G264, T265, S266T, G268, T270, T274, and E533, wherein all the amino acid positions correspond to a native VP1 sequence numbering of AAV rh10 or AAVrh74.

37. The population of claim 36, wherein the mutation is selected from the group consisting of N263S, G264A, T265S, S266T, G268A, T270del, T274H, and E533K.

38. The population of rational polyploid AAV virions of any of claims 34-37, wherein the mutated viral structural protein VP3 further comprises a mutation at an amino acid that corresponds to an amino acid selected from the group consisting of R727 and N737, wherein all the amino acid positions correspond to a native VP1 sequence numbering of AAVrh10.

39. The population of rational polyploid AAV virions of claim 38, wherein the mutation is selected from the group consisting of R727H and N737P.

40. The population of rational polyploid AAV virions of any of claims 34-37, wherein the mutated viral structural protein VP3 further comprises a mutation at an amino acid that corresponds to an amino acid selected from the group consisting of R726 and N736, wherein all the amino acid positions correspond to a native VP1 sequence numbering of AAV rh74.

41. The population of rational polyploid AAV virions of claim 40, wherein the mutation is selected from the group consisting of R726H and N736P.

42. The population of rational polyploid AAV virions of claim 41, wherein the mutated viral structural protein VP3 further comprises a mutation at an amino acid that corresponds to W at 581, wherein the W is replaced by two subsequent V residues (VV) and wherein all amino acid positions correspond to a native VP1 sequence numbering of AAV rh74.

43. The population of rational polyploid AAV virions of any of claims 34-42, wherein the AAV VP1 or VP2 viral structural protein is any AAV serotype selected from Table 1.

44. The population of rational polyploid AAV virions of any of claims 34-43, wherein the AAV VP1 or VP2 structural protein is AAV8.

45. Use of a population of rational polyploid AAV virions in the manufacturer of a medicament for use for delivering a transgene across a blood brain barrier, the medicament comprising a population of rational polyploid AAV virions of any of claims 1-15 or 18-19, 21, 23-27, 31-44.

46. The use of claim 45, wherein the population of rational polyploid AAV virions comprises VP1 and VP2 AAV viral structural proteins selected from an AAV8 viral serotype, and VP3 viral structural protein selected from an AAV rhesus monkey serotype AAV rh10 or AAVrh74.

47. The use of claim 45, wherein the population of rational polyploid AAV virions comprises VP1 and VP2 AAV viral structural proteins from an AAV8 viral serotype, and a VP3 structural protein from an AAV rhesus monkey serotype AAVrh74.

48. The use of claim 45, wherein the medicament is useful to treat a brain disease or brain disorder or a neurodegenerative disease, or a neurological disease.

49. The use of claim 45, wherein the medicament is useful to treat diseases of the central nervous system (CNS) or peripheral nervous system (PNS).

50. The use of claim 45, wherein the medicament is useful to treat a subject with a brain cancer or cancer in the brain.

51. The use of claim 45, wherein the medicament is useful to treat a subject with a disease or disorder selected from: Alzheimer's disease, Huntington's disease, Parkinson's disease, Amyotrophic Lateral sclerosis (ALS), and Dopamine transporter deficiency syndrome.

52. Use of a nucleic acid in the manufacturer of a medicament comprising a population of rational polyploid AAV virions for use for delivering a transgene across a blood brain barrier, the nucleic acid comprising any of claims 28 or 29.

53. Use of a population of rational polyploid AAV virions in the preparation of a first medicament and a second medicament for use in a method for repeat dosing of a first administration of the first medicament and second administration of the second medicament, wherein the repeat dosing comprises the first administration of the first medicament comprising a rational polyploid AAV virion from any of claims 1-15 or 18-19, and the second administration of the second medicament comprising a parental AAV serotypes of the at least one of VP1 or VP2 viral structural protein,
wherein the population of rational polyploid AAV virion elicits a reduced humoral response as compared to a humoral response as elicited by the parental AAV serotypes of the at least one of the VP1 or VP2 viral structural protein, and wherein the VP1 or VP2 is not from a Rhesus AAV serotype.

54. Use of a population of rational polyploid AAV virions in the preparation of a medicament for evading neutralizing antibodies against parental serotypes of AAV VP1, VP2, or VP3 the medicament comprising a population of rational polyploid AAV virions of any of claims 1-15 or 18-19, 21, 23-27 and 31-44.

55. Use of a population of rational polyploid AAV virions in the preparation of a medicament for delivering a transgene to the small intestine, the medicament comprising the population of rational polyploid AAV virions of any of claims 1-15 or 18-19, 21, 23-27 and 31-44.

56. Use of a population of rational polyploid AAV virions in the preparation of a medicament for the treatment of a gastrointestinal disease or disorder, the medicament comprising the population of rational polyploid AAV virions of any of claims 1-15 or 18-19, 21, 23-27 and 31-44.
